(19) United States
(12) Patent Application Publication
(10) Pub. No.: US 2026/0258000 A1
(43) Pub. Date: Sep. 3, 2026

(54) THYMIDYLATE SYNTHASE INHIBITORS AND USES THEREOF

(71) Applicant: University of Florida Research Foundation, Incorporated, Gainesville, FL (US)

(72) Inventors: Maria Zajac-Kaye, Gainesville, FL (US); Robert William Huigens, Gainesville, FL (US)

(73) Assignee: University of Florida Research Foundation, Incorporated, Gainesville, FL (US)

(21) Appl. No.: 18/706,496

(22) PCT Filed: Nov. 1, 2022
(86) PCT No.: PCT/US2022/048558

§ 371 Date: May 1, 2024

Related U.S. Application Data
(60) Provisional application No. 63/274,677, filed on Nov. 2, 2021.

(51) Int. Cl. C07D 239/48; A61K 31/505; A61K 31/506; A61P 35/00; C07D 403/12; C07D 405/12

(52) U.S. Cl. CPC C07D 239/48; A61K 31/505; A61K 31/506; A61P 35/00; C07D 403/12; C07D 405/12

(57) ABSTRACT

Provided herein are compounds of Formula (I) or (II), and pharmaceutically acceptable salts), solvates, hydrates, stereoisomers, polymorphs, tautomers, co-crystals, isotopically labeled derivatives, and prodrugs thereof, and compositions thereof. Also provided are methods and kits involving the compounds or compositions for treating and/or preventing diseases and/or conditions (e.g., proliferative disease, such as, cancers), or other diseases associated with a folate-dependent enzyme (e.g., thymidylate synthase, dihydrofolate reductase) in a subject. Provided are methods of inhibiting a folate-dependent enzyme (e.g., thymidylate synthase, dihydrofolate reductase) in a subject and/or biological sample.

### RELATED APPLICATIONS

This application claims priority under 35 U.S.C. § 119(e) to U.S. Provisional application, U.S. Ser. No. 63/274,677, filed on Nov. 2, 2021, which is incorporated herein by reference.

### GOVERNMENT SUPPORT

This invention was made with government support under Grant Numbers R01 CA188132 awarded by The National Institutes of Health. The government has certain rights in the invention.

### BACKGROUND OF THE INVENTION

Thymidylate synthase (TS) is an essential enzyme responsible for the reductive methylation of deoxyuridine monophosphate (dUMP) to deoxythymidine monophosphate (dTMP). This reductive methylation of dUMP requires the cofactor 5,10-methylenetetrahydrofolate (5,10-mTHF) as the methylene and hydride donor. The resulting dTMP is then phosphorylated to deoxythymidine triphosphate (dTTP), an essential precursor for DNA synthesis and repair. Importantly, this TS-catalyzed reaction is the sole intracellular source of de novo dTMP¹. Overexpression of TS is observed in a wide spectrum of tumor types and elevated TS levels are associated with increased cellular proliferation, tumor invasiveness and metastasis, drug resistance, and poor clinical outcomes²⁻¹¹. Additionally, ectopic overexpression of TS in primary mammalian cells exhibits oncogene-like activity by inducing parameters of the neoplastic phenotype including foci formation, anchor-independent growth, and tumor formation in nude mice¹². Therefore, inhibition of TS is an attractive target for intervention, especially given the spectrum of common adult tumor types that would benefit from TS inhibition. For these reasons, TS has been a target of cancer intervention since the 1950s and chemotherapy agents, such as 5-fluorouracil (5-FU), methotrexate, and pemetrexed, are still in use for patients with colon, pancreatic, and non-small cell lung cancer^{1,13}. The inhibition of TS results in depletion of dTMP followed by depletion of dTTP, which leads to a state of nucleotide pool imbalance^{1,14}. This nucleotide imbalance impairs DNA synthesis and repair, promoting cell cycle arrest, increased DNA damage, and thymine-less death^{14,15}.

Catalytically active TS is a homodimer where each subunit has an active site that accommodates both the dUMP substrate and the 5,10-mTHF cofactor. The nucleotide (dUMP) and the folate (5,10-mTHF) binding sites provide distinct opportunities to inhibit TS and targeting either site can inhibit TS catalytic activity. Classically, nucleotide or folate-based antimetabolites have been used to inhibit TS, each with their own distinct limitations.

A disclosed TS inhibitor is the fluoropyrimidine antimetabolite, 5-FU. As a prodrug, 5-FU is able to passively diffuse into the cell and is intracellularly converted to 5-fluoro-2′-deoxyuridine monophosphate (5-FdUrd) which competes with the dUMP substrate¹⁴. When bound in the nucleotide binding site in the presence of the 5,10-mTHF cofactor in the folate site, the 5-FdUrd will form a covalent complex with TS resulting in irreversible inhibition^{16,17}. However, the inhibition of TS by 5-FU treatment has been noted consistently to induce TS overexpression associated with acquired drug resistance^{1,18}. This mechanism for induction of drug resistance is hypothesized to explain the inability of 5-FU to induce durable complete remissions or cures in patients with locally advanced or metastatic disease¹⁹.

It has been more than 70 years since the first folate antimetabolites, aminopterin, was used in the early treatment of childhood lymphocytic leukemia²⁰. Prototypic antifolates are analogues of folic acid that compete with the 5,10-mTHF binding to directly inhibit TS²¹. Antifolates also indirectly block the TS-catalyzed conversion of dUMP to dTMP by inhibiting dihydrofolate reductase (DHFR)^{1,22,23}. DHFR is responsible for the reduction of DHF to THF, a first step in regenerating 5,10-mTHF. For example, methotrexate, a more selective antifolate DHFR inhibitor, suppresses TS due to depletion of 5,10-mTHF^{1,22}. As a result of the structural similarities to folic acid, classical antifolates are dependent on different folate metabolic pathways to be effective, presenting multiple pathway and feedback loops that cancer cells can utilize during the development of drug resistance^{21,24-28}. For example, cellular uptake of drug is dependent upon folate transporters, such as the reduced folate carrier and the proton-coupled folate transporter^{24,25}. Accordingly, impaired transport function affecting cellular uptake is one tactic exploited by cancer cells to yield resistance to classical antifolates^{27,29,30.} Once within the cell, antifolates must undergo polyglutamation by the enzyme folylpolyglutamate synthetase (FPGS), which increases cellular retention and efficiency against the target enzymes^{25-27,31,32}. Thus, impaired polyglutamation also results in antifolate resistance.

Thymidylate synthase (TS) inhibitors may provide chemotherapy for difficult-to-treat cancer subtypes. Despite initial therapeutic benefit, current inhibitors induce TS overexpression or alter folate transport/metabolism feedback pathways in ways that tumor cells exploit for drug resistance. There is therefore a need for new inhibitors of folate-dependent enzymes (e.g., thymidylate synthase, dihydrofolate reductase) which effectively treat proliferative diseases, such as cancers, without inducing drug resistance.

### SUMMARY OF THE INVENTION

Described herein are compounds of Formula (I) or (II), compounds of Table A, and salts (e.g., pharmaceutically acceptable salts), solvates, hydrates, stereoisomers, polymorphs, tautomers, co-crystals, isotopically labeled derivatives, and prodrugs thereof, and compositions thereof. The compounds of Formula (I) or (II),

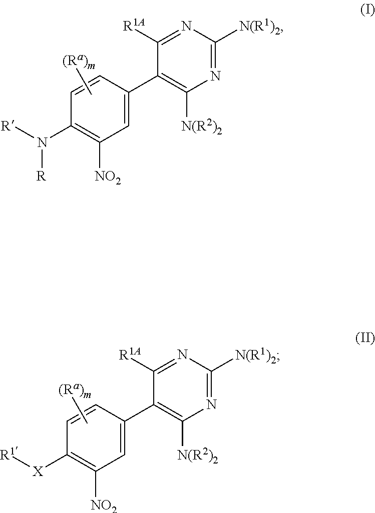

and the compounds of Table A:

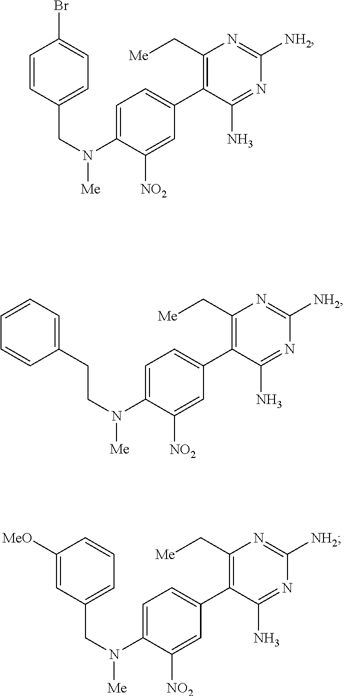 [SMILES: CCc1nc(N)nc(N)c1-c1ccc(N(C)Cc2ccc(Br)cc2)c([N+](=O)[O-])c1 ; CCc1nc(N)nc(N)c1-c1ccc(N(C)CCc2ccccc2)c([N+](=O)[O-])c1 ; CCc1nc(N)nc(N)c1-c1ccc(N(C)Cc2cccc(OC)c2)c([N+](=O)[O-])c1]

and pharmaceutically acceptable salts, solvates, hydrates, stereoisomers, polymorphs, tautomers, co-crystals, isotopically labeled derivatives, and prodrugs thereof are thought to act in some embodiments by inhibiting a folate-dependent enzyme (e.g., thymidylate synthase, dihydrofolate reductase). In some aspects, the compounds are believed to be small molecule inhibitors of thymidylate synthase (TS) and/or dihydrofolate reductase (DHFR) that exhibit: i) enhanced anti-tumor activity as compared to current fluoropyrimidines and antifolates without inducing TS overexpression, ii) without leading to resistance to traditional anti-cancer or anti-folate drugs (e.g., such as 5-fluorouracil); iii) extend survival in a subject, for example, in an animal model (e.g., pancreatic tumor mouse model), iv) are well tolerated; and/or v) having equal efficacy using either intraperitoneal or oral administration. The compounds are multifunctional antifolates, with structural features allowing direct TS inhibition while also having the ability to inhibit dihydrofolate reductase (DHFR). Described herein are new antifolates that also inhibit thymidylate biosynthesis with a favorable safety profile. Therefore, these compounds have potential as enhanced cancer therapeutics. Described herein are methods of using the compounds described herein, and pharmaceutically acceptable salts, solvates, hydrates, stereoisomers, polymorphs, tautomers, co-crystals, isotopically labeled derivatives, and prodrugs thereof, as well as compositions thereof. In some aspects, the compounds, and pharmaceutically acceptable salts, solvates, hydrates, stereoisomers, polymorphs, tautomers, co-crystals, isotopically labeled derivatives, and prodrugs thereof, and compositions thereof disclosed herein are used as therapeutics for the treatment of proliferative diseases, or diseases associated with the overexpression and/or aberrant (e.g., increased or unwanted) activity of a folate-dependent enzyme (e.g., thymidylate synthase, dihydrofolate reductase). In some aspects, the compounds, and pharmaceutically acceptable salts, solvates, hydrates, stereoisomers, polymorphs, tautomers, co-crystals, isotopically labeled derivatives, and prodrugs thereof, and compositions thereof are used to study the inhibition of a folate-dependent enzyme (e.g., thymidylate synthase, dihydrofolate reductase). The compounds described herein may be useful in treating and/or preventing a disease or condition (e.g., proliferative disease, such as cancer), in a subject in need thereof. Provided are uses including a compound described herein. Also provided are pharmaceutical compositions, and kits including a compound described herein.

In one aspect, the present disclosure provides compounds of Formula (I):

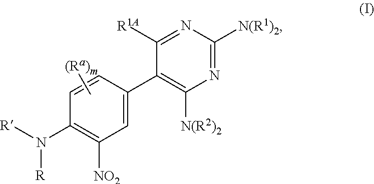

and pharmaceutically acceptable salts, solvates, hydrates, stereoisomers, polymorphs, tautomers, co-crystals, isotopically labeled derivatives, and prodrugs thereof, wherein R^{1A}, R¹, R², R^{a}, R′, R, and m are as defined herein.

In another aspect, the present disclosure provides compounds of Formula (II):

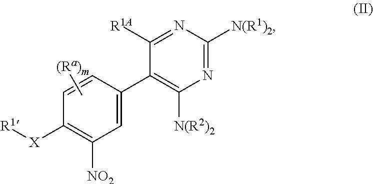

and pharmaceutically acceptable salts, solvates, hydrates, stereoisomers, polymorphs, tautomers, co-crystals, isotopically labeled derivatives, and prodrugs thereof, wherein R^{1A}, R¹, R², R^{a}, R^{1′}, X, and m are as defined herein.

Exemplary compounds of Formula (I) include, but are not limited to:

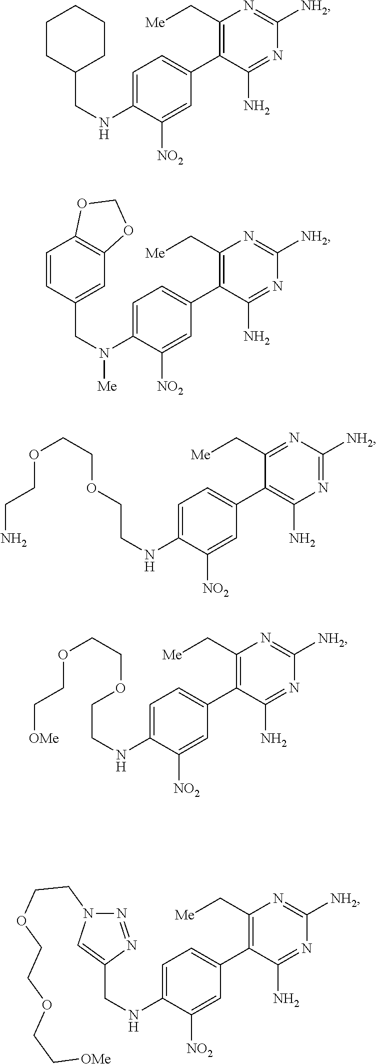 [SMILES: CCc1nc(N)nc(N)c1-c1ccc(NCC2CCCCC2)c([N+](=O)[O-])c1 ; CCc1nc(N)nc(N)c1-c1ccc(N(C)Cc2ccc3c(c2)OCO3)c([N+](=O)[O-])c1 ; CCc1nc(N)nc(N)c1-c1ccc(NCCOCCOCCN)c([N+](=O)[O-])c1 ; CCc1nc(N)nc(N)c1-c1ccc(NCCOCCOCCOC)c([N+](=O)[O-])c1 ; CCc1nc(N)nc(N)c1-c1ccc(NCc2cn(CCOCCOCCOC)nn2)c([N+](=O)[O-])c1]

and pharmaceutically acceptable salts, solvates, hydrates, stereoisomers, polymorphs, tautomers, co-crystals, isotopically labeled derivatives, and prodrugs thereof.

Also provided are compounds of the following formula in Table A:

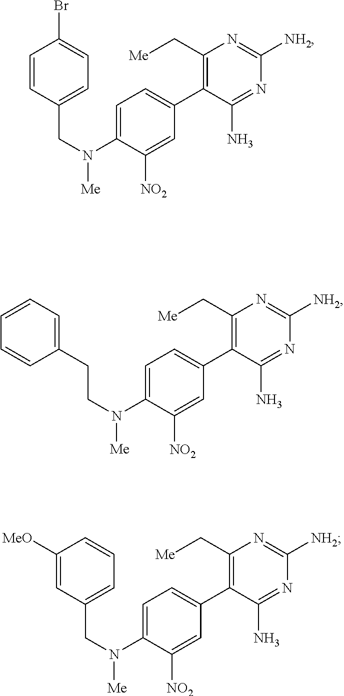 [SMILES: CCc1nc(N)nc(N)c1-c1ccc(N(C)Cc2ccc(Br)cc2)c([N+](=O)[O-])c1 ; CCc1nc(N)nc(N)c1-c1ccc(N(C)CCc2ccccc2)c([N+](=O)[O-])c1 ; CCc1nc(N)nc(N)c1-c1ccc(N(C)Cc2cccc(OC)c2)c([N+](=O)[O-])c1]

and pharmaceutically acceptable salts, solvates, hydrates, stereoisomers, polymorphs, tautomers, co-crystals, isotopically labeled derivatives, and thereof. In certain embodiments, the compounds of Table A are not encompassed by Formula (I) or (II).

Exemplary compounds of Formula (I) or (II) include, but are not limited to, the compounds disclosed in Examples 1-4 (e.g., the compounds disclosed in Table 1 of Examples 1-4).

In another aspect, the present disclosure provides pharmaceutical compositions including a compound described herein (e.g., compounds of Formula (I) or (II), compounds of Table A), and optionally a pharmaceutically acceptable excipient. In certain embodiments, a pharmaceutical composition described herein includes a therapeutically or prophylactically effective amount of a compound described herein. The pharmaceutical compositions may be useful in inhibiting a folate-dependent enzyme (e.g., thymidylate synthase, dihydrofolate reductase) in a subject, biological sample, or cell, in treating and/or preventing a disease (e.g., a proliferative disease, such as cancer, or other diseases associated with a folate-dependent enzyme (e.g., thymidylate synthase, dihydrofolate reductase)) in a subject in need thereof. In certain embodiments, the compound being administered or used (e.g., compounds of Formula (I) or (II), compounds of Table A) inhibits a folate-dependent enzyme (e.g., thymidylate synthase, dihydrofolate reductase) in a subject or cell, treats and/or prevents a disease (e.g., proliferative disease, such as cancers, or other diseases associated with a folate-dependent enzyme (e.g., thymidylate synthase, dihydrofolate reductase)) in a subject in need thereof.

In still another aspect, described herein are kits including a container with a compound or pharmaceutical composition described herein. A kit described herein may include a single dose or multiple doses of the compound or pharmaceutical composition. The kits may be useful in inhibiting a folate-dependent enzyme (e.g., thymidylate synthase, dihydrofolate reductase) in a subject, biological sample, or cell. The kits may be useful in inhibiting a thymidylate synthase and dihydrofolate reductase. The kits (e.g., including compounds of Formula (I) or (II), compounds of Table A) may be useful in treating and/or preventing a disease described herein (e.g., proliferative disease, such as, cancers, or other diseases associated with a folate-dependent enzyme (e.g., thymidylate synthase, dihydrofolate reductase)) in a subject in need thereof. The kits (e.g., including compounds of Formula (I) or (II), compounds of Table A) may be useful in treating and/or preventing a disease described herein (e.g., proliferative disease, such as, cancers, or other diseases associated with a folate-dependent enzyme (e.g., thymidylate synthase, dihydrofolate reductase)) in a subject in need thereof. In certain embodiments, a kit described herein further includes instructions for using the compound or pharmaceutical composition included in the kit. A kit described herein may also include information (e.g. prescribing information) as required by a regulatory agency, such as the U.S. Food and Drug Administration (FDA).

In yet another aspect, the present disclosure provides compounds and pharmaceutical compositions described herein for use in inhibiting a folate-dependent enzyme (e.g., thymidylate synthase, dihydrofolate reductase) in a subject, biological sample, or cell, and for treating and/or preventing a disease (e.g., proliferative disease, such as, cancers, or other diseases associated with a folate-dependent enzyme (e.g., thymidylate synthase, dihydrofolate reductase)) in a subject in need thereof. In yet another aspect, the present disclosure provides compounds (e.g., including compounds of Formula (I) or (II), compounds of Table A) and pharmaceutical compositions described herein for use in the disclosure (e.g., inhibiting a folate-dependent enzyme (e.g., thymidylate synthase, dihydrofolate reductase) in a subject or cell, and treating and/or preventing a disease (e.g., proliferative disease, such as, cancers, or other diseases associated with a folate-dependent enzyme (e.g., thymidylate synthase, dihydrofolate reductase)) in a subject in need thereof.

The present application refers to various issued patents, published patent applications, journal articles, and other publications, all of which are incorporated herein by reference. The details of one or more embodiments of the invention are set forth herein. Other features, objects, and advantages of the invention will be apparent from the Detailed Description, Examples, Figures, and Claims.

### Definitions

Definitions of specific functional groups and chemical terms are described in more detail below. The chemical elements are identified in accordance with the Periodic Table of the Elements, CAS version, Handbook of Chemistry and Physics, 75^{th} Ed., inside cover, and specific functional groups are generally defined as described therein. Additionally, general principles of organic chemistry, as well as specific functional moieties and reactivity, are described in Thomas Sorrell, Organic Chemistry, University Science Books, Sausalito, 1999; Michael B. Smith, March's Advanced Organic Chemistry, 7^{th} Edition, John Wiley & Sons, Inc., New York, 2013; Richard C. Larock, Comprehensive Organic Transformations, John Wiley & Sons, Inc., New York, 2018; and Carruthers, Some Modern Methods of Organic Synthesis, 3^{rd} Edition, Cambridge University Press, Cambridge, 1987.

Compounds described herein can comprise one or more asymmetric centers, and thus can exist in various stereoisomeric forms, e.g., enantiomers and/or diastereomers. For example, the compounds described herein can be in the form of an individual enantiomer, diastereomer, or geometric isomer, or can be in the form of a mixture of stereoisomers, including racemic mixtures and mixtures enriched in one or more stereoisomer. Isomers can be isolated from mixtures by methods known to those skilled in the art, including chiral high pressure liquid chromatography (HPLC) and the formation and crystallization of chiral salts; or preferred isomers can be prepared by asymmetric syntheses. See, for example, Jacques et al., Enantiomers, Racemates and Resolutions (Wiley Interscience, New York, 1981); Wilen et al., Tetrahedron 33:2725 (1977); Eliel, E. L. Stereochemistry of Carbon Compounds (McGraw-Hill, NY, 1962); and Wilen, S. H., Tables of Resolving Agents and Optical Resolutions p. 268 (E. L. Eliel, Ed., Univ. of Notre Dame Press, Notre Dame, IN 1972). The invention additionally encompasses compounds as individual isomers substantially free of other isomers, and alternatively, as mixtures of various isomers.

When a range of values is listed, it is intended to encompass each value and sub-range within the range. For example, “C₁₋₆” is intended to encompass C₁, C₂, C₃, C₄, C₅, C₆, C₁₋₆, C₁₋₅, C₁₋₄, C₁₋₃, C₁₋₂, C₂₋₆, C₂₋₅, C₂₋₄, C₂₋₃, C₃₋₆, C₃₋₅, C₃₋₄, C₄₋₆, C₄₋₅, and C₅₋₆. For example, “C₁₋₆ alkyl” encompasses, C₁, C₂, C₃, C₄, C₅, C₆, C₁₋₆, C₁₋₅, C₁₋₄, C₁₋₃, C₁₋₂, C₂₋₆, C₂₋₅, C₂₋₄, C₂₋₃, C₃₋₆, C₃₋₅, C₃₋₄, C₄₋₆, C₄₋₅, and C₅₋₆ alkyl.

The term “alkyl” refers to a radical of a straight-chain or branched saturated hydrocarbon group having from 1 to 20 carbon atoms (“C₁₋₂₀ alkyl”). In some embodiments, an alkyl group has 1 to 12 carbon atoms (“C₁₋₁₂ alkyl”). In some embodiments, an alkyl group has 1 to 10 carbon atoms (“C₁₋₁₀ alkyl”). In some embodiments, an alkyl group has 1 to 9 carbon atoms (“C₁₋₉ alkyl”). In some embodiments, an alkyl group has 1 to 8 carbon atoms (“C₁₋₈ alkyl”). In some embodiments, an alkyl group has 1 to 7 carbon atoms (“C₁₋₇ alkyl”). In some embodiments, an alkyl group has 1 to 6 carbon atoms (“C₁₋₆ alkyl”). In some embodiments, an alkyl group has 1 to 5 carbon atoms (“C₁₋₅ alkyl”). In some embodiments, an alkyl group has 1 to 4 carbon atoms (“C₁₋₄ alkyl”). In some embodiments, an alkyl group has 1 to 3 carbon atoms (“C₁₋₃ alkyl”). In some embodiments, an alkyl group has 1 to 2 carbon atoms (“C₁₋₂ alkyl”). In some embodiments, an alkyl group has 1 carbon atom (“C₁ alkyl”). In some embodiments, an alkyl group has 2 to 6 carbon atoms (“C₂₋₆ alkyl”). Examples of C₁₋₆ alkyl groups include methyl (C₁), ethyl (C₂), propyl (C₃) (e.g., n-propyl, isopropyl), butyl (C₄) (e.g., n-butyl, tert-butyl, sec-butyl, isobutyl), pentyl (C₅) (e.g., n-pentyl, 3-pentanyl, amyl, neopentyl, 3-methyl-2-butanyl, tert-amyl), and hexyl (C₆) (e.g., n-hexyl). Additional examples of alkyl groups include n-heptyl (C₇), n-octyl (C₈), n-dodecyl (C₁₂), and the like. Unless otherwise specified, each instance of an alkyl group is independently unsubstituted (an “unsubstituted alkyl”) or substituted (a “substituted alkyl”) with one or more substituents (e.g., halogen, such as F). In certain embodiments, the alkyl group is an unsubstituted C₁₋₁₂ alkyl (such as unsubstituted C₁₋₆ alkyl, e.g., —CH₃ (Me), unsubstituted ethyl (Et), unsubstituted propyl (Pr, e.g., unsubstituted n-propyl (n-Pr), unsubstituted isopropyl (i-Pr)), unsubstituted butyl (Bu, e.g., unsubstituted n-butyl (n-Bu), unsubstituted tert-butyl (tert-Bu or t-Bu), unsubstituted sec-butyl (sec-Bu or s-Bu), unsubstituted isobutyl (i-Bu)). In certain embodiments, the alkyl group is a substituted C₁₋₁₂ alkyl (such as substituted C₁₋₆ alkyl, e.g., —CH₂F, —CHF₂, —CF₃, —CH₂CH₂F, —CH₂CHF₂, —CH₂CF₃, or benzyl (Bn)).

The term “alkenyl” refers to a radical of a straight-chain or branched hydrocarbon group having from 1 to 20 carbon atoms and one or more carbon-carbon double bonds (e.g., 1, 2, 3, or 4 double bonds). In some embodiments, an alkenyl group has 1 to 20 carbon atoms (“C₁₋₂₀ alkenyl”). In some embodiments, an alkenyl group has 1 to 12 carbon atoms (“C₁₋₁₂ alkenyl”). In some embodiments, an alkenyl group has 1 to 11 carbon atoms (“C₁₋₁₂ alkenyl”). In some embodiments, an alkenyl group has 1 to 10 carbon atoms (“C₁₋₁₀ alkenyl”). In some embodiments, an alkenyl group has 1 to 9 carbon atoms (“C₁₋₉ alkenyl”). In some embodiments, an alkenyl group has 1 to 8 carbon atoms (“C₁₋₈ alkenyl”). In some embodiments, an alkenyl group has 1 to 7 carbon atoms (“C₁₋₇ alkenyl”). In some embodiments, an alkenyl group has 1 to 6 carbon atoms (“C₁₋₆ alkenyl”). In some embodiments, an alkenyl group has 1 to 5 carbon atoms (“C₁₋₅ alkenyl”). In some embodiments, an alkenyl group has 1 to 4 carbon atoms (“C₁₋₄ alkenyl”). In some embodiments, an alkenyl group has 1 to 3 carbon atoms (“C₁₋₃ alkenyl”). In some embodiments, an alkenyl group has 1 to 2 carbon atoms (“C₁₋₂ alkenyl”). In some embodiments, an alkenyl group has 1 carbon atom (“C₁ alkenyl”). The one or more carbon-carbon double bonds can be internal (such as in 2-butenyl) or terminal (such as in 1-butenyl). Examples of C₁₋₄ alkenyl groups include methylidenyl (C₁), ethenyl (C₂), 1-propenyl (C₃), 2-propenyl (C₃), 1-butenyl (C₄), 2-butenyl (C₄), butadienyl (C₄), and the like. Examples of C₁₋₆ alkenyl groups include the aforementioned C₂₋₄ alkenyl groups as well as pentenyl (C₅), pentadienyl (C₅), hexenyl (C₆), and the like. Additional examples of alkenyl include heptenyl (C₇), octenyl (C₈), octatrienyl (C₈), and the like. Unless otherwise specified, each instance of an alkenyl group is independently unsubstituted (an “unsubstituted alkenyl”) or substituted (a “substituted alkenyl”) with one or more substituents. In certain embodiments, the alkenyl group is an unsubstituted C₁₋₂₀ alkenyl. In certain embodiments, the alkenyl group is a substituted C₁₋₂₀ alkenyl. In an alkenyl group, a C═C double bond for which the stereochemistry is not specified (e.g., —CH═CHCH₃ or

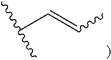

may be in the (E)- or (Z)-configuration.

The term “alkynyl” refers to a radical of a straight-chain or branched hydrocarbon group having from 1 to 20 carbon atoms and one or more carbon-carbon triple bonds (e.g., 1, 2, 3, or 4 triple bonds) (“C₁₋₂₀ alkynyl”). In some embodiments, an alkynyl group has 1 to 10 carbon atoms (“C₁₋₁₀ alkynyl”). In some embodiments, an alkynyl group has 1 to 9 carbon atoms (“C₁₋₉ alkynyl”). In some embodiments, an alkynyl group has 1 to 8 carbon atoms (“C₁₋₈ alkynyl”). In some embodiments, an alkynyl group has 1 to 7 carbon atoms (“C₁₋₇ alkynyl”). In some embodiments, an alkynyl group has 1 to 6 carbon atoms (“C₁₋₆ alkynyl”). In some embodiments, an alkynyl group has 1 to 5 carbon atoms (“C₁₋₅ alkynyl”). In some embodiments, an alkynyl group has 1 to 4 carbon atoms (“C₁₋₄ alkynyl”). In some embodiments, an alkynyl group has 1 to 3 carbon atoms (“C₁₋₃ alkynyl”). In some embodiments, an alkynyl group has 1 to 2 carbon atoms (“C₁₋₂ alkynyl”). In some embodiments, an alkynyl group has 2 to 10 carbon atoms (“C₂₋₁₀ alkynyl”). In some embodiments, an alkynyl group has 2 to 9 carbon atoms (“C₂₋₉ alkynyl”). In some embodiments, an alkynyl group has 2 to 8 carbon atoms (“C₂₋₈ alkynyl”). In some embodiments, an alkynyl group has 2 to 7 carbon atoms (“C₂₋₇ alkynyl”). In some embodiments, an alkynyl group has 2 to 6 carbon atoms (“C₂₋₆ alkynyl”). In some embodiments, an alkynyl group has 2 to 5 carbon atoms (“C₂₋₅ alkynyl”). In some embodiments, an alkynyl group has 2 to 4 carbon atoms (“C₂₋₄ alkynyl”). In some embodiments, an alkynyl group has 2 to 3 carbon atoms (“C₂₋₃ alkynyl”). In some embodiments, an alkynyl group has 2 carbon atoms (“C₂ alkynyl”). In some embodiments, an alkynyl group has 1 carbon atom (“C₁ alkynyl”). The one or more carbon-carbon triple bonds can be internal (such as in 2-butynyl) or terminal (such as in 1-butynyl). Examples of C₁_4 alkynyl groups include, without limitation, methylidynyl (C₁), ethynyl (C₂), 1-propynyl (C₃), 2-propynyl (C₃), 1-butynyl (C₄), 2-butynyl (C₄), and the like. Examples of C₁₋₆ alkenyl groups include the aforementioned C₂₋₄ alkynyl groups as well as pentynyl (C₅), hexynyl (C₆), and the like. Examples of C₂₋₄ alkynyl groups include, without limitation, ethynyl (C₂), 1-propynyl (C₃), 2-propynyl (C₃), 1-butynyl (C₄), 2-butynyl (C₄), and the like. Examples of C₂₋₆ alkenyl groups include the aforementioned C₂₋₄ alkynyl groups as well as pentynyl (C₅), hexynyl (C₆), and the like. Additional examples of alkynyl include heptynyl (C₇), octynyl (C₈), and the like. Unless otherwise specified, each instance of an alkynyl group is independently unsubstituted (an “unsubstituted alkynyl”) or substituted (a “substituted alkynyl”) with one or more substituents. In certain embodiments, the alkynyl group is an unsubstituted C₁₋₂₀ alkynyl. In certain embodiments, the alkynyl group is a substituted C₁₋₂₀ alkynyl. In certain embodiments, the alkynyl group is an optionally substituted C₂₋₂₀ alkynyl.

The term “carbocyclyl” or “carbocyclic” refers to a radical of a non-aromatic cyclic hydrocarbon group having from 3 to 14 ring carbon atoms (“C₃₋₁₄ carbocyclyl”) and zero heteroatoms in the non-aromatic ring system. In some embodiments, a carbocyclyl group has 3 to 14 ring carbon atoms (“C₃₋₁₄ carbocyclyl”). In some embodiments, a carbocyclyl group has 3 to 13 ring carbon atoms (“C₃₋₁₃ carbocyclyl”). In some embodiments, a carbocyclyl group has 3 to 12 ring carbon atoms (“C₃₋₁₂ carbocyclyl”). In some embodiments, a carbocyclyl group has 3 to 11 ring carbon atoms (“C₃₋₁₁ carbocyclyl”). In some embodiments, a carbocyclyl group has 3 to 10 ring carbon atoms (“C₃₋₁₀ carbocyclyl”). In some embodiments, a carbocyclyl group has 3 to 8 ring carbon atoms (“C₃₋₈ carbocyclyl”). In some embodiments, a carbocyclyl group has 3 to 7 ring carbon atoms (“C₃₋₇ carbocyclyl”). In some embodiments, a carbocyclyl group has 3 to 6 ring carbon atoms (“C₃₋₆ carbocyclyl”). In some embodiments, a carbocyclyl group has 4 to 6 ring carbon atoms (“C₄₋₆ carbocyclyl”). In some embodiments, a carbocyclyl group has 5 to 6 ring carbon atoms (“C₅₋₆ carbocyclyl”). In some embodiments, a carbocyclyl group has 5 to 10 ring carbon atoms (“C₅₋₁₀ carbocyclyl”). Exemplary C₃₋₆ carbocyclyl groups include cyclopropyl (C₃), cyclopropenyl (C₃), cyclobutyl (C₄), cyclobutenyl (C₄), cyclopentyl (C₅), cyclopentenyl (C₅), cyclohexyl (C₆), cyclohexenyl (C₆), cyclohexadienyl (C₆), and the like. Exemplary C₃₋₈ carbocyclyl groups include the aforementioned C₃₋₆ carbocyclyl groups as well as cycloheptyl (C₇), cycloheptenyl (C₇), cycloheptadienyl (C₇), cycloheptatrienyl (C₇), cyclooctyl (C₈), cyclooctenyl (C₈), bicyclo[2.2.1]heptanyl (C₇), bicyclo[2.2.2]octanyl (C₈), and the like. Exemplary C₃₋₁₀ carbocyclyl groups include the aforementioned C₃₋₈ carbocyclyl groups as well as cyclononyl (C₉), cyclononenyl (C₉), cyclodecyl (C₁₀), cyclodecenyl (C₁₀), octahydro-1H-indenyl (C₉), decahydronaphthalenyl (C₁₀), spiro[4.5]decanyl (C₁₀), and the like. Exemplary C₃₋₈ carbocyclyl groups include the aforementioned C₃₋₁₀ carbocyclyl groups as well as cycloundecyl (C₁₁), spiro[5.5]undecanyl (C₁₁), cyclododecyl (C₁₂), cyclododecenyl (C₁₂), cyclotridecane (C₁₃), cyclotetradecane (C₁₄), and the like. As the foregoing examples illustrate, in certain embodiments, the carbocyclyl group is either monocyclic (“monocyclic carbocyclyl”) or polycyclic (e.g., containing a fused, bridged or spiro ring system such as a bicyclic system (“bicyclic carbocyclyl”) or tricyclic system (“tricyclic carbocyclyl”)) and can be saturated or can contain one or more carbon-carbon double or triple bonds. “Carbocyclyl” also includes ring systems wherein the carbocyclyl ring, as defined above, is fused with one or more aryl or heteroaryl groups wherein the point of attachment is on the carbocyclyl ring, and in such instances, the number of carbons continue to designate the number of carbons in the carbocyclic ring system. Unless otherwise specified, each instance of a carbocyclyl group is independently unsubstituted (an “unsubstituted carbocyclyl”) or substituted (a “substituted carbocyclyl”) with one or more substituents. In certain embodiments, the carbocyclyl group is an unsubstituted C₃₋₁₄ carbocyclyl. In certain embodiments, the carbocyclyl group is a substituted C₃₋₁₄ carbocyclyl.

In some embodiments, “carbocyclyl” is a monocyclic, saturated carbocyclyl group having from 3 to 14 ring carbon atoms (“C₃₋₁₄ cycloalkyl”). In some embodiments, a cycloalkyl group has 3 to 10 ring carbon atoms (“C₃₋₁₀ cycloalkyl”). In some embodiments, a cycloalkyl group has 3 to 8 ring carbon atoms (“C₃_cycloalkyl”). In some embodiments, a cycloalkyl group has 3 to 6 ring carbon atoms (“C₃₋₆ cycloalkyl”). In some embodiments, a cycloalkyl group has 4 to 6 ring carbon atoms (“C₄₋₆ cycloalkyl”). In some embodiments, a cycloalkyl group has 5 to 6 ring carbon atoms (“C₅₋₆ cycloalkyl”). In some embodiments, a cycloalkyl group has 5 to 10 ring carbon atoms (“C₅₋₁₀ cycloalkyl”). Examples of C₅₋₆ cycloalkyl groups include cyclopentyl (C₅) and cyclohexyl (C₅). Examples of C₃₋₆ cycloalkyl groups include the aforementioned C₅₋₆ cycloalkyl groups as well as cyclopropyl (C₃) and cyclobutyl (C₄). Examples of C₃₋₈ cycloalkyl groups include the aforementioned C₃₋₆ cycloalkyl groups as well as cycloheptyl (C₇) and cyclooctyl (C₈). Unless otherwise specified, each instance of a cycloalkyl group is independently unsubstituted (an “unsubstituted cycloalkyl”) or substituted (a “substituted cycloalkyl”) with one or more substituents. In certain embodiments, the cycloalkyl group is an unsubstituted C₃₋₁₄ cycloalkyl. In certain embodiments, the cycloalkyl group is a substituted C₃₋₁₄ cycloalkyl. In certain embodiments, the carbocyclyl includes 0, 1, or 2 C═C double bonds in the carbocyclic ring system, as valency permits.

The term “heterocyclyl” or “heterocyclic” refers to a radical of a 3- to 14-membered non-aromatic ring system having ring carbon atoms and 1 to 4 ring heteroatoms, wherein each heteroatom is independently selected from nitrogen, oxygen, and sulfur (“3-14 membered heterocyclyl”). In heterocyclyl groups that contain one or more nitrogen atoms, the point of attachment can be a carbon or nitrogen atom, as valency permits. A heterocyclyl group can either be monocyclic (“monocyclic heterocyclyl”) or polycyclic (e.g., a fused, bridged or spiro ring system such as a bicyclic system (“bicyclic heterocyclyl”) or tricyclic system (“tricyclic heterocyclyl”)), and can be saturated or can contain one or more carbon-carbon double or triple bonds. Heterocyclyl polycyclic ring systems can include one or more heteroatoms in one or both rings. “Heterocyclyl” also includes ring systems wherein the heterocyclyl ring, as defined above, is fused with one or more carbocyclyl groups wherein the point of attachment is either on the carbocyclyl or heterocyclyl ring, or ring systems wherein the heterocyclyl ring, as defined above, is fused with one or more aryl or heteroaryl groups, wherein the point of attachment is on the heterocyclyl ring, and in such instances, the number of ring members continue to designate the number of ring members in the heterocyclyl ring system. Unless otherwise specified, each instance of heterocyclyl is independently unsubstituted (an “unsubstituted heterocyclyl”) or substituted (a “substituted heterocyclyl”) with one or more substituents. In certain embodiments, the heterocyclyl group is an unsubstituted 3-14 membered heterocyclyl. In certain embodiments, the heterocyclyl group is a substituted 3-14 membered heterocyclyl. In certain embodiments, the heterocyclyl is substituted or unsubstituted, 3- to 7-membered, monocyclic heterocyclyl, wherein 1, 2, or 3 atoms in the heterocyclic ring system are independently oxygen, nitrogen, or sulfur, as valency permits.

In some embodiments, a heterocyclyl group is a 5-10 membered non-aromatic ring system having ring carbon atoms and 1-4 ring heteroatoms, wherein each heteroatom is independently selected from nitrogen, oxygen, and sulfur (“5-10 membered heterocyclyl”). In some embodiments, a heterocyclyl group is a 5-8 membered non-aromatic ring system having ring carbon atoms and 1-4 ring heteroatoms, wherein each heteroatom is independently selected from nitrogen, oxygen, and sulfur (“5-8 membered heterocyclyl”). In some embodiments, a heterocyclyl group is a 5-6 membered non-aromatic ring system having ring carbon atoms and 1-4 ring heteroatoms, wherein each heteroatom is independently selected from nitrogen, oxygen, and sulfur (“5-6 membered heterocyclyl”). In some embodiments, the 5-6 membered heterocyclyl has 1-3 ring heteroatoms selected from nitrogen, oxygen, and sulfur. In some embodiments, the 5-6 membered heterocyclyl has 1-2 ring heteroatoms selected from nitrogen, oxygen, and sulfur. In some embodiments, the 5-6 membered heterocyclyl has 1 ring heteroatom selected from nitrogen, oxygen, and sulfur.

Exemplary 3-membered heterocyclyl groups containing 1 heteroatom include azirdinyl, oxiranyl, and thiiranyl. Exemplary 4-membered heterocyclyl groups containing 1 heteroatom include azetidinyl, oxetanyl, and thietanyl. Exemplary 5-membered heterocyclyl groups containing 1 heteroatom include tetrahydrofuranyl, dihydrofuranyl, tetrahydrothiophenyl, dihydrothiophenyl, pyrrolidinyl, dihydropyrrolyl, and pyrrolyl-2,5-dione. Exemplary 5-membered heterocyclyl groups containing 2 heteroatoms include dioxolanyl, oxathiolanyl and dithiolanyl. Exemplary 5-membered heterocyclyl groups containing 3 heteroatoms include triazolinyl, oxadiazolinyl, and thiadiazolinyl. Exemplary 6-membered heterocyclyl groups containing 1 heteroatom include piperidinyl, tetrahydropyranyl, dihydropyridinyl, and thianyl. Exemplary 6-membered heterocyclyl groups containing 2 heteroatoms include piperazinyl, morpholinyl, dithianyl, and dioxanyl. Exemplary 6-membered heterocyclyl groups containing 3 heteroatoms include triazinyl. Exemplary 7-membered heterocyclyl groups containing 1 heteroatom include azepanyl, oxepanyl, and thiepanyl. Exemplary 8-membered heterocyclyl groups containing 1 heteroatom include azocanyl, oxecanyl and thiocanyl. Exemplary bicyclic heterocyclyl groups include indolinyl, isoindolinyl, dihydrobenzofuranyl, dihydrobenzothienyl, tetra-hydrobenzothienyl, tetrahydrobenzofuranyl, tetrahydroindolyl, tetrahydroquinolinyl, tetrahydroisoquinolinyl, decahydroquinolinyl, decahydroisoquinolinyl, octahydrochromenyl, octahydroisochromenyl, decahydronaphthyridinyl, decahydro-1,8-naphthyridinyl, octahydropyrrolo[3,2-b]pyrrole, indolinyl, phthalimidyl, naphthalimidyl, chromanyl, chromenyl, 1H-benzo[e][1,4]diazepinyl, 1,4,5,7-tetrahydropyrano[3,4-b]pyrrolyl, 5,6-dihydro-4H-furo[3,2-b]pyrrolyl, 6,7-dihydro-5H-furo[3,2-b]pyranyl, 5,7-dihydro-4H-thieno[2,3-c]pyranyl, 2,3-dihydro-1H-pyrrolo[2,3-b]pyridinyl, 2,3-dihydrofuro[2,3-b]pyridinyl, 4,5,6,7-tetrahydro-1H-pyrrolo[2,3-b]pyridinyl, 4,5,6,7-tetrahydrofuro[3,2-c]pyridinyl, 4,5,6,7-tetrahydrothieno[3,2-b]pyridinyl, 1,2,3,4-tetrahydro-1,6-naphthyridinyl, and the like.

The term “aryl” refers to a radical of a monocyclic or polycyclic (e.g., bicyclic or tricyclic) 4n+2 aromatic ring system (e.g., having 6, 10, or 14 pi electrons shared in a cyclic array) having 6-14 ring carbon atoms and zero heteroatoms provided in the aromatic ring system (“C₆₋₁₄ aryl”). In some embodiments, an aryl group has 6 ring carbon atoms (“C₆ aryl”; e.g., phenyl). In some embodiments, an aryl group has 10 ring carbon atoms (“C₁₀ aryl”; e.g., naphthyl such as 1-naphthyl and 2-naphthyl). In some embodiments, an aryl group has 14 ring carbon atoms (“C₁₄ aryl”; e.g., anthracyl). “Aryl” also includes ring systems wherein the aryl ring, as defined above, is fused with one or more carbocyclyl or heterocyclyl groups wherein the radical or point of attachment is on the aryl ring, and in such instances, the number of carbon atoms continue to designate the number of carbon atoms in the aryl ring system. Unless otherwise specified, each instance of an aryl group is independently unsubstituted (an “unsubstituted aryl”) or substituted (a “substituted aryl”) with one or more substituents. In certain embodiments, the aryl group is an unsubstituted C₆₋₁₄ aryl. In certain embodiments, the aryl group is a substituted C₆₋₁₄ aryl.

“Aralkyl” is a subset of “alkyl” and refers to an alkyl group substituted by an aryl group, wherein the point of attachment is on the alkyl moiety.

The term “heteroaryl” refers to a radical of a 5-14 membered monocyclic or polycyclic (e.g., bicyclic, tricyclic) 4n+2 aromatic ring system (e.g., having 6, 10, or 14 □ electrons shared in a cyclic array) having ring carbon atoms and 1-4 ring heteroatoms provided in the aromatic ring system, wherein each heteroatom is independently selected from nitrogen, oxygen, and sulfur (“5-14 membered heteroaryl”). In heteroaryl groups that contain one or more nitrogen atoms, the point of attachment can be a carbon or nitrogen atom, as valency permits. Heteroaryl polycyclic ring systems can include one or more heteroatoms in one or both rings. “Heteroaryl” includes ring systems wherein the heteroaryl ring, as defined above, is fused with one or more carbocyclyl or heterocyclyl groups wherein the point of attachment is on the heteroaryl ring, and in such instances, the number of ring members continue to designate the number of ring members in the heteroaryl ring system. “Heteroaryl” also includes ring systems wherein the heteroaryl ring, as defined above, is fused with one or more aryl groups wherein the point of attachment is either on the aryl or heteroaryl ring, and in such instances, the number of ring members designates the number of ring members in the fused polycyclic (aryl/heteroaryl) ring system. Polycyclic heteroaryl groups wherein one ring does not contain a heteroatom (e.g., indolyl, quinolinyl, carbazolyl, and the like) the point of attachment can be on either ring, e.g., either the ring bearing a heteroatom (e.g., 2-indolyl) or the ring that does not contain a heteroatom (e.g., 5-indolyl). In certain embodiments, the heteroaryl is substituted or unsubstituted, 5- or 6-membered, monocyclic heteroaryl, wherein 1, 2, 3, or 4 atoms in the heteroaryl ring system are independently oxygen, nitrogen, or sulfur. In certain embodiments, the heteroaryl is substituted or unsubstituted, 9- or 10-membered, bicyclic heteroaryl, wherein 1, 2, 3, or 4 atoms in the heteroaryl ring system are independently oxygen, nitrogen, or sulfur.

In some embodiments, a heteroaryl group is a 5-10 membered aromatic ring system having ring carbon atoms and 1-4 ring heteroatoms provided in the aromatic ring system, wherein each heteroatom is independently selected from nitrogen, oxygen, and sulfur (“5-10 membered heteroaryl”). In some embodiments, a heteroaryl group is a 5-8 membered aromatic ring system having ring carbon atoms and 1-4 ring heteroatoms provided in the aromatic ring system, wherein each heteroatom is independently selected from nitrogen, oxygen, and sulfur (“5-8 membered heteroaryl”). In some embodiments, a heteroaryl group is a 5-6 membered aromatic ring system having ring carbon atoms and 1-4 ring heteroatoms provided in the aromatic ring system, wherein each heteroatom is independently selected from nitrogen, oxygen, and sulfur (“5-6 membered heteroaryl”). In some embodiments, the 5-6 membered heteroaryl has 1-3 ring heteroatoms selected from nitrogen, oxygen, and sulfur. In some embodiments, the 5-6 membered heteroaryl has 1-2 ring heteroatoms selected from nitrogen, oxygen, and sulfur. In some embodiments, the 5-6 membered heteroaryl has 1 ring heteroatom selected from nitrogen, oxygen, and sulfur. Unless otherwise specified, each instance of a heteroaryl group is independently unsubstituted (an “unsubstituted heteroaryl”) or substituted (a “substituted heteroaryl”) with one or more substituents. In certain embodiments, the heteroaryl group is an unsubstituted 5-14 membered heteroaryl. In certain embodiments, the heteroaryl group is a substituted 5-14 membered heteroaryl.

Exemplary 5-membered heteroaryl groups containing 1 heteroatom include pyrrolyl, furanyl, and thiophenyl. Exemplary 5-membered heteroaryl groups containing 2 heteroatoms include imidazolyl, pyrazolyl, oxazolyl, isoxazolyl, thiazolyl, and isothiazolyl. Exemplary 5-membered heteroaryl groups containing 3 heteroatoms include triazolyl, oxadiazolyl, and thiadiazolyl. Exemplary 5-membered heteroaryl groups containing 4 heteroatoms include tetrazolyl. Exemplary 6-membered heteroaryl groups containing 1 heteroatom include pyridinyl. Exemplary 6-membered heteroaryl groups containing 2 heteroatoms include pyridazinyl, pyrimidinyl, and pyrazinyl. Exemplary 6-membered heteroaryl groups containing 3 or 4 heteroatoms include triazinyl and tetrazinyl, respectively. Exemplary 7-membered heteroaryl groups containing 1 heteroatom include azepinyl, oxepinyl, and thiepinyl. Exemplary 5,6-bicyclic heteroaryl groups include indolyl, isoindolyl, indazolyl, benzotriazolyl, benzothiophenyl, isobenzothiophenyl, benzofuranyl, benzoisofuranyl, benzimidazolyl, benzoxazolyl, benzisoxazolyl, benzoxadiazolyl, benzthiazolyl, benzisothiazolyl, benzthiadiazolyl, indolizinyl, and purinyl. Exemplary 6,6-bicyclic heteroaryl groups include naphthyridinyl, pteridinyl, quinolinyl, isoquinolinyl, cinnolinyl, quinoxalinyl, phthalazinyl, and quinazolinyl. Exemplary tricyclic heteroaryl groups include phenanthridinyl, dibenzofuranyl, carbazolyl, acridinyl, phenothiazinyl, phenoxazinyl, and phenazinyl.

“Heteroaralkyl” is a subset of “alkyl” and refers to an alkyl group substituted by a heteroaryl group, wherein the point of attachment is on the alkyl moiety.

The term “unsaturated bond” refers to a double or triple bond.

The term “unsaturated” or “partially unsaturated” refers to a moiety that includes at least one double or triple bond.

The term “saturated” or “fully saturated” refers to a moiety that does not contain a double or triple bond, e.g., the moiety only contains single bonds.

Affixing the suffix “-ene” to a group indicates the group is a divalent moiety, e.g., alkylene is the divalent moiety of alkyl, alkenylene is the divalent moiety of alkenyl, alkynylene is the divalent moiety of alkynyl, heteroalkylene is the divalent moiety of heteroalkyl, heteroalkenylene is the divalent moiety of heteroalkenyl, heteroalkynylene is the divalent moiety of heteroalkynyl, carbocyclylene is the divalent moiety of carbocyclyl, heterocyclylene is the divalent moiety of heterocyclyl, arylene is the divalent moiety of aryl, and heteroarylene is the divalent moiety of heteroaryl.

A group is optionally substituted unless expressly provided otherwise. The term “optionally substituted” refers to being substituted or unsubstituted. In certain embodiments, alkyl, alkenyl, alkynyl, heteroalkyl, heteroalkenyl, heteroalkynyl, carbocyclyl, heterocyclyl, aryl, and heteroaryl groups are optionally substituted. “Optionally substituted” refers to a group which is substituted or unsubstituted (e.g., “substituted” or “unsubstituted” alkyl, “substituted” or “unsubstituted” alkenyl, “substituted” or “unsubstituted” alkynyl, “substituted” or “unsubstituted” heteroalkyl, “substituted” or “unsubstituted” heteroalkenyl, “substituted” or “unsubstituted” heteroalkynyl, “substituted” or “unsubstituted” carbocyclyl, “substituted” or “unsubstituted” heterocyclyl, “substituted” or “unsubstituted” aryl or “substituted” or “unsubstituted” heteroaryl group). In general, the term “substituted” means that at least one hydrogen present on a group is replaced with a permissible substituent, e.g., a substituent which upon substitution results in a stable compound, e.g., a compound which does not spontaneously undergo transformation such as by rearrangement, cyclization, elimination, or other reaction. Unless otherwise indicated, a “substituted” group has a substituent at one or more substitutable positions of the group, and when more than one position in any given structure is substituted, the substituent is either the same or different at each position. The term “substituted” is contemplated to include substitution with all permissible substituents of organic compounds, and includes any of the substituents described herein that results in the formation of a stable compound. The present invention contemplates any and all such combinations in order to arrive at a stable compound. For purposes of this invention, heteroatoms such as nitrogen may have hydrogen substituents and/or any suitable substituent as described herein which satisfy the valencies of the heteroatoms and results in the formation of a stable moiety. The invention is not limited in any manner by the exemplary substituents described herein.

Exemplary carbon atom substituents include halogen, —CN, —NO₂, —N₃, —SO₂H, —SO₃H, —OH, —OR^{aa}—, —ON(R^{bb})₂, —N(R^{bb})₂, —N(R^{bb})₃⁺X⁻, —N(OR^{cc})R^{bb}, —SH, —SR^{aa}—, —SSR^{cc}, —C(═O)R^{aa}, —CO₂H, —CHO, —C(OR^{cc})₂, —CO₂R^{aa}, —OC(═O)R^{aa}, —OCO₂R^{aa}, —C(═O)N(R^{bb})₂, —OC(═O)N(R^{bb})₂, —NR^{bb}C(═O)R^{aa}—, —NR^{bb}CO₂R^{aa}, —NR^{bb}C(═O)N(R^{bb})₂, —C(═NR^{bb})R^{aa}, —C(═NR^{bb})OR^{aa}—, —OC(═NR^{bb})R^{aa}—, —OC(═NR^{bb})OR^{aa}—, —C(═NR^{bb})N(R^{bb})₂, —OC(═NR^{bb})N(R^{bb})₂, —NR^{bb}C(═NR^{bb})N(R^{bb})₂, —C(═O)NR^{bb}SO₂R^{aa}, —NR^{bb}SO₂R^{aa}, —SO₂N(R^{bb})₂, —SO₂R^{aa}, —SO₂OR^{aa}, —OSO₂R^{aa}, —S(═O)R^{aa}, —OS(═O)R^{aa}, —Si(R^{aa})₃, —OSi(R^{aa})₃—C(═S)N(R^{bb})₂, —C(═O)SR^{aa}, —C(═S)SR^{aa}, —SC(═S)SR′, —SC(═O)SR′, —OC(═O)SR′, —SC(═O)OR′, —SC(═O)R′, —P(═O)(R′)₂, —P(═O)(OR^{cc})₂, —OP(═O)(R′)₂, —OP(═O)(OR^{cc})₂, —P(═O)(N(R^{bb})₂)₂, —OP(═O)(N(R^{bb})₂)₂, —NR^{bb}P(═O)(R^{aa})₂, —NR^{bb}P(═O)(OR^{cc})₂, —NR^{bb}P(═O)(N(R^{bb})₂)₂, —P(R^{cc})₂, —P(OR^{cc})₂, —P(R^{cc})₃⁺X⁻, —P(OR^{cc})₃⁺X⁻, —P(R^{cc})₄, —P(OR^{cc})₄, —OP(R^{cc})₂, —OP(R^{cc})₃⁺X⁻, —OP(OR^{cc})₂, —OP(OR^{cc})₃⁺X⁻, —OP(R^{cc})₄, —OP(OR^{cc})₄, —B(R^{cc})₂, —B(OR^{cc})₂, —BR^{aa}(OR^{cc}), C₁₋₂₀ alkyl, C₁₋₂₀ perhaloalkyl, C₁₋₂₀ alkenyl, C₁₋₂₀ alkynyl, heteroC₁₋₂₀ alkyl, heteroC₁₋₂₀ alkenyl, heteroC₁₋₂₀ alkynyl, C₃₋₁₀ carbocyclyl, 3-14 membered heterocyclyl, C₆₋₁₄ aryl, and 5-14 membered heteroaryl, wherein each alkyl, alkenyl, alkynyl, heteroalkyl, heteroalkenyl, heteroalkynyl, carbocyclyl, heterocyclyl, aryl, and heteroaryl is independently substituted with 0, 1, 2, 3, 4, or 5 R^{dd} groups; wherein X⁻ is a counterion;
or two geminal hydrogens on a carbon atom are replaced with the group ═O, ═S, ═NN(R^{bb})₂, ═NNR^{bb}C(═O)R^{aa}—, ═NNR^{bb}C(═O)OR^{aa}, ═NNR^{bb}S(═O)₂R^{aa}, ═NR^{bb}, or ═NOR^{cc}; wherein:
each instance of R^{aa} is, independently, selected from C₁₋₂₀ alkyl, C₁₋₂₀ perhaloalkyl, C₁₋₂₀ alkenyl, C₁₋₂₀ alkynyl, heteroC₁₋₂₀ alkyl, heteroC₁₋₂₀alkenyl, heteroC₁₋₂₀alkynyl, C₃₋₁₀ carbocyclyl, 3-14 membered heterocyclyl, C₆₋₁₄ aryl, and 5-14 membered heteroaryl, or two R^{bb} groups are joined to form a 3-14 membered heterocyclyl or 5-14 membered heteroaryl ring, wherein each of the alkyl, alkenyl, alkynyl, heteroalkyl, heteroalkenyl, heteroalkynyl, carbocyclyl, heterocyclyl, aryl, and heteroaryl is independently substituted with 0, 1, 2, 3, 4, or 5 R^{dd} groups;
each instance of R^{bb} is, independently, selected from hydrogen, —OH, —OR^{aa}, —N(R^{cc})₂, —CN, —C(═O)R^{aa}, —C(═O)N(R^{cc})₂, —CO₂R^{aa}, —SO₂R^{aa}, —C(═NR^{cc})OR^{aa}, —C(═NR^{cc})N(R^{cc})₂, —SO₂N(R^{cc})₂, —SO₂R^{cc}, —SO₂OR^{cc}, —SOR^{aa}, —C(═S)N(R^{cc})₂, —C(═O)SR^{cc}, —C(═S)SR^{cc}, —P(═O)(R^{aa})₂, —P(═O)(OR^{cc})₂, —P(═O)(N(R^{cc})₂)₂, C₁₋₂₀ alkyl, C₁₋₂₀ perhaloalkyl, C₁₋₂₀ alkenyl, C₁₋₂₀ alkynyl, heteroC₁₋₂₀ alkyl, heteroC₁₋₂₀ alkenyl, heteroC₁₋₂₀ alkynyl, C₃₋₁₀ carbocyclyl, 3-14 membered heterocyclyl, C₆₋₁₄ aryl, and 5-14 membered heteroaryl, or two R^{bb} groups are joined to form a 3-14 membered heterocyclyl or 5-14 membered heteroaryl ring, wherein each alkyl, alkenyl, alkynyl, heteroalkyl, heteroalkenyl, heteroalkynyl, carbocyclyl, heterocyclyl, aryl, and heteroaryl is independently substituted with 0, 1, 2, 3, 4, or 5 R^{dd} groups;
each instance of R^{cc} is, independently, selected from hydrogen, C₁₋₂₀ alkyl, C₁₋₂₀ perhaloalkyl, C₁₋₂₀ alkenyl, C₁₋₂₀ alkynyl, heteroC₁₋₂₀ alkyl, heteroC₁₋₂₀ alkenyl, heteroC₁₋₂₀ alkynyl, C₃₋₁₀ carbocyclyl, 3-14 membered heterocyclyl, C₆₋₁₄ aryl, and 5-14 membered heteroaryl, or two R^{cc} groups are joined to form a 3-14 membered heterocyclyl or 5-14 membered heteroaryl ring, wherein each alkyl, alkenyl, alkynyl, heteroalkyl, heteroalkenyl, heteroalkynyl, carbocyclyl, heterocyclyl, aryl, and heteroaryl is independently substituted with 0, 1, 2, 3, 4, or 5 R^{dd} groups;
each instance of R^{dd} is, independently, selected from halogen, —CN, —NO₂, —N₃, —SO₂H, —SO₃H, —OH, —OR^{ee}, —ON(R^{ff})₂, —N(R^{ff})₂, —N(R^{ff})₃⁺X⁻, —N(OR^{ee})R^{ff}, —SH, —SR^{ee}, —SSR^{ee}, —C(═O)R^{ee}, —CO₂H, —CO₂R^{ee}, —OC(═O)R^{ee}, —OCO₂R^{ee}, —C(═O)N(R^{ff})₂, —OC(═O)N(R^{ff})₂, —NR^{ff}C(═O)R^{ee}, —NR^{ff}CO₂R^{ee}, —NR^{ff}C(═O)N(R^{ff})₂, —C(═NR^{ff})OR^{ee}, —OC(═NR^{ff})R^{ee}, —OC(═NR^{ff})OR^{ee}, —C(═NR^{ee})N(R^{ff})₂, —OC(═NR^{ff})N(R^{ff})₂, —NR^{ff}C(═NR^{ff})N(R^{ff})₂, —NR^{ff}SO₂R^{ee}, —SO₂N(R^{ff})₂, —SO₂R^{ee}, —SO₂OR^{ee}, —OSO₂R^{ee}, —S(═O)R^{ee}, —Si(R^{ee})₃, —OSi(R^{ee})₃, —C(═S)N(R^{ff})₂, —C(═O)SR^{ee}, —C(═S)SR^{ee}, —SC(═S)SR^{ee}, —P(═O)(OR^{ee})₂, —P(═O)(R^{ee})₂, —OP(═O)(R^{ee})₂, —OP(═O)(OR^{ee})₂, C₁₋₁₀ alkyl, C₁₋₁₀ perhaloalkyl, C₁₋₁₀ alkenyl, C₁₋₁₀ alkynyl, heteroC₁₋₁₀ alkyl, heteroC₁₋₁₀ alkenyl, heteroC₁₋₁₀alkynyl, C₃₋₁₀ carbocyclyl, 3-10 membered heterocyclyl, C₆₋₁₀ aryl, and 5-10 membered heteroaryl, wherein each alkyl, alkenyl, alkynyl, heteroalkyl, heteroalkenyl, heteroalkynyl, carbocyclyl, heterocyclyl, aryl, and heteroaryl is independently substituted with 0, 1, 2, 3, 4, or 5 R^{gg} groups, or two geminal R^{dd} substituents are joined to form ═O or ═S; wherein X⁻ is a counterion;
each instance of R^{ee} is, independently, selected from C₁₋₁₀ alkyl, C₁₋₁₀ perhaloalkyl, C₁₋₁₀ alkenyl, C₁₋₁₀ alkynyl, heteroC₁₋₁₀ alkyl, heteroC₁₋₁₀ alkenyl, heteroC₁₋₁₀ alkynyl, C₃₋₁₀ carbocyclyl, C₆₋₁₀ aryl, 3-10 membered heterocyclyl, and 3-10 membered heteroaryl, wherein each alkyl, alkenyl, alkynyl, heteroalkyl, heteroalkenyl, heteroalkynyl, carbocyclyl, heterocyclyl, aryl, and heteroaryl is independently substituted with 0, 1, 2, 3, 4, or 5 R^{gg} groups;
each instance of R^{ff} is, independently, selected from hydrogen, C₁₋₁₀ alkyl, C₁₋₁₀ perhaloalkyl, C₁₋₁₀ alkenyl, C₁₋₁₀ alkynyl, heteroC₁₋₁₀ alkyl, heteroC₁₋₁₀ alkenyl, heteroC₁₋₁₀ alkynyl, C₃₋₁₀ carbocyclyl, 3-10 membered heterocyclyl, C₆₋₁₀ aryl, and 5-10 membered heteroaryl, or two R^{ff} groups are joined to form a 3-10 membered heterocyclyl or 5-10 membered heteroaryl ring, wherein each alkyl, alkenyl, alkynyl, heteroalkyl, heteroalkenyl, heteroalkynyl, carbocyclyl, heterocyclyl, aryl, and heteroaryl is independently substituted with 0, 1, 2, 3, 4, or 5 R^{gg} groups;
each instance of R^{gg} is, independently, halogen, —CN, —NO₂, —N₃, —SO₂H, —SO₃H, —OH, —OC₁₋₆ alkyl, —ON(C₁₋₆ alkyl)₂, —N(C₁₋₆ alkyl)₂, —N(C₁₋₆ alkyl)₃⁺X⁻, —NH(C₁₋₆ alkyl)₂⁺X⁻, —NH₂(C₁₋₆ alkyl)+X⁻, —NH₃⁺X⁻, —N(OC₁₋₆ alkyl)(C₁₋₆ alkyl), —N(OH)(C₁₋₆ alkyl), —NH(OH), —SH, —SC₁₋₆ alkyl, —SS(C₁₋₆ alkyl), —C(═O)(C₁₋₆ alkyl), —CO₂H, —CO₂(C₁₋₆ alkyl), —OC(═O)(C₁₋₆ alkyl), —OCO₂(C₁₋₆ alkyl), —C(═O)NH₂, —C(═O)N(C₁₋₆ alkyl)₂, —OC(═O)NH(C₁₋₆ alkyl), —NHC(═O)(C₁₋₆ alkyl), —N(C₁₋₆ alkyl)C(═O)(C₁₋₆ alkyl), —NHCO₂(C₁₋₆ alkyl), —NHC(═O)N(C₁₋₆ alkyl)₂, —NHC(═O)NH(C₁₋₆ alkyl), —NHC(═O)NH₂, —C(═NH)O(C₁₋₆ alkyl), —OC(═NH)(C₁₋₆ alkyl), —OC(═NH)OC₁₋₆ alkyl, —C(═NH)N(C₁₋₆ alkyl)₂, —C(═NH)NH(C₁₋₆ alkyl), —C(═NH)NH₂, —OC(═NH)N(C₁₋₆ alkyl)₂, —OC(NH)NH(C₁₋₆ alkyl), —OC(NH)NH₂, —NHC(NH)N(C₁₋₆ alkyl)₂, —NHC(═NH)NH₂, —NHSO₂(C₁₋₆ alkyl), —SO₂N(C₁₋₆ alkyl)₂, —SO₂NH(C₁₋₆ alkyl), —SO₂NH₂, —SO₂C₁₋₆ alkyl, —SO₂OC₁₋₆ alkyl, —OSO₂C₁₋₆ alkyl, —SOC₁₋₆ alkyl, —Si(C₁₋₆ alkyl)₃, —OSi(C₁₋₆ alkyl)₃ —C(═S)N(C₁₋₆ alkyl)₂, C(═S)NH(C₁₋₆ alkyl), C(═S)NH₂, —C(═O)S(C₁₋₆ alkyl), —C(═S)SC₁₋₆ alkyl, —SC(═S)SC₁₋₆ alkyl, —P(═O)(OC₁₋₆ alkyl)₂, —P(═O)(C₁₋₆ alkyl)₂, —OP(═O)(C₁₋₆ alkyl)₂, —OP(═O)(OC₁₋₆ alkyl)₂, C₁₋₁₀ alkyl, C₁₋₁₀ perhaloalkyl, C₁₋₁₀ alkenyl, C₁₋₁₀ alkynyl, heteroC₁₋₁₀ alkyl, heteroC₁₋₁₀ alkenyl, heteroC₁₋₁₀ alkynyl, C₃₋₁₀ carbocyclyl, C₆₋₁₀ aryl, 3-10 membered heterocyclyl, or 5-10 membered heteroaryl; or two geminal R⁹⁹ substituents can be joined to form O or ═S; and each X⁻ is a counterion.

In certain embodiments, each carbon atom substituent is independently halogen, substituted (e.g., substituted with one or more halogen) or unsubstituted C₁₋₆ alkyl, —OR^{aa}, —SR^{aa}—, —N(R^{bb})₂, —CN, —SCN, —NO₂, —C(═O)R^{aa}, —CO₂R^{aa}, —C(═O)N(R^{bb})₂, —OC(═O)R^{aa}, —OCO₂R^{aa}, —OC(═O)N(R^{bb})₂, —NR^{bb}C(═O)R^{aa}, —NR^{bb}CO₂R^{aa}, or —NR^{bb}C(═O)N(R^{bb})₂. In certain embodiments, each carbon atom substituent is independently halogen, substituted (e.g., substituted with one or more halogen) or unsubstituted C₁₋₁₀ alkyl, —OR^{aa}, SR^{aa}, —N(R^{bb})₂, —CN, —SCN, —NO₂, —C(═O)R^{aa}, —CO₂R′, —C(═O)N(R^{bb})₂, —OC(═O)R^{aa}, —OCO₂R^{aa}, —OC(═O)N(R^{bb})₂, —NR^{bb}C(═O)R^{aa}, —NR^{bb}CO₂R^{aa}, or —NR^{bb}C(═O)N(R^{bb})₂, wherein R^{aa} is hydrogen, substituted (e.g., substituted with one or more halogen) or unsubstituted C₁₋₁₀ alkyl, an oxygen protecting group (e.g., silyl, TBDPS, TBDMS, TIPS, TES, TMS, MOM, THP, t-Bu, Bn, allyl, acetyl, pivaloyl, or benzoyl) when attached to an oxygen atom, or a sulfur protecting group (e.g., acetamidomethyl, t-Bu, 3-nitro-2-pyridine sulfenyl, 2-pyridine-sulfenyl, or triphenylmethyl) when attached to a sulfur atom; and each R^{bb} is independently hydrogen, substituted (e.g., substituted with one or more halogen) or unsubstituted C₁₋₁₀ alkyl, or a nitrogen protecting group (e.g., Bn, Boc, Cbz, Fmoc, trifluoroacetyl, triphenylmethyl, acetyl, or Ts). In certain embodiments, each carbon atom substituent is independently halogen, substituted (e.g., substituted with one or more halogen) or unsubstituted C₁₋₆ alkyl, —OR^{aa}, —SR^{aa}, —N(R^{bb})₂, —CN, —SCN, or —NO₂. In certain embodiments, each carbon atom substituent is independently halogen, substituted (e.g., substituted with one or more halogen moieties) or unsubstituted C₁₋₁₀ alkyl, —OR^{aa}, —SR^{aa}, —N(R^{bb})₂, —CN, —SCN, or —NO₂, wherein R^{aa} is hydrogen, substituted (e.g., substituted with one or more halogen) or unsubstituted C₁₋₁₀ alkyl, an oxygen protecting group (e.g., silyl, TBDPS, TBDMS, TIPS, TES, TMS, MOM, THP, t-Bu, Bn, allyl, acetyl, pivaloyl, or benzoyl) when attached to an oxygen atom, or a sulfur protecting group (e.g., acetamidomethyl, t-Bu, 3-nitro-2-pyridine sulfenyl, 2-pyridine-sulfenyl, or triphenylmethyl) when attached to a sulfur atom; and each R^{bb} is independently hydrogen, substituted (e.g., substituted with one or more halogen) or unsubstituted C₁₋₁₀ alkyl, or a nitrogen protecting group (e.g., Bn, Boc, Cbz, Fmoc, trifluoroacetyl, triphenylmethyl, acetyl, or Ts).

In certain embodiments, the molecular weight of a carbon atom substituent is lower than 250, lower than 200, lower than 150, lower than 100, or lower than 50 g/mol. In certain embodiments, a carbon atom substituent consists of carbon, hydrogen, fluorine, chlorine, bromine, iodine, oxygen, sulfur, nitrogen, and/or silicon atoms. In certain embodiments, a carbon atom substituent consists of carbon, hydrogen, fluorine, chlorine, bromine, iodine, oxygen, sulfur, and/or nitrogen atoms. In certain embodiments, a carbon atom substituent consists of carbon, hydrogen, fluorine, chlorine, bromine, and/or iodine atoms. In certain embodiments, a carbon atom substituent consists of carbon, hydrogen, fluorine, and/or chlorine atoms.

The term “halo” or “halogen” refers to fluorine (fluoro, —F), chlorine (chloro, —Cl), bromine (bromo, —Br), or iodine (iodo, —I).

The term “hydroxyl” or “hydroxy” refers to the group —OH. The term “substituted hydroxyl” or “substituted hydroxyl,” by extension, refers to a hydroxyl group wherein the oxygen atom directly attached to the parent molecule is substituted with a group other than hydrogen, and includes groups selected from —OR^{aa}, —ON(R^{bb})₂, —OC(═O)SR^{aa}—, —OC(═O)R^{aa}, —OCO₂R^{aa}, —OC(═O)N(R^{bb})₂, —OC(═NR^{bb})R^{aa}, —OC(═NR^{bb})OR^{aa}, —OC(═NR^{bb})N(R^{bb})₂, —OS(═O)R^{aa}, —OSO₂R^{aa}, —OSi(R^{aa})₃, —OP(R^{aa})₂, —OP(R^{cc})₃⁺X⁻, —OP(OR^{cc})₂, —OP(OR^{cc})₃⁺X⁻, —OP(═O)(R^{cc})₂, —OP(═O)(OR^{cc})₂, and —OP(═O)(N(R^{bb}))₂, wherein X⁻, R^{aa}, R^{bb}, and R^{cc} are as defined herein.

The term “thiol” or “thio” refers to the group —SH. The term “substituted thiol” or “substituted thio,” by extension, refers to a thiol group wherein the sulfur atom directly attached to the parent molecule is substituted with a group other than hydrogen, and includes groups selected from —SR^{aa}, —S═SR^{cc}, —SC(═S)SR^{aa}, —SC(═S)OR^{aa}, —SC(═S)N(R^{bb})₂, —SC(═O)SR^{aa}, —SC(═O)OR^{aa}, —SC(═O)N(R^{bb})₂, and —SC(═O)R^{aa}, wherein R^{aa} and R^{cc} are as defined herein.

The term “amino” refers to the group —NH₂. The term “substituted amino,” by extension, refers to a monosubstituted amino, a disubstituted amino, or a trisubstituted amino. In certain embodiments, the “substituted amino” is a monosubstituted amino or a disubstituted amino group.

The term “monosubstituted amino” refers to an amino group wherein the nitrogen atom directly attached to the parent molecule is substituted with one hydrogen and one group other than hydrogen, and includes groups selected from —NH(R^{bb}), —NHC(═O)R^{aa}—, —NHCO₂R^{aa}, —NHC(═O)N(R^{bb})₂, —NHC(═NR^{bb})N(R^{bb})₂, —NHSO₂R^{aa}, —NHP(═O)(OR^{cc})₂, and —NHP(═O)(N(R^{bb})₂)₂, wherein R^{aa}, R^{bb} and R^{cc} are as defined herein, and wherein R^{bb} of the group —NH(R^{bb}) is not hydrogen.

The term “disubstituted amino” refers to an amino group wherein the nitrogen atom directly attached to the parent molecule is substituted with two groups other than hydrogen, and includes groups selected from —N(R^{bb})₂, —NR^{bb} C(═O)R^{aa}, —NR^{bb}CO₂R^{aa}, —NR^{bb}C(═O)N(R^{bb})₂, —NR^{bb}C(═NR^{bb})N(R^{bb})₂, —NR^{bb}SO₂R^{aa}, —NR^{bb}P(═O)(OR^{cc})₂, and —NR^{bb}P(═O)(N(R^{bb})₂)₂, wherein R^{aa}, R^{bb}, and R^{cc} are as defined herein, with the proviso that the nitrogen atom directly attached to the parent molecule is not substituted with hydrogen.

The term “trisubstituted amino” refers to an amino group wherein the nitrogen atom directly attached to the parent molecule is substituted with three groups, and includes groups selected from —N(R^{bb})₃ and —N(R^{bb})₃⁺X⁻, wherein R^{bb} and X⁻ are as defined herein.

The term “acyl” refers to a group having the general formula —C(═O)R^{X1}, —C(═O)OR^{X1}, —C(═O)—O—C(═O)R^{X1}, —C(═O)SR^{X1}, —C(═O)N(R^{X1})₂, —C(═S)R^{X1}, —C(═S)N(R^{X1})₂, and —C(═S)S(R^{X1}), —C(═NR^{X1})R^{X1}, —C(═NR^{X1})OR^{X1}, —C(═NR^{X1})SR^{X1}, and —C(═NR^{X1})N(R^{X1})₂, wherein R^{X1} is hydrogen; halogen; substituted or unsubstituted hydroxyl; substituted or unsubstituted thiol; substituted or unsubstituted amino; substituted or unsubstituted acyl, cyclic or acyclic, substituted or unsubstituted, branched or unbranched aliphatic; cyclic or acyclic, substituted or unsubstituted, branched or unbranched heteroaliphatic; cyclic or acyclic, substituted or unsubstituted, branched or unbranched alkyl; cyclic or acyclic, substituted or unsubstituted, branched or unbranched alkenyl; substituted or unsubstituted alkynyl; substituted or unsubstituted aryl, substituted or unsubstituted heteroaryl, aliphaticoxy, heteroaliphaticoxy, alkyloxy, heteroalkyloxy, aryloxy, heteroaryloxy, aliphaticthioxy, heteroaliphaticthioxy, alkylthioxy, heteroalkylthioxy, arylthioxy, heteroarylthioxy, mono- or di-aliphaticamino, mono- or di-heteroaliphaticamino, mono- or di-alkylamino, mono- or di-heteroalkylamino, mono- or di-arylamino, or mono- or di-heteroarylamino; or two R^{X1} groups taken together form a 5- to 6-membered heterocyclic ring. Exemplary acyl groups include aldehydes (—CHO), carboxylic acids (—CO₂H), ketones, acyl halides, esters, amides, imines, carbonates, carbamates, and ureas. Acyl substituents include, but are not limited to, any of the substituents described herein, that result in the formation of a stable moiety (e.g., aliphatic, alkyl, alkenyl, alkynyl, heteroaliphatic, heterocyclic, aryl, heteroaryl, acyl, oxo, imino, thiooxo, cyano, isocyano, amino, azido, nitro, hydroxyl, thiol, halo, aliphaticamino, heteroaliphaticamino, alkylamino, heteroalkylamino, arylamino, heteroarylamino, alkylaryl, arylalkyl, aliphaticoxy, heteroaliphaticoxy, alkyloxy, heteroalkyloxy, aryloxy, heteroaryloxy, aliphaticthioxy, heteroaliphaticthioxy, alkylthioxy, heteroalkylthioxy, arylthioxy, heteroarylthioxy, acyloxy, and the like, each of which may or may not be further substituted).

The term “carbonyl” refers to a group wherein the carbon directly attached to the parent molecule is sp² hybridized, and is substituted with an oxygen, nitrogen or sulfur atom, e.g., a group selected from ketones (—C(═O)R^{aa}), carboxylic acids (—CO₂H), aldehydes (—CHO), esters (—CO₂R^{aa}, —C(═O)SR^{aa}, —C(═S)SR^{aa}), amides (—C(═O)N(R^{bb})₂, —C(═O)NR^{bb}SO₂R^{aa}, C(═S)N(R^{bb})₂), and imines (—C(═NR^{bb})R^{aa}—, —C(═NR^{bb})OR^{aa}), —C(═NR^{bb})N(R^{bb})₂), wherein R^{aa} and R^{bb} are as defined herein.

The term “silyl” refers to the group —Si(R^{aa})₃, wherein R^{aa} is as defined herein.

The term “oxo” refers to the group ═O, and the term “thiooxo” refers to the group ═S.

Nitrogen atoms can be substituted or unsubstituted as valency permits, and include primary, secondary, tertiary, and quaternary nitrogen atoms. Exemplary nitrogen atom substituents include hydrogen, —OH, —OR^{aa}, —N(R^{cc})₂, —CN, —C(═O)R^{cc}, —C(═O)N(R^{cc})₂, —CO₂R^{aa}, —SO₂R^{aa}—C(═NR^{bb})R^{aa}—, —C(═NR^{cc})OR^{cc}, —C(═NR^{cc})N(R^{cc})₂, —SO₂N(R^{cc})₂, —SO₂R^{cc}, —SO₂OR^{cc}, —SOR^{aa}, —C(═S)N(R^{cc})₂, —C(═O)SR^{cc}, —C(═S)SR^{cc}, —P(═O)(OR^{cc})₂, —P(═O)(R^{aa})₂, —P(═O)(N(R^{cc})₂)₂, C₁₋₂₀ alkyl, C₁₋₂₀ perhaloalkyl, C₁₋₂₀ alkenyl, C₁₋₂₀ alkynyl, hetero C₁₋₂₀ alkyl, hetero C₁₋₂₀ alkenyl, hetero C₁₋₂₀ alkynyl, C₃₋₁₀ carbocyclyl, 3-14 membered heterocyclyl, C₆₋₁₄ aryl, and 5-14 membered heteroaryl, or two R^{cc} groups attached to an N atom are joined to form a 3-14 membered heterocyclyl or 5-14 membered heteroaryl ring, wherein each alkyl, alkenyl, alkynyl, heteroalkyl, heteroalkenyl, heteroalkynyl, carbocyclyl, heterocyclyl, aryl, and heteroaryl is independently substituted with 0, 1, 2, 3, 4, or 5 R^{dd} groups, and wherein R^{aa}, R^{bb}, R^{cc} and R^{dd} are as defined above.

In certain embodiments, each nitrogen atom substituent is independently substituted (e.g., substituted with one or more halogen) or unsubstituted C₁₋₆ alkyl, —C(═O)R^{aa}, —CO₂R^{aa}, —C(═O)N(R^{bb})₂, or a nitrogen protecting group. In certain embodiments, each nitrogen atom substituent is independently substituted (e.g., substituted with one or more halogen) or unsubstituted C₁₋₁₀ alkyl, —C(═O)R^{aa}, —CO₂R^{aa}, —C(═O)N(R^{bb})₂, or a nitrogen protecting group, wherein R^{aa} is hydrogen, substituted (e.g., substituted with one or more halogen) or unsubstituted C₁₋₁₀ alkyl, or an oxygen protecting group when attached to an oxygen atom; and each R^{bb} is independently hydrogen, substituted (e.g., substituted with one or more halogen) or unsubstituted C₁₋₁₀ alkyl, or a nitrogen protecting group. In certain embodiments, each nitrogen atom substituent is independently substituted (e.g., substituted with one or more halogen) or unsubstituted C₁₋₆ alkyl or a nitrogen protecting group.

In certain embodiments, the substituent present on the nitrogen atom is a nitrogen protecting group (also referred to herein as an “amino protecting group”). Nitrogen protecting groups include —OH, —OR^{aa}, —N(R^{cc})₂, —C(═O)R^{aa}, —C(═O)N(R^{cc})₂, —CO₂R^{aa}, —SO₂R^{aa}, —C(═NR^{cc})R^{aa}, —C(═NR^{cc})OR^{aa}, —C(═NR^{cc})N(R^{cc})₂, —SO₂N(R^{cc})₂, —SO₂R^{cc}, —SO₂OR^{cc}, —SOR^{aa}, —C(═S)N(R^{cc})₂, —C(═O)SR^{cc}, —C(═S)SR^{cc}, C₁₋₁₀ alkyl (e.g., aralkyl, heteroaralkyl), C₁₋₂₀ alkenyl, C₁₋₂₀ alkynyl, hetero C₁₋₂₀ alkyl, hetero C₁₋₂₀ alkenyl, hetero C₁₋₂₀ alkynyl, C₃₋₁₀ carbocyclyl, 3-14 membered heterocyclyl, C₆₋₁₄ aryl, and 5-14 membered heteroaryl groups, wherein each alkyl, alkenyl, alkynyl, heteroalkyl, heteroalkenyl, heteroalkynyl, carbocyclyl, heterocyclyl, aralkyl, aryl, and heteroaryl is independently substituted with 0, 1, 2, 3, 4, or 5 R^{dd} groups, and wherein R^{aa}, R^{bb}, R^{cc} and R^{dd} are as defined herein. Nitrogen protecting groups are well known in the art and include those described in detail in Protecting Groups in Organic Synthesis, T. W. Greene and P. G. M. Wuts, 3^{rd} edition, John Wiley & Sons, 1999, incorporated herein by reference.

For example, in certain embodiments, at least one nitrogen protecting group is an amide group (e.g., a moiety that include the nitrogen atom to which the nitrogen protecting groups (e.g., —C(═O)R^{aa}) is directly attached). In certain such embodiments, each nitrogen protecting group, together with the nitrogen atom to which the nitrogen protecting group is attached, is independently selected from the group consisting of formamide, acetamide, chloroacetamide, trichloroacetamide, trifluoroacetamide, phenylacetamide, 3-phenylpropanamide, picolinamide, 3-pyridylcarboxamide, N-benzoylphenylalanyl derivatives, benzamide, p-phenylbenzamide, o-nitophenylacetamide, o-nitrophenoxyacetamide, acetoacetamide, (N′-dithiobenzyloxyacylamino)acetamide, 3-(p-hydroxyphenyl)propanamide, 3-(o-nitrophenyl)propanamide, 2-methyl-2-(o-nitrophenoxy)propanamide, 2-methyl-2-(o-phenylazophenoxy)propanamide, 4-chlorobutanamide, 3-methyl-3-nitrobutanamide, o-nitrocinnamide, N-acetylmethionine derivatives, o-nitrobenzamide, and o-(benzoyloxymethyl)benzamide.

In certain embodiments, at least one nitrogen protecting group is a carbamate group (e.g., a moiety that include the nitrogen atom to which the nitrogen protecting groups (e.g., —C(═O)OR^{aa}) is directly attached). In certain such embodiments, each nitrogen protecting group, together with the nitrogen atom to which the nitrogen protecting group is attached, is independently selected from the group consisting of methyl carbamate, ethyl carbamate, 9-fluorenylmethyl carbamate (Fmoc), 9-(2-sulfo)fluorenylmethyl carbamate, 9-(2,7-dibromo)fluoroenylmethyl carbamate, 2,7-di-t-butyl-[9-(10,10-dioxo-10,10,10,10-tetrahydrothioxanthyl)]methyl carbamate (DBD-Tmoc), 4-methoxyphenacyl carbamate (Phenoc), 2,2,2-trichloroethyl carbamate (Troc), 2-trimethylsilylethyl carbamate (Teoc), 2-phenylethyl carbamate (hZ), 1-(1-adamantyl)-1-methylethyl carbamate (Adpoc), 1,1-dimethyl-2-haloethyl carbamate, 1,1-dimethyl-2,2-dibromoethyl carbamate (DB-t-BOC), 1,1-dimethyl-2,2,2-trichloroethyl carbamate (TCBOC), 1-methyl-1-(4-biphenylyl)ethyl carbamate (Bpoc), 1-(3,5-di-t-butylphenyl)-1-methylethyl carbamate (t-Bumeoc), 2-(2′- and 4′-pyridyl)ethyl carbamate (Pyoc), 2-(N,N-dicyclohexylcarboxamido)ethyl carbamate, t-butyl carbamate (BOC or Boc), 1-adamantyl carbamate (Adoc), vinyl carbamate (Voc), allyl carbamate (Alloc), 1-isopropylallyl carbamate (Ipaoc), cinnamyl carbamate (Coc), 4-nitrocinnamyl carbamate (Noc), 8-quinolyl carbamate, N-hydroxypiperidinyl carbamate, alkyldithio carbamate, benzyl carbamate (Cbz), p-methoxybenzyl carbamate (Moz), p-nitobenzyl carbamate, p-bromobenzyl carbamate, p-chlorobenzyl carbamate, 2,4-dichlorobenzyl carbamate, 4-methylsulfinylbenzyl carbamate (Msz), 9-anthrylmethyl carbamate, diphenylmethyl carbamate, 2-methylthioethyl carbamate, 2-methylsulfonylethyl carbamate, 2-(p-toluenesulfonyl)ethyl carbamate, [2-(1,3-dithianyl)]methyl carbamate (Dmoc), 4-methylthiophenyl carbamate (Mtpc), 2,4-dimethylthiophenyl carbamate (Bmpc), 2-phosphonioethyl carbamate (Peoc), 2-triphenylphosphonioisopropyl carbamate (Ppoc), 1,1-dimethyl-2-cyanoethyl carbamate, m-chloro-p-acyloxybenzyl carbamate, p-(dihydroxyboryl)benzyl carbamate, 5-benzisoxazolylmethyl carbamate, 2-(trifluoromethyl)-6-chromonylmethyl carbamate (Tcroc), m-nitrophenyl carbamate, 3,5-dimethoxybenzyl carbamate, o-nitrobenzyl carbamate, 3,4-dimethoxy-6-nitrobenzyl carbamate, phenyl(o-nitrophenyl)methyl carbamate, t-amyl carbamate, S-benzyl thiocarbamate, p-cyanobenzyl carbamate, cyclobutyl carbamate, cyclohexyl carbamate, cyclopentyl carbamate, cyclopropylmethyl carbamate, p-decyloxybenzyl carbamate, 2,2-dimethoxyacylvinyl carbamate, o-(N,N-dimethylcarboxamido)benzyl carbamate, 1,1-dimethyl-3-(N,N-dimethylcarboxamido)propyl carbamate, 1,1-dimethylpropynyl carbamate, di(2 -pyridyl)methyl carbamate, 2-furanylmethyl carbamate, 2-iodoethyl carbamate, isoborynl carbamate, isobutyl carbamate, isonicotinyl carbamate, p-(p′-methoxyphenylazo)benzyl carbamate, 1-methylcyclobutyl carbamate, 1-methylcyclohexyl carbamate, 1-methyl-1-cyclopropylmethyl carbamate, 1-methyl-1-(3,5-dimethoxyphenyl)ethyl carbamate, 1-methyl-1-(p-phenylazophenyl)ethyl carbamate, 1-methyl-1-phenylethyl carbamate, 1-methyl-1-(4-pyridyl)ethyl carbamate, phenyl carbamate, p-(phenylazo)benzyl carbamate, 2,4,6-tri-t-butylphenyl carbamate, 4-(trimethylammonium)benzyl carbamate, and 2,4,6-trimethylbenzyl carbamate.

In certain embodiments, at least one nitrogen protecting group is a sulfonamide group (e.g., a moiety that include the nitrogen atom to which the nitrogen protecting groups (e.g., —S(═O)₂R′) is directly attached). In certain such embodiments, each nitrogen protecting group, together with the nitrogen atom to which the nitrogen protecting group is attached, is independently selected from the group consisting of p-toluenesulfonamide (Ts), benzenesulfonamide, 2,3,6-trimethyl-4-methoxybenzenesulfonamide (Mtr), 2,4,6-trimethoxybenzenesulfonamide (Mtb), 2,6-dimethyl-4-methoxybenzenesulfonamide (Pme), 2,3,5,6-tetramethyl-4-methoxybenzenesulfonamide (Mte), 4-methoxybenzenesulfonamide (Mbs), 2,4,6-trimethylbenzenesulfonamide (Mts), 2,6-dimethoxy-4-methylbenzenesulfonamide (iMds), 2,2,5,7,8-pentamethylchroman-6-sulfonamide (Pmc), methanesulfonamide (Ms), β-trimethylsilylethanesulfonamide (SES), 9-anthracenesulfonamide, 4-(4′,8′-dimethoxynaphthylmethyl)benzenesulfonamide (DNMBS), benzylsulfonamide, trifluoromethylsulfonamide, and phenacylsulfonamide.

In certain embodiments, each nitrogen protecting group, together with the nitrogen atom to which the nitrogen protecting group is attached, is independently selected from the group consisting of phenothiazinyl-(10)-acyl derivatives, N′-p-toluenesulfonylaminoacyl derivatives, N′-phenylaminothioacyl derivatives, N-benzoylphenylalanyl derivatives, N-acetylmethionine derivatives, 4,5-diphenyl-3-oxazolin-2-one, N-phthalimide, N-dithiasuccinimide (Dts), N-2,3-diphenylmaleimide, N-2,5-dimethylpyrrole, N-1,1,4,4-tetramethyldisilylazacyclopentane adduct (STABASE), 5-substituted 1,3-dimethyl-1,3,5-triazacyclohexan-2-one, 5-substituted 1,3-dibenzyl-1,3,5-triazacyclohexan-2-one, 1-substituted 3,5-dinitro-4-pyridone, N-methylamine, N-allylamine, N-[2-(trimethylsilyl)ethoxy]methylamine (SEM), N-3-acetoxypropylamine, N-(1-isopropyl-4-nitro-2-oxo-3-pyroolin-3-yl)amine, quaternary ammonium salts, N-benzylamine, N-di(4-methoxyphenyl)methylamine, N-5-dibenzosuberylamine, N-triphenylmethylamine (Tr), N-[ (4-methoxyphenyl)diphenylmethyl]amine (MMTr), N-9-phenylfluorenylamine (PhF), N-2,7-dichloro-9-fluorenylmethyleneamine, N-ferrocenylmethylamino (Fcm), N-2-picolylamino N′-oxide, N-1,1-dimethylthiomethyleneamine, N-benzylideneamine, N-p-methoxybenzylideneamine, N-diphenylmethyleneamine, N-[(2-pyridyl)mesityl]methyleneamine, N—(N′,N′-dimethylaminomethylene)amine, N-p-nitrobenzylideneamine, N-salicylideneamine, N-5-chlorosalicylideneamine, N-(5-chloro-2-hydroxyphenyl)phenylmethyleneamine, N-cyclohexylideneamine, N-(5,5-dimethyl-3-oxo-1-cyclohexenyl)amine, N-borane derivatives, N-diphenylborinic acid derivatives, N-[phenyl(pentaacylchromium- or tungsten)acyl]amine, N-copper chelate, N-zinc chelate, N-nitroamine, N-nitrosoamine, amine N-oxide, diphenylphosphinamide (Dpp), dimethylthiophosphinamide (Mpt), diphenylthiophosphinamide (Ppt), dialkyl phosphoramidates, dibenzyl phosphoramidate, diphenyl phosphoramidate, benzenesulfenamide, o-nitrobenzenesulfenamide (Nps), 2,4-dinitrobenzenesulfenamide, pentachlorobenzenesulfenamide, 2-nitro-4-methoxybenzenesulfenamide, triphenylmethylsulfenamide, and 3-nitropyridinesulfenamide (Npys). In some embodiments, two instances of a nitrogen protecting group together with the nitrogen atoms to which the nitrogen protecting groups are attached are N,N′-isopropylidenediamine.

In certain embodiments, at least one nitrogen protecting group is Bn, Boc, Cbz, Fmoc, trifluoroacetyl, triphenylmethyl, acetyl, or Ts.

In certain embodiments, each oxygen atom substituent is independently substituted (e.g., substituted with one or more halogen) or unsubstituted C₁₋₁₀ alkyl, —C(═O)R^{aa}, —CO₂R^{aa}, —C(═O)N(R^{bb})₂, or an oxygen protecting group. In certain embodiments, each oxygen atom substituents is independently substituted (e.g., substituted with one or more halogen) or unsubstituted C₁₋₆ alkyl, —C(═O)R^{aa}, —CO₂R^{aa}, —C(═O)N(R^{bb})₂, or an oxygen protecting group, wherein R^{aa} is hydrogen, substituted (e.g., substituted with one or more halogen) or unsubstituted C₁₋₁₀ alkyl, or an oxygen protecting group when attached to an oxygen atom; and each R^{bb} is independently hydrogen, substituted (e.g., substituted with one or more halogen) or unsubstituted C₁₋₁₀ alkyl, or a nitrogen protecting group. In certain embodiments, each oxygen atom substituent is independently substituted (e.g., substituted with one or more halogen) or unsubstituted C₁₋₆ alkyl or an oxygen protecting group.

In certain embodiments, the substituent present on an oxygen atom is an oxygen protecting group (also referred to herein as an “hydroxyl protecting group”). Oxygen protecting groups include —R^{aa}, —N(R^{bb})₂, —C(═O)SR^{aa}, —C(═O)R^{aa}, —CO₂R^{aa}, —C(═O)N(R^{bb})₂, —C(═NR^{bb})R^{aa}, —C(═NR^{bb})OR^{aa}, —C(═NR^{bb})N(R^{bb})₂, —S(═O)R^{aa}, —SO₂R^{aa}, —Si(R^{aa})₃, —P(R^{cc})₂, —P(R^{cc})₃⁺X⁻, —P(OR^{cc})₂, —P(OR^{cc})₃⁺X⁻, —P(═O)(R^{aa})₂, —P(═O)(OR^{cc})₂, and —P(═O)(N(R^{bb})₂)₂, wherein X⁻, R^{aa}, R^{bb}, and R^{cc} are as defined herein. Oxygen protecting groups are well known in the art and include those described in detail in Protecting Groups in Organic Synthesis, T. W. Greene and P. G. M. Wuts, 3^{rd} edition, John Wiley & Sons, 1999, incorporated herein by reference.

In certain embodiments, each oxygen protecting group, together with the oxygen atom to which the oxygen protecting group is attached, is selected from the group consisting of methyl, methoxymethyl (MOM), methylthiomethyl (MTM), t-butylthiomethyl, (phenyldimethylsilyl)methoxymethyl (SMOM), benzyloxymethyl (BOM), p-methoxybenzyloxymethyl (PMBM), (4-methoxyphenoxy)methyl (p-AOM), guaiacolmethyl (GUM), t-butoxymethyl, 4-pentenyloxymethyl (POM), siloxymethyl, 2-methoxyethoxymethyl (MEM), 2,2,2-trichloroethoxymethyl, bis(2-chloroethoxy)methyl, 2-(trimethylsilyl)ethoxymethyl (SEMOR), tetrahydropyranyl (THP), 3-bromotetrahydropyranyl, tetrahydrothiopyranyl, 1-methoxycyclohexyl, 4-methoxytetrahydropyranyl (MTHP), 4-methoxytetrahydrothiopyranyl, 4-methoxytetrahydrothiopyranyl S,S-dioxide, 1-[(2-chloro-4-methyl)phenyl]-4-methoxypiperidin-4-yl (CTMP), 1,4-dioxan-2-yl, tetrahydrofuranyl, tetrahydrothiofuranyl, 2,3,3a,4,5,6,7,7a-octahydro-7,8,8-trimethyl-4,7-methanobenzofuran-2-yl, 1-ethoxyethyl, 1-(2-chloroethoxy)ethyl, 1-methyl-1-methoxyethyl, 1-methyl-1-benzyloxyethyl, 1-methyl-1-benzyloxy-2-fluoroethyl, 2,2,2-trichloroethyl, 2-trimethylsilylethyl, 2-(phenylselenyl)ethyl, t-butyl, allyl, p-chlorophenyl, p-methoxyphenyl, 2,4-dinitrophenyl, benzyl (Bn), p-methoxybenzyl (PMB), 3,4-dimethoxybenzyl, o-nitrobenzyl, p-nitrobenzyl, p-halobenzyl, 2,6-dichlorobenzyl, p-cyanobenzyl, p-phenylbenzyl, 2-picolyl, 4-picolyl, 3-methyl-2-picolyl N-oxido, diphenylmethyl, p,p′-dinitrobenzhydryl, 5-dibenzosuberyl, triphenylmethyl, α-naphthyldiphenylmethyl, p-methoxyphenyldiphenylmethyl, di(p-methoxyphenyl)phenylmethyl, tri(p-methoxyphenyl)methyl, 4-(4′-bromophenacyloxyphenyl)diphenylmethyl, 4,4′,4″-tris(4,5-dichlorophthalimidophenyl)methyl, 4,4′,4″-tris(levulinoyloxyphenyl)methyl, 4,4′,4″-tris(benzoyloxyphenyl)methyl, 4,4′-Dimethoxy-3″′-[N-(imidazolylmethyl)]trityl Ether (IDTr-OR), 4,4′-Dimethoxy-3″′-[N-(imidazolylethyl)carbamoyl]trityl Ether (IETr-OR), 1,1-bis(4-methoxyphenyl)-1′-pyrenylmethyl, 9-anthryl, 9-(9-phenyl)xanthenyl, 9-(9-phenyl-10-oxo)anthryl, 1,3-benzodithiolan-2-yl, benzisothiazolyl S,S-dioxido, trimethylsilyl (TMS), triethylsilyl (TES), triisopropylsilyl (TIPS), dimethylisopropylsilyl (IPDMS), diethylisopropylsilyl (DEIPS), dimethylthexylsilyl, t-butyldimethylsilyl (TBDMS), t-butyldiphenylsilyl (TBDPS), tribenzylsilyl, tri-p-xylylsilyl, triphenylsilyl, diphenylmethylsilyl (DPMS), t-butylmethoxyphenylsilyl (TBMPS), formate, benzoylformate, acetate, chloroacetate, dichloroacetate, trichloroacetate, trifluoroacetate, methoxyacetate, triphenylmethoxyacetate, phenoxyacetate, p-chlorophenoxyacetate, 3-phenylpropionate, 4-oxopentanoate (levulinate), 4,4-(ethylenedithio)pentanoate (levulinoyldithioacetal), pivaloate, adamantoate, crotonate, 4-methoxycrotonate, benzoate, p-phenylbenzoate, 2,4,6-trimethylbenzoate (mesitoate), methyl carbonate, 9-fluorenylmethyl carbonate (Fmoc), ethyl carbonate, 2,2,2-trichloroethyl carbonate (Troc), 2-(trimethylsilyl)ethyl carbonate (TMSEC), 2-(phenylsulfonyl) ethyl carbonate (Psec), 2-(triphenylphosphonio) ethyl carbonate (Peoc), isobutyl carbonate, vinyl carbonate, allyl carbonate, t-butyl carbonate (BOC or Boc), p-nitrophenyl carbonate, benzyl carbonate, p-methoxybenzyl carbonate, 3,4-dimethoxybenzyl carbonate, o-nitrobenzyl carbonate, p-nitrobenzyl carbonate, S-benzyl thiocarbonate, 4-ethoxy-1-napththyl carbonate, methyl dithiocarbonate, 2-iodobenzoate, 4-azidobutyrate, 4-nitro-4-methylpentanoate, o-(dibromomethyl)benzoate, 2-formylbenzenesulfonate, 2-(methylthiomethoxy)ethyl carbonate (MTMEC-OR), 4-(methylthiomethoxy)butyrate, 2-(methylthiomethoxymethyl)benzoate, 2,6-dichloro-4-methylphenoxyacetate, 2,6-dichloro-4-(1,1,3,3-tetramethylbutyl)phenoxyacetate, 2,4-bis(1,1-dimethylpropyl)phenoxyacetate, chlorodiphenylacetate, isobutyrate, monosuccinoate, (E)-2-methyl-2-butenoate, o-(methoxyacyl)benzoate, α-naphthoate, nitrate, alkyl N,N,N′,N′-tetramethylphosphorodiamidate, alkyl N-phenylcarbamate, borate, dimethylphosphinothioyl, alkyl 2,4-dinitrophenylsulfenate, sulfate, methanesulfonate (mesylate), benzylsulfonate, and tosylate (Ts).

In certain embodiments, at least one oxygen protecting group is silyl, TBDPS, TBDMS, TIPS, TES, TMS, MOM, THP, t-Bu, Bn, allyl, acetyl, pivaloyl, or benzoyl.

In certain embodiments, each sulfur atom substituent is independently substituted (e.g., substituted with one or more halogen) or unsubstituted C₁₋₁₀ alkyl, —C(═O)R^{aa}, —CO₂R^{aa}, —C(═O)N(R^{bb})₂, or a sulfur protecting group. In certain embodiments, each sulfur atom substituent is independently substituted (e.g., substituted with one or more halogen) or unsubstituted C₁₋₁₀ alkyl, —C(═O)R^{aa}, —CO₂R^{aa}, —C(═O)N(R^{bb})₂, or a sulfur protecting group, wherein R^{aa} is hydrogen, substituted (e.g., substituted with one or more halogen) or unsubstituted C₁₋₁₀ alkyl, or an oxygen protecting group when attached to an oxygen atom; and each R^{bb} is independently hydrogen, substituted (e.g., substituted with one or more halogen) or unsubstituted C₁₋₁₀ alkyl, or a nitrogen protecting group. In certain embodiments, each sulfur atom substituent is independently substituted (e.g., substituted with one or more halogen) or unsubstituted C₁₋₆ alkyl or a sulfur protecting group.

In certain embodiments, the substituent present on a sulfur atom is a sulfur protecting group (also referred to as a “thiol protecting group”). In some embodiments, each sulfur protecting group is selected from the group consisting of —R^{aa}, —N(R^{bb})₂, —C(═O)SR^{aa}, —C(═O)R^{aa}, —CO₂R^{aa}, —C(═O)N(R^{bb})₂, —C(═NR^{bb})R^{aa}, —C(═NR^{bb})OR^{aa}, —C(═NR^{bb})N(R^{bb})₂, —S(═O)R^{aa}, —SO₂R^{a}, —Si(R^{aa})₃, —P(R^{cc})₂, —P(R^{cc})₃⁺X⁻, —P(OR^{cc})₂, —P(OR^{cc})₃⁺X, —P(═O)(R^{cc})₂, —P(═O)(OR^{cc})₂, and —P(═O)(N(R^{bb})₂)₂, wherein R^{aa}, R^{bb}, and R^{cc} are as defined herein. Sulfur protecting groups are well known in the art and include those described in detail in Protecting Groups in Organic Synthesis, T. W. Greene and P. G. M. Wuts, 3^{rd} edition, John Wiley & Sons, 1999, incorporated herein by reference.

In certain embodiments, the molecular weight of a substituent is lower than 250, lower than 200, lower than 150, lower than 100, or lower than 50 g/mol. In certain embodiments, a substituent consists of carbon, hydrogen, fluorine, chlorine, bromine, iodine, oxygen, sulfur, nitrogen, and/or silicon atoms. In certain embodiments, a substituent consists of carbon, hydrogen, fluorine, chlorine, bromine, iodine, oxygen, sulfur, and/or nitrogen atoms. In certain embodiments, a substituent consists of carbon, hydrogen, fluorine, chlorine, bromine, and/or iodine atoms. In certain embodiments, a substituent consists of carbon, hydrogen, fluorine, and/or chlorine atoms. In certain embodiments, a substituent comprises 0, 1, 2, or 3 hydrogen bond donors. In certain embodiments, a substituent comprises 0, 1, 2, or 3 hydrogen bond acceptors.

A “counterion” or “anionic counterion” is a negatively charged group associated with a positively charged group in order to maintain electronic neutrality. An anionic counterion may be monovalent (e.g., including one formal negative charge). An anionic counterion may also be multivalent (e.g., including more than one formal negative charge), such as divalent or trivalent. Exemplary counterions include halide ions (e.g., F⁻, Cl⁻, Br⁻, I⁻), NO₃⁻, ClO₄⁻, OH⁻, H₂PO₄⁻, HCO₃⁻, HSO₄⁻, sulfonate ions (e.g., methansulfonate, trifluoromethanesulfonate, p-toluenesulfonate, benzenesulfonate, 10-camphor sulfonate, naphthalene-2-sulfonate, naphthalene-1-sulfonic acid-5-sulfonate, ethan-1-sulfonic acid-2-sulfonate, and the like), carboxylate ions (e.g., acetate, propanoate, benzoate, glycerate, lactate, tartrate, glycolate, gluconate, and the like), BF₄⁻, PF₄⁻, PF₆⁻, AsF₆⁻, SbF₆⁻, B[3,5-(CF₃)₂C₆H₃]₄]⁻, B(C₆F₅)₄⁻, BPh₄⁻, Al(OC(CF₃)₃)₄⁻, and carborane anions (e.g., CB₁₁H₁₂⁻ or (HCB₁₁Me₅Br₆)⁻). Exemplary counterions which may be multivalent include CO₃²⁻, HPO₄²⁻, PO₄³⁻, B₄O₇²⁻, SO₄²⁻, S₂O₃²⁻, carboxylate anions (e.g., tartrate, citrate, fumarate, maleate, malate, malonate, gluconate, succinate, glutarate, adipate, pimelate, suberate, azelate, sebacate, salicylate, phthalates, aspartate, glutamate, and the like), and carboranes.

Use of the phrase “at least one instance” refers to 1, 2, 3, 4, or more instances, but also encompasses a range, e.g., for example, from 1 to 4, from 1 to 3, from 1 to 2, from 2 to 4, from 2 to 3, or from 3 to 4 instances, inclusive.

A “non-hydrogen group” refers to any group that is defined for a particular variable that is not hydrogen.

The term “pharmaceutically acceptable salt” refers to those salts which are, within the scope of sound medical judgment, suitable for use in contact with the tissues of humans and lower animals without undue toxicity, irritation, allergic response, and the like, and are commensurate with a reasonable benefit/risk ratio. Pharmaceutically acceptable salts are well known in the art. For example, Berge et al. describe pharmaceutically acceptable salts in detail in J. Pharmaceutical Sciences, 1977, 66, 1-19, incorporated herein by reference. Pharmaceutically acceptable salts of the compounds of this invention include those derived from suitable inorganic and organic acids and bases. Examples of pharmaceutically acceptable, nontoxic acid addition salts are salts of an amino group formed with inorganic acids, such as hydrochloric acid, hydrobromic acid, phosphoric acid, sulfuric acid, and perchloric acid or with organic acids, such as acetic acid, oxalic acid, maleic acid, tartaric acid, citric acid, succinic acid, or malonic acid or by using other methods known in the art such as ion exchange. Other pharmaceutically acceptable salts include adipate, alginate, ascorbate, aspartate, benzenesulfonate, benzoate, bisulfate, borate, butyrate, camphorate, camphorsulfonate, citrate, cyclopentanepropionate, digluconate, dodecylsulfate, ethanesulfonate, formate, fumarate, glucoheptonate, glycerophosphate, gluconate, hemisulfate, heptanoate, hexanoate, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, picrate, pivalate, propionate, stearate, succinate, sulfate, tartrate, thiocyanate, p-toluenesulfonate, undecanoate, valerate salts, and the like. Salts derived from appropriate bases include alkali metal, alkaline earth metal, ammonium, and N⁺(C₁₋₄ alkyl)₄ salts. Representative alkali or alkaline earth metal salts include sodium, lithium, potassium, calcium, magnesium, and the like. Further pharmaceutically acceptable salts include, when appropriate, nontoxic ammonium, quaternary ammonium, and amine cations formed using counterions such as halide, hydroxide, carboxylate, sulfate, phosphate, nitrate, lower alkyl sulfonate, and aryl sulfonate.

The term “solvate” refers to forms of the compound, or a salt thereof, that are associated with a solvent, usually by a solvolysis reaction. This physical association may include hydrogen bonding. Conventional solvents include water, methanol, ethanol, acetic acid, DMSO, THF, diethyl ether, and the like. The compounds described herein may be prepared, e.g., in crystalline form, and may be solvated. Suitable solvates include pharmaceutically acceptable solvates and further include both stoichiometric solvates and non-stoichiometric solvates. In certain instances, the solvate will be capable of isolation, for example, when one or more solvent molecules are incorporated in the crystal lattice of a crystalline solid. “Solvate” encompasses both solution-phase and isolatable solvates. Representative solvates include hydrates, ethanolates, and methanolates.

The term “stoichiometric solvate” refers to a solvate, which comprises a compound (e.g., a compound disclosed herein) and a solvent, wherein the solvent molecules are an integral part of the crystal lattice, in which they interact strongly with the compound and each other. The removal of the solvent molecules will cause instability of the crystal network, which subsequently collapses into an amorphous phase or recrystallizes as a new crystalline form with reduced solvent content.

The term “non-stoichiometric solvate” refers to a solvate, which comprises a compound (e.g., a compound disclosed herein) and a solvent, wherein the solvent content may vary without major changes in the crystal structure. The amount of solvent in the crystal lattice only depends on the partial pressure of solvent in the surrounding atmosphere. In the fully solvated state, non-stoichiometric solvates may, but not necessarily have to, show an integer molar ratio of solvent to the compound. During drying of a non-stoichiometric solvate, a portion of the solvent may be removed without significantly disturbing the crystal network, and the resulting solvate can subsequently be resolvated to give the initial crystalline form. Unlike stoichiometric solvates, the desolvation and resolvation of non-stoichiometric solvates is not accompanied by a phase transition, and all solvation states represent the same crystal form.

The term “hydrate” refers to a compound that is associated with water. Typically, the number of the water molecules contained in a hydrate of a compound is in a definite ratio to the number of the compound molecules in the hydrate. Therefore, a hydrate of a compound may be represented, for example, by the general formula R·x H₂O, wherein R is the compound, and x is a number greater than 0. A given compound may form more than one type of hydrate, including, e.g., monohydrates (x is 1), lower hydrates (x is a number greater than 0 and smaller than 1, e.g., hemihydrates (R·0.5 H₂O)), and polyhydrates (x is a number greater than 1, e.g., dihydrates (R·2H₂O) and hexahydrates (R·6H₂O)).

The term “tautomers” or “tautomeric” refers to two or more interconvertible compounds resulting from at least one formal migration of a hydrogen atom and at least one change in valency (e.g., a single bond to a double bond, a triple bond to a single bond, or vice versa). The exact ratio of the tautomers depends on several factors, including temperature, solvent, and pH. Tautomerizations (i.e., the reaction providing a tautomeric pair) may catalyzed by acid or base. Exemplary tautomerizations include keto-to-enol, amide-to-imide, lactam-to-lactim, enamine-to-imine, and enamine-to-(a different enamine) tautomerizations.

It is also to be understood that compounds that have the same molecular formula but differ in the nature or sequence of bonding of their atoms or the arrangement of their atoms in space are termed “isomers”. Isomers that differ in the arrangement of their atoms in space are termed “stereoisomers”.

Stereoisomers that are not mirror images of one another are termed “diastereomers” and those that are non-superimposable mirror images of each other are termed “enantiomers”. When a compound has an asymmetric center, for example, it is bonded to four different groups, a pair of enantiomers is possible. An enantiomer can be characterized by the absolute configuration of its asymmetric center and is described by the R- and S-sequencing rules of Cahn and Prelog, or by the manner in which the molecule rotates the plane of polarized light and designated as dextrorotatory or levorotatory (i.e., as (+) or (−)-isomers respectively). A chiral compound can exist as either individual enantiomer or as a mixture thereof. A mixture containing equal proportions of the enantiomers is called a “racemic mixture”.

The term “crystalline” or “crystalline form” refers to a solid form substantially exhibiting three-dimensional order. In certain embodiments, a crystalline form of a solid is a solid form that is substantially not amorphous. In certain embodiments, the X-ray powder diffraction (XRPD) pattern of a crystalline form includes one or more sharply defined peaks.

The term “co-crystal” refers to a crystalline structure comprising at least two different components (e.g., a compound disclosed herein and an acid), wherein each of the components is independently an atom, ion, or molecule. In certain embodiments, none of the components is a solvent. In certain embodiments, at least one of the components is a solvent. A co-crystal of a compound disclosed herein and an acid is different from a salt formed from a compound disclosed herein and the acid. In the salt, a compound disclosed herein is complexed with the acid in a way that proton transfer (e.g., a complete proton transfer) from the acid to a compound disclosed herein easily occurs at room temperature. In the co-crystal, however, a compound disclosed herein is complexed with the acid in a way that proton transfer from the acid to a compound disclosed herein does not easily occur at room temperature. In certain embodiments, in the co-crystal, there is no proton transfer from the acid to a compound disclosed herein. In certain embodiments, in the co-crystal, there is partial proton transfer from the acid to a compound disclosed herein. Co-crystals may be useful to improve the properties (e.g., solubility, stability, and ease of formulation) of a compound disclosed herein.

The term “polymorph” refers to a crystalline form of a compound (or a salt, hydrate, or solvate thereof). All polymorphs have the same elemental composition. Different crystalline forms usually have different X-ray diffraction patterns, infrared spectra, melting points, density, hardness, crystal shape, optical and electrical properties, stability, and solubility. Recrystallization solvent, rate of crystallization, storage temperature, and other factors may cause one crystal form to dominate. Various polymorphs of a compound can be prepared by crystallization under different conditions.

The term “prodrugs” refers to compounds, including derivatives of the compounds of Formula (I), (II), or Table A, that have cleavable groups and become by solvolysis or under physiological conditions the compounds described herein (e.g., the compounds of Formula (I) or (II), compounds of Table A), which are pharmaceutically active in vivo. Such examples include, but are not limited to, choline ester derivatives and the like, N-alkylmorpholine esters and the like. Other derivatives of the compounds described herein have activity in both their acid and acid derivative forms, but in the acid sensitive form often offer advantages of solubility, tissue compatibility, or delayed release in the mammalian organism (see, Bundgard, H., Design of Prodrugs, pp. 7-9, 21-24, Elsevier, Amsterdam 1985). Prodrugs include acid derivatives well known to practitioners of the art, such as, for example, esters prepared by reaction of the parent acid with a suitable alcohol, or amides prepared by reaction of the parent acid compound with a substituted or unsubstituted amine, or acid anhydrides, or mixed anhydrides. Simple aliphatic or aromatic esters, amides, and anhydrides derived from acidic groups pendant on the compounds described herein are particular prodrugs. In some cases it is desirable to prepare double ester type prodrugs such as (acyloxy)alkyl esters or ((alkoxycarbonyl)oxy)alkylesters. C₁-C₈ alkyl, C₂-C₈ alkenyl, C₂-C₈ alkynyl, aryl, C₇-C₁₂ substituted aryl, and C₇-C₁₂ arylalkyl esters of the compounds described herein may be preferred.

A “subject” to which administration is contemplated includes, but is not limited to, humans (i.e., a male or female of any age group, e.g., a pediatric subject (e.g., infant, child, adolescent) or adult subject (e.g., young adult, middle-aged adult, or senior adult)) and/or other non-human animals, for example, mammals (e.g., primates (e.g., cynomolgus monkeys, rhesus monkeys); commercially relevant mammals such as cattle, pigs, horses, sheep, goats, cats, and/or dogs) and birds (e.g., commercially relevant birds such as chickens, ducks, geese, and/or turkeys). In certain embodiments, the animal is a mammal. The animal may be a male or female and at any stage of development. A non-human animal may be a transgenic animal.

The term “biological sample” refers to any sample including tissue samples (such as tissue sections and needle biopsies of a tissue); cell samples (e.g., cytological smears (such as Pap or blood smears) or samples of cells obtained by microdissection); samples of whole organisms (such as samples of yeasts or bacteria); or cell fractions, fragments, organelles (such as obtained by lysing cells and separating the components thereof by centrifugation or otherwise). Other examples of biological samples include blood, serum, urine, semen, fecal matter, cerebrospinal fluid, interstitial fluid, mucus, tears, sweat, pus, biopsied tissue (e.g., obtained by a surgical biopsy or needle biopsy), nipple aspirates, milk, vaginal fluid, saliva, swabs (such as buccal swabs), or any material containing biomolecules that is derived from a first biological sample. Biological samples also include those biological samples that are transgenic, such as a transgenic oocyte, sperm cell, blastocyst, embryo, fetus, donor cell, or cell nucleus, or cells or cell lines derived from biological samples.

The term “tissue” refers to any biological tissue of a subject (including a group of cells, a body part, or an organ) or a part thereof, including blood and/or lymph vessels, which is the object to which a compound, particle, and/or composition of the invention is delivered. A tissue may be an abnormal or unhealthy tissue, which may need to be treated. A tissue may also be a normal or healthy tissue that is under a higher than normal risk of becoming abnormal or unhealthy, which may need to be prevented. In certain embodiments, the tissue is the central nervous system. In certain embodiments, the tissue is the brain.

The terms “administer,” “administering,” or “administration” refer to implanting, absorbing, ingesting, injecting, inhaling, or otherwise introducing a compound, or a pharmaceutical composition thereof.

The terms “treatment,” “treat,” and “treating” refer to reversing, alleviating, delaying the onset of, or inhibiting the progress of a “pathological condition” (e.g., a disease, disorder, or condition, or one or more signs or symptoms thereof) described herein. In some embodiments, treatment may be administered after one or more signs or symptoms have developed or have been observed. In other embodiments, treatment may be administered in the absence of signs or symptoms of the disease or condition. For example, treatment may be administered to a susceptible individual prior to the onset of symptoms (e.g., in light of a history of symptoms and/or in light of genetic or other susceptibility factors). Treatment may also be continued after symptoms have resolved, for example, to delay or prevent recurrence.

The terms “condition,” “disease,” and “disorder” are used interchangeably.

An “effective amount” of a compound of Formula (I) or (II), or a compound of Table A, refers to an amount sufficient to elicit the desired biological response, i.e., treating the condition. As will be appreciated by those of ordinary skill in this art, the effective amount of a compound of Formula (I) or (II), or a compound of Table A, may vary depending on such factors as the desired biological endpoint, the pharmacokinetics of the compound, the condition being treated, the mode of administration, and the age and health of the subject. An effective amount encompasses therapeutic and prophylactic treatment. For example, in treating cancer, an effective amount of a compound may reduce the tumor burden or stop the growth or spread of a tumor.

A “therapeutically effective amount” of a compound of Formula (I) or (II), or a compound of Table A, is an amount sufficient to provide a therapeutic benefit in the treatment of a condition or to delay or minimize one or more symptoms associated with the condition. A therapeutically effective amount of a compound means an amount of therapeutic agent, alone or in combination with other therapies, which provides a therapeutic benefit in the treatment of the condition. The term “therapeutically effective amount” can encompass an amount that improves overall therapy, reduces, or avoids symptoms or causes of the condition, or enhances the therapeutic efficacy of another therapeutic agent.

A “prophylactically effective amount” of a compound described herein is an amount sufficient to prevent a condition, or one or more signs or symptoms associated with the condition, or prevent its recurrence. A prophylactically effective amount of a compound means an amount of a therapeutic agent, alone or in combination with other agents, which provides a prophylactic benefit in the prevention of the condition. The term “prophylactically effective amount” can encompass an amount that improves overall prophylaxis or enhances the prophylactic efficacy of another prophylactic agent. In certain embodiments, a prophylactically effective amount is an amount sufficient for binding a folate-dependent enzyme (e.g., thymidylate synthase, dihydrofolate reductase) and/or inhibiting a folate-dependent enzyme. In certain embodiments, a prophylactically effective amount is an amount sufficient for binding a folate-dependent enzyme (e.g., thymidylate synthase, dihydrofolate reductase) and/or inhibiting a folate-dependent enzyme (e.g., thymidylate synthase, dihydrofolate reductase). In certain embodiments, a prophylactically effective amount is an amount sufficient for preventing a disease and/or condition (e.g., proliferative disease, such as, cancers, or other diseases associated with a folate-dependent enzyme (e.g., thymidylate synthase, dihydrofolate reductase)).

In certain embodiments, a prophylactically effective amount is an amount sufficient for binding a folate-dependent enzyme (e.g., thymidylate synthase, dihydrofolate reductase) and/or inhibiting a folate-dependent enzyme (e.g., thymidylate synthase, dihydrofolate reductase), and preventing a disease and/or condition (e.g., proliferative disease, such as, cancers, or other diseases associated with a folate-dependent enzyme (e.g., thymidylate synthase, dihydrofolate reductase)).

A “proliferative disease” refers to a disease that occurs due to abnormal growth or extension by the multiplication of cells (Walker, Cambridge Dictionary of Biology; Cambridge University Press: Cambridge, UK, 1990). A proliferative disease may be associated with: 1) the pathological proliferation of normally quiescent cells; 2) the pathological migration of cells from their normal location (e.g., metastasis of neoplastic cells); 3) the pathological expression of proteolytic enzymes such as the matrix metalloproteinases (e.g., collagenases, gelatinases, and elastases); or 4) the pathological angiogenesis as in proliferative retinopathy and tumor metastasis. Exemplary proliferative diseases include cancers (i.e., “malignant neoplasms”), benign neoplasms, carcinoma, lung cancer, breast cancer, liver cancer, pancreatic cancer, gastric cancer, ovarian cancer, cervical cancer, colon cancer, colorectal cancer, bladder cancer, hematopoietic cancer, and/or drug-resistant cancer. Exemplary proliferative diseases include cancers (i.e., “malignant neoplasms”), benign neoplasms, angiogenesis, inflammatory diseases, autoinflammatory diseases, and autoimmune diseases.

The terms “neoplasm” and “tumor” are used herein interchangeably and refer to an abnormal mass of tissue wherein the growth of the mass surpasses and is not coordinated with the growth of a normal tissue. A neoplasm or tumor may be “benign” or “malignant,” depending on the following characteristics: degree of cellular differentiation (including morphology and functionality), rate of growth, local invasion, and metastasis. A “benign neoplasm” is generally well differentiated, has characteristically slower growth than a malignant neoplasm, and remains localized to the site of origin. In addition, a benign neoplasm does not have the capacity to infiltrate, invade, or metastasize to distant sites. Exemplary benign neoplasms include, but are not limited to, lipoma, chondroma, adenomas, acrochordon, senile angiomas, seborrheic keratoses, lentigos, and sebaceous hyperplasias. In some cases, certain “benign” tumors may later give rise to malignant neoplasms, which may result from additional genetic changes in a subpopulation of the tumor's neoplastic cells, and these tumors are referred to as “pre-malignant neoplasms.” An exemplary pre-malignant neoplasm is a teratoma. In contrast, a “malignant neoplasm” is generally poorly differentiated (anaplasia) and has characteristically rapid growth accompanied by progressive infiltration, invasion, and destruction of the surrounding tissue. Furthermore, a malignant neoplasm generally has the capacity to metastasize to distant sites. The term “metastasis,” “metastatic,” or “metastasize” refers to the spread or migration of cancerous cells from a primary original tumor to another organ or tissue and is typically identifiable by the presence of a “secondary tumor” or “secondary cell mass” of the tissue type of the primary original tumor and not of that of the organ or tissue in which the secondary (metastatic) tumor is located. For example, a prostate cancer that has migrated to bone is said to be metastasized prostate cancer and includes cancerous prostate cancer cells growing in bone tissue.

The term “cancer” refers to a malignant neoplasm (Stedman's Medical Dictionary, 25th ed.; Hensyl ed.; Williams & Wilkins: Philadelphia, 1990). Exemplary cancers include, but are not limited to, acoustic neuroma; adenocarcinoma; adrenal gland cancer; anal cancer; angiosarcoma (e.g., lymphangiosarcoma, lymphangioendotheliosarcoma, hemangiosarcoma); appendix cancer; benign monoclonal gammopathy; biliary cancer (e.g., cholangiocarcinoma); bladder cancer; breast cancer (e.g., adenocarcinoma of the breast, papillary carcinoma of the breast, mammary cancer, medullary carcinoma of the breast); brain cancer (e.g., meningioma, glioblastomas, glioma (e.g., astrocytoma, oligodendroglioma), medulloblastoma); bronchus cancer; carcinoid tumor; cervical cancer (e.g., cervical adenocarcinoma); choriocarcinoma; chordoma; craniopharyngioma; colorectal cancer (e.g., colon cancer, rectal cancer, colorectal adenocarcinoma); connective tissue cancer; epithelial carcinoma; ependymoma; endotheliosarcoma (e.g., Kaposi's sarcoma, multiple idiopathic hemorrhagic sarcoma); endometrial cancer (e.g., uterine cancer, uterine sarcoma); esophageal cancer (e.g., adenocarcinoma of the esophagus, Barrett's adenocarcinoma); Ewing's sarcoma; eye cancer (e.g., intraocular melanoma, retinoblastoma); familiar hypereosinophilia; gall bladder cancer; gastric cancer (e.g., stomach adenocarcinoma); gastrointestinal stromal tumor (GIST); germ cell cancer; head and neck cancer (e.g., head and neck squamous cell carcinoma, oral cancer (e.g., oral squamous cell carcinoma), throat cancer (e.g., laryngeal cancer, pharyngeal cancer, nasopharyngeal cancer, oropharyngeal cancer)); hematopoietic cancers (e.g., leukemia such as acute lymphocytic leukemia (ALL) (e.g., B-cell ALL, T-cell ALL), acute myelocytic leukemia (AML) (e.g., B-cell AML, T-cell AML), chronic myelocytic leukemia (CML) (e.g., B-cell CML, T-cell CML), acute monocytic leukemia (AMOL), hairy cell leukemia, and chronic lymphocytic leukemia (CLL) (e.g., B-cell CLL, T-cell CLL)); lymphoma such as Hodgkin lymphoma (HL) (e.g., B-cell HL, T-cell HL) and non-Hodgkin lymphoma (NHL) (e.g., B-cell NHL such as diffuse large cell lymphoma (DLCL) (e.g., diffuse large B-cell lymphoma), follicular lymphoma, chronic lymphocytic leukemia/small lymphocytic lymphoma (CLL/SLL), immunoblastic large cell lymphoma, Hodgkin's lymphoma, non-Hodgkin's lymphoma, precursor B-lymphoblastic lymphoma and primary central nervous system (CNS) lymphoma; and T-cell NHL such as precursor T-lymphoblastic lymphoma/leukemia, peripheral T-cell lymphoma (PTCL) (e.g., cutaneous T-cell lymphoma (CTCL) (e.g., mycosis fungoides, Sezary syndrome), angioimmunoblastic T-cell lymphoma, extranodal natural killer T-cell lymphoma, enteropathy type T-cell lymphoma, subcutaneous panniculitis-like T-cell lymphoma, and anaplastic large cell lymphoma); a mixture of one or more leukemia/lymphoma as described above; and multiple myeloma (MM)), plasmacytoma, heavy chain disease (e.g., alpha chain disease, gamma chain disease, mu chain disease); hemangioblastoma; hypopharynx cancer; inflammatory myofibroblastic tumors; immunocytic amyloidosis; kidney cancer (e.g., nephroblastoma a.k.a. Wilms' tumor, renal cell carcinoma); liver cancer (e.g., hepatocellular cancer (HCC), malignant hepatoma); lung cancer (e.g., bronchogenic carcinoma, small cell lung cancer (SCLC), non-small cell lung cancer (NSCLC), adenocarcinoma of the lung); leiomyosarcoma (LMS); mastocytosis (e.g., systemic mastocytosis); muscle cancer; myelodysplastic syndrome (MDS); mesothelioma; myeloproliferative disorder (MPD) (e.g., polycythemia vera (PV), essential thrombocytosis (ET), agnogenic myeloid metaplasia (AMM) a.k.a. myelofibrosis (MF), chronic idiopathic myelofibrosis, chronic myelocytic leukemia (CML), chronic neutrophilic leukemia (CNL), hypereosinophilic syndrome (HES)); neuroblastoma; neurofibroma (e.g., neurofibromatosis (NF) type 1 or type 2, schwannomatosis); neuroendocrine cancer (e.g., gastroenteropancreatic neuroendocrinetumor (GEP-NET), carcinoid tumor); osteosarcoma (e.g., bone cancer); ovarian cancer (e.g., cystadenocarcinoma, ovarian embryonal carcinoma, ovarian adenocarcinoma); papillary adenocarcinoma; pancreatic cancer (e.g., pancreatic andenocarcinoma, intraductal papillary mucinous neoplasm (IPMN), Islet cell tumors); penile cancer (e.g., Paget's disease of the penis and scrotum); pinealoma; primitive neuroectodermal tumor (PNT); plasma cell neoplasia; paraneoplastic syndromes; intraepithelial neoplasms; prostate cancer (e.g., prostate adenocarcinoma); rectal cancer; rhabdomyosarcoma; salivary gland cancer; skin cancer (e.g., squamous cell carcinoma (SCC), keratoacanthoma (KA), melanoma, basal cell carcinoma (BCC)); small bowel cancer (e.g., appendix cancer); soft tissue sarcoma (e.g., malignant fibrous histiocytoma (MFH), liposarcoma, malignant peripheral nerve sheath tumor (MPNST), chondrosarcoma, fibrosarcoma, myxosarcoma); sebaceous gland carcinoma; small intestine cancer; sweat gland carcinoma; synovioma; testicular cancer (e.g., seminoma, testicular embryonal carcinoma); thyroid cancer (e.g., papillary carcinoma of the thyroid, papillary thyroid carcinoma (PTC), medullary thyroid cancer); urethral cancer; vaginal cancer; and vulvar cancer (e.g., Paget's disease of the vulva).

The term “inflammatory disease” refers to a disease caused by, resulting from, or resulting in inflammation. The term “inflammatory disease” may also refer to a dysregulated inflammatory reaction that causes an exaggerated response by macrophages, granulocytes, and/or T-lymphocytes leading to abnormal tissue damage and/or cell death. An inflammatory disease can be either an acute or chronic inflammatory condition and can result from infections or non-infectious causes. Inflammatory diseases include, without limitation, atherosclerosis, arteriosclerosis, autoimmune disorders, multiple sclerosis, systemic lupus erythematosus, polymyalgia rheumatica (PMR), gouty arthritis, degenerative arthritis, tendonitis, bursitis, psoriasis, cystic fibrosis, arthrosteitis, rheumatoid arthritis, inflammatory arthritis, Sjogren's syndrome, giant cell arteritis, progressive systemic sclerosis (scleroderma), ankylosing spondylitis, polymyositis, dermatomyositis, pemphigus, pemphigoid, diabetes (e.g., Type I), myasthenia gravis, Hashimoto's thyroiditis, Graves' disease, Goodpasture's disease, mixed connective tissue disease, sclerosing cholangitis, inflammatory bowel disease, Crohn's disease, ulcerative colitis, pernicious anemia, inflammatory dermatoses, usual interstitial pneumonitis (UIP), asbestosis, silicosis, bronchiectasis, berylliosis, talcosis, pneumoconiosis, sarcoidosis, desquamative interstitial pneumonia, lymphoid interstitial pneumonia, giant cell interstitial pneumonia, cellular interstitial pneumonia, extrinsic allergic alveolitis, Wegener's granulomatosis and related forms of angiitis (temporal arteritis and polyarteritis nodosa), inflammatory dermatoses, hepatitis, delayed-type hypersensitivity reactions (e.g., poison ivy dermatitis), pneumonia, respiratory tract inflammation, Adult Respiratory Distress Syndrome (ARDS), encephalitis, immediate hypersensitivity reactions, asthma, hayfever, allergies, acute anaphylaxis, rheumatic fever, glomerulonephritis, pyelonephritis, cellulitis, cystitis, chronic cholecystitis, ischemia (ischemic injury), reperfusion injury, allograft rejection, host-versus-graft rejection, appendicitis, arteritis, blepharitis, bronchiolitis, bronchitis, cervicitis, cholangitis, chorioamnionitis, conjunctivitis, dacryoadenitis, dermatomyositis, endocarditis, endometritis, enteritis, enterocolitis, epicondylitis, epididymitis, fasciitis, fibrositis, gastritis, gastroenteritis, gingivitis, ileitis, iritis, laryngitis, myelitis, myocarditis, nephritis, omphalitis, oophoritis, orchitis, osteitis, otitis, pancreatitis, parotitis, pericarditis, pharyngitis, pleuritis, phlebitis, pneumonitis, proctitis, prostatitis, rhinitis, salpingitis, sinusitis, stomatitis, synovitis, testitis, tonsillitis, urethritis, urocystitis, uveitis, vaginitis, vasculitis, vulvitis, vulvovaginitis, angitis, chronic bronchitis, osteomyelitis, optic neuritis, temporal arteritis, transverse myelitis, necrotizing fasciitis, and necrotizing enterocolitis. An ocular inflammatory disease includes, but is not limited to, post-surgical inflammation.

Thymidylate synthase is an enzyme that catalyzes the conversion of deoxyuridine monophosphate (dUMP) to thymidine monophosphate (dTMP or thymidylate). This reaction provides the sole de novo pathway for thymidylate production, so that thymidylate synthase is essential for DNA synthesis and repair. The absence of thymidylate synthase blocks proliferation and causes cell death. An exemplary sequence of thymidylate synthase from GenBank is BAB83677.1 (Homo sapiens), with accession number BAB83677.

Dihydrofolate reductase (DHFR) catalyzes the NADPH-dependent reduction of 7,8-dihydrofolate (FH2) to (6s)-5,6,7,8-tetrahydrofolate (FH4). DHFR also reduces folic acid to dihydrofolate. DHFR is an important enzyme in folate metabolism, as well as the synthesis of tetrahydrofolate cofactors, purines, thymidine, glycine, and methionine, and thymidylate, and subsequently, of DNA. For DHFR, an exemplary sequence from GenBank is: AAA58485.1 (Homo sapiens), with accession number AAA58485.

### BRIEF DESCRIPTION OF THE DRAWINGS

FIGS. 1A-1D show compound 19-S inhibits TS catalytic activity, shows cytotoxicity in vitro and does not increase TS levels. (FIG. 1A) Diagram illustrating the TS tritium assay and conversion of tritiated dUMP to dTMP generating tritiated water for quantification of TS activity. (FIG. 1B) TS tritium assay showing TS activity for control reactions and reactions performed in the presence of 5-fluorouridine (FUrd), 5-fluoro-2′-deoxyuridine (FdUrd), and compound 19-S. Drug concentrations were 250 μM using 10 μg/mL bacterially expressed TS protein. Data is expressed as mean SD. (FIG. 1C) Viability assays comparing disclosed TS inhibitor 5-FU to compound 19-S in the indicated cell lines following a 72 hour drug incubation. Data is expressed as mean SD of technical triplicated. (FIG. 1D) Immunoblot analysis showing TS overexpression following 5-FU treatment and stable TS expression levels following compound 19-S treatment. PANC-1 cells were treated for 72 hours with the indicated 5-FU and 19-S concentrations. Quantification shows mean SD of 2 independent experiments.

FIGS. 2A-2H show that compound 19-S inhibits tumor growth and progression. FIG. (2A) Experimental timeline for the subcutaneous Luc-PANC-1 (human bladder carcinoma) cell line derived tumor model treated with compound 19-S or vehicle control. Timeline indicates the subcutaneous injection of Luc-PANC-1 cells to generate tumors and compound 19-S (25 mg/kg) or vehicle control treatment cycles administered by IP injection. For each cycle animals were treated once a day for 5 continuous days then allowed 2 days rest when no treatments were administered. Tumor bearing mice received a total of 4 treatment cycles before endpoint (n=4 per cohort). (FIG. 2B) Effect of compound 19-S and vehicle control administered by IP injection on body weight for Luc-PANC-1 tumor bearing NSG mice. Data is expressed as mean+SD/SEM of 4 animals per cohort. (FIG. 2C) Bioluminescence imaging of Luc-PANC-1 derived tumors and quantification of bioluminescence photon flux over time for animals treated with compound 19-S or vehicle control (n=4 per cohort). Data is expressed as mean+SD/SEM Statistics. (FIG. 2D) Luc-PANC-1 tumor volumes for animals treated with compound 19-S or vehicle control. Data is expressed as mean+SD/Standard Error of the Mean (SEM) Statistics. (FIG. 2E) Final excised Luc-PANC-1 tumor weight for animals treated with compound 19-S or vehicle control. Data is expressed as mean+SD/SEM Statistics. (FIG. 2F) Timeline indicating the IP injection of Luc-CM cells to generate a disseminated tumor model and compound 19-S (25 mg/kg) or vehicle control treatment cycles administered by IP injection. For each cycle animals were treated once a day for 5 continuous days then allowed 2 days rest when no treatments were administered. Animals received 3 treatment cycles before endpoint. (FIG. 2G) Effect of compound 19-S and vehicle control administered by IP injection on body weight for Luc-CM tumor bearing NSG mice. Data are represented as mean body weight of 8 animals per cohort +SEM. (FIG. 2H) Bioluminescence imaging of Luc-CM tumor bearing NSG mice and quantification of bioluminescence photon flux from the abdominal region to quantify tumor burden over time for animals treated with compound 19-S (n=8) or vehicle control. (n=8). Data is expressed as mean+SD/SEM Statistics.

FIGS. 3A-3F show compound 19-S prolongs survival. (FIG. 3A) Experimental timeline indicating the subcutaneous Luc-PANC-1 cell line derived tumor model treated with compound 19-S or vehicle control. Timeline indicates the subcutaneous injection of Luc-PANC-1 cells in NSG mice to generate tumors and compound 19-S (10 mg/kg or 25 mg/kg) or vehicle control treatment cycles administered orally (PO). For each cycle animals were treated once a day for 5 continuous days then allowed 2 days rest when no treatments were administered. Animals received treatment until survival endpoint. (FIG. 3B) Effect of compound 19-S and vehicle control administered PO on body weight for Luc-PANC-1 tumor NSG bearing mice. Data are presented as mean body weight of 5 animals per group ±SEM. (FIG. 3C) Kaplan-Meier survival analysis for Luc-PANC-1 injected NSG mice treated with compound 19-S (10 mg/kg or 25 mg/kg, n=5 per group) or vehicle control (n=5). Log-rank (Mantel-Cox) test was used to calculate p-values. (FIG. 3D and FIG. 3E) Bioluminescence imaging of Luc-PANC-1 derived tumors and quantification of bioluminescence photon flux over time for animals treated with compound 19-S (10 mg/kg or 25 mg/kg) or vehicle control. Data are presented as mean total flux of 5 animals per group ±SEM and indicated p values by 2-tailed Mann-Whitney t-test. (FIG. 3F) Luc-PANC-1 tumor volumes for animals treated with compound 19-S (10 mg/kg or 25 mg/kg) or vehicle control. Data are presented as mean tumor volume of 5 animals per group ±SEM and indicated p values by 2-tailed Mann-Whitney t-test.

FIGS. 4A-4E show select compound 19-S series analogues show dual TS and DHFR inhibition. (FIG. 4A) Tritium based TS activity assay screen of compound 19-S series analogues. Compounds were initially screened at 250 μM to determine analogues showing TS inhibition. (FIG. 4B) Absorbance based DHFR activity assay screen of compound 19-S series analogues, all compounds were initially screened at 1 μM concentration. (FIG. 4C) Comparison showing TS activity utilizing the tritium-based activity assay in the presence of pemetrexed (Pem), methotrexate (MTX), compounds 19-S, 19-S5, and 19-S7 at the indicated concentrations. Data is expressed as mean+SD/SEM Statistics. (FIG. 4D) Comparison showing DHFR activity utilizing the absorbance-based activity assay in the presence of Pem, MTX, compound 19-S, 19-S5, and 19-S7 at the indicated concentrations. (FIG. 4E) MIA PaCa-2 (pancreatic ductal adenocarcinoma) cell line viability assays following 72 hour drug treatment with compound 19-S and 19-S series analogues 19-S5 and 19-S7 and known control anti-folate inhibitors Pem and MXT.

FIGS. 5A-5F show the mechanism of TS inhibition reveals non-classical anti-folate inhibitors. (FIG. 5A) Illustration outlining the drug displacement from TS with increased substrate concentrations to highlight how increased molar ratios of the competing substrate will displace the pre-bound drug and shift the equilibrium to the enzyme:substrate complex resulting in an increased conversion of tritiated dUMP. (FIG. 5B) Tritium assay for TS activity showing the actual scintillation counts recorded from the indicated reaction conditions to demonstrate how the increased substrate concentrations do not impact the total conversion of tritiated dUMP since the standard 1× substrate concentrations already provide saturating substrate conditions. (FIGS. 5C-5F) Tritium assay for TS activity with increasing 5,10-mTHF or dUMP ratios for compound 19-S (FIG. 5C), compound 19-55 (FIG. 5D), compound 19-S7 (FIG. 5E), and control classical anti-folate Pem (FIG. 5F). TS was pre-incubated with each compound using the indicated concentration, for each compound the concentration required for a 50% reduction in dUMP conversion after a 30 minutes reaction was utilized to allow changes in dUMP conversion to be observed. Increasing TS activity with increasing 5,10-mTHF ratios was observed for all compounds including the control anti-folate Pem, while increasing dUMP ratios had no significant effect on TS activity indicating compounds 19-S, 19-55, and 19-S7 act as non-classical anti-folate inhibitors by competing only with the 5,10-mTHF substrate.

FIGS. 6A-6D show the proposed binding mode for compound 19-S. FIGS. 6A-6C and 6B-6D show compound 19-S at canonical binding sites of TS and DHFR, respectively. FIGS. 6A-6B shows the protein backbone as grey and purple ribbons for TS and DHFR, respectively. Cofactors, key residues, and compound 19-S are depicted as sticks and colored by element with carbons, oxygens, nitrogens, and phosphorous in orange, red, blue, and brown respectively (except compound 19-S with carbons in cyan). FIGS. 2C-2D shows a 2D diagram of the protein-ligand interactions between the 19-S compound and TS/DHFR, respectively. Key residues and 19-S are colored by elements with carbons, oxygens, and nitrogens in black, red, and blue, respectively. Polar and aromatic interactions are presented as a black and green dotted dashed line, respectively. Non-polar interactions are presented as a continuous green line surrounding the ligand functional groups.

FIGS. 7A-7H show compounds 19-S and 19-S7 prevent tumor progression without signs of toxicity following oral delivery. (FIG. 7A) Timeline indicating the subcutaneous injection (SQ) of Luc-PANC-1 cells to generate subcutaneous tumors and the treatment cycles for compound 19-S (25 mg/kg), compound 19-S7 (25 mg/kg), or vehicle control administered by oral gavage (PO). For each cycle animals were treated once a day for 5 continuous days then allowed 2 days rest when no treatments were administered. NSG mice received 4 treatment cycles until endpoint. (FIG. 7B) Effect of compound 19-S and 19-S7 treatment with matched vehicle control treatment on body weight for Luc-PANC-1 tumor bearing NSG mice. Data is express as mean+SD/SEM of compound 19-S (n=7, 19S-7 (n=6) and controls (n=7 and n=6, respectively. (FIG. 7C) Bioluminescence imaging of Luc-PANC-1 derived tumors and quantification of bioluminescence photon flux over time for animals treated with compound 19-S or vehicle control administered PO. (FIG. 7D) Luc-PANC-1 tumor volumes for animals treated with compound 19-S or vehicle control administered PO. (FIG. 7E) Final excised Luc-PANC-1 tumor weight for animals treated with compound 19-S or vehicle control administered PO. (FIG. 7F) Bioluminescence imaging of Luc-PANC-1 derived tumors and quantification of bioluminescence photon flux over time for animals treated with compound 19-S7 or vehicle control administered PO. (FIG. 7G) Luc-PANC-1 tumor volumes for animals treated with compound 19-S7 or vehicle control administered PO. (FIG. 7H) Final excised Luc-PANC-1 tumor weight for animals treated with compound 19-S7 or vehicle control administered PO. For both compound 19-S and 19-S7 these studies confirm the treatment is well tolerated and show potent anti-tumor effects using the preferred PO administration route.

FIG. 8 shows the viability assay screen with compound 19-S in cancer cell lines. Viability assay performed in a panel of cancer cell lines including H1048 (small cell lung cancer), CM (pancreatic neuroendocrine tumor), MIA PaCa-2 and PANC-1 (pancreatic ductal adenocarcinoma), Luc-PANC-1 (pancreatic ductal adenocarcinoma cells expressing firefly luciferase). Initial viability assay screens were performed with single drug concentration (10 μM) or DMSO control (0.25%). Each cell line was treated with either compound 19-S (right/red bars) or DMSO control (left/black bars) for 72 hours. The threshold of 10 μM was selected since compounds showing under 50% viability at 10 μM would indicate greater cytotoxicity than disclosed TS inhibitor 5-FU and present a promising lead candidate for further study. Data is presented as mean±SD of 3 technical replicates except for PANC-1 that shows mean±SD of 3 experiments with 3 biological replicates each.

FIGS. 9A-9C show the tritium assay optimization to detect thymidylate synthase catalytic activity. 2 μg is the optimal amount of purified human thymidylate synthase (hTS) to be used as a control in the tritium-based catalytic activity assay. (FIG. 9A) hTS catalytic activity was determined using a range of purified hTS, from 0.00625 μg to 100 μg. hTS catalytic activity was around 20,000 counts/minute between 100 μg and 0.5 μg hTS, but activity dropped one-fold when using from 0.1 μg to 0.00625 μg hTS. (FIG. 9B) hTS catalytic activity was determined, as in FIG. 9A, using a smaller hTS μg range (from 5 μg to 0.00625 μg). hTS catalytic activity dropped one-fold when using 0.1 μg to 0.00625 μg purified hTS, as previously observed. (FIG. 9C) No hTS catalytic activity is observed when no 5,10-methelyne tetrahydrofolate (mTHF) was present in the reaction in the tritium-based catalytic assay. This confirmed the importance of mTHF as methylene and hydride donor in the methylation of deoxyuridine monophosphate (dUMP) in each reaction carried. In addition, we determined the number of counts per minute present in tritiated dUMP added to each reaction. This was determined by adding 20 μL of diluted tritiated dUMP to 5 mL of liquid scintillation cocktail which was then measured on the liquid scintillation counter. Data presented as mean±SD of two independent experiments, n=2 technical replicates per experiment.

FIGS. 10A-10C show compound 19-S is not toxic at a dose of 25 mg/kg for IP treatment in vivo. (FIG. 10A) Timeline indicating IP treatment schedule for compound 19-S in NSG mice to determine maximum tolerated dose (MTD). Compound 19-S was administered IP 5 days a week for 3 weeks and animals rested (no treatment) for one extra week before experimental endpoint. (FIG. 10B) Effect of compound 19-S [25 mg/kg (n=3), 50 mg/kg (n=3), or 100 mg/kg (n=2)] on BW following the treatment schedule shown in FIG. 10A, compared to vehicle control (n=2). Individual mice weight is shown. 100 mg/kg treated animals (n=2) were found dead after 1st and 3rd dose, respectively. 50 mg/kg treated animals (n=3) were found dead after 12th, 13th and 14th dose, respectively. All 25 mg/kg treated animals reached endpoint. (FIG. 10C) Effect of compound 19-S [10 mg/kg (n=3), 25 mg/kg (n=3), 50 mg/kg (n=3) and 100 mg/kg (n=3)] on BW following the treatment schedule shown in FIG. 10A, compared to vehicle control (n=3). Individual mice weight is represented. All 100 mg/kg treated mice were found dead after 1st dose. Out of n=3 animals treated with 50 mg/kg, 2 were found dead after 1st dose and one animal reached endpoint. Mice treated with 10 and 25 mg/kg reached endpoint.

FIG. 11 shows bioluminescence imaging of Luc-PANC-1 derived tumors (IP treatment). Bioluminescence imaging of Luc-PANC-1 derived tumors showing the bioluminescence photon flux over time for animals treated via IP injection with compound 19-S (n=4) or vehicle control (n=4).

FIG. 12 show bioluminescence imaging of Luc-CM derived tumors (IP treatment). Bioluminescence imaging of Luc-CM tumor bearing NSG mice show the bioluminescence photon flux from the abdominal region to quantify tumor burden over time for animals treated via IP injection with compound 19-S (n=8) or vehicle control (n=8).

FIGS. 13A-13D shows compound 19-S inhibits tumor growth and progression at 25 mg/kg in a Luc-CM disseminated tumor model (PO treatment). (FIG. 13A) Timeline indicating IP injection of Luc-CM cells to generate disseminated tumor model and compound 19-S (25 mg/kg) or vehicle control treatment cycles administered PO. For each cycle animals were treated once a day for 5 consecutive days followed by 2 days rest (no treatment administered). After 3 treatment cycles, mice rested for one extra week without treatment before endpoint. (FIG. 13B) Effect of compound 19-S on BW compared to matched vehicle control (administered as indicated in FIG. 13A) to Luc-CM tumor bearing NSG mice. Data are expressed as mean±SEM of n=8 controls and n=7 compound 19-S treated mice. Arrow indicates day when treatment started. (FIG. 13C) Bioluminescence quantification of total photon flux of abdominal region of Luc-CM tumor bearing NSG mice treated PO with compound 19-S (n=7) or vehicle control (n=8). Data are expressed as mean±SEM. A two-way ANOVA was used to assess differences between control and treated mice over time. Arrow indicates day when treatment started. (FIG. 13D) Bioluminescence images of Luc-CM tumor bearing NSG mice to determine tumor burden over time in animals treated with compound 19-S compared to vehicle control mice. Red bar over days after cell injection indicates days of treatment.

FIGS. 14A-14B shows a pharmacophoric interaction used as a restraint for biased docking simulations. FIGS. 14A and 14B show interactions used as pharmacophoric restraints for TS (PDB ID 1HVY) and DHFR (PDB ID 4KAK) respectively. Polar and aromatic restraints are presented as black and green dotted dashed lines respectively.

FIG. 15 shows the pharmacophoric interaction used as a restraint for biased docking simulations.

FIGS. 16A-16B show compound 19-S7 is not toxic at 25 mg/kg for PO treatment. (FIG. 16A) Timeline indicating PO administration of compound 19-S7 in NSG mice to determine MTD. 25 mg/kg, 50 mg/kg or vehicle control treatment was administered PO to 6-8 weeks old NSG mice. Schedule was once a day for five continuous days, then allowed 2 days to rest when no treatment was administered. After 3 treatment cycles, animals rest for 1 week with no treatment before endpoint. (FIG. 16B) Effect of compound 19-S7 [25 mg/kg (n=3) and 50 mg/kg (n=3)] compared to vehicle control (n=3) on NSG BW. Individual mice BW is represented. 50 mg/kg treated mice (n=3) were found dead after 12th (n=1) and 15th (n=2) dose, respectively. Animals treated with 25 mg/kg (n=3) reached endpoint.

FIGS. 17A-17B show bioluminescence imaging of Luc-PANC-1 derived tumors (PO treatment). (FIG. 17A) Bioluminescence imaging of Luc-PANC-1 derived tumors over time for animals treated with compound 19-S (n=7) or vehicle control (n=7). (FIG. 17B) Bioluminescence imaging of Luc-PANC-1 derived tumors over time for animals treated with compound 19-S7 (n=6) or vehicle control (n=6). Red bars and blue bars over the “weeks after cell injection” label indicates the treatment phase.

FIG. 18 shows HSQC NMR spectra for 19-S7.

FIG. 19 shows HSQC NMR spectra for 19-S8.

### DETAILED DESCRIPTION OF CERTAIN EMBODIMENTS OF THE INVENTION

The present disclosure provides inhibitors of a folate-dependent enzyme (e.g., thymidylate synthase, dihydrofolate reductase). In certain embodiments, the compounds inhibit the activity of a folate-dependent enzyme (e.g., thymidylate synthase, dihydrofolate reductase). In certain embodiments, the compounds inhibit the activity of dihydrofolate reductase. In certain embodiments, the compounds inhibit the activity of thymidylate synthase and dihydrofolate reductase. The present disclosure further provides methods of using the compounds described herein, e.g., as biological probes to study the inhibition of the activity of a folate-dependent enzyme (e.g., thymidylate synthase, dihydrofolate reductase), and as therapeutics, e.g., in the treatment and/or prevention of proliferative diseases, or diseases associated with the overexpression and/or aberrant activity of the folate-dependent enzyme (e.g., thymidylate synthase, dihydrofolate reductase). In certain embodiments, the diseases treated and/or prevented include, but are not limited to, proliferative disease, such as, cancers, or other diseases associated with folate-dependent enzymes (e.g., thymidylate synthase, dihydrofolate reductase). The proliferative diseases include, but are not limited to, cancer. Also provided by the present disclosure are pharmaceutical compositions, kits, methods, and uses of a compound of Formula (I) or (II), or a compound of Table A, as described herein.

### Compounds

Certain aspects of the present disclosure relate to the compounds described herein. The compounds described herein may be useful in treating and/or preventing diseases and/or conditions (e.g., proliferative disease, such as, cancers, or other diseases associated with a folate-dependent enzyme (e.g., thymidylate synthase, dihydrofolate reductase)), or diseases associated with the activity of a folate-dependent enzyme (e.g., thymidylate synthase, dihydrofolate reductase) in a subject or biological sample, or inhibiting the activity of a folate-dependent enzyme (e.g., thymidylate synthase, dihydrofolate reductase) in a subject or biological sample. In certain embodiments, a compound described herein is a compound of Formula (I) or (II), or a compound of Table A, or a pharmaceutically acceptable salt, solvate, hydrate, stereoisomer, polymorph, tautomer, isotopically enriched form (e.g., isotopically labeled derivative), or prodrug thereof. In certain embodiments, a compound described herein is a compound of Formula (I) or (II), or a compound of Table A, or a pharmaceutically acceptable salt thereof.

In certain embodiments, a compound described herein is of Formula (I):

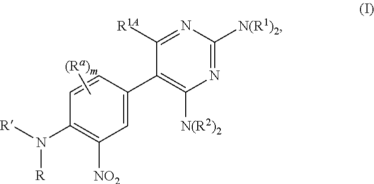

or a pharmaceutically acceptable salt, solvate, hydrate, stereoisomer, polymorph, tautomer, isotopically enriched form, or prodrug thereof, wherein:
R^{1A} is hydrogen or optionally substituted alkyl;
each instance of R¹ is independently hydrogen, optionally substituted acyl, optionally substituted alkyl, or a nitrogen protecting group; or optionally two instances of R¹ are taken together with their intervening atoms to form a substituted or unsubstituted heterocyclic or substituted or unsubstituted heteroaryl ring;
each instance of R² is independently hydrogen, optionally substituted acyl, optionally substituted alkyl, or a nitrogen protecting group; or optionally two instances of R² are taken together with their intervening atoms to form a substituted or unsubstituted heterocyclic or substituted or unsubstituted heteroaryl ring;
each instance of R^{a} is independently halogen, optionally substituted acyl, optionally substituted alkyl, optionally substituted alkenyl, optionally substituted alkynyl, optionally substituted carbocyclyl, optionally substituted heterocyclyl, optionally substituted aryl, optionally substituted heteroaryl, —CN, —NO₂, —OR^{D1}, —N(R^{D1a})₂ or —SR^{D1}, wherein R^{D1} is independently hydrogen, optionally substituted acyl, optionally substituted alkyl, optionally substituted alkenyl, optionally substituted alkynyl, optionally substituted carbocyclyl, optionally substituted heterocyclyl, optionally substituted aryl, optionally substituted heteroaryl, or an oxygen protecting group when attached to an oxygen atom, or a sulfur protecting group when attached to a sulfur atom;
wherein each occurrence of R^{D1a} is independently hydrogen, optionally substituted acyl, optionally substituted alkyl, optionally substituted alkenyl, optionally substituted alkynyl, optionally substituted carbocyclyl, optionally substituted heterocyclyl, optionally substituted aryl, optionally substituted heteroaryl, or a nitrogen protecting group; or optionally two instances of R^{D1a} are taken together with their intervening atoms to form a substituted or unsubstituted heterocyclic or substituted or unsubstituted heteroaryl ring;
R is hydrogen, unsubstituted alkyl, or a nitrogen protecting group;
R′ is

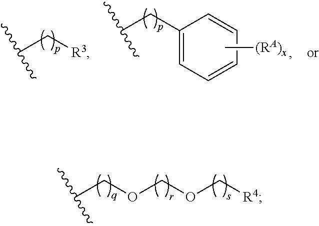

R³ is optionally substituted carbocyclyl, optionally substituted heterocyclyl, or optionally substituted heteroaryl;
each occurrence of R^{A} is independently —SO₂, —SR^{D1}, —NO₂, —N₃, or —CN, or optionally two instances of R^{A} are taken together with their intervening atoms to form a substituted or unsubstituted heterocyclic or substituted or unsubstituted heteroaryl ring;
R⁴ is —OR^{4A} or —N(R^{4B})₂;
R^{4A} is hydrogen, optionally substituted acyl, optionally substituted alkyl, optionally substituted alkenyl, optionally substituted alkynyl, optionally substituted carbocyclyl, optionally substituted heterocyclyl, optionally substituted aryl, optionally substituted heteroaryl, or an oxygen protecting group;
each occurrence of R^{4B} is independently hydrogen, optionally substituted acyl, optionally substituted alkyl, optionally substituted alkenyl, optionally substituted alkynyl, optionally substituted carbocyclyl, optionally substituted heterocyclyl, optionally substituted aryl, optionally substituted heteroaryl, or a nitrogen protecting group; or optionally two instances of R^{4B} are taken together with their intervening atoms to form a substituted or unsubstituted heterocyclic or substituted or unsubstituted heteroaryl ring;
m is 0, 1, 2, or 3;
p is 1, 2, 3, 4, 5, or 6;
q is 1, 2, 3, 4, 5, or 6;
r is 1, 2, 3, 4, 5, or 6;
s is 1, 2, 3, 4, 5, or 6; and
x is 1, 2, 3, 4, or 5.

In certain embodiments, a compound of Formula (I) is of Formula (I-A):

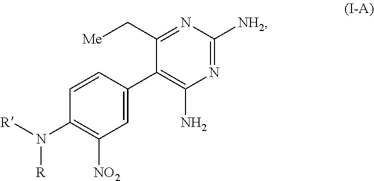

or a pharmaceutically acceptable salt, solvate, hydrate, stereoisomer, polymorph, tautomer, isotopically enriched form, or prodrug thereof.

In certain embodiments, a compound described herein is a compound of Formula (I) or a pharmaceutically acceptable salt, solvate, hydrate, stereoisomer, polymorph, tautomer, isotopically enriched form, or prodrug thereof. In certain embodiments, a compound described herein is a compound of Formula (I-A) or a pharmaceutically acceptable salt, solvate, hydrate, stereoisomer, polymorph, tautomer, isotopically enriched form, or prodrug thereof. In certain embodiments, a compound described herein is a compound of Formula (I), or a pharmaceutically acceptable salt thereof. In certain embodiments, a compound described herein is a compound of Formula (I-A), or a pharmaceutically acceptable salt thereof.

In certain embodiments, a compound described herein is of Formula (II):

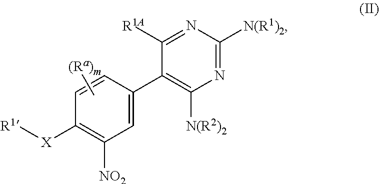

or a pharmaceutically acceptable salt, solvate, hydrate, stereoisomer, polymorph, tautomer, isotopically enriched form, or prodrug thereof, wherein:
X is —O— or —S—;
R^{1A} is hydrogen or optionally substituted alkyl;
each instance of R¹ is independently hydrogen, optionally substituted acyl, optionally substituted alkyl, or a nitrogen protecting group; or optionally two instances of R¹ are taken together with their intervening atoms to form a substituted or unsubstituted heterocyclic or substituted or unsubstituted heteroaryl ring;
each instance of R² is independently hydrogen, optionally substituted acyl, optionally substituted alkyl, or a nitrogen protecting group; or optionally two instances of R² are taken together with their intervening atoms to form a substituted or unsubstituted heterocyclic or substituted or unsubstituted heteroaryl ring;
each instance of R^{a} is independently halogen, optionally substituted acyl, optionally substituted alkyl, optionally substituted alkenyl, optionally substituted alkynyl, optionally substituted carbocyclyl, optionally substituted heterocyclyl, optionally substituted aryl, optionally substituted heteroaryl, —CN, —NO₂, —OR^{D1}, —N(R^{D1a})₂ or —SR^{D1}, wherein R^{D1} is independently hydrogen, optionally substituted acyl, optionally substituted alkyl, optionally substituted alkenyl, optionally substituted alkynyl, optionally substituted carbocyclyl, optionally substituted heterocyclyl, optionally substituted aryl, optionally substituted heteroaryl, or an oxygen protecting group when attached to an oxygen atom, or a sulfur protecting group when attached to a sulfur atom;
wherein each occurrence of R^{D1a} is independently hydrogen, optionally substituted acyl, optionally substituted alkyl, optionally substituted alkenyl, optionally substituted alkynyl, optionally substituted carbocyclyl, optionally substituted heterocyclyl, optionally substituted aryl, optionally substituted heteroaryl, or a nitrogen protecting group; or optionally two instances of R^{D1a} are taken together with their intervening atoms to form a substituted or unsubstituted heterocyclic or substituted or unsubstituted heteroaryl ring;

R¹′ is

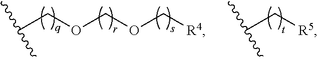

optionally substituted carbocyclyl, optionally substituted heterocyclyl, optionally substituted aryl, or optionally substituted heteroaryl;
R⁴ is —OR^{4A} or —N(R^{4B})₂,
R⁵ is hydrogen, —OR^{D1}, optionally substituted acyl, optionally substituted alkyl, optionally substituted alkenyl, optionally substituted alkynyl, optionally substituted carbocyclyl, optionally substituted heterocyclyl, optionally substituted aryl, or optionally substituted heteroaryl;
R^{4A} is hydrogen, optionally substituted acyl, optionally substituted alkyl, optionally substituted alkenyl, optionally substituted alkynyl, optionally substituted carbocyclyl, optionally substituted heterocyclyl, optionally substituted aryl, optionally substituted heteroaryl, or an oxygen protecting group;
each occurrence of R^{4B} is independently hydrogen, optionally substituted acyl, optionally substituted alkyl, optionally substituted alkenyl, optionally substituted alkynyl, optionally substituted carbocyclyl, optionally substituted heterocyclyl, optionally substituted aryl, optionally substituted heteroaryl, or a nitrogen protecting group; or optionally two instances of R^{4B} are taken together with their intervening atoms to form a substituted or unsubstituted heterocyclic or substituted or unsubstituted heteroaryl ring;
m is 0, 1, 2, or 3;
q is 1, 2, 3, 4, 5, or 6;
r is 1, 2, 3, 4, 5, or 6;
s is 1, 2, 3, 4, 5, or 6; and
t is 1, 2, 3, 4, 5, or 6.

In certain embodiments, a compound described herein is a compound of Formula (II) or a pharmaceutically acceptable salt, solvate, hydrate, stereoisomer, polymorph, tautomer, isotopically enriched form, or prodrug thereof. In certain embodiments, a compound described herein is a compound of Formula (II-A) or a pharmaceutically acceptable salt, solvate, hydrate, stereoisomer, polymorph, tautomer, isotopically enriched form, or prodrug thereof. In certain embodiments, a compound described herein is a compound of Formula (II), or a pharmaceutically acceptable salt thereof. In certain embodiments, a compound described herein is a compound of Formula (II-A), or a pharmaceutically acceptable salt thereof.

In certain embodiments, a compound of Formula (II) is of Formula (II-A):

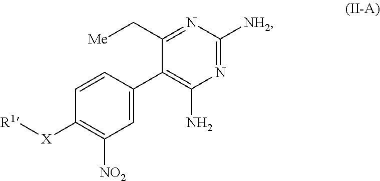

or a pharmaceutically acceptable salt, solvate, hydrate, stereoisomer, polymorph, tautomer, isotopically enriched form, or prodrug thereof.

Compounds of Formula (I) or (II) include substituent R^{1A}. In certain embodiments, R^{1A} is hydrogen. In certain embodiments, R^{1A} is substituted or unsubstituted alkyl (e.g., substituted or unsubstituted C₁₋₆ alkyl). In certain embodiments, R^{1A} is unsubstituted C₁₋₆ alkyl (e.g., methyl, ethyl, n-propyl). In certain embodiments, R^{1A} is ethyl.

Compounds of Formula (I) or (II) include one or more instances of substituent R¹. In certain embodiments, at least one instance of R¹ is hydrogen. In certain embodiments, both instances of R¹ are hydrogen. In certain embodiments, at least one instance of R¹ is optionally substituted acyl (e.g., —C(═O)Me). In certain embodiments, at least one instance of R¹ is optionally substituted alkyl (e.g., substituted or unsubstituted C₁₋₆ alkyl). In certain embodiments, at least one instance of R¹ is substituted or unsubstituted methyl. In certain embodiments, at least one instance of R¹ is substituted methyl (e.g., —CF₃). In certain embodiments, at least one instance of R¹ is unsubstituted methyl. In certain embodiments, at least one instance of R¹ is substituted or unsubstituted ethyl. In certain embodiments, at least one instance of R¹ is substituted or unsubstituted propyl. In certain embodiments, at least one instance of R¹ is substituted or unsubstituted butyl (e.g., t-butyl, n-butyl). In certain embodiments, at least one instance of R¹ is optionally substituted alkenyl (e.g., substituted or unsubstituted C₂₋₆ alkenyl). In certain embodiments, at least one instance of R¹ is optionally substituted alkynyl (e.g., substituted or unsubstituted C₂₋₆ alkynyl). In certain embodiments, at least one instance of R¹ is optionally substituted carbocyclyl (e.g., substituted or unsubstituted, 3- to 10-membered, monocyclic carbocyclyl comprising zero, one, or two double bonds in the carbocyclic ring system). In certain embodiments, at least one instance of R¹ is optionally substituted heterocyclyl (e.g., substituted or unsubstituted, 5- to 10-membered monocyclic or bicyclic heterocyclic ring, wherein one or two atoms in the heterocyclic ring are independently nitrogen, oxygen, or sulfur). In certain embodiments, at least one instance of R¹ is optionally substituted aryl (e.g., substituted or unsubstituted, 6- to 10-membered aryl). In certain embodiments, at least one instance R¹ is benzyl. In certain embodiments, at least one instance of R¹ is substituted or unsubstituted phenyl. In certain embodiments, at least one instance of R¹ is optionally substituted heteroaryl (e.g., substituted or unsubstituted, 5- to 6-membered, monocyclic heteroaryl, wherein one, two, three, or four atoms in the heteroaryl ring system are independently nitrogen, oxygen, or sulfur; or substituted or unsubstituted, 9- to 10-membered, bicyclic heteroaryl, wherein one, two, three, or four atoms in the heteroaryl ring system are independently nitrogen, oxygen, or sulfur). In certain embodiments, two instances of R¹ are taken together with their intervening atoms to form a optionally substituted heterocyclic or optionally substituted heteroaryl ring (e.g., substituted or unsubstituted, 5- to 10-membered monocyclic or bicyclic heterocyclic ring, wherein one or two atoms in the heterocyclic ring are independently nitrogen, oxygen, or sulfur; or substituted or unsubstituted, 5- to 6-membered, monocyclic heteroaryl, wherein one, two, three, or four atoms in the heteroaryl ring system are independently nitrogen, oxygen, or sulfur; or substituted or unsubstituted, 9- to 10-membered, bicyclic heteroaryl, wherein one, two, three, or four atoms in the heteroaryl ring system are independently nitrogen, oxygen, or sulfur).

Compounds of Formula (I) or (II) include one or more instances of substituent R². In certain embodiments, at least one instance of R² is hydrogen. In certain embodiments, both instances of R² are hydrogen. In certain embodiments, at least one instance of R² is optionally substituted acyl (e.g., —C(═O)Me). In certain embodiments, at least one instance of R² is optionally substituted alkyl (e.g., substituted or unsubstituted C₁₋₆ alkyl). In certain embodiments, at least one instance of R² is substituted or unsubstituted methyl. In certain embodiments, at least one instance of R² is substituted methyl (e.g., —CF₃). In certain embodiments, at least one instance of R² is unsubstituted methyl. In certain embodiments, at least one instance of R² is substituted or unsubstituted ethyl. In certain embodiments, at least one instance of R² is substituted or unsubstituted propyl. In certain embodiments, at least one instance of R² is substituted or unsubstituted butyl (e.g., t-butyl, n-butyl). In certain embodiments, at least one instance of R² is optionally substituted alkenyl (e.g., substituted or unsubstituted C₂₋₆ alkenyl). In certain embodiments, at least one instance of R² is optionally substituted alkynyl (e.g., substituted or unsubstituted C₂₋₆ alkynyl). In certain embodiments, at least one instance of R² is optionally substituted carbocyclyl (e.g., substituted or unsubstituted, 3- to 10-membered, monocyclic carbocyclyl comprising zero, one, or two double bonds in the carbocyclic ring system). In certain embodiments, at least one instance of R² is optionally substituted heterocyclyl (e.g., substituted or unsubstituted, 5- to 10-membered monocyclic or bicyclic heterocyclic ring, wherein one or two atoms in the heterocyclic ring are independently nitrogen, oxygen, or sulfur). In certain embodiments, at least one instance of R² is optionally substituted aryl (e.g., substituted or unsubstituted, 6- to 10-membered aryl). In certain embodiments, at least one instance R² is benzyl. In certain embodiments, at least one instance of R² is substituted or unsubstituted phenyl. In certain embodiments, at least one instance of R² is optionally substituted heteroaryl (e.g., substituted or unsubstituted, 5- to 6-membered, monocyclic heteroaryl, wherein one, two, three, or four atoms in the heteroaryl ring system are independently nitrogen, oxygen, or sulfur; or substituted or unsubstituted, 9- to 10-membered, bicyclic heteroaryl, wherein one, two, three, or four atoms in the heteroaryl ring system are independently nitrogen, oxygen, or sulfur). In certain embodiments, two instances of R² are taken together with their intervening atoms to form a optionally substituted heterocyclic or optionally substituted heteroaryl ring (e.g., substituted or unsubstituted, 5- to 10-membered monocyclic or bicyclic heterocyclic ring, wherein one or two atoms in the heterocyclic ring are independently nitrogen, oxygen, or sulfur; or substituted or unsubstituted, 5- to 6-membered, monocyclic heteroaryl, wherein one, two, three, or four atoms in the heteroaryl ring system are independently nitrogen, oxygen, or sulfur; or substituted or unsubstituted, 9- to 10-membered, bicyclic heteroaryl, wherein one, two, three, or four atoms in the heteroaryl ring system are independently nitrogen, oxygen, or sulfur)

In certain embodiments, in compounds of Formula (I) or (II), both instances of R¹ are hydrogen, and both instances of R² are hydrogen. In certain embodiments, in compounds of Formula (I) or (II), R^{1A} is unsubstituted C₁₋₆ alkyl (e.g., methyl, ethyl, n-propyl), both instances of R¹ are hydrogen, and both instances of R² are hydrogen. In certain embodiments, in compounds of Formula (I) or (II), R^{1A} is ethyl, both instances of R¹ are hydrogen, and both instances of R² are hydrogen.

Compounds of Formula (I) or (II) include zero or more instances of R^{a}. In certain embodiments, m is 0. In certain embodiments, m is 1. In certain embodiments, m is 2. In certain embodiments, m is 3. In certain embodiments, m is 0 or 1. In certain embodiments, at least one instance of R^{a} is halogen (e.g., F, Cl, Br, or I). In certain embodiments, at least one instance of R^{a} is optionally substituted acyl (e.g., —C(═O)Me). In certain embodiments, at least one instance of R^{a} is optionally substituted alkyl (e.g., substituted or unsubstituted C₁_6 alkyl). In certain embodiments, at least one instance of R^{a} is substituted or unsubstituted methyl. In certain embodiments, at least one instance of R^{a} is substituted or unsubstituted ethyl. In certain embodiments, at least one instance of R^{a} is substituted or unsubstituted propyl. In certain embodiments, at least one instance of R^{a} is optionally substituted alkenyl (e.g., substituted or unsubstituted C₂₋₆ alkenyl). In certain embodiments, at least one instance of R^{a} is optionally substituted alkynyl (e.g., substituted or unsubstituted C₂₋₆ alkynyl). In certain embodiments, at least one instance of R^{a} is optionally substituted carbocyclyl (e.g., substituted or unsubstituted, 3- to 7-membered, monocyclic carbocyclyl comprising zero, one, or two double bonds in the carbocyclic ring system). In certain embodiments, at least one instance of R^{a} is optionally substituted heterocyclyl (e.g., substituted or unsubstituted, 5- to 10-membered monocyclic or bicyclic heterocyclic ring, wherein one or two atoms in the heterocyclic ring are independently nitrogen, oxygen, or sulfur). In certain embodiments, at least one instance of R^{a} is optionally substituted aryl (e.g., substituted or unsubstituted, 6- to 10-membered aryl). In certain embodiments, at least one instance of R^{a} is benzyl. In certain embodiments, at least one instance of R^{a} is substituted or unsubstituted phenyl. In certain embodiments, at least one instance of R^{a} is optionally substituted heteroaryl (e.g., substituted or unsubstituted, 5- to 6-membered, monocyclic heteroaryl, wherein one, two, three, or four atoms in the heteroaryl ring system are independently nitrogen, oxygen, or sulfur; or substituted or unsubstituted, 9- to 10-membered, bicyclic heteroaryl, wherein one, two, three, or four atoms in the heteroaryl ring system are independently nitrogen, oxygen, or sulfur). In certain embodiments, at least one instance of R^{a} is —CN. In certain embodiments, at least one instance of R^{a} is —NO₂. In certain embodiments, at least one instance of R^{a} is —OR^{D1} (e.g., —OH or —OMe). In certain embodiments, at least one instance of R^{a} is —N(R^{D1a})₂ (e.g., —NMe₂). In certain embodiments, at least one instance of R^{a} is —SR^{D1} (e.g., —SMe).

In certain embodiments, at least one instance of R^{a} is —OR^{D1}, —N(R^{D1a})₂ or —SR^{D1} and R^{D1} and R^{D1a} are as defined herein. In certain embodiments, R^{D1} is hydrogen. In certain embodiments, R^{D1} is optionally substituted acyl (e.g., —C(═O)Me). In certain embodiments, R^{D1} is optionally substituted alkyl (e.g., substituted or unsubstituted C₁₋₆ alkyl). In certain embodiments, R^{D1} is substituted or unsubstituted methyl. In certain embodiments, R^{D1} is substituted or unsubstituted ethyl. In certain embodiments, R^{D1} is substituted or unsubstituted propyl. In certain embodiments, R^{D1} is optionally substituted alkenyl (e.g., substituted or unsubstituted C₂₋₆ alkenyl). In certain embodiments, R^{D1} is optionally substituted alkynyl (e.g., substituted or unsubstituted C₂₋₆ alkynyl). In certain embodiments, R^{D1} is optionally substituted carbocyclyl (e.g., substituted or unsubstituted, 3- to 7-membered, monocyclic carbocyclyl comprising zero, one, or two double bonds in the carbocyclic ring system). In certain embodiments, R^{D1} is optionally substituted heterocyclyl (e.g., substituted or unsubstituted, 5- to 10-membered monocyclic or bicyclic heterocyclic ring, wherein one or two atoms in the heterocyclic ring are independently nitrogen, oxygen, or sulfur). In certain embodiments, R^{D1} is optionally substituted aryl (e.g., substituted or unsubstituted, 6- to 10-membered aryl). In certain embodiments, R^{D1} is benzyl. In certain embodiments, R^{D1} is substituted or unsubstituted phenyl. In certain embodiments, R^{D1} is optionally substituted heteroaryl (e.g., substituted or unsubstituted, 5- to 6-membered, monocyclic heteroaryl, wherein one, two, three, or four atoms in the heteroaryl ring system are independently nitrogen, oxygen, or sulfur; or substituted or unsubstituted, 9- to 10-membered, bicyclic heteroaryl, wherein one, two, three, or four atoms in the heteroaryl ring system are independently nitrogen, oxygen, or sulfur). In certain embodiments, R^{D1} is an oxygen protecting group when attached to an oxygen atom. In certain embodiments, R^{D1} is a sulfur protecting group when attached to a sulfur atom.

In certain embodiments, at least one instance of R^{D1a} is hydrogen. In certain embodiments, at least one instance of R^{D1a} is optionally substituted acyl (e.g., —C(═O)Me). In certain embodiments, at least one R^{D1a} is optionally substituted alkyl (e.g., substituted or unsubstituted C₁₋₆ alkyl). In certain embodiments, at least one instance of R^{D1a} is substituted or unsubstituted methyl. In certain embodiments, at least one instance of R^{D1a} is substituted or unsubstituted ethyl. In certain embodiments, at least one instance of R^{D1a} is substituted or unsubstituted propyl. In certain embodiments, at least one instance of R^{D1a} is optionally substituted alkenyl (e.g., substituted or unsubstituted C₂₋₆ alkenyl). In certain embodiments, at least one instance of R^{D1a} is optionally substituted alkynyl (e.g., substituted or unsubstituted C₂₋₆ alkynyl). In certain embodiments, at least one instance of R^{D1a} is optionally substituted carbocyclyl (e.g., substituted or unsubstituted, 3- to 7-membered, monocyclic carbocyclyl comprising zero, one, or two double bonds in the carbocyclic ring system). In certain embodiments, at least one instance of R^{D1a} is optionally substituted heterocyclyl (e.g., substituted or unsubstituted, 5- to 10-membered monocyclic or bicyclic heterocyclic ring, wherein one or two atoms in the heterocyclic ring are independently nitrogen, oxygen, or sulfur). In certain embodiments, at least one instance of R^{D1a} is optionally substituted aryl (e.g., substituted or unsubstituted, 6- to 10-membered aryl). In certain embodiments, at least one instance of R^{D1a} is benzyl. In certain embodiments, at least one instance of R^{D1a} is substituted or unsubstituted phenyl. In certain embodiments, at least one instance of R^{D1a} is optionally substituted heteroaryl (e.g., substituted or unsubstituted, 5- to 6-membered, monocyclic heteroaryl, wherein one, two, three, or four atoms in the heteroaryl ring system are independently nitrogen, oxygen, or sulfur; or substituted or unsubstituted, 9- to 10 -membered, bicyclic heteroaryl, wherein one, two, three, or four atoms in the heteroaryl ring system are independently nitrogen, oxygen, or sulfur). In certain embodiments, at least one instance of R^{D1}a is a nitrogen protecting group (e.g., benzyl (Bn), t-butyl carbonate (BOC or Boc), benzyl carbamate (Cbz), 9-fluorenylmethyl carbonate (Fmoc), trifluoroacetyl, triphenylmethyl, acetyl, or p-toluenesulfonamide (Ts)). In certain embodiments, two instances of R^{D1}a are taken together with their intervening atoms to form a optionally substituted heterocyclic ring (e.g., substituted or unsubstituted, 5- to 10-membered monocyclic or bicyclic heterocyclic ring, wherein one or two atoms in the heterocyclic ring are independently nitrogen, oxygen, or sulfur) or optionally substituted heteroaryl ring (e.g., substituted or unsubstituted, 5- to 6-membered, monocyclic heteroaryl, wherein one, two, three, or four atoms in the heteroaryl ring system are independently nitrogen, oxygen, or sulfur; or substituted or unsubstituted, 9- to 10-membered, bicyclic heteroaryl, wherein one, two, three, or four atoms in the heteroaryl ring system are independently nitrogen, oxygen, or sulfur).

Compounds of Formula (I) include substituent R. In certain embodiments, R is hydrogen. In certain embodiments, R is unsubstituted alkyl (e.g., unsubstituted C₁₋₆ alkyl). In certain embodiments, R is unsubstituted C₁₋₆ alkyl. In certain embodiments, R is methyl. In certain embodiments, R is ethyl. In certain embodiments, R is unsubstituted propyl. In certain embodiments, R is n-propyl. In certain embodiments, R is methyl or isopropyl. In certain embodiments, R is a nitrogen protecting group (e.g., benzyl (Bn), t-butyl carbonate (BOC or Boc), benzyl carbamate (Cbz), 9-fluorenylmethyl carbonate (Fmoc), trifluoroacetyl, triphenylmethyl, acetyl, or p-toluenesulfonamide (Ts)).

Compounds of Formula (I) include substituent R′. In certain embodiments, R′ is of the formula:

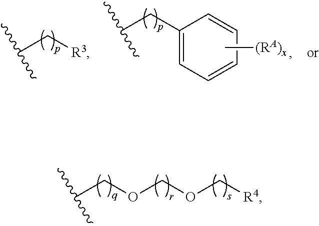

wherein p, q, r, s, R^{A}, R³, and R⁴ are as defined herein. In certain embodiments, R′ is of the formula:

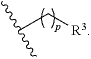

In certain embodiments, p is 1. In certain embodiments, p is 2. In certain embodiments, p is 3. In certain embodiments, p is 4. In certain embodiments, p is 5. In certain embodiments, p is 6. In certain embodiments, R³ is optionally substituted carbocyclyl, optionally substituted heterocyclyl, or optionally substituted heteroaryl. In certain embodiments, R³ is optionally substituted carbocyclyl (e.g., substituted or unsubstituted, 3- to 7-membered, monocyclic carbocyclyl comprising zero, one, or two double bonds in the carbocyclic ring system). In certain embodiments, R³ is optionally substituted, 3- to 7-membered, monocyclic carbocyclyl comprising zero, one, or two double bonds in the carbocyclic ring system. In certain embodiments, R³ is optionally substituted C₃₋₆ monocyclic carbocyclyl (e.g., cyclopentyl, cyclohexyl, cycloheptyl). In certain embodiments, R³ is optionally substituted C₅₋₆ monocyclic carbocyclyl (e.g., cyclopentyl, cyclohexyl). In certain embodiments, R³ is substituted or unsubstituted cyclohexyl. In certain embodiments, R³ is unsubstituted cyclohexyl. In certain embodiments, R³ is an optionally substituted heterocyclyl (e.g., substituted or unsubstituted, 5- to 10-membered monocyclic or bicyclic heterocyclic ring, wherein one or two atoms in the heterocyclic ring are independently nitrogen, oxygen, or sulfur), for example, optionally substituted tetrahydrofuranyl or optionally substituted pyrrolidinyl. In certain embodiments, R³ is optionally substituted heteroaryl (e.g., substituted or unsubstituted, 5- to 6-membered, monocyclic heteroaryl, wherein one, two, three, or four atoms in the heteroaryl ring system are independently nitrogen, oxygen, or sulfur; or substituted or unsubstituted, 9- to 10-membered, bicyclic heteroaryl, wherein one, two, three, or four atoms in the heteroaryl ring system are independently nitrogen, oxygen, or sulfur). In certain embodiments, R³ is optionally substituted, 5- to 6-membered, monocyclic heteroaryl, wherein one, two, three, or four atoms in the heteroaryl ring system are independently nitrogen, oxygen, or sulfur. In certain embodiments, R³ is optionally substituted 5-membered heteroaryl (e.g., optionally substituted furanyl or optionally substituted triazolyl). In certain embodiments, R³ is optionally substituted furanyl. In certain embodiments, R³ is triazolyl (e.g., 1, 2, 3,-triazolyl) optionally substituted with —OR^{3A}, wherein R^{3A} is hydrogen, optionally substituted acyl, or optionally substituted alkyl. In certain embodiments, R³ is optionally substituted 1, 2, 3,-triazolyl. In certain embodiments, R³ is 1, 2, 3,-triazolyl optionally substituted with optionally substituted alkyl, optionally substituted alkenyl, hydroxyl, or optionally substituted alkoxyl. In certain embodiments, R³ is

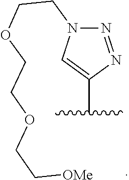 [SMILES: *c1cn(CCOCCOCCOC)nn1]

In certain embodiments, R³ is optionally substituted 1, 2, 4,-triazolyl. In certain embodiments, R³ is optionally substituted 6-membered heteroaryl (e.g., optionally substituted pyridinyl, optionally substituted pyrimidinyl, optionally substituted pyridazinyl, optionally substituted pyrazinyl). In certain embodiments, R³ is optionally substituted pyridinyl or optionally substituted pyrimidinyl. In certain embodiments, R³ is not aryl (e.g., phenyl).

In certain embodiments, R′ is of the formula:

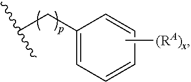

wherein p, x, and R^{A} are as defined herein. In certain embodiments, p is 1. In certain embodiments, p is 2. In certain embodiments, p is 3. In certain embodiments, p is 4. In certain embodiments, p is 5. In certain embodiments, p is 6. In certain embodiments, x is 1. In certain embodiments, x is 2. In certain embodiments, x is 3. In certain embodiments, x is 4. In certain embodiments, x is 5. In certain embodiments, at least one instance of R^{A} is —SO₂. In certain embodiments, at least one instance of R^{A} is —SR^{D1} (e.g., —SMe). In certain embodiments, at least one instance of R^{A} is —NO₂. In certain embodiments, at least one instance of R^{A} is —N₃. In certain embodiments, at least one instance of R^{A} is —CN. In certain embodiments, at least one instance of R^{A} is —SO₂, —SR^{D1}, —NO₂, —N₃, or —CN. In certain embodiments, x is 2, and two instances of R^{A} are taken together with their intervening atoms to form a optionally substituted heterocyclic or optionally substituted heteroaryl ring (e.g., substituted or unsubstituted, 5- to 6-membered, monocyclic heteroaryl, wherein one, two, three, or four atoms in the heteroaryl ring system are independently nitrogen, oxygen, or sulfur; or substituted or unsubstituted, 9- to 10-membered, bicyclic heteroaryl, wherein one, two, three, or four atoms in the heteroaryl ring system are independently nitrogen, oxygen, or sulfur). In certain embodiments, two instances of R^{A} are taken together with their intervening atoms to form a optionally substituted heterocyclic or optionally substituted heteroaryl ring (e.g., substituted or unsubstituted, 5- to 6-membered, monocyclic heteroaryl, wherein one, two, three, or four atoms in the heteroaryl ring system are independently nitrogen, oxygen, or sulfur; or substituted or unsubstituted, 9- to 10-membered, bicyclic heteroaryl, wherein one, two, three, or four atoms in the heteroaryl ring system are independently nitrogen, oxygen, or sulfur). In certain embodiments, two instances of R^{A} are taken together with their intervening atoms to form a optionally substituted 5- to 6-membered, monocyclic heterocyclic or optionally substituted heteroaryl ring, wherein at least 1-2 atom(s) in the heteroaryl ring system are independently oxygen. In certain embodiments, R′ is of the formula:

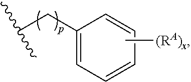

x is 2, and two instances of R^{A} are taken together with their intervening atoms to form a optionally substituted heterocyclic or optionally substituted heteroaryl ring (e.g., substituted or unsubstituted, 5- to 6-membered, monocyclic heteroaryl, wherein one, two, three, or four atoms in the heteroaryl ring system are independently nitrogen, oxygen, or sulfur; or substituted or unsubstituted, 9- to 10-membered, bicyclic heteroaryl, wherein one, two, three, or four atoms in the heteroaryl ring system are independently nitrogen, oxygen, or sulfur). In certain embodiments, R′ is of the formula:

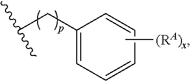

x is 2, and two instances of R^{A} are taken together with their intervening atoms to form a optionally substituted heterocyclic or optionally substituted heteroaryl ring (e.g., substituted or unsubstituted, 5- to 6-membered, monocyclic heteroaryl, wherein one, two, three, or four atoms in the heteroaryl ring system are independently nitrogen, oxygen, or sulfur). In certain embodiments, R′ is of the formula:

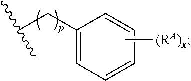

p is 1, 2, or 3; x is 2; and two instances of R^{A} are taken together with their intervening atoms to form a optionally substituted heterocyclic or optionally substituted heteroaryl ring (e.g., substituted or unsubstituted, 5- to 6-membered, monocyclic heteroaryl, wherein one, two, three, or four atoms in the heteroaryl ring system are independently nitrogen, oxygen, or sulfur). In certain embodiments, R′ is of the formula:

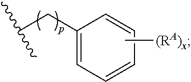

p is 1, 2, 3, 4, 5, or 6; x is 2; and two instances of R^{A} are taken together with their intervening atoms to form a optionally substituted heterocyclic or optionally substituted heteroaryl ring (e.g., substituted or unsubstituted, 5- to 6-membered, monocyclic heteroaryl, wherein one, two, three, or four atoms in the heteroaryl ring system are independently nitrogen, oxygen, or sulfur). In certain embodiments, R′ is of the formula:

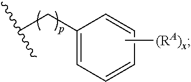

p is 1; x is 2; and two instances of R^{A} are taken together with their intervening atoms to form a optionally substituted heterocyclic or optionally substituted heteroaryl ring (e.g., substituted or unsubstituted, 5- to 6-membered, monocyclic heteroaryl, wherein one, two, three, or four atoms in the heteroaryl ring system are independently nitrogen, oxygen, or sulfur). In certain embodiments, R′ is of the formula:

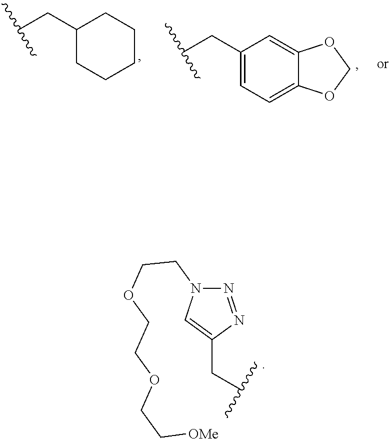 [SMILES: *CC1CCCCC1 ; *Cc1ccc2c(c1)OCO2 ; *Cc1cn(CCOCCOCCOC)nn1]

In certain embodiments, R′ is of the formula:

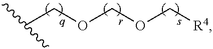

wherein q, r, s, and R⁴ are as defined herein. In certain embodiments, q is 1. In certain embodiments, q is 2. In certain embodiments, q is 3. In certain embodiments, q is 4. In certain embodiments, q is 5. In certain embodiments, q is 6. In certain embodiments, r is 1. In certain embodiments, r is 2. In certain embodiments, r is 3. In certain embodiments, r is 4. In certain embodiments, r is 5. In certain embodiments, r is 6. In certain embodiments, s is 1. In certain embodiments, s is 2. In certain embodiments, s is 3. In certain embodiments, s is 4. In certain embodiments, s is 5. In certain embodiments, s is 6. In certain embodiments, R⁴ is hydrogen. In certain embodiments, R⁴ is optionally substituted acyl (e.g., —C(═O)Me). In certain embodiments, R⁴ is optionally substituted alkyl (e.g., substituted or unsubstituted C₁₋₆ alkyl). In certain embodiments, R⁴ is substituted or unsubstituted methyl. In certain embodiments, R⁴ is substituted methyl (e.g., —CF₃). In certain embodiments, R⁴ is unsubstituted methyl. In certain embodiments, R⁴ is substituted or unsubstituted ethyl. In certain embodiments, R⁴ is substituted or unsubstituted propyl. In certain embodiments, R⁴ is substituted or unsubstituted butyl (e.g., t-butyl, n-butyl). In certain embodiments, R⁴ is optionally substituted alkenyl (e.g., substituted or unsubstituted C₂₋₆ alkenyl). In certain embodiments, R⁴ is optionally substituted alkynyl (e.g., substituted or unsubstituted C₂₋₆ alkynyl). In certain embodiments, R⁴ is optionally substituted carbocyclyl (e.g., substituted or unsubstituted, 3- to 10-membered, monocyclic carbocyclyl comprising zero, one, or two double bonds in the carbocyclic ring system). In certain embodiments, R⁴ is optionally substituted heterocyclyl (e.g., substituted or unsubstituted, 5- to 10-membered monocyclic or bicyclic heterocyclic ring, wherein one or two atoms in the heterocyclic ring are independently nitrogen, oxygen, or sulfur). In certain embodiments, R⁴ is optionally substituted aryl (e.g., substituted or unsubstituted, 6- to 10-membered aryl). In certain embodiments, R⁴ is benzyl. In certain embodiments, R⁴ is substituted or unsubstituted phenyl. In certain embodiments, R⁴ is optionally substituted heteroaryl (e.g., substituted or unsubstituted, 5- to 6-membered, monocyclic heteroaryl, wherein one, two, three, or four atoms in the heteroaryl ring system are independently nitrogen, oxygen, or sulfur; or substituted or unsubstituted, 9- to 10-membered, bicyclic heteroaryl, wherein one, two, three, or four atoms in the heteroaryl ring system are independently nitrogen, oxygen, or sulfur). In certain embodiments, R′ is of the formula:

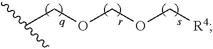

q is 1, 2, or 3; r is 1, 2, or 3; s is 1, 2, or 3; R⁴ is —OR^{4A} or —N(R^{4B})₂; R^{4A} is hydrogen, optionally substituted acyl, optionally substituted alkyl, optionally substituted alkenyl, optionally substituted alkynyl, optionally substituted carbocyclyl, optionally substituted heterocyclyl, optionally substituted aryl, or optionally substituted heteroaryl; and each occurrence of R^{4B} is independently hydrogen, optionally substituted acyl, optionally substituted alkyl, optionally substituted alkenyl, optionally substituted alkynyl, optionally substituted carbocyclyl, optionally substituted heterocyclyl, optionally substituted aryl, or optionally substituted heteroaryl; or optionally two instances of R^{4B} are taken together with their intervening atoms to form a optionally substituted heterocyclic or optionally substituted heteroaryl ring. In certain embodiments, R′ is of the formula:

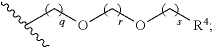

q is 1 or 2; r is 1 or 2; s is 1 or 2; R⁴ is —OR^{4A} or —N(R^{4B})₂. In certain embodiments, R′ is of the formula:

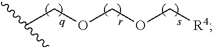

q is 2; r is 2; s is 2; R⁴ is —OR^{4A} or N(R^{4B})₂. In certain embodiments, R′ is of the formula:

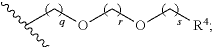

q is 2; r is 2; and s is 2. In certain embodiments, R′ is of the formula:

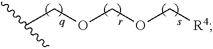

and q is 2.

In certain embodiments, R′ is of the formula:

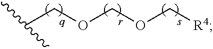

and r is 2. In certain embodiments, R′ is of the formula:

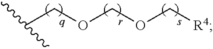

and s is 2. In certain embodiments, R⁴ is —OR^{4A}, —N(R^{4B})₂, or —SR^{4A}, and R^{4A} and R^{4B} are as defined herein. In certain embodiments, R⁴ is —OR^{4A} or —N(R^{4B})₂, and R^{4A} and R^{4B} are as defined herein. In certain embodiments, R⁴ is —OR^{4A} (e.g., —OH or —OMe). In certain embodiments, R⁴ is C₁_s alkoxyl. In certain embodiments, R⁴ is —OR^{4A}; and R^{4A} is optionally substituted acyl or optionally substituted C₁₋₆ alkyl. In certain embodiments, R⁴ is —OMe. In certain embodiments, R⁴ is —N(R^{4B})₂ (e.g., —NMe₂). In certain embodiments, R⁴ is —N(R^{4B})₂; and each occurrence of R^{4B} is independently hydrogen, optionally substituted acyl, optionally substituted alkyl, or optionally substituted alkenyl. In certain embodiments, R⁴ is —NH₂. In certain embodiments, R⁴ is —SR^{4A} (e.g., —SMe). In certain embodiments, R^{4A} is hydrogen. In certain embodiments, R^{4A} is optionally substituted acyl (e.g., —C(═O)Me). In certain embodiments, R^{4A} is optionally substituted alkyl (e.g., substituted or unsubstituted C₁_s alkyl). In certain embodiments, R^{4A} is substituted or unsubstituted methyl. In certain embodiments, R^{4A} is substituted or unsubstituted ethyl. In certain embodiments, R^{4A} is substituted or unsubstituted propyl. In certain embodiments, R^{4A} is optionally substituted alkenyl (e.g., substituted or unsubstituted C₂₋₆ alkenyl). In certain embodiments, R^{4A} is optionally substituted alkynyl (e.g., substituted or unsubstituted C₂₋₆ alkynyl). In certain embodiments, R^{4A} is optionally substituted carbocyclyl (e.g., substituted or unsubstituted, 3- to 7-membered, monocyclic carbocyclyl comprising zero, one, or two double bonds in the carbocyclic ring system). In certain embodiments, R^{4A} is optionally substituted heterocyclyl (e.g., substituted or unsubstituted, 5- to 10-membered monocyclic or bicyclic heterocyclic ring, wherein one or two atoms in the heterocyclic ring are independently nitrogen, oxygen, or sulfur). In certain embodiments, R^{4A} is optionally substituted aryl (e.g., substituted or unsubstituted, 6- to 10-membered aryl). In certain embodiments, R^{4A} is benzyl. In certain embodiments, R^{4A} is substituted or unsubstituted phenyl. In certain embodiments, R^{4A} is optionally substituted heteroaryl (e.g., substituted or unsubstituted, 5- to 6-membered, monocyclic heteroaryl, wherein one, two, three, or four atoms in the heteroaryl ring system are independently nitrogen, oxygen, or sulfur; or substituted or unsubstituted, 9- to 10-membered, bicyclic heteroaryl, wherein one, two, three, or four atoms in the heteroaryl ring system are independently nitrogen, oxygen, or sulfur). In certain embodiments, R^{4A} is an oxygen protecting group when attached to an oxygen atom. In certain embodiments, R^{4A} is a sulfur protecting group when attached to a sulfur atom.

In certain embodiments, at least one instance of R^{4B} is hydrogen. In certain embodiments, at least one instance of R^{4B} is optionally substituted acyl (e.g., —C(═O)Me). In certain embodiments, at least one R^{4B} is optionally substituted alkyl (e.g., substituted or unsubstituted C₁₋₆ alkyl). In certain embodiments, at least one instance of R^{4B} is substituted or unsubstituted methyl. In certain embodiments, at least one instance of R^{4B} is substituted or unsubstituted ethyl. In certain embodiments, at least one instance of R^{4B} is substituted or unsubstituted propyl. In certain embodiments, at least one instance of R^{4B} is optionally substituted alkenyl (e.g., substituted or unsubstituted C₂₋₆ alkenyl). In certain embodiments, at least one instance of R^{4B} is optionally substituted alkynyl (e.g., substituted or unsubstituted C₂₋₆ alkynyl). In certain embodiments, at least one instance of R^{4B} is optionally substituted carbocyclyl (e.g., substituted or unsubstituted, 3- to 7-membered, monocyclic carbocyclyl comprising zero, one, or two double bonds in the carbocyclic ring system). In certain embodiments, at least one instance of R^{4B} is optionally substituted heterocyclyl (e.g., substituted or unsubstituted, 5- to 10-membered monocyclic or bicyclic heterocyclic ring, wherein one or two atoms in the heterocyclic ring are independently nitrogen, oxygen, or sulfur). In certain embodiments, at least one instance of R^{4B} is optionally substituted aryl (e.g., substituted or unsubstituted, 6- to 10-membered aryl). In certain embodiments, at least one instance of R^{4B} is benzyl. In certain embodiments, at least one instance of R^{4B} is substituted or unsubstituted phenyl. In certain embodiments, at least one instance of R^{4B} is optionally substituted heteroaryl (e.g., substituted or unsubstituted, 5- to 6-membered, monocyclic heteroaryl, wherein one, two, three, or four atoms in the heteroaryl ring system are independently nitrogen, oxygen, or sulfur; or substituted or unsubstituted, 9- to 10-membered, bicyclic heteroaryl, wherein one, two, three, or four atoms in the heteroaryl ring system are independently nitrogen, oxygen, or sulfur). In certain embodiments, at least one instance of R^{4B} is a nitrogen protecting group (e.g., benzyl (Bn), t-butyl carbonate (BOC or Boc), benzyl carbamate (Cbz), 9-fluorenylmethyl carbonate (Fmoc), trifluoroacetyl, triphenylmethyl, acetyl, or p-toluenesulfonamide (Ts)). In certain embodiments, two instances of R^{4B} are taken together with their intervening atoms to form a optionally substituted heterocyclic ring (e.g., substituted or unsubstituted, 5- to 10-membered monocyclic or bicyclic heterocyclic ring, wherein one or two atoms in the heterocyclic ring are independently nitrogen, oxygen, or sulfur) or optionally substituted heteroaryl ring (e.g., substituted or unsubstituted, 5- to 6-membered, monocyclic heteroaryl, wherein one, two, three, or four atoms in the heteroaryl ring system are independently nitrogen, oxygen, or sulfur; or substituted or unsubstituted, 9- to 10-membered, bicyclic heteroaryl, wherein one, two, three, or four atoms in the heteroaryl ring system are independently nitrogen, oxygen, or sulfur). In certain embodiments, R′ is of the formula:

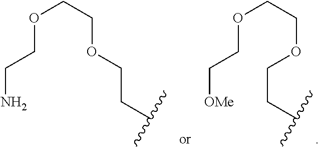 [SMILES: *CCOCCOCCN ; *CCOCCOCCOC]

In certain embodiments, R′ is of the formula:

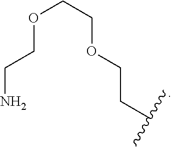 [SMILES: *CCOCCOCCN]

In certain embodiments, R′ is of the formula:

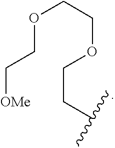 [SMILES: *CCOCCOCCOC]

In certain embodiments, R is hydrogen or unsubstituted C₁₋₆ alkyl; and R′ is of the formula:

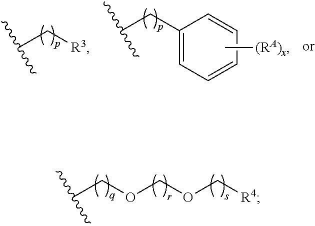

p is 1, 2, or 3; x is 2; q is 1, 2, or 3; r is 1, 2, or 3; s is 1, 2, or 3; two instances of R^{A} are taken together with their intervening atoms to form a optionally substituted heterocyclic or optionally substituted heteroaryl ring; R³ is optionally substituted carbocyclyl, optionally substituted heterocyclyl, or 5-membered or 6-membered optionally substituted heteroaryl; R⁴ is —OR^{4A} or —N(R^{4B})₂, R^{4A} is hydrogen, optionally substituted acyl, optionally substituted alkyl, optionally substituted alkenyl, or optionally substituted alkynyl; and each occurrence of R^{4B} is independently hydrogen, optionally substituted acyl, optionally substituted alkyl, optionally substituted alkenyl, or optionally substituted alkynyl; or optionally two instances of R^{4B} are taken together with their intervening atoms to form a optionally substituted heterocyclic or optionally substituted heteroaryl ring.

In certain embodiments, the compound of Formula (I) is of the formula:

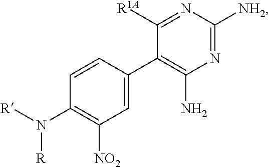

or a pharmaceutically acceptable salt, solvate, hydrate, stereoisomer, polymorph, tautomer, isotopically enriched form, or prodrug thereof, wherein R^{1A} is unsubstituted C₁₋₆ alkyl; R is hydrogen or unsubstituted C₁₋₆ alkyl; and R′ is of the formula:

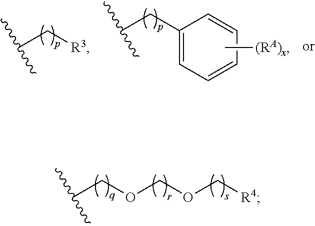

p is 1, 2, or 3; x is 2; q is 1, 2, or 3; r is 1, 2, or 3; s is 1, 2, or 3; two instances of R^{A} are taken together with their intervening atoms to form a optionally substituted heterocyclic or optionally substituted heteroaryl ring; R³ is optionally substituted carbocyclyl, optionally substituted heterocyclyl, or 5-membered or 6-membered optionally substituted heteroaryl; R⁴ is —OR^{4A} or —N(R^{4B})₂; R^{4A} is hydrogen, optionally substituted acyl, optionally substituted alkyl, optionally substituted alkenyl, or optionally substituted alkynyl; and each occurrence of R^{4B} is independently hydrogen, optionally substituted acyl, optionally substituted alkyl, optionally substituted alkenyl, or optionally substituted alkynyl; or optionally two instances of R^{4B} are taken together with their intervening atoms to form a optionally substituted heterocyclic or optionally substituted heteroaryl ring.

In certain embodiments, the compound of Formula (I) is of Formula (I-A):

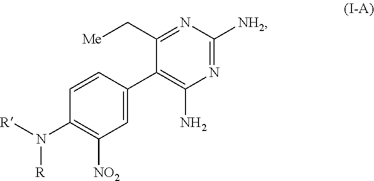

or a pharmaceutically acceptable salt, solvate, hydrate, stereoisomer, polymorph, tautomer, isotopically enriched form, or prodrug thereof, wherein R is hydrogen or unsubstituted C₁₋₆ alkyl; and R′ is of the formula:

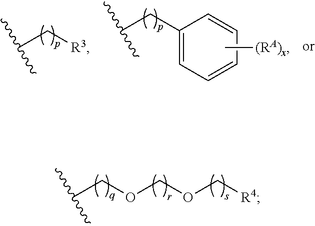

p is 1, 2, or 3; x is 2; q is 1, 2, or 3; r is 1, 2, or 3; s is 1, 2, or 3; two instances of R^{A} are taken together with their intervening atoms to form a optionally substituted heterocyclic or optionally substituted heteroaryl ring; R³ is optionally substituted carbocyclyl, optionally substituted heterocyclyl, or 5-membered or 6-membered optionally substituted heteroaryl; R⁴ is —OR^{4A} or —N(R^{4B})₂; R^{4A} is hydrogen, optionally substituted acyl, optionally substituted alkyl, optionally substituted alkenyl, or optionally substituted alkynyl; and each occurrence of R^{4B} is independently hydrogen, optionally substituted acyl, optionally substituted alkyl, optionally substituted alkenyl, or optionally substituted alkynyl; or optionally two instances of R^{4B} are taken together with their intervening atoms to form a optionally substituted heterocyclic or optionally substituted heteroaryl ring.

In certain embodiments, the compound of Formula (I) is of the formula:

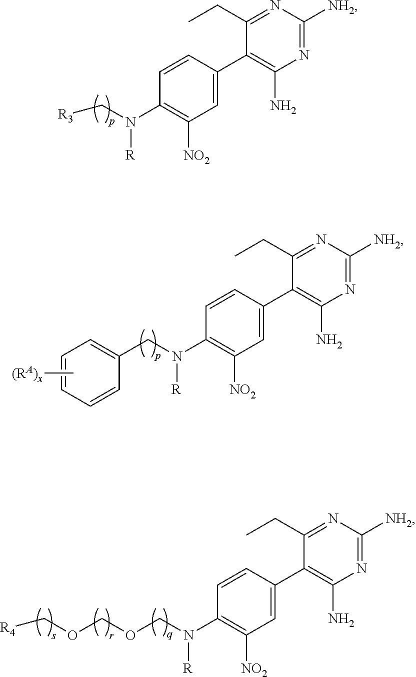

or a pharmaceutically acceptable salt, solvate, hydrate, stereoisomer, polymorph, tautomer, isotopically enriched form, or prodrug thereof.

In certain embodiments, the compound of Formula (I) is not a compound disclosed in PCT Application Publication No. WO 1998/030550, published Jul. 16, 1998, or a compound disclosed in PCT Application Publication No. WO 1988/004293, published Jun. 16, 1988. In certain embodiments, a compound of Formula (I) or a compound described herein is not of the formula:

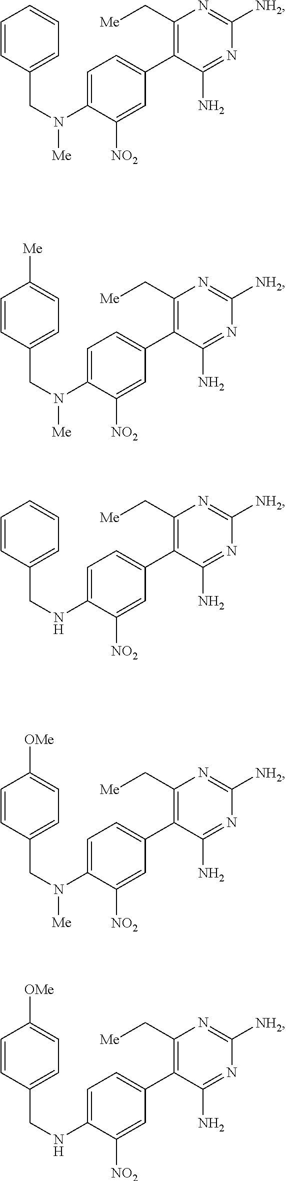 [SMILES: CCc1nc(N)nc(N)c1-c1ccc(N(C)Cc2ccccc2)c([N+](=O)[O-])c1 ; CCc1nc(N)nc(N)c1-c1ccc(N(C)Cc2ccc(C)cc2)c([N+](=O)[O-])c1 ; CCc1nc(N)nc(N)c1-c1ccc(NCc2ccccc2)c([N+](=O)[O-])c1 ; CCc1nc(N)nc(N)c1-c1ccc(N(C)Cc2ccc(OC)cc2)c([N+](=O)[O-])c1 ; CCc1nc(N)nc(N)c1-c1ccc(NCc2ccc(OC)cc2)c([N+](=O)[O-])c1]

or a pharmaceutically acceptable salt, solvate, hydrate, stereoisomer, polymorph, tautomer, isotopically enriched form, or prodrug thereof.

In certain embodiments, the compound of Formula (I) is of the formula:

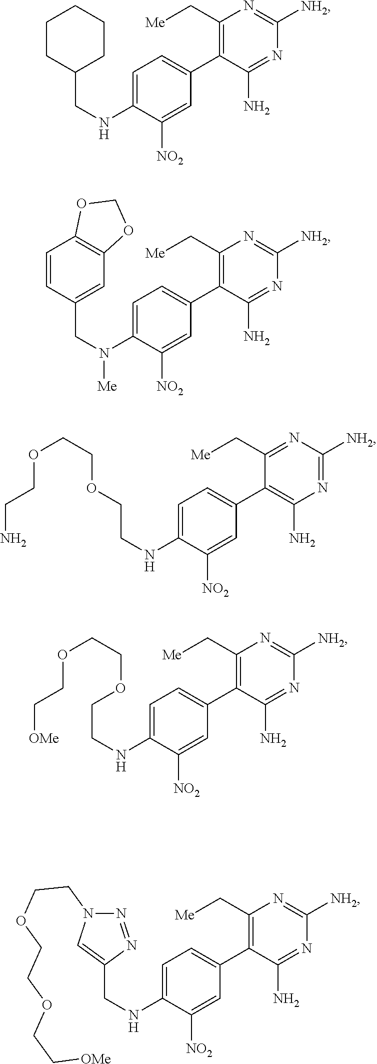 [SMILES: CCc1nc(N)nc(N)c1-c1ccc(NCC2CCCCC2)c([N+](=O)[O-])c1 ; CCc1nc(N)nc(N)c1-c1ccc(N(C)Cc2ccc3c(c2)OCO3)c([N+](=O)[O-])c1 ; CCc1nc(N)nc(N)c1-c1ccc(NCCOCCOCCN)c([N+](=O)[O-])c1 ; CCc1nc(N)nc(N)c1-c1ccc(NCCOCCOCCOC)c([N+](=O)[O-])c1 ; CCc1nc(N)nc(N)c1-c1ccc(NCc2cn(CCOCCOCCOC)nn2)c([N+](=O)[O-])c1]

or a pharmaceutically acceptable salt, solvate, hydrate, stereoisomer, polymorph, tautomer, isotopically enriched form, or prodrug thereof.

In certain embodiments, the compound of Formula (II) has the substituent X. In certain embodiments, X is —O—. In certain embodiments, X is —S—.

In certain embodiments, the compound of Formula (II) has the substituent R¹ of the formula:

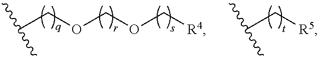

optionally substituted carbocyclyl, optionally substituted heterocyclyl, optionally substituted aryl, or optionally substituted heteroaryl. In certain embodiments, R¹′ is optionally substituted alkyl. In certain embodiments, R¹′ is of the formula:

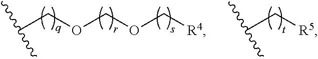

wherein R⁴, R⁵, q, r, s, and t are as defined herein.

In certain embodiments, R¹′ is of the formula:

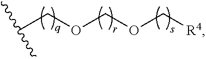

wherein q, r, s, and R⁴ are as defined herein. In certain embodiments, q is 1. In certain embodiments, q is 2. In certain embodiments, q is 3. In certain embodiments, q is 4. In certain embodiments, q is 5. In certain embodiments, q is 6. In certain embodiments, r is 1. In certain embodiments, r is 2. In certain embodiments, r is 3. In certain embodiments, r is 4. In certain embodiments, r is 5. In certain embodiments, r is 6. In certain embodiments, s is 1. In certain embodiments, s is 2. In certain embodiments, s is 3. In certain embodiments, s is 4. In certain embodiments, s is 5. In certain embodiments, s is 6. In certain embodiments, R⁴ is hydrogen. In certain embodiments, R⁴ is optionally substituted acyl (e.g., —C(═O)Me). In certain embodiments, R⁴ is optionally substituted alkyl (e.g., substituted or unsubstituted C₁₋₆ alkyl). In certain embodiments, R⁴ is substituted or unsubstituted methyl. In certain embodiments, R⁴ is substituted methyl (e.g., —CF₃). In certain embodiments, R⁴ is unsubstituted methyl. In certain embodiments, R⁴ is substituted or unsubstituted ethyl. In certain embodiments, R⁴ is substituted or unsubstituted propyl. In certain embodiments, R⁴ is substituted or unsubstituted butyl (e.g., t-butyl, n-butyl). In certain embodiments, R⁴ is optionally substituted alkenyl (e.g., substituted or unsubstituted C₂₋₆ alkenyl). In certain embodiments, R⁴ is optionally substituted alkynyl (e.g., substituted or unsubstituted C₂₋₆ alkynyl). In certain embodiments, R⁴ is optionally substituted carbocyclyl (e.g., substituted or unsubstituted, 3- to 10-membered, monocyclic carbocyclyl comprising zero, one, or two double bonds in the carbocyclic ring system). In certain embodiments, R⁴ is optionally substituted heterocyclyl (e.g., substituted or unsubstituted, 5- to 10-membered monocyclic or bicyclic heterocyclic ring, wherein one or two atoms in the heterocyclic ring are independently nitrogen, oxygen, or sulfur). In certain embodiments, R⁴ is optionally substituted aryl (e.g., substituted or unsubstituted, 6- to 10-membered aryl). In certain embodiments, R⁴ is benzyl. In certain embodiments, R⁴ is substituted or unsubstituted phenyl. In certain embodiments, R⁴ is optionally substituted heteroaryl (e.g., substituted or unsubstituted, 5- to 6-membered, monocyclic heteroaryl, wherein one, two, three, or four atoms in the heteroaryl ring system are independently nitrogen, oxygen, or sulfur; or substituted or unsubstituted, 9- to 10-membered, bicyclic heteroaryl, wherein one, two, three, or four atoms in the heteroaryl ring system are independently nitrogen, oxygen, or sulfur). In certain embodiments, R¹′ is of the formula:

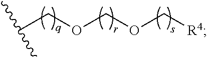

q is 1, 2, or 3; r is 1, 2, or 3; s is 1, 2, or 3; R⁴ is —OR^{4A} or —N(R^{4B})₂; R^{4A} is hydrogen, optionally substituted acyl, optionally substituted alkyl, optionally substituted alkenyl, optionally substituted alkynyl, optionally substituted carbocyclyl, optionally substituted heterocyclyl, optionally substituted aryl, or optionally substituted heteroaryl; and each occurrence of R^{4B} is independently hydrogen, optionally substituted acyl, optionally substituted alkyl, optionally substituted alkenyl, optionally substituted alkynyl, optionally substituted carbocyclyl, optionally substituted heterocyclyl, optionally substituted aryl, or optionally substituted heteroaryl; or optionally two instances of R^{4B} are taken together with their intervening atoms to form a substituted or unsubstituted heterocyclic or substituted or unsubstituted heteroaryl ring. In certain embodiments, R¹′ is of the formula:

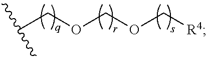

q is 1 or 2; r is 1 or 2; s is 1 or 2; R⁴ is —OR^{4A} or —N(R^{4B})₂.

In certain embodiments, R¹′ is of the formula:

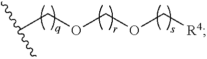

q is 2; r is 2; s is 2; R⁴ is —OR^{4A} or —N(R^{4B})₂. In certain embodiments, R¹′ is of the formula:

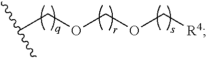

q is 2; r is 2; and s is 2. In certain embodiments, R^{1′} is of the formula:

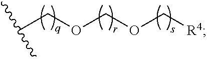

and q is 2.

In certain embodiments, R^{1′} is of the formula:

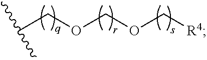

and r is 2. In certain embodiments, R¹′ is of the formula:

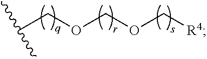

and s is 2. In certain embodiments, R⁴ is —OR^{4A}, —N(R^{4B})₂, or —SR^{4A}, and R^{4A} and R^{4B} are as defined herein. In certain embodiments, R⁴ is —OR^{4A} or —N(R^{4B})₂, and R^{4A} and R^{4B} are as defined herein. In certain embodiments, R⁴ is —OR^{4A} (e.g., —OH or —OMe). In certain embodiments, R⁴ is C₁₋₆ alkoxyl. In certain embodiments, R⁴ is —OR^{4A}; and R^{4A} is optionally substituted acyl or optionally substituted C₁₋₆ alkyl. In certain embodiments, R⁴ is —OMe. In certain embodiments, R⁴ is —N(R^{4B})₂ (e.g., —NMe₂). In certain embodiments, R⁴ is —N(R^{4B})₂; and each occurrence of R^{4B} is independently hydrogen, optionally substituted acyl, optionally substituted alkyl, or optionally substituted alkenyl. In certain embodiments, R⁴ is —NH₂. In certain embodiments, R⁴ is —SR^{4A} (e.g., —SMe). In certain embodiments, R^{4A} is hydrogen. In certain embodiments, R^{4A} is optionally substituted acyl (e.g., —C(═O)Me). In certain embodiments, R^{4A} is substituted or unsubstituted alkyl (e.g., substituted or unsubstituted C₁₋₆ alkyl). In certain embodiments, R^{4A} is substituted or unsubstituted methyl. In certain embodiments, R^{4A} is substituted or unsubstituted ethyl. In certain embodiments, R^{4A} is substituted or unsubstituted propyl. In certain embodiments, R^{4A} is substituted or unsubstituted alkenyl (e.g., substituted or unsubstituted C₂₋₆ alkenyl). In certain embodiments, R^{4A} is substituted or unsubstituted alkynyl (e.g., substituted or unsubstituted C₂₋₆ alkynyl). In certain embodiments, R^{4A} is substituted or unsubstituted carbocyclyl (e.g., substituted or unsubstituted, 3- to 7-membered, monocyclic carbocyclyl comprising zero, one, or two double bonds in the carbocyclic ring system). In certain embodiments, R^{4A} is substituted or unsubstituted heterocyclyl (e.g., substituted or unsubstituted, 5- to 10-membered monocyclic or bicyclic heterocyclic ring, wherein one or two atoms in the heterocyclic ring are independently nitrogen, oxygen, or sulfur). In certain embodiments, R^{4A} is substituted or unsubstituted aryl (e.g., substituted or unsubstituted, 6- to 10-membered aryl). In certain embodiments, R^{4A} is benzyl. In certain embodiments, R^{4A} is substituted or unsubstituted phenyl. In certain embodiments, R^{4A} is substituted or unsubstituted heteroaryl (e.g., substituted or unsubstituted, 5- to 6-membered, monocyclic heteroaryl, wherein one, two, three, or four atoms in the heteroaryl ring system are independently nitrogen, oxygen, or sulfur; or substituted or unsubstituted, 9- to 10-membered, bicyclic heteroaryl, wherein one, two, three, or four atoms in the heteroaryl ring system are independently nitrogen, oxygen, or sulfur). In certain embodiments, R^{4A} is an oxygen protecting group when attached to an oxygen atom. In certain embodiments, R^{4A} is a sulfur protecting group when attached to a sulfur atom. In certain embodiments, at least one instance of R^{4B} is hydrogen. In certain embodiments, at least one instance of R^{4B} is optionally substituted acyl (e.g., —C(═O)Me). In certain embodiments, at least one R^{4B} is substituted or unsubstituted alkyl (e.g., substituted or unsubstituted C₁₋₆ alkyl). In certain embodiments, at least one instance of R^{4B} is substituted or unsubstituted methyl. In certain embodiments, at least one instance of R^{4B} is substituted or unsubstituted ethyl. In certain embodiments, at least one instance of R^{4B} is substituted or unsubstituted propyl. In certain embodiments, at least one instance of R^{4B} is substituted or unsubstituted alkenyl (e.g., substituted or unsubstituted C₂₋₆ alkenyl). In certain embodiments, at least one instance of R^{4B} is substituted or unsubstituted alkynyl (e.g., substituted or unsubstituted C₂₋₆ alkynyl). In certain embodiments, at least one instance of R^{4B} is substituted or unsubstituted carbocyclyl (e.g., substituted or unsubstituted, 3- to 7-membered, monocyclic carbocyclyl comprising zero, one, or two double bonds in the carbocyclic ring system). In certain embodiments, at least one instance of R^{4B} is substituted or unsubstituted heterocyclyl (e.g., substituted or unsubstituted, 5- to 10-membered monocyclic or bicyclic heterocyclic ring, wherein one or two atoms in the heterocyclic ring are independently nitrogen, oxygen, or sulfur). In certain embodiments, at least one instance of R^{4B} is substituted or unsubstituted aryl (e.g., substituted or unsubstituted, 6- to 10-membered aryl). In certain embodiments, at least one instance of R^{4B} is benzyl. In certain embodiments, at least one instance of R^{4B} is substituted or unsubstituted phenyl. In certain embodiments, at least one instance of R^{4B} is substituted or unsubstituted heteroaryl (e.g., substituted or unsubstituted, 5- to 6-membered, monocyclic heteroaryl, wherein one, two, three, or four atoms in the heteroaryl ring system are independently nitrogen, oxygen, or sulfur; or substituted or unsubstituted, 9- to 10-membered, bicyclic heteroaryl, wherein one, two, three, or four atoms in the heteroaryl ring system are independently nitrogen, oxygen, or sulfur). In certain embodiments, at least one instance of R^{4B} is a nitrogen protecting group (e.g., benzyl (Bn), t-butyl carbonate (BOC or Boc), benzyl carbamate (Cbz), 9-fluorenylmethyl carbonate (Fmoc), trifluoroacetyl, triphenylmethyl, acetyl, or p-toluenesulfonamide (Ts)). In certain embodiments, two instances of R^{4B} are taken together with their intervening atoms to form a substituted or unsubstituted heterocyclic ring (e.g., substituted or unsubstituted, 5- to 10-membered monocyclic or bicyclic heterocyclic ring, wherein one or two atoms in the heterocyclic ring are independently nitrogen, oxygen, or sulfur) or substituted or unsubstituted heteroaryl ring (e.g., substituted or unsubstituted, 5- to 6-membered, monocyclic heteroaryl, wherein one, two, three, or four atoms in the heteroaryl ring system are independently nitrogen, oxygen, or sulfur; or substituted or unsubstituted, 9- to 10-membered, bicyclic heteroaryl, wherein one, two, three, or four atoms in the heteroaryl ring system are independently nitrogen, oxygen, or sulfur). In certain embodiments, R¹′ is of the formula:

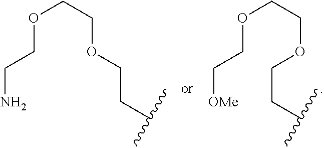 [SMILES: *CCOCCOCCN ; *CCOCCOCCOC]

In certain embodiments, R¹′ is of the formula:

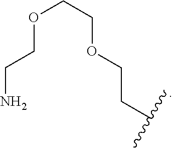 [SMILES: *CCOCCOCCN]

In certain embodiments, R¹′ is of the formula:

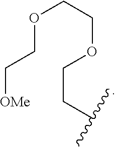 [SMILES: *CCOCCOCCOC]

In certain embodiments, R¹′ is of the formula:

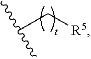

wherein t and R⁵ are as defined herein. In certain embodiments, t is 1, 2, 3, 4, 5, or 6. In certain embodiments, t is 1. In certain embodiments, t is 2. In certain embodiments, t is 3. In certain embodiments, t is 4. In certain embodiments, t is 5. In certain embodiments, t is 6. In certain embodiments, R¹′ is of the formula:

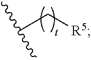

t is 1, 2, or 3; and R⁵ is optionally substituted acyl, optionally substituted alkyl, optionally substituted alkenyl, optionally substituted alkynyl, optionally substituted carbocyclyl, optionally substituted heterocyclyl, optionally substituted aryl, or optionally substituted heteroaryl. In certain embodiments, R⁵ is hydrogen. In certain embodiments, R⁵ is —OR^{D1} (e.g., —OH or —OMe). In certain embodiments, R⁵ is optionally substituted acyl (e.g., —C(═O)Me). In certain embodiments, R⁵ is optionally substituted alkyl (e.g., substituted or unsubstituted C₁₋₆ alkyl). In certain embodiments, R⁵ is substituted or unsubstituted methyl. In certain embodiments, R⁵ is substituted or unsubstituted ethyl. In certain embodiments, R⁵ is substituted or unsubstituted propyl. In certain embodiments, R⁵ is optionally substituted alkenyl (e.g., substituted or unsubstituted C₂₋₆ alkenyl). In certain embodiments, R⁵ is optionally substituted alkynyl (e.g., substituted or unsubstituted C₂₋₆ alkynyl). In certain embodiments, R⁵ is optionally substituted carbocyclyl (e.g., substituted or unsubstituted, 3- to 7-membered, monocyclic carbocyclyl comprising zero, one, or two double bonds in the carbocyclic ring system). In certain embodiments, R⁵ is optionally substituted, 3- to 7-membered, monocyclic carbocyclyl comprising zero, one, or two double bonds in the carbocyclic ring system. In certain embodiments, R⁵ is optionally substituted C₃₋₆ monocyclic carbocyclyl (e.g., cyclopentyl, cyclohexyl, cycloheptyl). In certain embodiments, R⁵ is optionally substituted C₅₋₆ monocyclic carbocyclyl (e.g., cyclopentyl, cyclohexyl). In certain embodiments, R⁵ is optionally substituted cyclohexyl. In certain embodiments, R⁵ is optionally substituted heterocyclyl (e.g., substituted or unsubstituted, 5- to 10-membered monocyclic or bicyclic heterocyclic ring, wherein one or two atoms in the heterocyclic ring are independently nitrogen, oxygen, or sulfur). In certain embodiments, R⁵ is optionally substituted aryl (e.g., substituted or unsubstituted, 6- to 10-membered aryl). In certain embodiments, R⁵ is benzyl. In certain embodiments, R⁵ is optionally substituted phenyl. In certain embodiments, R⁵ is phenyl. In certain embodiments, R⁵ is optionally substituted heteroaryl (e.g., substituted or unsubstituted, 5- to 6-membered, monocyclic heteroaryl, wherein one, two, three, or four atoms in the heteroaryl ring system are independently nitrogen, oxygen, or sulfur; or substituted or unsubstituted, 9- to 10 -membered, bicyclic heteroaryl, wherein one, two, three, or four atoms in the heteroaryl ring system are independently nitrogen, oxygen, or sulfur). In certain embodiments, R⁵ is optionally substituted, 5- to 6-membered, monocyclic heteroaryl, wherein one, two, three, or four atoms in the heteroaryl ring system are independently nitrogen, oxygen, or sulfur.

In certain embodiments, R¹′ is optionally substituted carbocyclyl (e.g., substituted or unsubstituted, 3- to 7-membered, monocyclic carbocyclyl comprising zero, one, or two double bonds in the carbocyclic ring system). In certain embodiments, R¹′ is optionally substituted heterocyclyl (e.g., substituted or unsubstituted, 5- to 10-membered monocyclic or bicyclic heterocyclic ring, wherein one or two atoms in the heterocyclic ring are independently nitrogen, oxygen, or sulfur). In certain embodiments, R¹′ is optionally substituted aryl (e.g., substituted or unsubstituted, 6- to 10-membered aryl). In certain embodiments, R¹′ is benzyl. In certain embodiments, R¹′ is substituted or unsubstituted phenyl. In certain embodiments, R⁵ is optionally substituted heteroaryl (e.g., substituted or unsubstituted, 5- to 6-membered, monocyclic heteroaryl, wherein one, two, three, or four atoms in the heteroaryl ring system are independently nitrogen, oxygen, or sulfur; or substituted or unsubstituted, 9- to 10-membered, bicyclic heteroaryl, wherein one, two, three, or four atoms in the heteroaryl ring system are independently nitrogen, oxygen, or sulfur).

In certain embodiments, the compound of Formula (II) is of the formula:

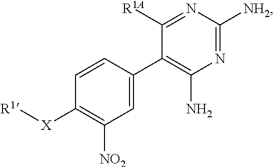

or a pharmaceutically acceptable salt, solvate, hydrate, stereoisomer, polymorph, tautomer, isotopically enriched form, or prodrug thereof, wherein R^{1A} is unsubstituted C₁₋₆ alkyl; R is hydrogen or unsubstituted C₁₋₆ alkyl; and R¹′ is

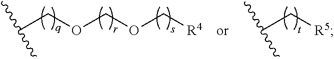

t is 1, 2, or 3; q is 1, 2, or 3; r is 1, 2, or 3; s is 1, 2, or 3; and R⁵ is optionally substituted acyl, optionally substituted alkyl, optionally substituted alkenyl, optionally substituted alkynyl, optionally substituted carbocyclyl, optionally substituted heterocyclyl, optionally substituted aryl, or optionally substituted heteroaryl.

In certain embodiments, the compound of Formula (II) is of Formula (II-A):

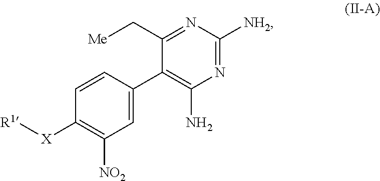

or a pharmaceutically acceptable salt, solvate, hydrate, stereoisomer, polymorph, tautomer, isotopically enriched form, or prodrug thereof, wherein R^{1′} is

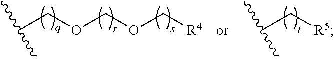

t is 1, 2, or 3; q is 1, 2, or 3; r is 1, 2, or 3; s is 1, 2, or 3; and R⁵ is optionally substituted acyl, optionally substituted alkyl, optionally substituted alkenyl, optionally substituted alkynyl, optionally substituted carbocyclyl, optionally substituted heterocyclyl, optionally substituted aryl, or optionally substituted heteroaryl.

In certain embodiments, the compound of Formula (II) is of the formula:

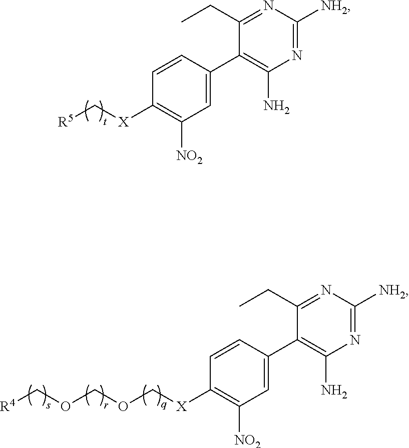

or a pharmaceutically acceptable salt, solvate, hydrate, stereoisomer, polymorph, tautomer, isotopically enriched form, or prodrug thereof.

In certain embodiments, a compound described herein is of the formula:

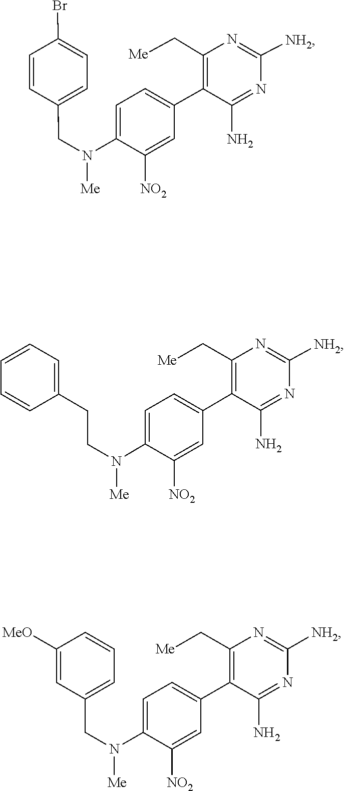 [SMILES: CCc1nc(N)nc(N)c1-c1ccc(N(C)Cc2ccc(Br)cc2)c([N+](=O)[O-])c1 ; CCc1nc(N)nc(N)c1-c1ccc(N(C)CCc2ccccc2)c([N+](=O)[O-])c1 ; CCc1nc(N)nc(N)c1-c1ccc(N(C)Cc2cccc(OC)c2)c([N+](=O)[O-])c1]

or a pharmaceutically acceptable salt, solvate, hydrate, stereoisomer, polymorph, tautomer, isotopically enriched form, or prodrug thereof.

In certain embodiments, the compound of Formula (I) or (II) is a compound provided in any one of the Examples below. In certain embodiments, a compound described herein is a compound of Formula (I) or (II), or a pharmaceutically acceptable salt thereof. In certain embodiments, the compound described herein is a compound of Table A.

Certain compounds described herein bind, modify, and/or inhibit a thymidylate synthase, dihydrofolate reductase. In certain embodiments, the compounds described herein irreversibly inhibit a thymidylate synthase, dihydrofolate reductase. In certain embodiments, the compounds described herein reversibly inhibit a thymidylate synthase, dihydrofolate reductase. In certain embodiments, the compounds described herein covalently bind to the thymidylate synthase, dihydrofolate reductase. In certain embodiments, the compounds described herein reversibly bind to the thymidylate synthase, dihydrofolate reductase. In certain embodiments, the compounds described herein non-reversibly bind to the thymidylate synthase, dihydrofolate reductase. In certain embodiments, the compounds described herein modulate the activity of a thymidylate synthase, dihydrofolate reductase. In certain embodiments, the compounds described herein inhibit the thymidylate synthase, dihydrofolate reductase. In certain embodiments, the compounds described herein reversibly inhibit the activity of a thymidylate synthase, dihydrofolate reductase.

The binding affinity of a compound described herein to a thymidylate synthase, dihydrofolate reductase may be measured by the dissociation constant (K_{d}) value of an adduct of the compound and the thymidylate synthase, dihydrofolate reductase using methods known in the art (e.g., isothermal titration calorimetry (ITC)). In certain embodiments, the K_{d} value of the adduct is not more than about 100 μM, not more than about 10 μM, not more than about 1 μM, not more than about 100 nM, not more than about 10 nM, or not more than about 1 nM.

In certain embodiments, the activity of a thymidylate synthase, dihydrofolate reductase is inhibited by a compound described herein. The inhibition of the activity of a thymidylate synthase, dihydrofolate reductase by a compound described herein may be measured by determining the half maximal inhibitory concentration (IC₅₀) of the compound when the compound, or a pharmaceutical composition thereof, is contacted with the thymidylate synthase, dihydrofolate reductase. The IC₅₀ values may be obtained using methods known in the art (e.g., by a competition binding assay). In certain embodiments, the IC₅₀ value of a compound described herein is not more than about 1 mM, not more than about 100 μM, not more than about 10 μM, not more than about 1 μM, not more than about 100 nM, not more than about 10 nM, or not more than about 1 nM.

The compounds described herein may selectively modulate the activity of a thymidylate synthase, dihydrofolate reductase. In certain embodiments, the compounds selectively decrease the activity of a thymidylate synthase, dihydrofolate reductase.

The selectivity of a compound described herein in inhibiting the activity of a first folate-dependent enzyme (e.g., thymidylate synthase) over a second folate-dependent enzyme may be measured by the quotient of the IC₅₀ value of the compound in inhibiting the activity of the second folate-dependent enzyme over the IC₅₀ value of the compound in inhibiting the activity of the first folate-dependent enzyme. The selectivity of a compound described herein in modulating the activity of a first folate-dependent enzyme (e.g., thymidylate synthase) over a second folate-dependent enzyme may also be measured by the quotient of the K_{d} value of an adduct of the compound and the second folate-dependent enzyme over the K_{d} value of an adduct of the compound and the first folate-dependent enzyme (e.g., thymidylate synthase). In certain embodiments, the selectivity is at least about 1-fold, at least about 3-fold, at least about 10-fold, at least about 30-fold, at least about 100-fold, at least about 300-fold, at least about 1,000-fold, at least about 3,000-fold, at least about 10,000-fold, at least about 30,000-fold, or at least about 100,000-fold. In certain embodiments, the selectivity is at least about 1 at least 2-fold, 5-fold, 10-fold, or more. In certain embodiments, the compounds of Formula (I) or (II), or compounds of Table A, are selective for a first folate-dependent enzyme (e.g., thymidylate synthase) compared to other folate-dependent enzymes (e.g., at least 2-fold, 5-fold, 10-fold, or more selective for thymidylate synthase). In certain embodiments, the compounds of Formula (I) or (II), or compounds of Table A, are selective for a first folate-dependent enzyme (e.g., thymidylate synthase) compared to other folate-dependent enzymes (e.g., at least 2-fold, 5-fold, 10-fold, or more selective for thymidylate synthase). In certain embodiments, the compounds of Formula (I) or (II), or compounds of Table A, bind both thymidylate synthase and dihydrofolate reductase.

It is expected that the compounds described herein may be useful in treating and/or preventing diseases associated with aberrant activity (e.g., increased activity, undesired activity, abnormal activity) of a thymidylate synthase, dihydrofolate reductase. It is known in the art that thymidylate synthase, dihydrofolate reductase are implicated in a wide range of diseases and conditions, such as proliferative disease (e.g., cancers). Therefore, the compounds described herein are expected to be useful in treating and/or preventing diseases (e.g., proliferative disease, such as, cancers).

### Pharmaceutical Compositions, Kits, and Administration

The present disclosure also provides pharmaceutical compositions comprising a compound described herein and optionally a pharmaceutically acceptable excipient. In certain embodiments, a compound described herein is a compound of Formula (I) or (II), or a compound of Table A, or a pharmaceutically acceptable salt thereof, and a pharmaceutically acceptable excipient. In certain embodiments, a compound described herein in a pharmaceutical composition for treating the diseases and/or conditions described herein is a compound of Formula (I) or (II), a compound of Table A, or a pharmaceutically acceptable salt thereof, and a pharmaceutically acceptable excipient.

In certain embodiments, the compound described herein is provided in an effective amount in the pharmaceutical composition. In certain embodiments, the effective amount is a therapeutically effective amount. In certain embodiments, the effective amount is a prophylactically effective amount. In certain embodiments, a therapeutically effective amount is an amount effective for treating a disease (e.g., proliferative disease, such as, cancers); or for treating other diseases associated with folate-dependent enzyme (e.g., thymidylate synthase, dihydrofolate reductase). In certain embodiments, a therapeutically effective amount is an amount effective for inhibiting the aberrant activity of a thymidylate synthase, dihydrofolate reductase. In certain embodiments, a therapeutically effective amount is an amount effective for inhibiting the aberrant activity of a thymidylate synthase, dihydrofolate reductase and treating a disease (e.g., a disease associated with aberrant activity of a folate-dependent enzyme (e.g., thymidylate synthase, dihydrofolate reductase). In certain embodiments, a prophylactically effective amount is an amount effective for inhibiting the aberrant activity of a thymidylate synthase, dihydrofolate reductase. In certain embodiments, a prophylactically effective amount is an amount effective for preventing or keeping a subject in need thereof in remission of a disease (e.g., a disease associated with aberrant activity of a folate-dependent enzyme (e.g., thymidylate synthase, dihydrofolate reductase) (e.g., proliferative disease, such as, cancers), and other diseases associated with folate-dependent enzyme (e.g., thymidylate synthase, dihydrofolate reductase). In certain embodiments, a prophylactically effective amount is an amount effective for inhibiting the aberrant activity of a folate-dependent enzyme (e.g., thymidylate synthase, dihydrofolate reductase), and preventing or keeping a subject in need thereof in remission of a disease (e.g., a disease associated with aberrant activity of a thymidylate synthase, dihydrofolate reductase (e.g., proliferative disease, such as, cancers); and other diseases associated with a folate-dependent enzyme (e.g., thymidylate synthase, dihydrofolate reductase). In certain embodiments, a compound of Formula (I) or (II), or Table A, and salts (e.g., pharmaceutically acceptable salts), solvates, hydrates, stereoisomers, polymorphs, tautomers, co-crystals, isotopically labeled derivatives, and prodrugs thereof, is used for treating diseases and/or conditions disclosed herein.

In certain embodiments, the effective amount is an amount effective for inhibiting the activity of a thymidylate synthase, dihydrofolate reductase by at least 10%, at least 20%, at least 30%, at least 40%, at least 50%, at least 60%, at least 70%, at least 80%, at least 90%, at least 95%, or at least 98%. In certain embodiments, the effective amount is an amount effective for inhibiting the activity of a thymidylate synthase, dihydrofolate reductase by not more than 10%, not more than 20%, not more than 30%, not more than 40%, not more than 50%, not more than 60%, not more than 70%, not more than 80%, not more than 90%, not more than 95%, or not more than 98%.

In certain embodiments, the subject is an animal. The animal may be of either sex and may be at any stage of development. In certain embodiments, the subject described herein is a human. In certain embodiments, the subject is a non-human animal. In certain embodiments, the subject is a mammal. In certain embodiments, the subject is a non-human mammal. In certain embodiments, the subject is a domesticated animal, such as a dog, cat, cow, pig, horse, sheep, or goat. In certain embodiments, the subject is a companion animal, such as a dog or cat. In certain embodiments, the subject is a livestock animal, such as a cow, pig, horse, sheep, or goat. In certain embodiments, the subject is a zoo animal. In another embodiment, the subject is a research animal, such as a rodent (e.g., mouse, rat), dog, pig, or non-human primate. In certain embodiments, the animal is a genetically engineered animal. In certain embodiments, the animal is a transgenic animal (e.g., transgenic mice and transgenic pigs). In certain embodiments, the subject is a fish or reptile.

In certain embodiments, the cell being contacted with a compound or composition described herein is in vitro. In certain embodiments, the cell being contacted with a compound or composition described herein is in vivo.

Pharmaceutical compositions described herein can be prepared by any method known in the art of pharmacology. In general, such preparatory methods include bringing the compound described herein (i.e., the “active ingredient”) into association with a carrier or excipient, and/or one or more other accessory ingredients, and then, if necessary and/or desirable, shaping, and/or packaging the product into a desired single- or multi-dose unit.

Pharmaceutical compositions can be prepared, packaged, and/or sold in bulk, as a single unit dose, and/or as a plurality of single unit doses. A “unit dose” is a discrete amount of the pharmaceutical composition comprising a predetermined amount of the active ingredient. The amount of the active ingredient is generally equal to the dosage of the active ingredient which would be administered to a subject and/or a convenient fraction of such a dosage, such as one-half or one-third of such a dosage.

Relative amounts of the active ingredient, the pharmaceutically acceptable excipient, and/or any additional ingredients in a pharmaceutical composition described herein will vary, depending upon the identity, size, and/or condition of the subject treated and further depending upon the route by which the composition is to be administered. The composition may comprise between 0.1% and 100% (w/w) active ingredient.

Pharmaceutically acceptable excipients used in the manufacture of provided pharmaceutical compositions include inert diluents, dispersing and/or granulating agents, surface active agents and/or emulsifiers, disintegrating agents, binding agents, preservatives, buffering agents, lubricating agents, and/or oils. Excipients such as cocoa butter and suppository waxes, coloring agents, coating agents, sweetening, flavoring, and perfuming agents may also be present in the composition.

Exemplary diluents include calcium carbonate, sodium carbonate, calcium phosphate, dicalcium phosphate, calcium sulfate, calcium hydrogen phosphate, sodium phosphate lactose, sucrose, cellulose, microcrystalline cellulose, kaolin, mannitol, sorbitol, inositol, sodium chloride, dry starch, cornstarch, powdered sugar, and mixtures thereof.

Exemplary granulating and/or dispersing agents include potato starch, corn starch, tapioca starch, sodium starch glycolate, clays, alginic acid, guar gum, citrus pulp, agar, bentonite, cellulose, and wood products, natural sponge, cation-exchange resins, calcium carbonate, silicates, sodium carbonate, cross-linked poly(vinyl-pyrrolidone) (crospovidone), sodium carboxymethyl starch (sodium starch glycolate), carboxymethyl cellulose, cross-linked sodium carboxymethyl cellulose (croscarmellose), methylcellulose, pregelatinized starch (starch 1500), microcrystalline starch, water insoluble starch, calcium carboxymethyl cellulose, magnesium aluminum silicate (Veegum), sodium lauryl sulfate, quaternary ammonium compounds, and mixtures thereof.

Exemplary surface active agents and/or emulsifiers include natural emulsifiers (e.g., acacia, agar, alginic acid, sodium alginate, tragacanth, chondrux, cholesterol, xanthan, pectin, gelatin, egg yolk, casein, wool fat, cholesterol, wax, and lecithin), colloidal clays (e.g., bentonite (aluminum silicate) and Veegum (magnesium aluminum silicate)), long chain amino acid derivatives, high molecular weight alcohols (e.g., stearyl alcohol, cetyl alcohol, oleyl alcohol, triacetin monostearate, ethylene glycol distearate, glyceryl monostearate, and propylene glycol monostearate, polyvinyl alcohol), carbomers (e.g., carboxy polymethylene, polyacrylic acid, acrylic acid polymer, and carboxyvinyl polymer), carrageenan, cellulosic derivatives (e.g., carboxymethylcellulose sodium, powdered cellulose, hydroxymethyl cellulose, hydroxypropyl cellulose, hydroxypropyl methylcellulose, methylcellulose), sorbitan fatty acid esters (e.g., polyoxyethylene sorbitan monolaurate (Tween® 20), polyoxyethylene sorbitan (Tween® 60), polyoxyethylene sorbitan monooleate (Tween® 80), sorbitan monopalmitate (Span® 40), sorbitan monostearate (Span® 60), sorbitan tristearate (Span® 65), glyceryl monooleate, sorbitan monooleate (Span® 80), polyoxyethylene esters (e.g., polyoxyethylene monostearate (Myrj® 45), polyoxyethylene hydrogenated castor oil, polyethoxylated castor oil, polyoxymethylene stearate, and Solutol®), sucrose fatty acid esters, polyethylene glycol fatty acid esters (e.g., Cremophor®), polyoxyethylene ethers, (e.g., polyoxyethylene lauryl ether (Brij® 30)), poly(vinyl-pyrrolidone), diethylene glycol monolaurate, triethanolamine oleate, sodium oleate, potassium oleate, ethyl oleate, oleic acid, ethyl laurate, sodium lauryl sulfate, Pluronic® F-68, poloxamer P-188, cetrimonium bromide, cetylpyridinium chloride, benzalkonium chloride, docusate sodium, and/or mixtures thereof.

Exemplary binding agents include starch (e.g., cornstarch and starch paste), gelatin, sugars (e.g., sucrose, glucose, dextrose, dextrin, molasses, lactose, lactitol, mannitol, etc.), natural and synthetic gums (e.g., acacia, sodium alginate, extract of Irish moss, panwar gum, ghatti gum, mucilage of isapol husks, carboxymethylcellulose, methylcellulose, ethylcellulose, hydroxyethylcellulose, hydroxypropyl cellulose, hydroxypropyl methylcellulose, microcrystalline cellulose, cellulose acetate, poly(vinyl-pyrrolidone), magnesium aluminum silicate (Veegum®), and larch arabogalactan), alginates, polyethylene oxide, polyethylene glycol, inorganic calcium salts, silicic acid, polymethacrylates, waxes, water, alcohol, and/or mixtures thereof.

Exemplary preservatives include antioxidants, chelating agents, antimicrobial preservatives, antifungal preservatives, antiprotozoan preservatives, alcohol preservatives, acidic preservatives, and other preservatives. In certain embodiments, the preservative is an antioxidant. In other embodiments, the preservative is a chelating agent.

Exemplary antioxidants include alpha tocopherol, ascorbic acid, acorbyl palmitate, butylated hydroxyanisole, butylated hydroxytoluene, monothioglycerol, potassium metabisulfite, propionic acid, propyl gallate, sodium ascorbate, sodium bisulfite, sodium metabisulfite, and sodium sulfite.

Exemplary chelating agents include ethylenediaminetetraacetic acid (EDTA) and salts and hydrates thereof (e.g., sodium edetate, disodium edetate, trisodium edetate, calcium disodium edetate, dipotassium edetate, and the like), citric acid and salts and hydrates thereof (e.g., citric acid monohydrate), fumaric acid and salts and hydrates thereof, malic acid and salts and hydrates thereof, phosphoric acid and salts and hydrates thereof, and tartaric acid and salts and hydrates thereof. Exemplary antimicrobial preservatives include benzalkonium chloride, benzethonium chloride, benzyl alcohol, bronopol, cetrimide, cetylpyridinium chloride, chlorhexidine, chlorobutanol, chlorocresol, chloroxylenol, cresol, ethyl alcohol, glycerin, hexetidine, imidurea, phenol, phenoxyethanol, phenylethyl alcohol, phenylmercuric nitrate, propylene glycol, and thimerosal.

Exemplary antifungal preservatives include butyl paraben, methyl paraben, ethyl paraben, propyl paraben, benzoic acid, hydroxybenzoic acid, potassium benzoate, potassium sorbate, sodium benzoate, sodium propionate, and sorbic acid.

Exemplary alcohol preservatives include ethanol, polyethylene glycol, phenol, phenolic compounds, bisphenol, chlorobutanol, hydroxybenzoate, and phenylethyl alcohol.

Exemplary acidic preservatives include vitamin A, vitamin C, vitamin E, beta-carotene, citric acid, acetic acid, dehydroacetic acid, ascorbic acid, sorbic acid, and phytic acid.

Other preservatives include tocopherol, tocopherol acetate, deteroxime mesylate, cetrimide, butylated hydroxyanisol (BHA), butylated hydroxytoluened (BHT), ethylenediamine, sodium lauryl sulfate (SLS), sodium lauryl ether sulfate (SLES), sodium bisulfite, sodium metabisulfite, potassium sulfite, potassium metabisulfite, Glydant® Plus, Phenonip®, methylparaben, Germall® 115, Germaben® II, Neolone®, Kathon®, and Euxyl®.

Exemplary buffering agents include citrate buffer solutions, acetate buffer solutions, phosphate buffer solutions, ammonium chloride, calcium carbonate, calcium chloride, calcium citrate, calcium glubionate, calcium gluceptate, calcium gluconate, D-gluconic acid, calcium glycerophosphate, calcium lactate, propanoic acid, calcium levulinate, pentanoic acid, dibasic calcium phosphate, phosphoric acid, tribasic calcium phosphate, calcium hydroxide phosphate, potassium acetate, potassium chloride, potassium gluconate, potassium mixtures, dibasic potassium phosphate, monobasic potassium phosphate, potassium phosphate mixtures, sodium acetate, sodium bicarbonate, sodium chloride, sodium citrate, sodium lactate, dibasic sodium phosphate, monobasic sodium phosphate, sodium phosphate mixtures, tromethamine, magnesium hydroxide, aluminum hydroxide, alginic acid, pyrogen-free water, isotonic saline, Ringer's solution, ethyl alcohol, and mixtures thereof.

Exemplary lubricating agents include magnesium stearate, calcium stearate, stearic acid, silica, talc, malt, glyceryl behanate, hydrogenated vegetable oils, polyethylene glycol, sodium benzoate, sodium acetate, sodium chloride, leucine, magnesium lauryl sulfate, sodium lauryl sulfate, and mixtures thereof.

Exemplary natural oils include almond, apricot kernel, avocado, babassu, bergamot, black current seed, borage, cade, camomile, canola, caraway, carnauba, castor, cinnamon, cocoa butter, coconut, cod liver, coffee, corn, cotton seed, emu, eucalyptus, evening primrose, fish, flaxseed, geraniol, gourd, grape seed, hazel nut, hyssop, isopropyl myristate, jojoba, kukui nut, lavandin, lavender, lemon, litsea cubeba, macademia nut, mallow, mango seed, meadowfoam seed, mink, nutmeg, olive, orange, orange roughy, palm, palm kernel, peach kernel, peanut, poppy seed, pumpkin seed, rapeseed, rice bran, rosemary, safflower, sandalwood, sasquana, savoury, sea buckthorn, sesame, shea butter, silicone, soybean, sunflower, tea tree, thistle, tsubaki, vetiver, walnut, and wheat germ oils. Exemplary synthetic oils include, but are not limited to, butyl stearate, caprylic triglyceride, capric triglyceride, cyclomethicone, diethyl sebacate, dimethicone 360, isopropyl myristate, mineral oil, octyldodecanol, oleyl alcohol, silicone oil, and mixtures thereof.

Liquid dosage forms foral and parenteral administration include pharmaceutically acceptable emulsions, microemulsions, solutions, suspensions, syrups and elixirs. In addition to the active ingredients, the liquid dosage forms may comprise inert diluents commonly used in the art such as, for example, water or other solvents, solubilizing agents and emulsifiers such as ethyl alcohol, isopropyl alcohol, ethyl carbonate, ethyl acetate, benzyl alcohol, benzyl benzoate, propylene glycol, 1,3-butylene glycol, dimethylformamide, oils (e.g., cottonseed, groundnut, corn, germ, olive, castor, and sesame oils), glycerol, tetrahydrofurfuryl alcohol, polyethylene glycols and fatty acid esters of sorbitan, and mixtures thereof. Besides inert diluents, the oral compositions can include adjuvants such as wetting agents, emulsifying and suspending agents, sweetening, flavoring, and perfuming agents. In certain embodiments for parenteral administration, the conjugates described herein are mixed with solubilizing agents such as Cremophor®, alcohols, oils, modified oils, glycols, polysorbates, cyclodextrins, polymers, and mixtures thereof.

Injectable preparations, for example, sterile injectable aqueous or oleaginous suspensions can be formulated according to the known art using suitable dispersing or wetting agents and suspending agents. The sterile injectable preparation can be a sterile injectable solution, suspension, or emulsion in a nontoxic parenterally acceptable diluent or solvent, for example, as a solution in 1,3-butanediol. Among the acceptable vehicles and solvents that can be employed are water, Ringer's solution, U.S.P., and isotonic sodium chloride solution. In addition, sterile, fixed oils are conventionally employed as a solvent or suspending medium. For this purpose any bland fixed oil can be employed including synthetic mono- or di-glycerides. In addition, fatty acids such as oleic acid are used in the preparation of injectables.

The injectable formulations can be sterilized, for example, by filtration through a bacterial-retaining filter, or by incorporating sterilizing agents in the form of sterile solid compositions which can be dissolved or dispersed in sterile water or other sterile injectable medium prior to use.

In order to prolong the effect of a drug, it is often desirable to slow the absorption of the drug from subcutaneous or intramuscular injection. This can be accomplished by the use of a liquid suspension of crystalline or amorphous material with poor water solubility. The rate of absorption of the drug then depends upon its rate of dissolution, which, in turn, may depend upon crystal size and crystalline form. Alternatively, delayed absorption of a parenterally administered drug form may be accomplished by dissolving or suspending the drug in an oil vehicle.

Compositions for rectal or vaginal administration are typically suppositories which can be prepared by mixing the conjugates described herein with suitable non-irritating excipients or carriers such as cocoa butter, polyethylene glycol, or a suppository wax which are solid at ambient temperature but liquid at body temperature and therefore melt in the rectum or vaginal cavity and release the active ingredient.

Solid dosage forms foral administration include capsules, tablets, pills, powders, and granules. In such solid dosage forms, the active ingredient is mixed with at least one inert, pharmaceutically acceptable excipient or carrier such as sodium citrate or dicalcium phosphate and/or (a) fillers or extenders such as starches, lactose, sucrose, glucose, mannitol, and silicic acid, (b) binders such as, for example, carboxymethylcellulose, alginates, gelatin, polyvinylpyrrolidinone, sucrose, and acacia, (c) humectants such as glycerol, (d) disintegrating agents such as agar, calcium carbonate, potato or tapioca starch, alginic acid, certain silicates, and sodium carbonate, (e) solution retarding agents such as paraffin, (f) absorption accelerators such as quaternary ammonium compounds, (g) wetting agents such as, for example, cetyl alcohol and glycerol monostearate, (h) absorbents such as kaolin and bentonite clay, and (i) lubricants such as talc, calcium stearate, magnesium stearate, solid polyethylene glycols, sodium lauryl sulfate, and mixtures thereof. In the case of capsules, tablets, and pills, the dosage form may include a buffering agent.

Solid compositions of a similar type can be employed as fillers in soft and hard-filled gelatin capsules using such excipients as lactose or milk sugar as well as high molecular weight polyethylene glycols and the like. The solid dosage forms of tablets, dragees, capsules, pills, and granules can be prepared with coatings and shells such as enteric coatings and other coatings well known in the art of pharmacology. They may optionally comprise opacifying agents and can be of a composition that they release the active ingredient(s) only, or preferentially, in a certain part of the intestinal tract, optionally, in a delayed manner. Examples of encapsulating compositions which can be used include polymeric substances and waxes. Solid compositions of a similar type can be employed as fillers in soft and hard-filled gelatin capsules using such excipients as lactose or milk sugar as well as high molecular weight polyethylene glycols and the like.

The active ingredient can be in a micro-encapsulated form with one or more excipients as noted above. The solid dosage forms of tablets, dragees, capsules, pills, and granules can be prepared with coatings and shells such as enteric coatings, release controlling coatings, and other coatings well known in the pharmaceutical formulating art. In such solid dosage forms the active ingredient can be admixed with at least one inert diluent such as sucrose, lactose, or starch. Such dosage forms may comprise, as is normal practice, additional substances other than inert diluents, e.g., tableting lubricants and other tableting aids such a magnesium stearate and microcrystalline cellulose. In the case of capsules, tablets and pills, the dosage forms may comprise buffering agents. They may optionally comprise opacifying agents and can be of a composition that they release the active ingredient(s) only, or preferentially, in a certain part of the intestinal tract, optionally, in a delayed manner. Examples of encapsulating agents which can be used include polymeric substances and waxes.

Dosage forms for topical and/or transdermal administration of a compound described herein may include ointments, pastes, creams, lotions, gels, powders, solutions, sprays, inhalants, and/or patches. Generally, the active ingredient is admixed under sterile conditions with a pharmaceutically acceptable carrier or excipient and/or any needed preservatives and/or buffers as can be required. Additionally, the present disclosure contemplates the use of transdermal patches, which often have the added advantage of providing controlled delivery of an active ingredient to the body. Such dosage forms can be prepared, for example, by dissolving and/or dispensing the active ingredient in the proper medium. Alternatively or additionally, the rate can be controlled by either providing a rate controlling membrane and/or by dispersing the active ingredient in a polymer matrix and/or gel.

Suitable devices for use in delivering intradermal pharmaceutical compositions described herein include short needle devices. Intradermal compositions can be administered by devices which limit the effective penetration length of a needle into the skin. Alternatively or additionally, conventional syringes can be used in the classical mantoux method of intradermal administration. Jet injection devices which deliver liquid formulations to the dermis via a liquid jet injector and/or via a needle which pierces the stratum corneum and produces a jet which reaches the dermis are suitable. Ballistic powder/particle delivery devices which use compressed gas to accelerate the compound in powder form through the outer layers of the skin to the dermis are suitable.

Formulations suitable for topical administration include, but are not limited to, liquid and/or semi-liquid preparations such as liniments, lotions, oil-in-water and/or water-in-oil emulsions such as creams, ointments, and/or pastes, and/or solutions and/or suspensions. Topically administrable formulations may, for example, comprise from about 1% to about 10% (w/w) active ingredient, although the concentration of the active ingredient can be as high as the solubility limit of the active ingredient in the solvent. Formulations for topical administration may further comprise one or more of the additional ingredients described herein.

A pharmaceutical composition described herein can be prepared, packaged, and/or sold in a formulation suitable for pulmonary administration via the buccal cavity. Such a formulation may comprise dry particles which comprise the active ingredient and which have a diameter in the range from about 0.5 to about 7 nanometers, or from about 1 to about 6 nanometers. Such compositions are conveniently in the form of dry powders for administration using a device comprising a dry powder reservoir to which a stream of propellant can be directed to disperse the powder and/or using a self-propelling solvent/powder dispensing container such as a device comprising the active ingredient dissolved and/or suspended in a low-boiling propellant in a sealed container. Such powders comprise particles wherein at least 98% of the particles by weight have a diameter greater than 0.5 nanometers and at least 95% of the particles by number have a diameter less than 7 nanometers. Alternatively, at least 95% of the particles by weight have a diameter greater than 1 nanometer and at least 90% of the particles by number have a diameter less than 6 nanometers. Dry powder compositions may include a solid fine powder diluent such as sugar and are conveniently provided in a unit dose form.

Low boiling propellants generally include liquid propellants having a boiling point of below 65° F. at atmospheric pressure. Generally, the propellant may constitute 50 to 99.9% (w/w) of the composition, and the active ingredient may constitute 0.1 to 20% (w/w) of the composition. The propellant may further comprise additional ingredients such as a liquid non-ionic and/or solid anionic surfactant and/or a solid diluent (which may have a particle size of the same order as particles comprising the active ingredient).

Pharmaceutical compositions described herein formulated for pulmonary delivery may provide the active ingredient in the form of droplets of a solution and/or suspension. Such formulations can be prepared, packaged, and/or sold as aqueous and/or dilute alcoholic solutions and/or suspensions, optionally sterile, comprising the active ingredient, and may conveniently be administered using any nebulization and/or atomization device. Such formulations may further comprise one or more additional ingredients including, but not limited to, a flavoring agent such as saccharin sodium, a volatile oil, a buffering agent, a surface active agent, and/or a preservative such as methylhydroxybenzoate. The droplets provided by this route of administration may have an average diameter in the range from about 0.1 to about 200 nanometers.

Formulations described herein as being useful for pulmonary delivery are useful for intranasal delivery of a pharmaceutical composition described herein. Another formulation suitable for intranasal administration is a coarse powder comprising the active ingredient and having an average particle from about 0.2 to 500 micrometers. Such a formulation is administered by rapid inhalation through the nasal passage from a container of the powder held close to the nares.

Formulations for nasal administration may, for example, comprise from about as little as 0.1% (w/w) to as much as 100% (w/w) of the active ingredient, and may comprise one or more of the additional ingredients described herein. A pharmaceutical composition described herein can be prepared, packaged, and/or sold in a formulation for buccal administration. Such formulations may, for example, be in the form of tablets and/or lozenges made using conventional methods, and may contain, for example, 0.1 to 20% (w/w) active ingredient, the balance comprising an orally dissolvable and/or degradable composition and, optionally, one or more of the additional ingredients described herein. Alternately, formulations for buccal administration may comprise a powder and/or an aerosolized and/or atomized solution and/or suspension comprising the active ingredient. Such powdered, aerosolized, and/or aerosolized formulations, when dispersed, may have an average particle and/or droplet size in the range from about 0.1 to about 200 nanometers, and may further comprise one or more of the additional ingredients described herein.

A pharmaceutical composition described herein can be prepared, packaged, and/or sold in a formulation for ophthalmic administration. Such formulations may, for example, be in the form of eye drops including, for example, a 0.1-1.0% (w/w) solution and/or suspension of the active ingredient in an aqueous or oily liquid carrier or excipient. Such drops may further comprise buffering agents, salts, and/or one or more other of the additional ingredients described herein. Other ophthalmically-administrable formulations which are useful include those which comprise the active ingredient in microcrystalline form and/or in a liposomal preparation. Ear drops and/or eye drops are also contemplated as being within the scope of this disclosure.

Although the descriptions of pharmaceutical compositions provided herein are principally directed to pharmaceutical compositions which are suitable for administration to humans, it will be understood by the skilled artisan that such compositions are generally suitable for administration to animals of all sorts. Modification of pharmaceutical compositions suitable for administration to humans in order to render the compositions suitable for administration to various animals is well understood, and the ordinarily skilled veterinary pharmacologist can design and/or perform such modification with ordinary experimentation.

Compounds provided herein are typically formulated in dosage unit form for ease of administration and uniformity of dosage. It will be understood, however, that the total daily usage of the compositions described herein will be decided by a physician within the scope of sound medical judgment. The specific therapeutically effective dose level for any particular subject organism will depend upon a variety of factors including the disease being treated and the severity of the disorder; the activity of the specific active ingredient employed; the specific composition employed; the age, body weight, general health, sex, and diet of the subject; the time of administration, route of administration, and rate of excretion of the specific active ingredient employed; the duration of the treatment; drugs used in combination or coincidental with the specific active ingredient employed; and like factors well known in the medical arts.

The compounds and compositions provided herein can be administered by any route, including enteral (e.g., oral), parenteral, intravenous, intramuscular, intra-arterial, intramedullary, intrathecal, subcutaneous, intraventricular, transdermal, interdermal, rectal, intravaginal, intraperitoneal, topical (as by powders, ointments, creams, and/or drops), mucosal, nasal, bucal, sublingual; by intratracheal instillation, bronchial instillation, and/or inhalation; and/or as an oral spray, nasal spray, and/or aerosol. Specifically contemplated routes are oral administration, intravenous administration (e.g., systemic intravenous injection), regional administration via blood and/or lymph supply, and/or direct administration to an affected site. In general, the most appropriate route of administration will depend upon a variety of factors including the nature of the agent (e.g., its stability in the environment of the gastrointestinal tract), and/or the condition of the subject (e.g., whether the subject is able to tolerate oral administration). In certain embodiments, the compound or pharmaceutical composition described herein is suitable for topical administration to the eye of a subject.

The exact amount of a compound required to achieve an effective amount will vary from subject to subject, depending, for example, on species, age, and general condition of a subject, severity of the side effects or disorder, identity of the particular compound, mode of administration, and the like. An effective amount may be included in a single dose (e.g., single oral dose) or multiple doses (e.g., multiple oral doses). In certain embodiments, when multiple doses are administered to a subject or applied to a biological sample, tissue, or cell, any two doses of the multiple doses include different or substantially the same amounts of a compound described herein. In certain embodiments, when multiple doses are administered to a subject or applied to a biological sample, tissue, or cell, the frequency of administering the multiple doses to the subject or applying the multiple doses to the biological sample, tissue, or cell is three doses a day, two doses a day, one dose a day, one dose every other day, one dose every third day, one dose every week, one dose every two weeks, one dose every three weeks, or one dose every four weeks. In certain embodiments, the frequency of administering the multiple doses to the subject or applying the multiple doses to the biological sample, tissue, or cell is one dose per day. In certain embodiments, the frequency of administering the multiple doses to the subject or applying the multiple doses to the biological sample, tissue, or cell is two doses per day. In certain embodiments, the frequency of administering the multiple doses to the subject or applying the multiple doses to the biological sample, tissue, or cell is three doses per day. In certain embodiments, when multiple doses are administered to a subject or applied to a biological sample, tissue, or cell, the duration between the first dose and last dose of the multiple doses is one day, two days, four days, one week, two weeks, three weeks, one month, two months, three months, four months, six months, nine months, one year, two years, three years, four years, five years, seven years, ten years, fifteen years, twenty years, or the lifetime of the subject, or biological sample (e.g., tissue, or cell). In certain embodiments, the duration between the first dose and last dose of the multiple doses is three months, six months, or one year. In certain embodiments, the duration between the first dose and last dose of the multiple doses is the lifetime of the subject or biological sample (e.g., tissue, or cell). In certain embodiments, a dose (e.g., a single dose, or any dose of multiple doses) described herein includes independently between 0.1 μg and 1 μg, between 0.001 mg and 0.01 mg, between 0.01 mg and 0.1 mg, between 0.1 mg and 1 mg, between 1 mg and 3 mg, between 3 mg and 10 mg, between 10 mg and 30 mg, between 30 mg and 100 mg, between 100 mg and 300 mg, between 300 mg and 1,000 mg, or between 1 g and 10 g, inclusive, of a compound described herein. In certain embodiments, a dose described herein includes independently between 1 mg and 3 mg, inclusive, of a compound described herein. In certain embodiments, a dose described herein includes independently between 3 mg and 10 mg, inclusive, of a compound described herein. In certain embodiments, a dose described herein includes independently between 10 mg and 30 mg, inclusive, of a compound described herein. In certain embodiments, a dose described herein includes independently between 30 mg and 100 mg, inclusive, of a compound described herein.

Dose ranges as described herein provide guidance for the administration of provided pharmaceutical compositions to an adult. The amount to be administered to, for example, a child or an adolescent can be determined by a medical practitioner or person skilled in the art and can be lower or the same as that administered to an adult.

A compound or composition, as described herein, can be administered in combination with one or more additional pharmaceutical agents (e.g., therapeutically and/or prophylactically active agents). The compounds or compositions can be administered in combination with additional pharmaceutical agents that improve their activity (e.g., activity (e.g., potency and/or efficacy) in treating a disease in a subject in need thereof, in preventing a disease in a subject in need thereof, in inhibiting the activity of a thymidylate synthase, dihydrofolate reductase in a subject, biological sample, tissue, or cell), improve bioavailability, improve safety, reduce drug resistance, reduce and/or modify metabolism, inhibit excretion, and/or modify distribution in a subject, biological sample, tissue, or cell. It will also be appreciated that the therapy employed may achieve a desired effect for the same disorder, and/or it may achieve different effects. In certain embodiments, a pharmaceutical composition described herein including a compound described herein and an additional pharmaceutical agent shows a synergistic effect that is absent in a pharmaceutical composition including one of the compound and the additional pharmaceutical agent, but not both.

The compound or composition can be administered concurrently with, prior to, or subsequent to one or more additional pharmaceutical agents, which may be useful as, e.g., combination therapies. Pharmaceutical agents include therapeutically active agents. Pharmaceutical agents also include prophylactically active agents. Pharmaceutical agents include small organic molecules such as drug compounds (e.g., compounds approved for human or veterinary use by the U.S. Food and Drug Administration as provided in the Code of Federal Regulations (CFR)), peptides, proteins, carbohydrates, monosaccharides, oligosaccharides, polysaccharides, nucleoproteins, mucoproteins, lipoproteins, synthetic polypeptides or proteins, small molecules linked to proteins, glycoproteins, steroids, nucleic acids, DNAs, RNAs, nucleotides, nucleosides, oligonucleotides, antisense oligonucleotides, lipids, hormones, vitamins, and cells. In certain embodiments, the additional pharmaceutical agent is a pharmaceutical agent useful for treating and/or preventing a disease (e.g., proliferative disease, such as, cancers, or other diseases associated with a folate-dependent enzyme (e.g., thymidylate synthase, dihydrofolate reductase)). In certain embodiments, the additional pharmaceutical agent is a pharmaceutical agent useful for treating diseases associated with a folate-dependent enzyme (e.g., thymidylate synthase, dihydrofolate reductase). In certain embodiments, the additional pharmaceutical agent is a chemotherapeutic agent. In certain embodiments, the additional pharmaceutical agent is a thymidylate synthase inhibitor. In certain embodiments, the additional pharmaceutical agent is an inhibitor of phosphoinositide-3-kinase (PI-3K); an inhibitor of serine-threonine protein kinase B (AKT); an inhibitor of mammalian target of rapamycin (mTOR). Each additional pharmaceutical agent may be administered at a dose and/or on a time schedule determined for that pharmaceutical agent. The additional pharmaceutical agents may also be administered together with each other and/or with the compound or composition described herein in a single dose or administered separately in different doses. The particular combination to employ in a regimen will take into account compatibility of the compound described herein with the additional pharmaceutical agent(s) and/or the desired therapeutic and/or prophylactic effect to be achieved. In general, it is expected that the additional pharmaceutical agent(s) in combination be utilized at levels that do not exceed the levels at which they are utilized individually. In some embodiments, the levels utilized in combination will be lower than those utilized individually.

The additional pharmaceutical agents include, but are not limited to, anti-proliferative agents, anti-cancer agents, anti-angiogenesis agents, anti-inflammatory agents, immunosuppressants, anti-bacterial agents, anti-viral agents, cardiovascular agents, cholesterol-lowering agents, anti-diabetic agents, anti-allergic agents, contraceptive agents, pain-relieving agents, and a combination thereof. In certain embodiments, the additional pharmaceutical agent is an anti-proliferative agent (e.g., anti-cancer agent). In certain embodiments, the additional pharmaceutical agent is an anti-leukemia agent. In certain embodiments, the additional pharmaceutical agent is ABITREXATE (methotrexate), ADE, Adriamycin RDF (doxorubicin hydrochloride), Ambochlorin (chlorambucil), ARRANON (nelarabine), ARZERRA (ofatumumab), BOSULIF (bosutinib), BUSULFEX (busulfan), CAMPATH (alemtuzumab), CERUBIDINE (daunorubicin hydrochloride), CLAFEN (cyclophosphamide), CLOFAREX (clofarabine), CLOLAR (clofarabine), CVP, CYTOSAR-U (cytarabine), CYTOXAN (cyclophosphamide), ERWINAZE (Asparaginase Erwinia chrysanthemi), FLUDARA (fludarabine phosphate), FOLEX (methotrexate), FOLEX PFS (methotrexate), GAZYVA (obinutuzumab), GLEEVEC (imatinib mesylate), Hyper-CVAD, ICLUSIG (ponatinib hydrochloride), IMBRUVICA (ibrutinib), LEUKERAN (chlorambucil), LINFOLIZIN (chlorambucil), MARQIBO (vincristine sulfate liposome), METHOTREXATE LPF (methorexate), MEXATE (methotrexate), MEXATE-AQ (methotrexate), mitoxantrone hydrochloride, MUSTARGEN (mechlorethamine hydrochloride), MYLERAN (busulfan), NEOSAR (cyclophosphamide), ONCASPAR (Pegaspargase), PURINETHOL (mercaptopurine), PURIXAN (mercaptopurine), Rubidomycin (daunorubicin hydrochloride), SPRYCEL (dasatinib), SYNRIBO (omacetaxine mepesuccinate), TARABINE PFS (cytarabine), TASIGNA (nilotinib), TREANDA (bendamustine hydrochloride), TRISENOX (arsenic trioxide), VINCASAR PFS (vincristine sulfate), ZYDELIG (idelalisib), or a combination thereof. In certain embodiments, the additional pharmaceutical agent is an anti-lymphoma agent. In certain embodiments, the additional pharmaceutical agent is ABITREXATE (methotrexate), ABVD, ABVE, ABVE-PC, ADCETRIS (brentuximab vedotin), ADRIAMYCIN PFS (doxorubicin hydrochloride), ADRIAMYCIN RDF (doxorubicin hydrochloride), AMBOCHLORIN (chlorambucil), AMBOCLORIN (chlorambucil), ARRANON (nelarabine), BEACOPP, BECENUM (carmustine), BELEODAQ (belinostat), BEXXAR (tositumomab and iodine 1131 tositumomab), BICNU (carmustine), BLENOXANE (bleomycin), CARMUBRIS (carmustine), CHOP, CLAFEN (cyclophosphamide), COPP, COPP-ABV, CVP, CYTOXAN (cyclophosphamide), DEPOCYT (liposomal cytarabine), DTIC-DOME (dacarbazine), EPOCH, FOLEX (methotrexate), FOLEX PFS (methotrexate), FOLOTYN (pralatrexate), HYPER-CVAD, ICE, IMBRUVICA (ibrutinib), INTRON A (recombinant interferon alfa-2b), ISTODAX (romidepsin), LEUKERAN (chlorambucil), LINFOLIZIN (chlorambucil), Lomustine, MATULANE (procarbazine hydrochloride), METHOTREXATE LPF (methotrexate), MEXATE (methotrexate), MEXATE-AQ (methotrexate), MOPP, MOZOBIL (plerixafor), MUSTARGEN (mechlorethamine hydrochloride), NEOSAR (cyclophosphamide), OEPA, ONTAK (denileukin diftitox), OPPA, R—CHOP, REVLIMID (lenalidomide), RITUXAN (rituximab), STANFORD V, TREANDA (bendamustine hydrochloride), VAMP, VELBAN (vinblastine sulfate), VELCADE (bortezomib), VELSAR (vinblastine sulfate), VINCASAR PFS (vincristine sulfate), ZEVALIN (ibritumomab tiuxetan), ZOLINZA (vorinostat), ZYDELIG (idelalisib), or a combination thereof. In certain embodiments, the additional pharmaceutical agent is REVLIMID (lenalidomide), DACOGEN (decitabine), VIDAZA (azacitidine), CYTOSAR-U (cytarabine), IDAMYCIN (idarubicin), CERUBIDINE (daunorubicin), LEUKERAN (chlorambucil), NEOSAR (cyclophosphamide), FLUDARA (fludarabine), LEUSTATIN (cladribine), or a combination thereof. In certain embodiments, the additional pharmaceutical agent is ABITREXATE (methotrexate), ABRAXANE (paclitaxel albumin-stabilized nanoparticle formulation), AC, AC-T, ADE, ADRIAMYCIN PFS (doxorubicin hydrochloride), ADRUCIL (fluorouracil), AFINITOR (everolimus), AFINITOR DISPERZ (everolimus), ALDARA (imiquimod), ALIMTA (pemetrexed disodium), AREDIA (pamidronate disodium), ARIMIDEX (anastrozole), AROMASIN (exemestane), AVASTIN (bevacizumab), BECENUM (carmustine), BEP, BICNU (carmustine), BLENOXANE (bleomycin), CAF, CAMPTOSAR (irinotecan hydrochloride), CAPOX, CAPRELSA (vandetanib), CARBOPLATIN-TAXOL, CARMUBRIS (carmustine), CASODEX (bicalutamide), CEENU (lomustine), CERUBIDINE (daunorubicin hydrochloride), CERVARIX (recombinant HPV bivalent vaccine), CLAFEN (cyclophosphamide), CMF, COMETRIQ (cabozantinib-s-malate), COSMEGEN (dactinomycin), CYFOS (ifosfamide), CYRAMZA (ramucirumab), CYTOSAR-U (cytarabine), CYTOXAN (cyclophosphamide), DACOGEN (decitabine), DEGARELIX, DOXIL (doxorubicin hydrochloride liposome), DOXORUBICIN HYDROCHLORIDE, DOX-SL (doxorubicin hydrochloride liposome), DTIC-DOME (dacarbazine), EFUDEX (fluorouracil), ELLENCE (epirubicin hydrochloride), ELOXATIN (oxaliplatin), ERBITUX (cetuximab), ERIVEDGE (vismodegib), ETOPOPHOS (etoposide phosphate), EVACET (doxorubicin hydrochloride liposome), FARESTON (toremifene), FASLODEX (fulvestrant), FEC, FEMARA (letrozole), FLUOROPLEX (fluorouracil), FOLEX (methotrexate), FOLEX PFS (methotrexate), FOLFIRI, FOLFIRI-BEVACIZUMAB, FOLFIRI-CETUXIMAB, FOLFIRINOX, FOLFOX, FU-LV, GARDASIL (recombinant human papillomavirus (HPV) quadrivalent vaccine), GEMCITABINE-CISPLATIN, GEMCITABINE-OXALIPLATIN, GEMZAR (gemcitabine hydrochloride), GILOTRIF (afatinib dimaleate), GLEEVEC (imatinib mesylate), GLIADEL (carmustine implant), GLIADEL WAFER (carmustine implant), HERCEPTIN (trastuzumab), HYCAMTIN (topotecan hydrochloride), IFEX (ifosfamide), IFOSFAMIDUM (ifosfamide), INLYTA (axitinib), INTRON A (recombinant interferon alfa-2b), IRESSA (gefitinib), IXEMPRA (ixabepilone), JAKAFI (ruxolitinib phosphate), JEVTANA (cabazitaxel), KADCYLA (ado-trastuzumab emtansine), KEYTRUDA (pembrolizumab), KYPROLIS (carfilzomib), LIPODOX (doxorubicin hydrochloride liposome), LUPRON (leuprolide acetate), LUPRON DEPOT (leuprolide acetate), LUPRON DEPOT-3 MONTH (leuprolide acetate), LUPRON DEPOT-4 MONTH (leuprolide acetate), LUPRON DEPOT-PED (leuprolide acetate), MEGACE (megestrol acetate), MEKINIST (trametinib), METHAZOLASTONE (temozolomide), METHOTREXATE LPF (methotrexate), MEXATE (methotrexate), MEXATE-AQ (methotrexate), MITOXANTRONE HYDROCHLORIDE, MITOZYTREX (mitomycin c), MOZOBIL (plerixafor), MUSTARGEN (mechlorethamine hydrochloride), MUTAMYCIN (mitomycin c), MYLOSAR (azacitidine), NAVELBINE (vinorelbine tartrate), NEOSAR (cyclophosphamide), NEXAVAR (sorafenib tosylate), NOLVADEX (tamoxifen citrate), NOVALDEX (tamoxifen citrate), OFF, PAD, PARAPLAT (carboplatin), PARAPLATIN (carboplatin), PEG-INTRON (peginterferon alfa-2b), PEMETREXED DISODIUM, PERJETA (pertuzumab), PLATINOL (cisplatin), PLATINOL-AQ (cisplatin), POMALYST (pomalidomide), prednisone, PROLEUKIN (aldesleukin), PROLIA (denosumab), PROVENGE (sipuleucel-t), REVLIMID (lenalidomide), RUBIDOMYCIN (daunorubicin hydrochloride), SPRYCEL (dasatinib), STIVARGA (regorafenib), SUTENT (sunitinib malate), SYLATRON (peginterferon alfa-2b), SYLVANT (siltuximab), SYNOVIR (thalidomide), TAC, TAFINLAR (dabrafenib), TARABINE PFS (cytarabine), TARCEVA (erlotinib hydrochloride), TASIGNA (nilotinib), TAXOL (paclitaxel), TAXOTERE (docetaxel), TEMODAR (temozolomide), THALOMID (thalidomide), TOPOSAR (etoposide), TORISEL (temsirolimus), TPF, TRISENOX (arsenic trioxide), TYKERB (lapatinib ditosylate), VECTIBIX (panitumumab), VEIP, VELBAN (vinblastine sulfate), VELCADE (bortezomib), VELSAR (vinblastine sulfate), VEPESID (etoposide), VIADUR (leuprolide acetate), VIDAZA (azacitidine), VINCASAR PFS (vincristine sulfate), VOTRIENT (pazopanib hydrochloride), WELLCOVORIN (leucovorin calcium), XALKORI (crizotinib), XELODA (capecitabine), XELOX, XGEVA (denosumab), XOFIGO (radium 223 dichloride), XTANDI (enzalutamide), YERVOY (ipilimumab), ZALTRAP (ziv-aflibercept), ZELBORAF (vemurafenib), ZOLADEX (goserelin acetate), ZOMETA (zoledronic acid), ZYKADIA (ceritinib), ZYTIGA (abiraterone acetate), ENMD-2076, PCI-32765, AC220, dovitinib lactate (TK1258, CHIR-258), BIBW 2992 (TOVOK™), SGX523, PF-04217903, PF-02341066, PF-299804, BMS-777607, ABT-869, MP470, BIBF 1120 (VARGATEF®), AP24534, JNJ-26483327, MGCD265, DCC-2036, BMS-690154, CEP-11981, tivozanib (AV-951), OSI-930, MM-121, XL-184, XL-647, and/or XL228), proteasome inhibitors (e.g., bortezomib (Velcade)), mTOR inhibitors (e.g., rapamycin, temsirolimus (CCI-779), everolimus (RAD-001), ridaforolimus, AP23573 (Ariad), AZD8055 (AstraZeneca), BEZ235 (Novartis), BGT226 (Norvartis), XL765 (Sanofi Aventis), PF-4691502 (Pfizer), GDC0980 (Genetech), SF1126 (Semafoe) and OSI-027 (OSI)), oblimersen, gemcitabine, carminomycin, leucovorin, pemetrexed, cyclophosphamide, dacarbazine, procarbizine, prednisolone, dexamethasone, campathecin, plicamycin, asparaginase, aminopterin, methopterin, porfiromycin, melphalan, leurosidine, leurosine, chlorambucil, trabectedin, procarbazine, discodermolide, carminomycin, aminopterin, and hexamethyl melamine, or a combination thereof. In certain embodiments, the additional pharmaceutical agent is ibrutinib. In certain embodiments, the additional pharmaceutical agent is a protein kinase inhibitor (e.g., tyrosine protein kinase inhibitor). In certain embodiments, the additional pharmaceutical agent is a binder or inhibitor of Bruton's tyrosine kinase (BTK). In certain embodiments, the additional pharmaceutical agent is selected from the group consisting of epigenetic or transcriptional modulators (e.g., DNA methyltransferase inhibitors, histone deacetylase inhibitors (HDAC inhibitors), lysine methyltransferase inhibitors), antimitotic drugs (e.g., taxanes and vinca alkaloids), hormone receptor modulators (e.g., estrogen receptor modulators and androgen receptor modulators), cell signaling pathway inhibitors (e.g., tyrosine protein kinase inhibitors), modulators of protein stability (e.g., proteasome inhibitors), Hsp90 inhibitors, glucocorticoids, all-trans retinoic acids, and other agents that promote differentiation. In certain embodiments, the compounds described herein or pharmaceutical compositions can be administered in combination with an anti-cancer therapy including, but not limited to, surgery, radiation therapy, transplantation (e.g., stem cell transplantation, bone marrow transplantation), immunotherapy, and chemotherapy. In certain embodiments, the compounds described herein or pharmaceutical compositions can be administered in combination with a pharmaceutical agent useful for treating and/or preventing a proliferative disease (e.g., cancer).

Also encompassed by the disclosure are kits (e.g., pharmaceutical packs). The kits provided may comprise a pharmaceutical composition or compound described herein and a container (e.g., a vial, ampule, bottle, syringe, and/or dispenser package, or other suitable container). In some embodiments, provided kits may optionally further include a second container comprising a pharmaceutical excipient for dilution or suspension of a pharmaceutical composition or compound described herein. In some embodiments, the pharmaceutical composition or compound described herein provided in the first container and the second container are combined to form one unit dosage form.

Thus, in one aspect, provided are kits including a first container comprising a compound or pharmaceutical composition described herein. In certain embodiments, the kits are useful for treating a disease (e.g., proliferative disease) in a subject in need thereof. In certain embodiments, the kits are useful for preventing a disease (e.g., proliferative disease, such as, cancers, or other diseases associated with a folate-dependent enzyme (e.g., thymidylate synthase, dihydrofolate reductase)) in a subject in need thereof. In certain embodiments, the kits are useful for inhibiting the activity (e.g., aberrant or unwanted activity, such as increased activity) of a thymidylate synthase, dihydrofolate reductase in a subject, biological sample, tissue, or cell.

In certain embodiments, a kit described herein further includes instructions for using the compound or pharmaceutical composition included in the kit. A kit described herein may also include information as required by a regulatory agency such as the U.S. Food and Drug Administration (FDA). In certain embodiments, the information included in the kits is prescribing information. In certain embodiments, the kits and instructions provide for treating a disease (e.g., proliferative disease, such as, cancers, or other diseases associated with a folate-dependent enzyme (e.g., thymidylate synthase, dihydrofolate reductase)) in a subject in need thereof. In certain embodiments, the kits and instructions provide for preventing a disease (e.g., proliferative disease, such as, cancers, or other diseases associated with a folate-dependent enzyme (e.g., thymidylate synthase, dihydrofolate reductase)) in a subject in need thereof. In certain embodiments, the kits and instructions provide for modulating (e.g., inhibiting) the activity (e.g., aberrant activity, such as increased activity) of a folate-dependent enzyme (e.g., thymidylate synthase, dihydrofolate reductase) in a subject, biological sample, tissue, or cell. A kit described herein may include one or more additional pharmaceutical agents described herein as a separate composition.

### Methods of Treatment and Uses

The present disclosure provides methods of modulating (e.g., inhibiting or increasing) the activity (e.g., aberrant activity, such as increased or decreased activity) of a thymidylate synthase, dihydrofolate reductase. The present disclosure provides methods of modulating (e.g., inhibiting or increasing) the activity (e.g., aberrant activity, such as increased or decreased activity) of a thymidylate synthase, dihydrofolate reductase in a subject, biological sample, or cell. The present disclosure also provides methods for the treatment of a wide range of diseases, such as diseases associated with the aberrant activity (e.g., increased activity) of a thymidylate synthase, dihydrofolate reductase, e.g., proliferative disease, such as, cancers, or other diseases associated with a folate-dependent enzyme (e.g., thymidylate synthase, dihydrofolate reductase)) in a subject in need thereof. The present disclosure provides methods for the treatment and/or prevention of a proliferative disease, such as, cancers, or other diseases associated with a folate-dependent enzyme (e.g., thymidylate synthase, dihydrofolate reductase)).

The present disclosure also provides a compound of Formula (I) or (II), a compound of Table A, or a pharmaceutically acceptable salt, co-crystal, tautomer, stereoisomer, solvate, hydrate, polymorph, isotopically enriched derivative, or prodrug, or composition thereof, for use in the treatment of a disease, such as proliferative disease, such as, cancers, or other diseases associated with folate-dependent enzymes (e.g., thymidylate synthase, dihydrofolate reductase), in a subject in need thereof. The present disclosure also provides a compound of Formula (I) or (II), a compound of Table A, or a pharmaceutically acceptable salt, co-crystal, tautomer, stereoisomer, solvate, hydrate, polymorph, isotopically enriched derivative, or prodrug, or composition thereof, for use in the treatment of a disease, such as proliferative disease, such as, cancers, or other diseases associated with folate-dependent enzymes (e.g., thymidylate synthase, dihydrofolate reductase), in a subject in need thereof.

The present disclosure also provides uses of a compound of Formula (I) or (II), a compound of Table A, or a pharmaceutically acceptable salt, co-crystal, tautomer, stereoisomer, solvate, hydrate, polymorph, isotopically enriched derivative, or prodrug, or composition thereof, in the manufacture of a medicament for the treatment of a disease, such as proliferative disease, such as, cancers, or other diseases associated with folate-dependent enzymes (e.g., thymidylate synthase, dihydrofolate reductase), in a subject in need thereof. The present disclosure also provides uses of a compound of Formula (I) or (II), a compound of Table A, or a pharmaceutically acceptable salt, co-crystal, tautomer, stereoisomer, solvate, hydrate, polymorph, isotopically enriched derivative, or prodrug, or composition thereof, in the manufacture of a medicament for the treatment of a disease, such as proliferative disease, such as, cancers, or other diseases associated with folate-dependent enzymes (e.g., thymidylate synthase, dihydrofolate reductase), in a subject in need thereof.

In another aspect, the present disclosure provides treating a disease, such as proliferative disease, such as, cancers, or other diseases associated with folate-dependent enzymes (e.g., thymidylate synthase, dihydrofolate reductase), the methods comprising administering to the subject an effective amount of a compound of Formula (I) or (II), or a compound of formula:

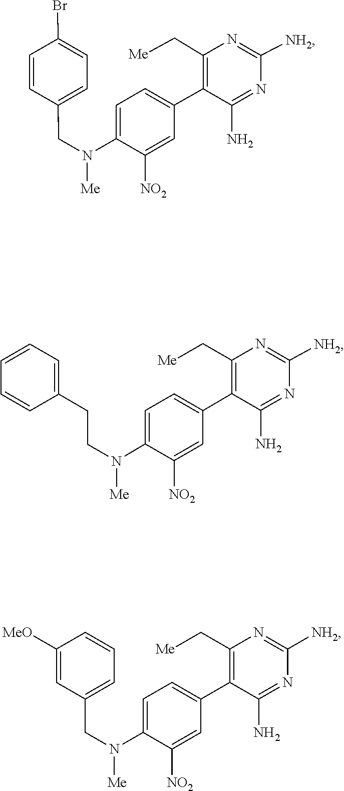 [SMILES: CCc1nc(N)nc(N)c1-c1ccc(N(C)Cc2ccc(Br)cc2)c([N+](=O)[O-])c1 ; CCc1nc(N)nc(N)c1-c1ccc(N(C)CCc2ccccc2)c([N+](=O)[O-])c1 ; CCc1nc(N)nc(N)c1-c1ccc(N(C)Cc2cccc(OC)c2)c([N+](=O)[O-])c1]

or a pharmaceutically acceptable salt, solvate, hydrate, polymorph, co-crystal, tautomer, stereoisomer, isotopically labeled derivative, prodrug, composition, or mixture thereof, or pharmaceutical composition thereof, as described herein.

In another aspect, the present disclosure provides methods of modulating the activity of a thymidylate synthase, dihydrofolate reductase in a subject, biological sample, or cell. In certain embodiments, provided are methods of inhibiting a folate dependent enzyme (e.g., thymidylate synthase, dihydrofolate reductase) in a subject. In certain embodiments, provided are methods of inhibiting a thymidylate synthase, dihydrofolate reductase in a cell. In certain embodiments, provided are methods of inhibiting the activity of a thymidylate synthase, dihydrofolate reductase in a subject. In certain embodiments, provided are methods of inhibiting the activity of a thymidylate synthase, dihydrofolate reductase in a cell. The compounds described herein may exhibit thymidylate synthase, dihydrofolate reductase inhibitory activity; the ability to inhibit a thymidylate synthase, dihydrofolate reductase; a therapeutic effect and/or preventative effect in the treatment of proliferative disease (e.g., cancers), or other diseases associated with a folate dependent enzyme (e.g., thymidylate synthase, dihydrofolate reductase) in a subject in need thereof. In certain embodiments, the compound being administered or used inhibits a folate-dependent enzyme (e.g., thymidylate synthase, dihydrofolate reductase) in a subject or cell, treats and/or prevents a disease, such as proliferative disease, such as, cancers, or other diseases associated with a folate-dependent enzyme (e.g., thymidylate synthase, dihydrofolate reductase)); and/or a therapeutic profile (e.g., optimum safety and curative effect) that is superior to existing chemotherapeutic agents, or agents for proliferative disease, such as, cancers, or other diseases associated with a folate-dependent enzyme (e.g., thymidylate synthase, dihydrofolate reductase)) in a subject in need thereof. In certain embodiments, the compound being administered or used inhibits a folate-dependent enzyme (e.g., thymidylate synthase, dihydrofolate reductase) in a subject or cell, treats and/or prevents a disease proliferative disease, such as, cancers, or other diseases associated with a folate-dependent enzyme (e.g., thymidylate synthase, dihydrofolate reductase)).

In certain embodiments, provided are methods of decreasing the activity of a folate-dependent enzyme (e.g., thymidylate synthase, dihydrofolate reductase) in a subject or biological sample (e.g., cell, tissue) by a method described herein by at least about 1%, at least about 3%, at least about 10%, at least about 20%, at least about 30%, at least about 40%, at least about 50%, at least about 60%, at least about 70%, at least about 80%, or at least about 90%. In certain embodiments, the activity of a folate-dependent enzyme (e.g., thymidylate synthase, dihydrofolate reductase) in a subject or cell is decreased by a method described herein by at least about 1%, at least about 3%, at least about 10%, at least about 20%, at least about 30%, at least about 40%, at least about 50%, at least about 60%, at least about 70%, at least about 80%, or at least about 90%. In some embodiments, the activity of a folate-dependent enzyme (e.g., thymidylate synthase, dihydrofolate reductase) in a subject or cell is selectively inhibited by the method. In some embodiments, the activity of a folate-dependent enzyme (e.g., thymidylate synthase, dihydrofolate reductase) in a subject or cell is selectively decreased by the method.

Without wishing to be bound by any particular theory, the compounds described herein are able to bind (e.g., modify) the folate-dependent enzyme (e.g., thymidylate synthase, dihydrofolate reductase) being inhibited. In certain embodiments, a compound described herein is able to bind (e.g., covalently modify) the folate-dependent enzyme (e.g., thymidylate synthase, dihydrofolate reductase). In certain embodiments, the compound described herein is able to bind in the deoxyuridine monophosphate (dUMP) binding site of thymidylate synthase. In certain embodiments, the compound is capable of binding in the folate (5,10-mTHF cofactor) binding site of thymidylate synthase. In certain embodiments, the compound is capable of binding Asp218 of the folate binding site for thymidylate synthase. In certain embodiments, the compound is capable of binding in the folate (5,10-mTHF cofactor) binding site of dihydrofolate reductase. In certain embodiments, the compound is capable of binding Ser59, Glu30, and/or Val8, and/or Phe34 of dihydrofolate reductase. In certain embodiments, the compound is capable of binding Ser59, Glu30, Val8, and Phe34 of dihydrofolate reductase.

In certain embodiments, the compound is capable of covalently binding a folate-dependent enzyme (e.g., thymidylate synthase, dihydrofolate reductase). In certain embodiments, the compound is capable of covalently binding thymidylate synthase, dihydrofolate reductase, In certain embodiments, the compound is capable of covalently modifying thymidylate synthase, dihydrofolate reductase.

In certain embodiments, the compound is capable of binding a folate-dependent enzyme (e.g., thymidylate synthase, dihydrofolate reductase). In certain embodiments, the compound is capable of binding thymidylate synthase and dihydrofolate reductase. In certain embodiments, the compound is capable of non-covalently modifying a folate-dependent enzyme (e.g., thymidylate synthase, dihydrofolate reductase). In certain embodiments, the compound is capable of non-covalently inhibiting a folate-dependent enzyme (e.g., thymidylate synthase, dihydrofolate reductase). In certain embodiments, the compound is capable of non-covalently modifying thymidylate synthase, dihydrofolate reductase. In certain embodiments, the compound is capable of non-covalently inhibiting thymidylate synthase, dihydrofolate reductase.

In another aspect, the present disclosure provides methods of inhibiting the activity of a folate-dependent enzyme (e.g., thymidylate synthase, dihydrofolate reductase) in a subject, the methods comprising administering to the subject an effective amount (e.g., therapeutically effective amount) of a compound, or pharmaceutical composition thereof, as described herein. In another aspect, the present disclosure provides methods of inhibiting the activity of a folate-dependent enzyme (e.g., thymidylate synthase, dihydrofolate reductase) in a biological sample, the methods comprising contacting the biological sample with an effective amount of a compound, or pharmaceutical composition thereof, as described herein. In another aspect, the present disclosure provides methods of inhibiting the activity of a folate-dependent enzyme (e.g., thymidylate synthase, dihydrofolate reductase) in a biological sample (e.g., tissue or cell), the methods comprising contacting the biological sample (e.g., tissue or cell) with an effective amount of a compound, or pharmaceutical composition thereof, as described herein.

In another aspect, the present disclosure provides methods of inhibiting the activity of a folate-dependent enzyme (e.g., thymidylate synthase, dihydrofolate reductase) in a biological sample (e.g., tissue or cell), the methods comprising contacting the biological sample (e.g., tissue or cell) with an effective amount of a compound, or pharmaceutical composition thereof, as described herein. In another aspect, the present disclosure provides methods of inhibiting (e.g., inhibiting the activity of) a folate-dependent enzyme (e.g., thymidylate synthase, dihydrofolate reductase) in a subject or biological sample, the methods comprising administering to the subject or contacting the biological sample (e.g., tissue or cell) with an effective amount of a compound of Formula (I) or (II), or a compound of formula:

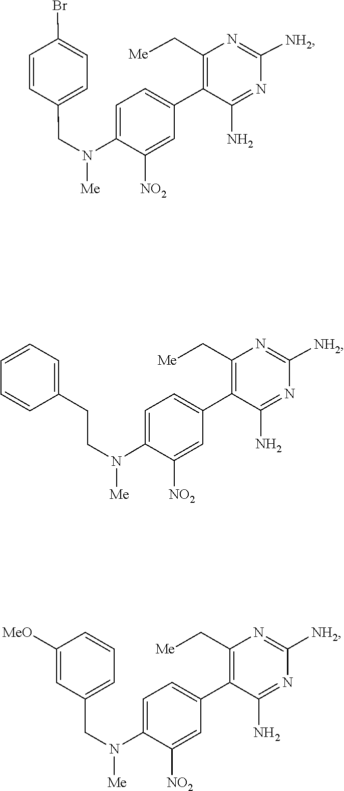 [SMILES: CCc1nc(N)nc(N)c1-c1ccc(N(C)Cc2ccc(Br)cc2)c([N+](=O)[O-])c1 ; CCc1nc(N)nc(N)c1-c1ccc(N(C)CCc2ccccc2)c([N+](=O)[O-])c1 ; CCc1nc(N)nc(N)c1-c1ccc(N(C)Cc2cccc(OC)c2)c([N+](=O)[O-])c1]

or a pharmaceutically acceptable salt, solvate, hydrate, polymorph, co-crystal, tautomer, stereoisomer, isotopically labeled derivative, prodrug, composition, or mixture thereof, or pharmaceutical composition thereof, as described herein. In certain embodiments, the subject being treated is a mammal. In certain embodiments, the subject is a human. In certain embodiments, the subject is a domesticated animal, such as a dog, cat, cow, pig, horse, sheep, or goat. In certain embodiments, the subject is a companion animal, such as a dog or cat. In certain embodiments, the subject is a livestock animal, such as a cow, pig, horse, sheep, or goat. In certain embodiments, the subject is a zoo animal. In another embodiment, the subject is a research animal such as a rodent, dog, or non-human primate. In certain embodiments, the subject is a non-human transgenic animal such as a transgenic mouse or transgenic pig.

In certain embodiments, the biological sample being contacted with the compound or composition is breast tissue, bone marrow, lymph node, lymph tissue, spleen, or blood. In certain embodiments, the biological sample being contacted with the compound or composition is a tumor cancerous tissue. In certain embodiments, the biological sample being contacted with the compound or composition is serum, cerebrospinal fluid, interstitial fluid, mucous, tears, sweat, pus, biopsied tissue (e.g., obtained by a surgical biopsy or needle biopsy), nipple aspirates, milk, vaginal fluid, saliva, swabs (such as buccal swabs), or any material containing biomolecules that is derived from a first biological sample.

In certain embodiments, the cell or tissue being contacted with the compound or composition is present in vitro. In certain embodiments, the cell or tissue being contacted with the compound or composition is present in vivo. In certain embodiments, the cell or tissue being contacted with the compound or composition is present ex vivo. In certain embodiments, the cell or tissue being contacted with the compound or composition is a malignant cell (e.g., malignant blood cell). In certain embodiments, the cell being contacted with the compound or composition is a malignant hematopoietic stem cell (e.g., malignant myeloid cell or malignant lymphoid cell). In certain embodiments, the cell being contacted with the compound or composition is a malignant lymphocyte (e.g., malignant T-cell or malignant B-cell). In certain embodiments, the cell being contacted with the compound or composition is a malignant white blood cell. In certain embodiments, the cell being contacted with the compound or composition is a malignant neutrophil, malignant macrophage, or malignant plasma cell. In certain embodiments, the cell being contacted with the compound or composition is a carcinoma cell. In certain embodiments, the cell being contacted with the compound or composition is a breast carcinoma cell. In certain embodiments, the cell being contacted with the compound or composition is a sarcoma cell. In certain embodiments, the cell being contacted with the compound or composition is a sarcoma cell from breast tissue.

In certain embodiments, the disease to be treated or prevented using the compounds described herein is a proliferative disease, such as cancer, for example, colon, pancreatic, or lung cancer. The disease (e.g., proliferative disease, such as cancers, for example, colon, pancreatic, and lung cancer), to be treated or prevented using the compounds described herein may be associated with increased activity of a folate-dependent enzyme (e.g., thymidylate synthase, dihydrofolate reductase). The disease (e.g., proliferative disease, such as, cancers) to be treated or prevented using the compounds described herein may be associated with the overexpression of a folate-dependent enzyme (e.g., thymidylate synthase, dihydrofolate reductase).

In certain embodiments, the disease (e.g., proliferative disease, such as, cancers), to be treated or prevented using the compounds described herein may be associated with the overexpression of a folate-dependent enzyme (e.g., thymidylate synthase, dihydrofolate reductase). In certain embodiments, a disease (e.g., proliferative disease, such as, cancers), may be associated with aberrant activity of a folate-dependent enzyme (e.g., thymidylate synthase, dihydrofolate reductase). Aberrant activity of a folate-dependent enzyme (e.g., thymidylate synthase, dihydrofolate reductase) may be elevated and/or inappropriate or undesired activity of the folate-dependent enzyme. The compounds described herein, and pharmaceutically acceptable salts, solvates, hydrates, polymorphs, co-crystals, tautomers, stereoisomers, isotopically labeled derivatives, prodrugs, and compositions thereof, may inhibit the activity of a folate-dependent enzyme (e.g., thymidylate synthase, dihydrofolate reductase) and be useful in treating and/or preventing diseases (e.g., proliferative disease, such as, cancers). The compounds described herein, and pharmaceutically acceptable salts, solvates, hydrates, polymorphs, co-crystals, tautomers, stereoisomers, isotopically labeled derivatives, prodrugs, and compositions thereof, may inhibit the activity of a folate-dependent enzyme (e.g., thymidylate synthase, dihydrofolate reductase) and be useful in treating and/or preventing diseases (e.g., proliferative disease, such as, cancers). The compounds described herein, and pharmaceutically acceptable salts, solvates, hydrates, polymorphs, co-crystals, tautomers, stereoisomers, isotopically labeled derivatives, prodrugs, and compositions thereof, may inhibit the activity of a folate-dependent enzyme (e.g., thymidylate synthase, dihydrofolate reductase) and be useful in treating and/or preventing diseases (e.g., proliferative disease, such as, cancers).

All types of biological samples described herein or known in the art are contemplated as being within the scope of the invention. In certain embodiments, the proliferative disease to be treated or prevented using the compounds described herein is cancer. All types of cancers disclosed herein or known in the art are contemplated as being within the scope of the invention. In certain embodiments, the cancer is a cancer treated by a standard anti-folate drug. In certain embodiments, the cancer is a cancer treated by 5-fluorouracil (5-FU), methotrexate, and/or pemetrexed. In certain embodiments, the proliferative disease is a hematological malignancy. In certain embodiments, the proliferative disease is a blood cancer. In certain embodiments, the proliferative disease is a hematological malignancy. In certain embodiments, the cancer is a carcinoma, lung cancer, breast cancer, liver cancer, pancreatic cancer, gastric cancer, ovarian cancer, cervical cancer, colon cancer, colorectal cancer, bladder cancer, hematopoietic cancer, and/or drug-resistant cancer. In certain embodiments, the cancer is a colon, pancreatic, or lung cancer. In certain embodiments, the cancer is hematopoietic cancer. In certain embodiments, the cancer is leukemia or lymphoma. In certain embodiments, the proliferative disease is leukemia. In certain embodiments, the leukemia is acute lymphoblastic leukemia (ALL), acute myelogenous leukemia (AML), acute promyelocytic leukemia, chronic lymphocytic leukemia (CLL), chronic myelogenous leukemia (CML), acute monocytic leukemia (AMOL), or hairy cell leukemia. In certain embodiments, the lymphoma is large cell immunoblastic lymphoma, Hodgkin's lymphoma, or non-Hodgkin's lymphoma. In certain embodiments, the hematopoietic cancer is plasmacytoma or multiple myeloma. In certain embodiments, the proliferative disease is chronic lymphocytic leukemia (CLL). In certain embodiments, the proliferative disease is acute lymphoblastic leukemia (ALL). In certain embodiments, the proliferative disease is T-cell acute lymphoblastic leukemia (T-ALL). In certain embodiments, the proliferative disease is chronic myelogenous leukemia (CML). In certain embodiments, the proliferative disease is acute myeloid leukemia (AML). In certain embodiments, the proliferative disease is acute monocytic leukemia (AMoL). In certain embodiments, the proliferative disease is a carcinoma. In certain embodiments, the proliferative disease is lymphoma. In certain embodiments, the proliferative disease is T-cell lymphoma. In some embodiments, the proliferative disease is Burkitt's lymphoma. In certain embodiments, the proliferative disease is a Hodgkin's lymphoma. In certain embodiments, the proliferative disease is a non-Hodgkin's lymphoma. In certain embodiments, the proliferative disease is multiple myeloma. In certain embodiments, the proliferative disease is melanoma. In certain embodiments, the proliferative disease is colorectal cancer. In certain embodiments, the proliferative disease is colon cancer. In certain embodiments, the proliferative disease is breast cancer. In certain embodiments, the proliferative disease is recurring breast cancer. In certain embodiments, the proliferative disease is mutant breast cancer. In certain embodiments, the proliferative disease is HER2+ breast cancer. In certain embodiments, the proliferative disease is HER2− breast cancer. In certain embodiments, the proliferative disease is triple-negative breast cancer (TNBC). In certain embodiments, the proliferative disease is a bone cancer. In certain embodiments, the proliferative disease is osteosarcoma. In certain embodiments, the proliferative disease is Ewing's sarcoma. In some embodiments, the proliferative disease is a brain cancer. In some embodiments, the proliferative disease is neuroblastoma. In some embodiments, the proliferative disease is a lung cancer. In some embodiments, the proliferative disease is small cell lung cancer (SCLC). In some embodiments, the proliferative disease is non-small cell lung cancer. In some embodiments, the proliferative disease is liver cancer. In some embodiments, the proliferative disease is pancreatic cancer. In some embodiments, the proliferative disease is gastric cancer. In some embodiments, the proliferative disease is ovarian cancer. In some embodiments, the proliferative disease is ovarian cancer. In certain embodiments, the proliferative disease is cervical cancer. In some embodiments, the proliferative disease is a benign neoplasm. All types of benign neoplasms disclosed herein or known in the art are contemplated as being within the scope of the invention. In some embodiments, the proliferative disease is associated with angiogenesis. All types of angiogenesis disclosed herein or known in the art are contemplated as being within the scope of the invention. In some embodiments, the cancer is resistant to a drug (e.g., 5-fluorouracil (5-FU)). In some embodiments, the cancer is resistant to standard chemotherapeutics (e.g., standard classical antifolate drugs related to folate transport and/or metabolism pathways).

Another aspect of the disclosure relates to methods of inhibiting the activity of a folate-dependent enzyme (e.g., thymidylate synthase, dihydrofolate reductase) in a biological sample (e.g., tissue, cell), or subject. In certain embodiments, the folate-dependent enzyme is a thymidylate synthase, dihydrofolate reductase. In certain embodiments, the folate-dependent enzyme is a thymidylate synthase. In certain embodiments, the folate-dependent enzyme is dihydrofolate reductase. In certain embodiments, the folate-dependent enzymes are thymidylate synthase and dihydrofolate reductase. In certain embodiments, the activity of the folate-dependent enzyme is aberrant activity of the folate-dependent enzyme (e.g., thymidylate synthase, dihydrofolate reductase). In certain embodiments, the activity of the folate-dependent enzyme (e.g., thymidylate synthase, dihydrofolate reductase) is increased activity of the folate-dependent enzyme (e.g., thymidylate synthase, dihydrofolate reductase). In certain embodiments, the inhibition of the activity of the folate-dependent enzyme (e.g., thymidylate synthase, dihydrofolate reductase) is irreversible. In other embodiments, the inhibition of the activity of the folate-dependent enzyme (e.g., thymidylate synthase, dihydrofolate reductase) is reversible. In certain embodiments, the methods of inhibiting the activity of the folate-dependent enzyme (e.g., thymidylate synthase, dihydrofolate reductase) include attaching a compound described herein to the folate-dependent enzyme (e.g., thymidylate synthase, dihydrofolate reductase). In certain embodiments, the methods comprise covalently inhibiting a folate-dependent enzyme (e.g., thymidylate synthase, dihydrofolate reductase). In certain embodiments, the methods comprise covalently inhibiting a folate-dependent enzyme (e.g., thymidylate synthase, dihydrofolate reductase). In certain embodiments, the methods comprise reversibly inhibiting a folate-dependent enzyme (e.g., thymidylate synthase, dihydrofolate reductase).

In certain embodiments, the methods described herein include administering to a subject or contacting a biological sample with an effective amount of a compound described herein, or a pharmaceutically acceptable salt, solvate, hydrate, polymorph, co-crystal, tautomer, stereoisomer, isotopically labeled derivative, or prodrug thereof, or a pharmaceutical composition thereof. In certain embodiments, the methods described herein include administering to a subject or contacting a biological sample with an effective amount of a compound described herein, or a pharmaceutically acceptable salt thereof, or a pharmaceutical composition thereof. In certain embodiments, the compound is contacted with a biological sample. In certain embodiments, the compound is administered to a subject. In certain embodiments, the compound is administered in combination with one or more additional pharmaceutical agents described herein. In certain embodiments, the additional pharmaceutical agent is a targeted therapy, for example, an FDA-approved targeted therapy (e.g., small molecules targeting molecular targets associated with cancers; monoclonal antibodies, for example, targeted therapies such as hormone therapies, signal transduction inhibitors, gene expression modulators, apoptosis inducers, angiogenesis inhibitors, immunotherapies, toxin delivery molecules) and/or chemotherapeutic agent (e.g., an anti-cancer agent). In certain embodiments, the additional pharmaceutical agent is a targeted therapy, for example, an FDA-approved targeted therapy (e.g., small molecules targeting molecular targets associated with cancers; monoclonal antibodies, for example, targeted therapies such as hormone therapies, signal transduction inhibitors, gene expression modulators, apoptosis inducers, angiogenesis inhibitors, immunotherapies, toxin delivery molecules). In certain embodiments, the additional pharmaceutical agent is a chemotherapeutic agent (e.g., an anti-cancer agent). The additional pharmaceutical agent may also be an inhibitor of a folate-dependent enzyme (e.g., thymidylate synthase, dihydrofolate reductase). In certain embodiments, the additional pharmaceutical agent is an inhibitor of a thymidylate synthase. In certain embodiments, the additional pharmaceutical agent is an inhibitor of dihydrofolate reductase. In certain embodiments, the additional pharmaceutical agent is an inhibitor of phosphoinositide-3-kinase (PI-3K); an inhibitor of serine-threonine protein kinase B (AKT); an inhibitor of mammalian target of rapamycin (mTOR). In some embodiments, the additional pharmaceutical agent is a topoisomerase inhibitor, a MCL1 inhibitor, a BCL-2 inhibitor, a BCL-xL inhibitor, a BRD4 inhibitor, a BRCA1 inhibitor, BRCA2 inhibitor, HER1 inhibitor, HER2 inhibitor, a CDK9 inhibitor, a Jumonji histone demethylase inhibitor, or a DNA damage inducer. In some embodiments, the additional pharmaceutical agent is etoposide, obatoclax, navitoclax, JQ1, 4-(((5′-chloro-2′-(((1R,4R)-4-(((R)-1-methoxypropan-2-yl)amino)cyclohexyl)amino)-[2,4′-bipyridin]-6-yl)amino)methyl)tetrahydro-2H-pyran-4-carbonitrile, JIB04, or cisplatin. Exemplary chemotherapeutic agents include alkylating agents such as nitrogen mustards, ethylenimines, methylmelamines, alkyl sulfonates, nitrosuoureas, and triazenes; antimetabolites such as folic acid analogs, pyrimidine analogs, in particular fluorouracil and cytosine arabinoside, and purine analogs; natural products such as vinca alkaloids epi-podophyllotoxins, antibiotics, enzymes, and biological response modifiers; and miscellaneous products such as platinum coordination complexes, anthracenedione, substituted urea such as hydroxyurea, methyl hydrazine derivatives, and adrenocorticoid suppressant. Exemplary chemotherapeutic agents also include anthracycline antibiotics, actinomycin D, plicamycin, puromycin, gramicidin D, paclitaxel, colchicine, cytochalasin B, emetine, maytansine, amsacrine, cisplatin, carboplatin, mitomycin, altretamine, cyclophosphamide, lomustine, and carmustine. In certain embodiments, a pharmaceutical composition described herein further comprises a combination of the additional pharmaceutical agents described herein.

The compounds or compositions may synergistically augment inhibition of a folate-dependent enzyme (e.g., thymidylate synthase, dihydrofolate reductase) induced by the additional pharmaceutical agent(s) in the biological sample or subject. Thus, the combination of the compounds or compositions and the additional pharmaceutical agent(s) may be useful in treating proliferative diseases resistant to a treatment using the additional pharmaceutical agent(s) without the compounds or compositions.

In some embodiments, the activity of a folate-dependent enzyme (e.g., thymidylate synthase, dihydrofolate reductase) is non-selectively inhibited by the compounds or pharmaceutical compositions described herein. In some embodiments, the activity of the folate-dependent enzyme (e.g., thymidylate synthase, dihydrofolate reductase) being inhibited is selectively inhibited by the compounds or pharmaceutical compositions described herein, compared to the activity of a folate-dependent enzyme (e.g., thymidylate synthase, dihydrofolate reductase). In certain embodiments, the activity of a folate-dependent enzyme (e.g., thymidylate synthase, dihydrofolate reductase) is selectively inhibited by a compound or pharmaceutical composition described herein, compared to the activity of a different protein. In certain embodiments, the activity of thymidylate synthase is selectively inhibited by a compound or pharmaceutical composition described herein, compared to the activity of another folate-dependent enzyme (e.g., thymidylate synthase, dihydrofolate reductase).

The selectivity of a compound or pharmaceutical composition described herein in inhibiting the activity of a thymidylate synthase over a different protein (e.g., a different folate-dependent enzyme) may be measured by the quotient of the IC₅₀ value of the compound or pharmaceutical composition in inhibiting the activity of the different protein over the IC₅₀ value of the compound or pharmaceutical composition in inhibiting the activity of the folate-dependent enzyme. The selectivity of a compound or pharmaceutical composition described herein for a folate-dependent enzyme over a different protein may also be measured by the quotient of the K_{d} value of an adduct of the compound or pharmaceutical composition and the different protein over the K_{d} value of an adduct of the compound or pharmaceutical composition and the folate-dependent enzyme. In certain embodiments, the selectivity is at least 2-fold, at least 3-fold, at least 5-fold, at least 10-fold, at least 30-fold, at least 100-fold, at least 300-fold, at least 1,000-fold, at least 3,000-fold, at least 10,000-fold, at least 30,000-fold, or at least 100,000-fold. In certain embodiments, the selectivity is not more than 100,000-fold, not more than 10,000-fold, not more than 1,000-fold, not more than 100-fold, not more than 10-fold, or not more than 2-fold. Combinations of the above-referenced ranges (e.g., at least 2-fold and not more than 10,000-fold) are also within the scope of the disclosure.

In certain embodiments, a kit described herein includes a first container comprising a compound or pharmaceutical composition described herein. In certain embodiments, a kit described herein is useful in treating and/or preventing a disease, such as a proliferative disease, such as, cancers, or other diseases associated with a folate-dependent enzyme (e.g., thymidylate synthase, dihydrofolate reductase)), in a subject in need thereof, inhibiting the activity of a folate-dependent enzyme (e.g., thymidylate synthase, dihydrofolate reductase) in a subject, biological sample, tissue, or cell.

In certain embodiments, a kit described herein further includes instructions for using the compound or pharmaceutical composition included in the kit. A kit described herein may also include information as required by a regulatory agency such as the U.S. Food and Drug Administration (FDA). In certain embodiments, the information included in the kits is prescribing information. In certain embodiments, the kits and instructions provide for treating and/or preventing a proliferative disease in a subject in need thereof, or other diseases associated with a folate-dependent enzyme (e.g., thymidylate synthase, dihydrofolate reductase) in a subject in need thereof, inhibiting the activity of a folate-dependent enzyme (e.g., thymidylate synthase, dihydrofolate reductase) in a subject, biological sample, tissue, or cell. A kit described herein may include one or more additional pharmaceutical agents described herein as a separate composition.

### EXAMPLES

In order that the present disclosure may be more fully understood, the following examples are set forth. The synthetic and biological examples described in this application are offered to illustrate the compounds, pharmaceutical compositions, and methods provided herein and are not to be construed in any way as limiting their scope. The compounds provided herein can be prepared from readily available starting materials using the following general methods and procedures or methods known in the art. It will be appreciated that where typical or preferred process conditions (i.e., reaction temperatures, times, mole ratios of reactants, solvents, pressures, etc.) are given, other process conditions can also be used unless otherwise stated. Optimum reaction conditions may vary with the particular reactants or solvents used, but such conditions can be determined by those skilled in the art by routine optimization procedures.

In the Examples, the data shows the development of TS inhibitors that are structurally distinct from folic acid and other classical antifolates, e.g., antifolates that require specific transporters or intracellular conversion/metabolism. These novel antifolates directly inhibit TS without inducing the TS overexpression associated with drug resistance. Using analogs based on an initial TS inhibitor scaffold, compound 19-S, the structural features associated with direct TS inhibition while maintaining the ability to inhibit DHFR were determined. These features enabled both direct TS inhibition and also indirect inhibition of thymidine synthesis, by preventing regeneration of the required 5,10-mTHF cofactor. The multifunctional non-classical antifolates were well tolerated when administered either by intraperitoneal (IP) injection or oral gavage (PO) and were effective at inhibiting tumor progression and extending survival using mouse pancreatic cancer tumor models.

### Example 1

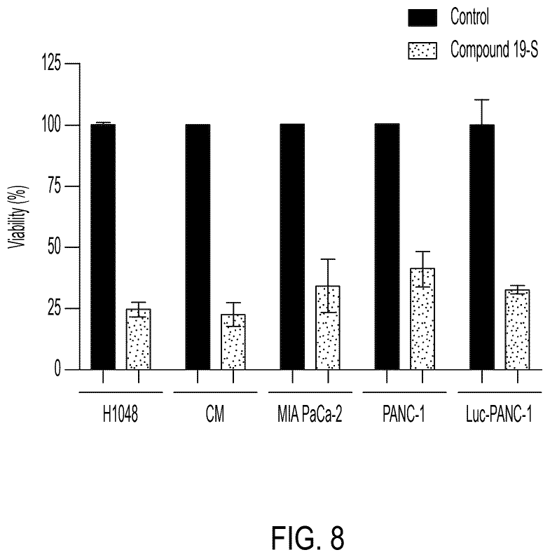

To identify potential TS small molecule inhibitors, molecular docking was used to computationally screen compounds with the ability to bind TS. The details of the procedure are presented in the Methods section. The top ranked 26 compounds were acquired and designated 1-A through 26-Z, then used to determine the cytotoxicity at a 10 uM concentration in several cancer cell lines. Compound 19-S (NSC 382035) exhibited cytotoxicity in the 5 cell lines examined (FIG. 8) and presented a promising lead candidate for further study and comparison against established TS inhibitors. This initial computational screen provided a predictive model that allowed a preliminary assessment for possible drug candidates. Subsequent experiments were pursued to validate biological activity and to define mechanisms for TS inhibition.

Small Molecule Compound Inhibits TS Catalytic Activity, Shows Cytotoxicity In Vitro and does not Increase TS Levels

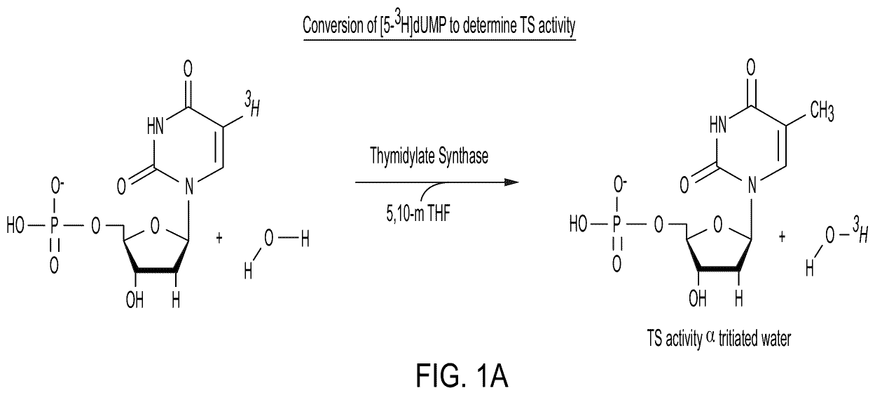

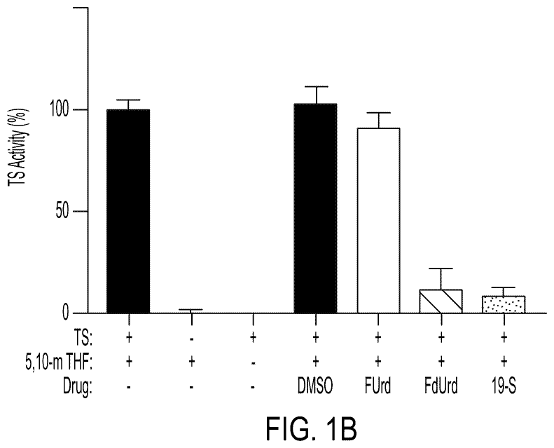

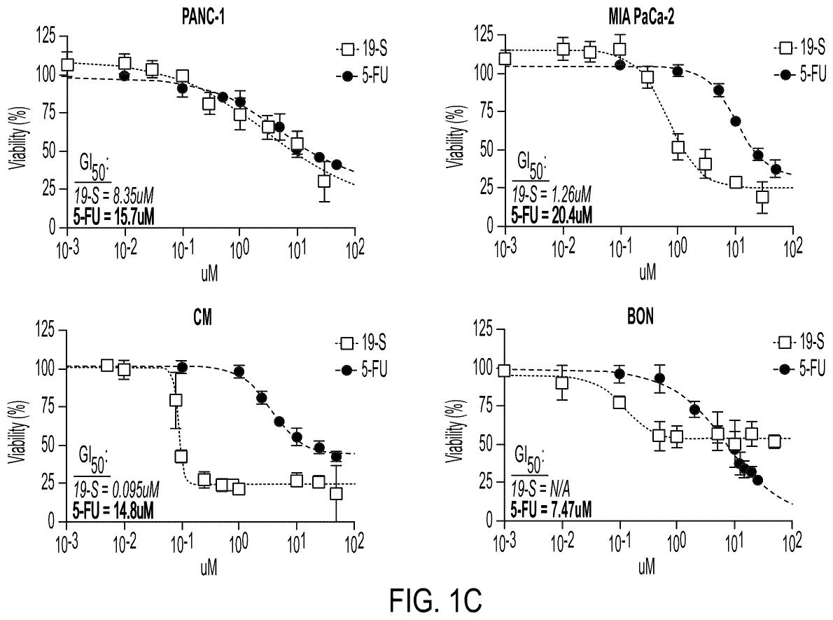

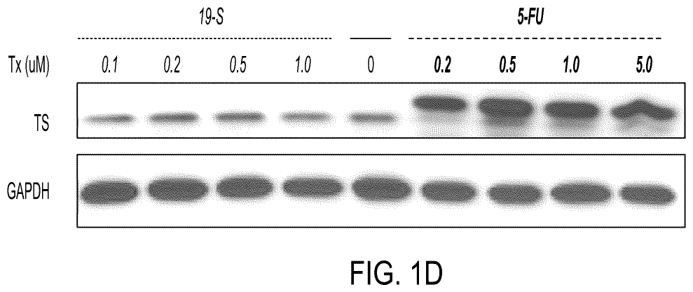

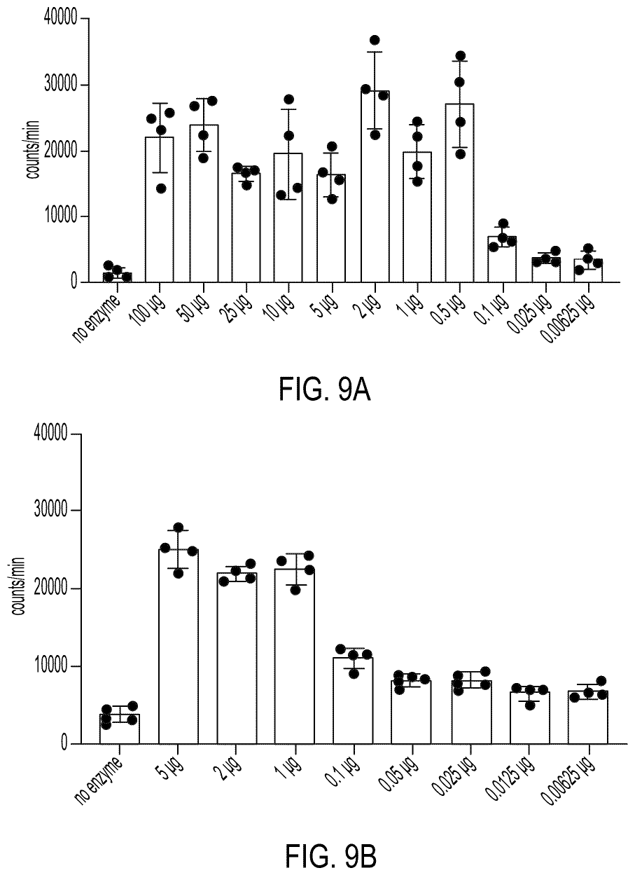

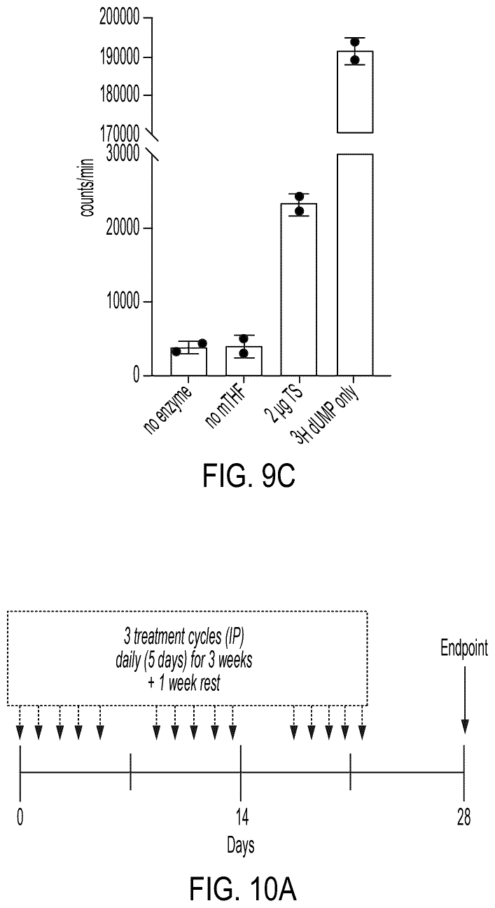

To determine the inhibition of TS activity, a tritium-based TS catalytic assay was utilized to quantify the conversion of dUMP which is proportional to the TS activity33 (FIG. 1A & FIGS. 9A-9C). Reactions were performed in the presence of compound 19-S or 5-FU metabolites controls, including 5-fluoro-2′-deoxyuridine (FdUrd) which is recognized to inhibit TS activity, and 5-fluorouridine (FUrd) that does not inhibit TS activity14. As expected, the 5-FU metabolite FdUrd reduced conversion of dUMP while FUrd was inactive (FIG. 1B). Compound 19-S yielded comparable TS inhibition as seen with the 5-FU metabolite FdUrd (FIG. 1B), confirming compound 19-S shows inhibition of TS activity.

To compare the cytotoxicity of compound 19-S relative to the TS inhibitor 5-FU, a panel of four established pancreatic cancer cell lines including two pancreatic ductal adenocarcinoma (PDAC) cells lines (PANC-1 and MIA PaCa-2) and two pancreatic neuroendocrine tumor (PanNET) cell lines (CM and BON) was utilized. Treatment with compound 19-S resulted in potent cytotoxicity in all cell lines examined (FIG. 1C). In MIA PaCa-2 the GI50 for compound 19-S was 16-fold lower than 5-FU (1.26 M vs 20.4 M), and in CM cells the GI50 was over 150-fold lower than 5-FU (0.095 M vs 14.8 M). In PANC-1, the GI50 for compound 19-S was 1.9-fold lower than observed for 5-FU (8.35 M vs 15.7 M, respectively), and the difference in GI50 was further magnified by the broad hill slope observed in both dose response curves (FIG. 1C). Since 50% viability was not reached for compound 19-S in BON cells the GI50 could not be calculated, although a more potent IC50 of 0.12 M with 19-S was observed as compared to an IC50 of 7.1 M for 5-FU (FIG. 1C).

Earlier studies have reported that the sustained therapeutic benefits of 5-FU therapy are limited by a common molecular mechanism: drug-induced elevation of TS expression that contributes to increased drug resistance and limiting 5-FU effectiveness. Therefore, treatment with compound 19-S was tested to determine if this exemplary compound also induced TS protein expression as compared to 5-FU treatment (FIG. 1D). 5-FU induced TS expression with the characteristic band shift caused by the covalently bound 5-FU that slows the migration of TS. In contrast, there was no increase in steady-state TS levels after treatment with compound 19-S (FIG. 1D), suggesting compound 19-S treatment can escape this drug-induced resistance mechanism.

### Compound 19-S Inhibits Tumor Growth in Xenograft Tumor Models

Based on the in vitro studies demonstrating the ability of 19-S to inhibit TS catalytic activity (FIG. 1B) and causing cytotoxicity in cancer cell lines (FIGS. 1C and 8), the in vivo effects of compound 19-S in two distinct xenograft tumor models were studied. It was demonstrated that compound 19-S inhibited tumor growth and progression in vivo. First a subcutaneous tumor model was tested with the PANC-1 cell line expressing firefly luciferase (Luc-PANC-1) and then tested with a disseminated tumor model with the CM cell line expressing firefly luciferase (Luc-CM).

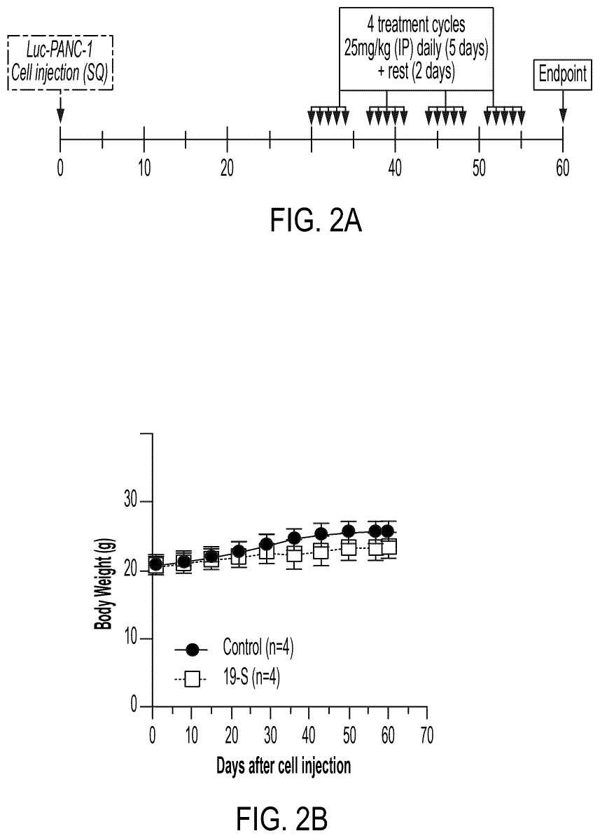

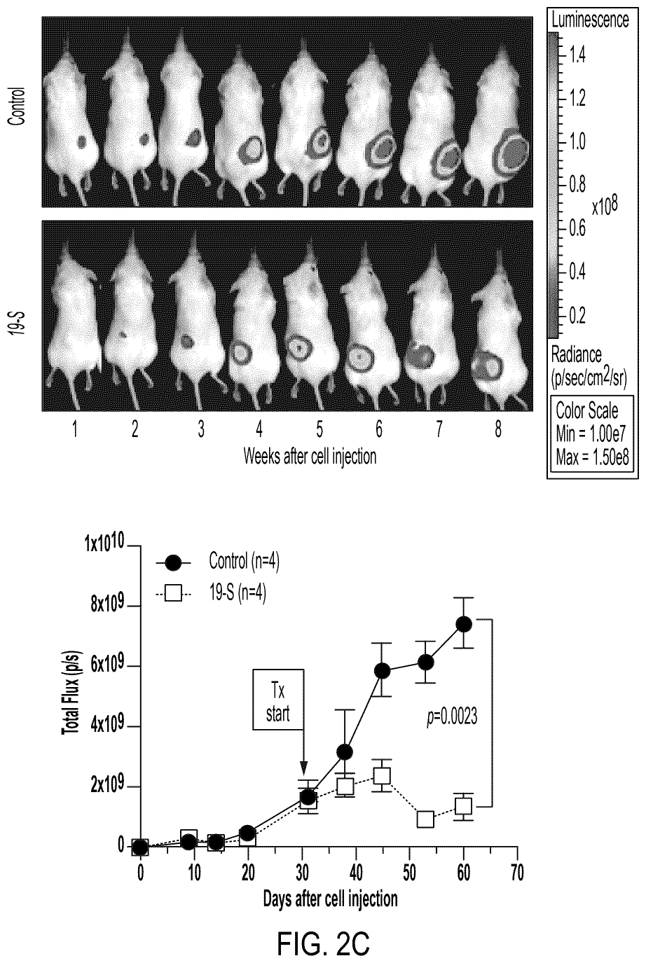

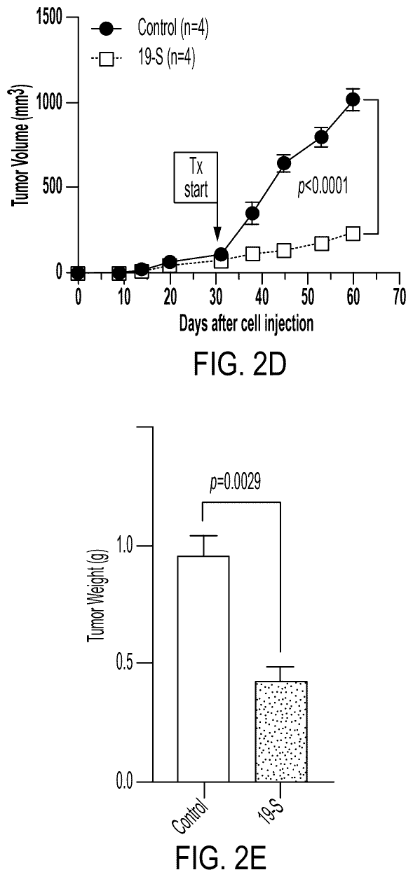

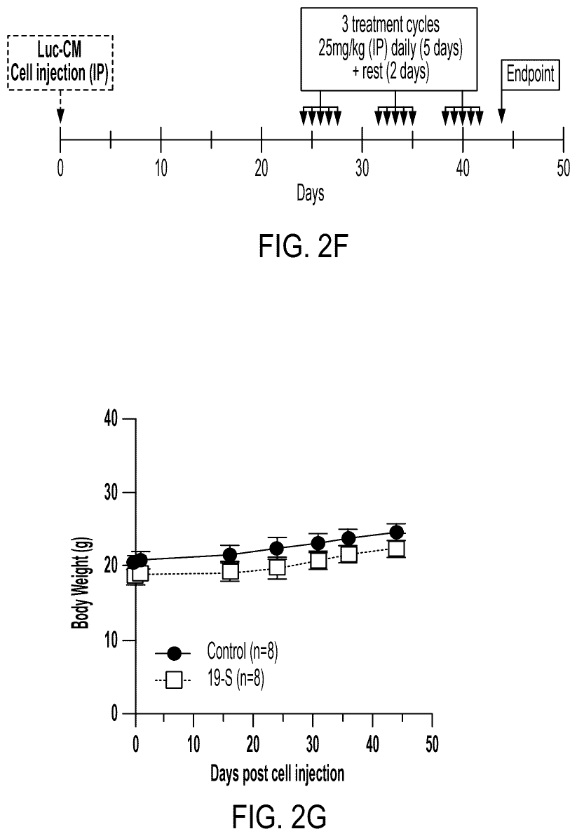

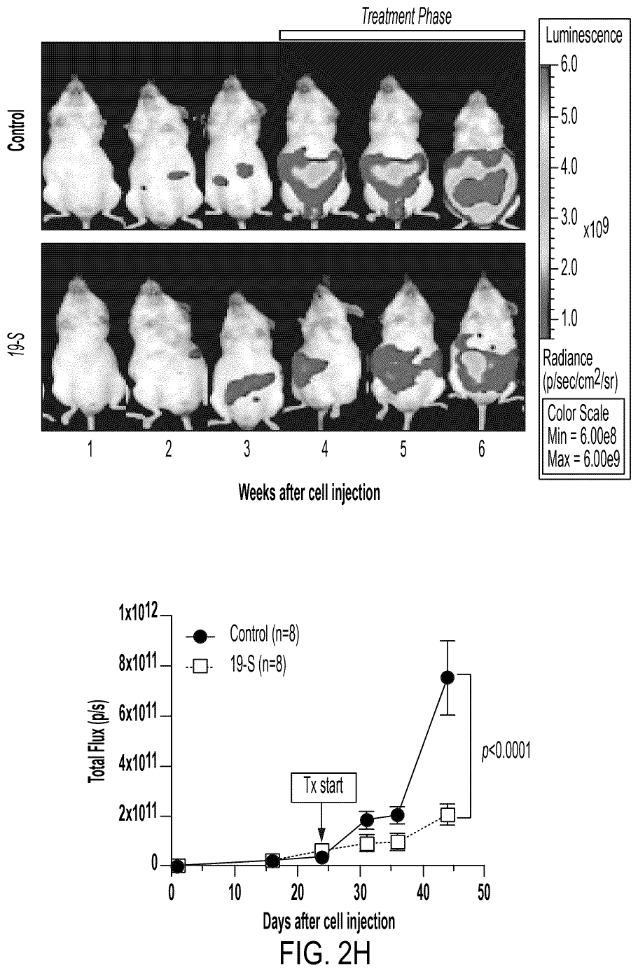

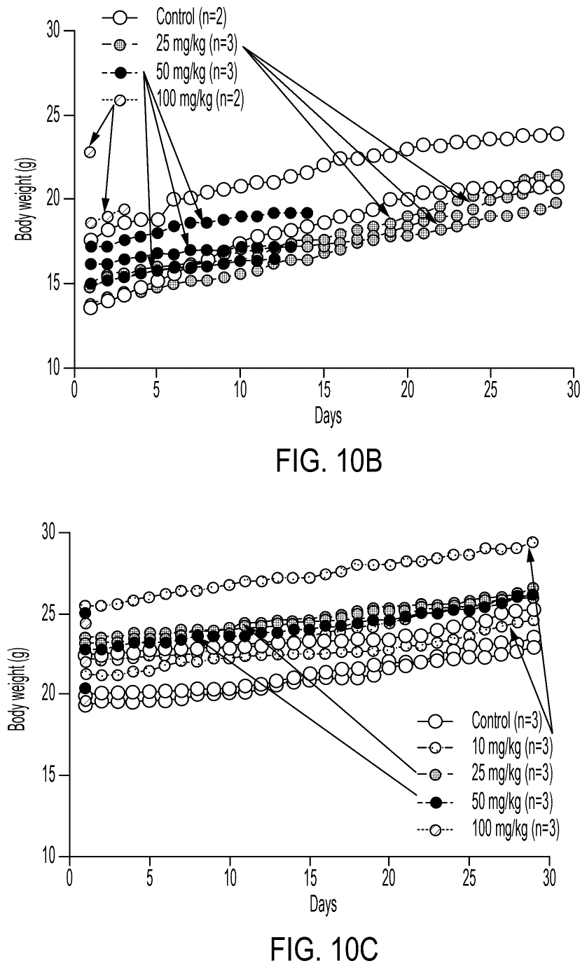

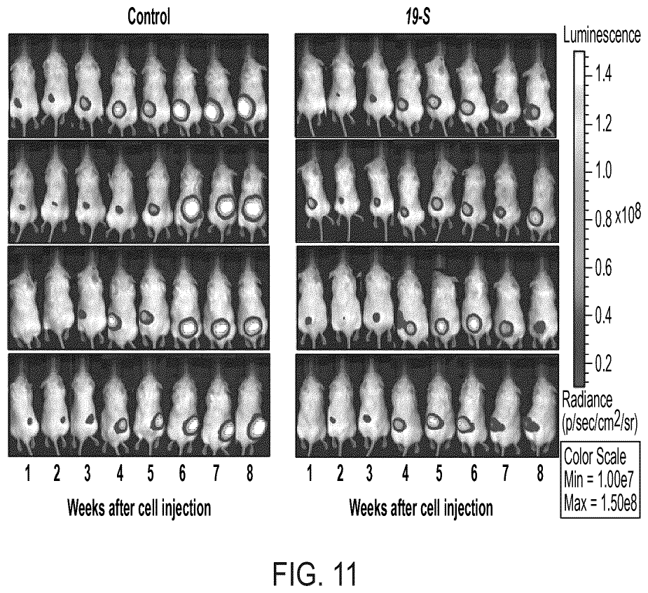

In the subcutaneous tumor model, the resulting Luc-PANC-1 xenograft tumors were monitored by direct tumor measurements and bioluminescence photon flux that is proportional to tumor size. The experimental timeline (FIG. 2A) illustrates the treatment cycles used for compound 19-S and the vehicle control delivered by intraperitoneal (IP) injections to tumor bearing NSG mice. Treatment was initiated 30 days after Luc-Panc-1 cell line subcutaneous injections (5×106 cells), when tumors reached 100 mm3 in volume generating a bioluminescence photon flux of 3-4×109 photons/second. Animals were randomly assigned into treatment groups receiving a total of 4 cycles with each cycle defined as either 25 mg/kg compound 19-S treated once a day for 5 continuous days then allowed 2 days rest without treatment or an equivalent volume of the vehicle control delivered by IP injection. Toxicity studies demonstrated the 25 mg/kg treatment dose was the maximum tolerated dosage (MTD), determined as the dose when animals were alive following treatment (FIG. 2 and FIG. 10A-10C). Additionally, the body weight was monitored for all animals throughout the experiment showing no significant deviations between the compound 19-S or control vehicle treated animals at the 25 mg/kg treatment dose (FIG. 2B), and no adverse animal behavior was observed for both treatment groups. While vehicle control treated tumors rapidly progressed, treatment with compound 19-S inhibited tumor progression as illustrated in the bioluminescence photon flux (p=0.0023, FIG. 2C and FIG. 11) and the tumor volume (p<0.0001, FIG. 2D). After completion of the 4th treatment cycle animals were given a 5-day rest without treatment, then all animals were euthanized due to the control tumors reaching experimental endpoint. Tumors from both treatment groups were excised and tumor mass weighted (FIG. 2E). Consistent with the bioluminescence photon flux and tumor volume, the final tumor mass for the compound 19-S treatment group was significantly reduced compared to the tumors from vehicle control group (p=0.0029). These data from the subcutaneous xenograft pancreatic tumor model demonstrate the ability of compound 19-S to prevent tumor progression of localized solid tumors.

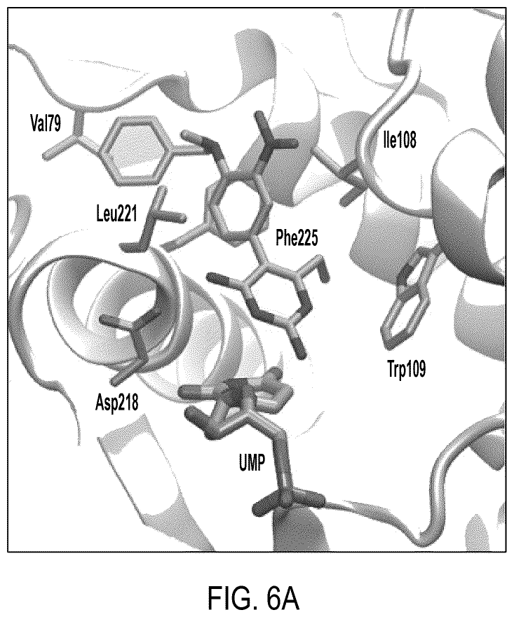

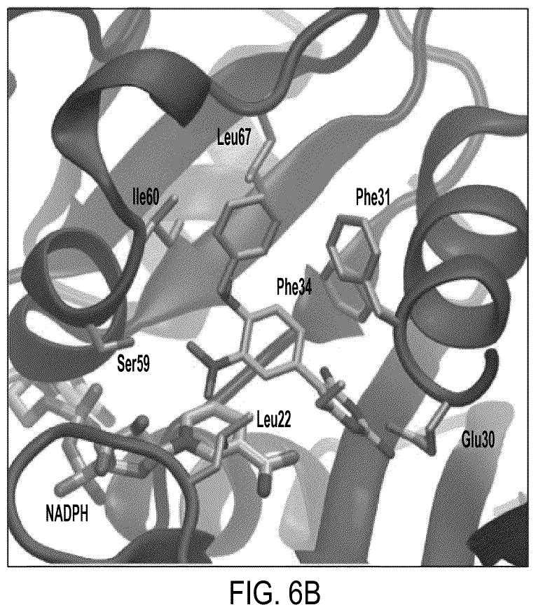

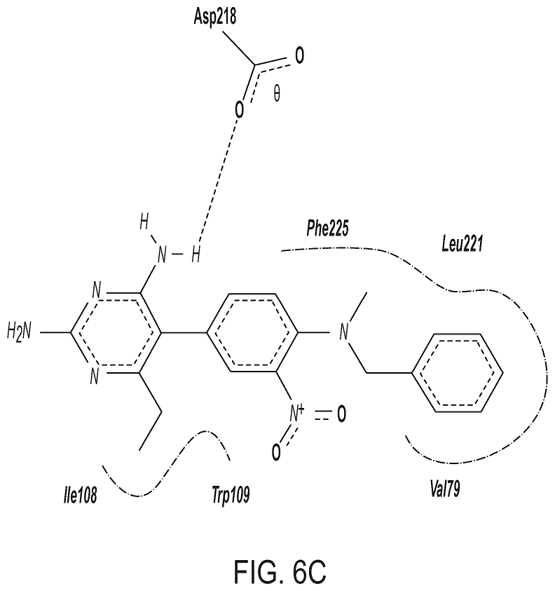

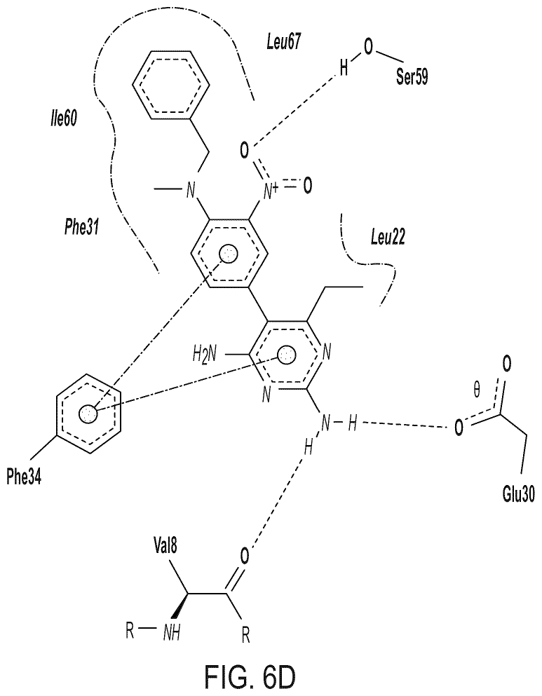

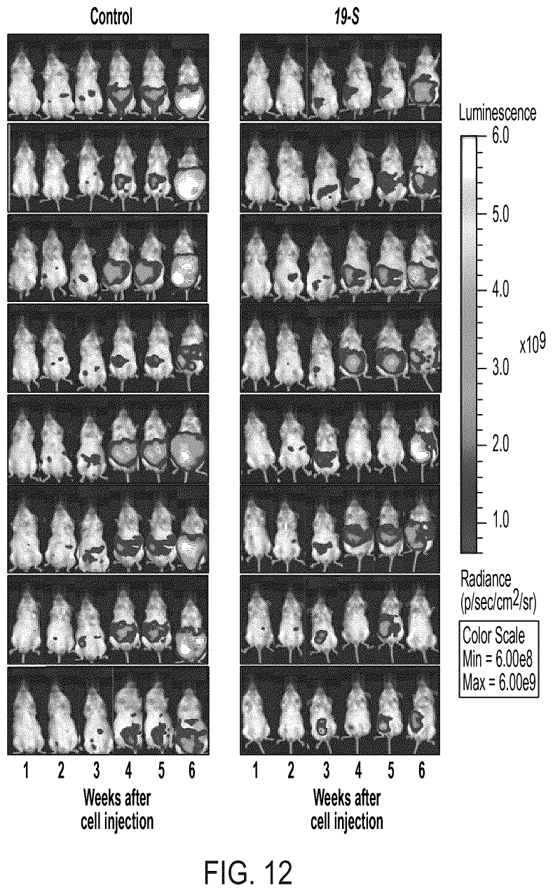

To further test the effect of compound 19-S on tumor growth and progression, a Luc-CM disseminated xenograft tumor model was utilized. Luc-CM cancer cells were delivered by IP injection allowing distribution throughout the abdominal cavity. Tumor progression was then monitored by the bioluminescence photon flux from the abdominal region, and treatment was initiated after 24 days when the abdominal region bioluminescence photon flux was in the range of 5×1010 photons/second. Animals were randomly assigned into treatment groups receiving either 25 mg/kg compound 19-S or an equivalent volume of the vehicle control delivered by IP injection. A total of 3 treatment cycles were administered and for each treatment cycle animals were treated once a day for 5 continuous days and then allowed 2 days rest without treatment (FIG. 2F). Similar to the data observed with the subcutaneous tumor model, the animals with Luc-CM-derived disseminated tumors did not show significant deviations in body weight or display adverse behavior as a result of the compound 19-S treatment (FIG. 2G). Although this disseminated tumor model does not allow direct tumor measurements, the bioluminescence photon flux from the abdominal region is proportional to the tumor burden. Once treatment was initiated, the vehicle control group experienced rapid tumor progression indicated by the increasing bioluminescence photon flux from the abdominal region, while compound 19-S treatment efficiently inhibited tumor progression (p=0.0032, FIGS. 2H & FIG. 12). In addition, this disseminated tumor model with Luc-CM cells was also used to determine if oral delivery of compound 19-S was well tolerated and resulted in similar inhibition of tumor progression. When treated with the same dose of compound 19-S (25 mg/kg) by oral gavage instead of IP injections, no significant deviations in body weight and similar inhibition of tumor progression were observed as with compound 19-S delivered by IP injections (FIGS. 6A-6D). Collectively, both xenograft tumor models demonstrate compound 19-S is tolerated while inhibiting tumor growth and its progression.

### Compound 19-S Prolongs Survival

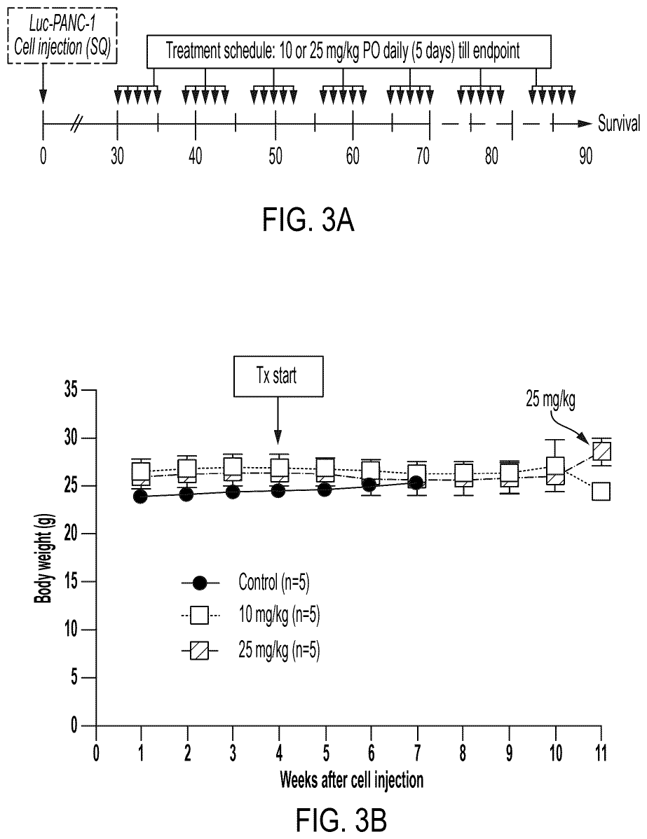

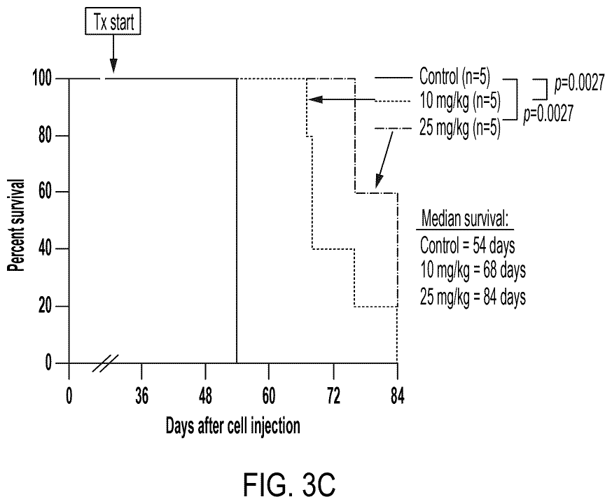

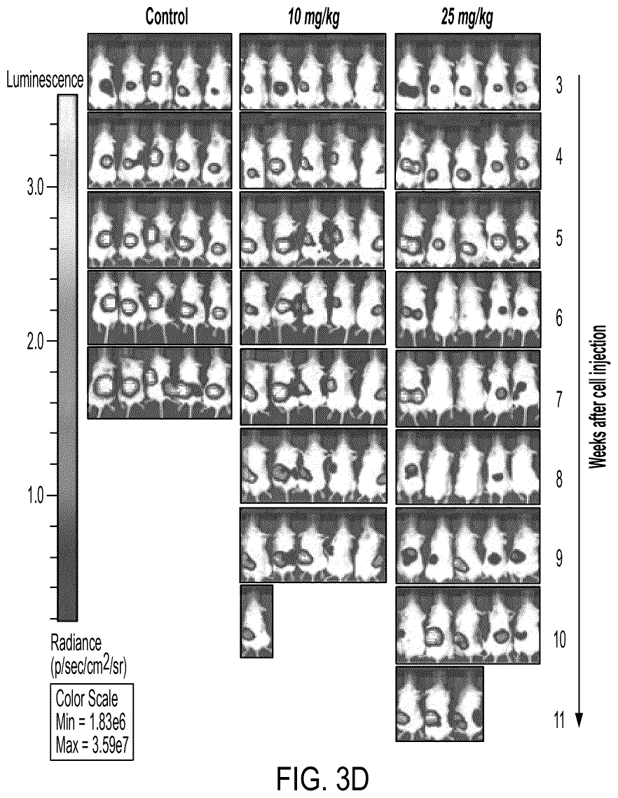

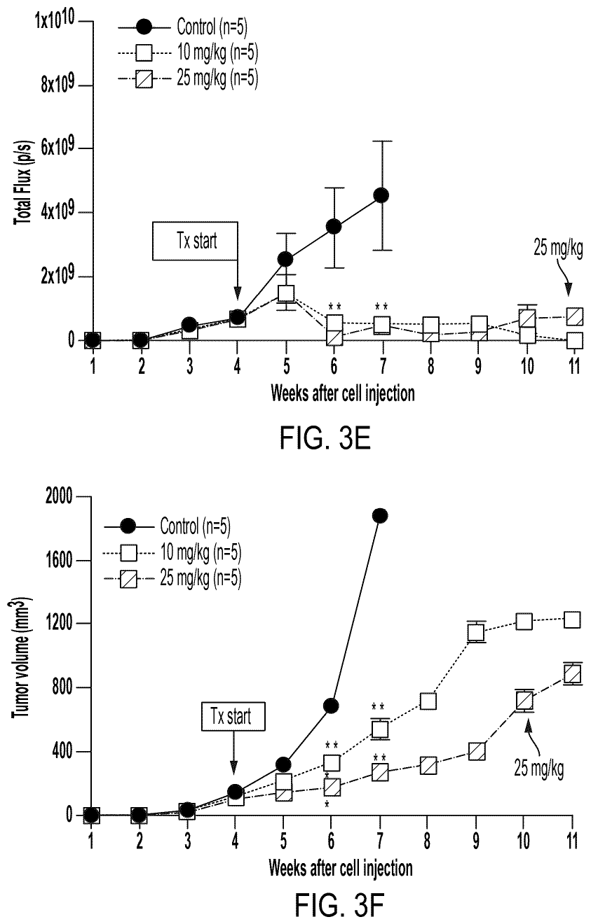

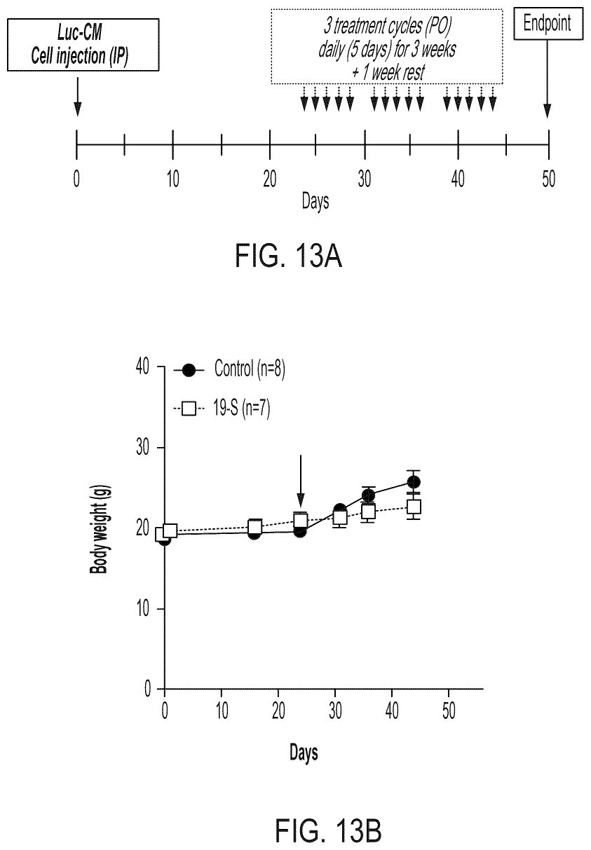

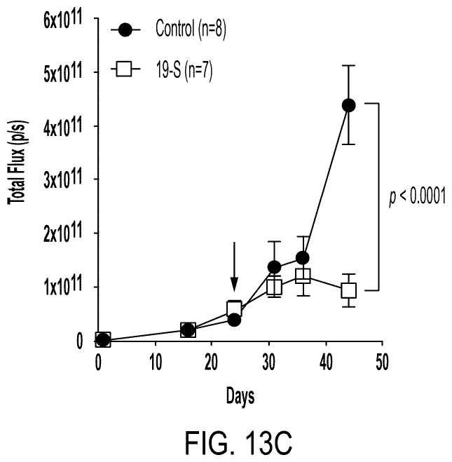

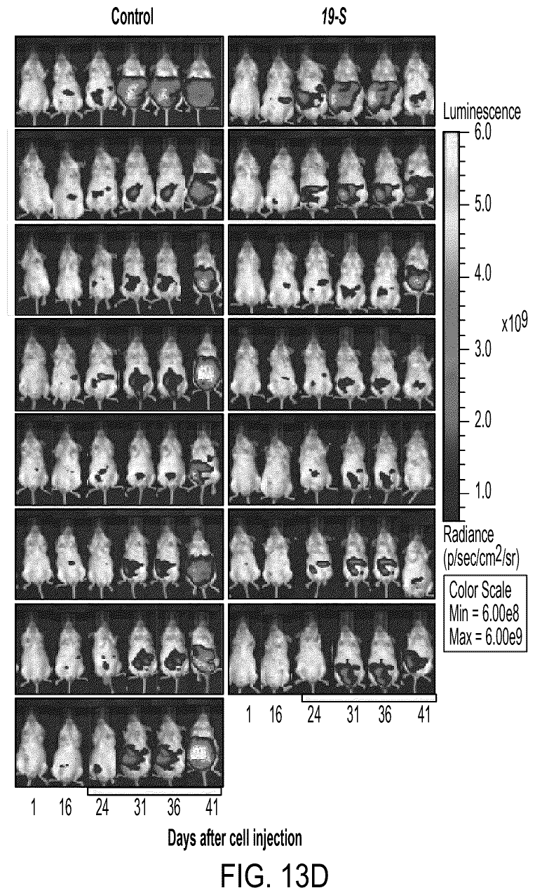

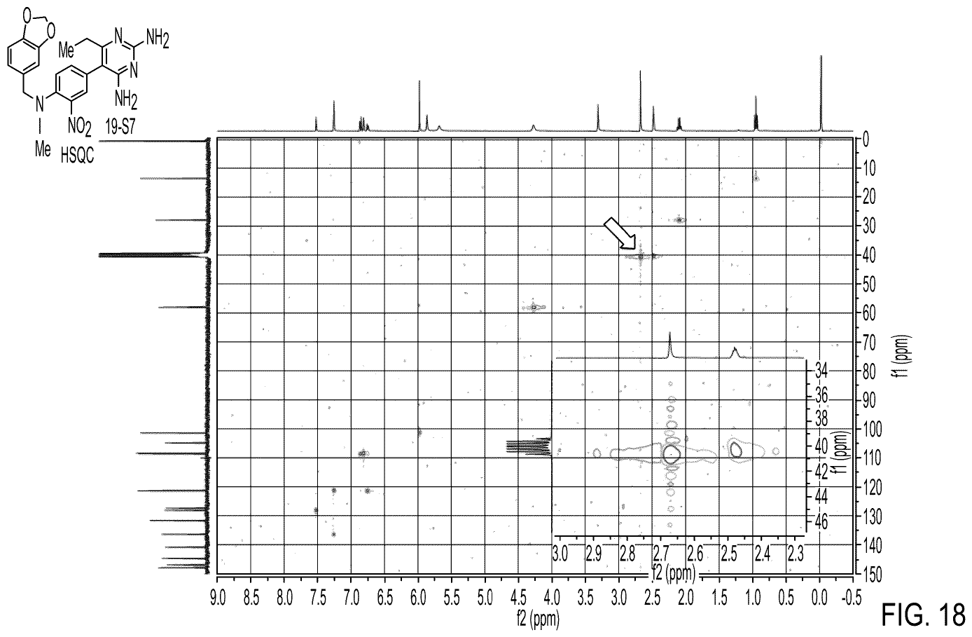

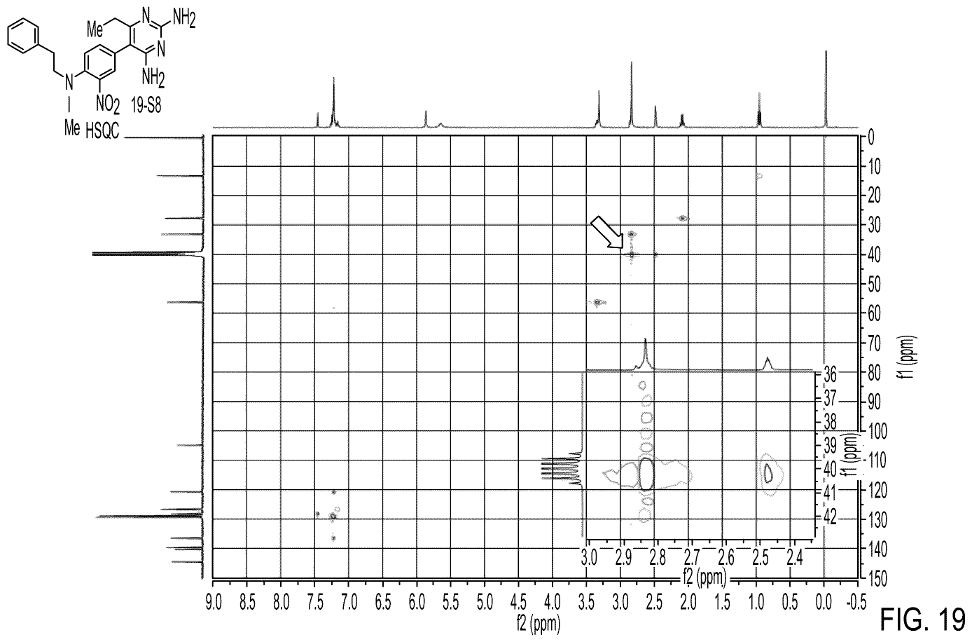

Since it was observed that compound 19-S inhibited tumor growth in two distinct xenograft tumor models (FIGS. 2 and 6A-61), the next question was whether the antitumoral effect of compound 19-S would prolong survival. The Luc-PANC-1 subcutaneous tumor model was tested with the PANC-1 cell line expressing firefly luciferase and followed the experimental timeline shown in FIG. 3A. Treatment was initiated 30 days after Luc-PANC-1 cell injections (5×106 cells) and was administered till survival endpoint. Animals were randomly assigned into treatment groups receiving 10 or 25 mg/kg compound 19-S by oral gavage (PO) once a day for 5 consecutive days, and then allowed 2 days to rest without treatment; control animals received vehicle (corn oil) on the same schedule. Treatment by oral gavage (PO) was chosen over intraperitoneal (IP) injection since it was observed that a similar antitumoral effect occurred by either route (FIG. 2 and FIGS. 6A-6D). Body weight was monitored throughout the experiment showing no significant differences between compound 19-S groups and vehicle control treated animals (FIG. 3B). Treatment with compound 19-S at either dose resulted in a statistically significant (p=0.0027) prolonged survival compared to vehicle control (FIG. 3C). While controls had to be sacrificed at 7 weeks due to tumor size, compound 19-S treated animals reached endpoint between 3 and 4 weeks after controls due to tumor ulceration. Thus, median survival increased from 54 days in control treated animals to 68 and 84 days in 10 mg/kg or 25 mg/kg compound 19-S treated animals, respectively. Compound 19-S independently of the concentration, inhibit tumor progression as determined by the reduced abdominal bioluminescence photon flux. At 5 weeks after cell injection (when animals have been treated for only one week) compound 19-S slowed down tumor growth. As a consequence, treatment induced a significant reduction of total flux compared to vehicle control at 6 weeks (p=00079, FIG. 3D and FIG. 3E) consistent with a significant inhibition of tumor volume (p=0.0079) that was maintained till endpoint (FIG. 3F). These data demonstrate the effectiveness of daily oral compound 19-S to prolong survival and control disease progression.

### Example 2. Synthesis of Exemplary Compound 19-S Analog Compounds

After confirming the safety and activity of compound 19-S, a series of compound 19-S analogues were synthesized. These analogues introduced structural diversity with some analogues designed to increase aqueous solubility. Generating the diverse series of 19-S analogues provides the ability to determine structural features with increased potency, while also understanding which structural features contribute to the observed biological activities.

19-5 and related analogues were synthesized from pyrimethamine using a two-step route that involved: (1) nitration of the p-chlorobenzene ring of pyrimethamine, and (2) subsequent nucleophilic aromatic substitution with several primary or secondary amines, as shown in scheme 1 below:

### Scheme 1. Synthetic Pathway to 19-S Analog Compounds

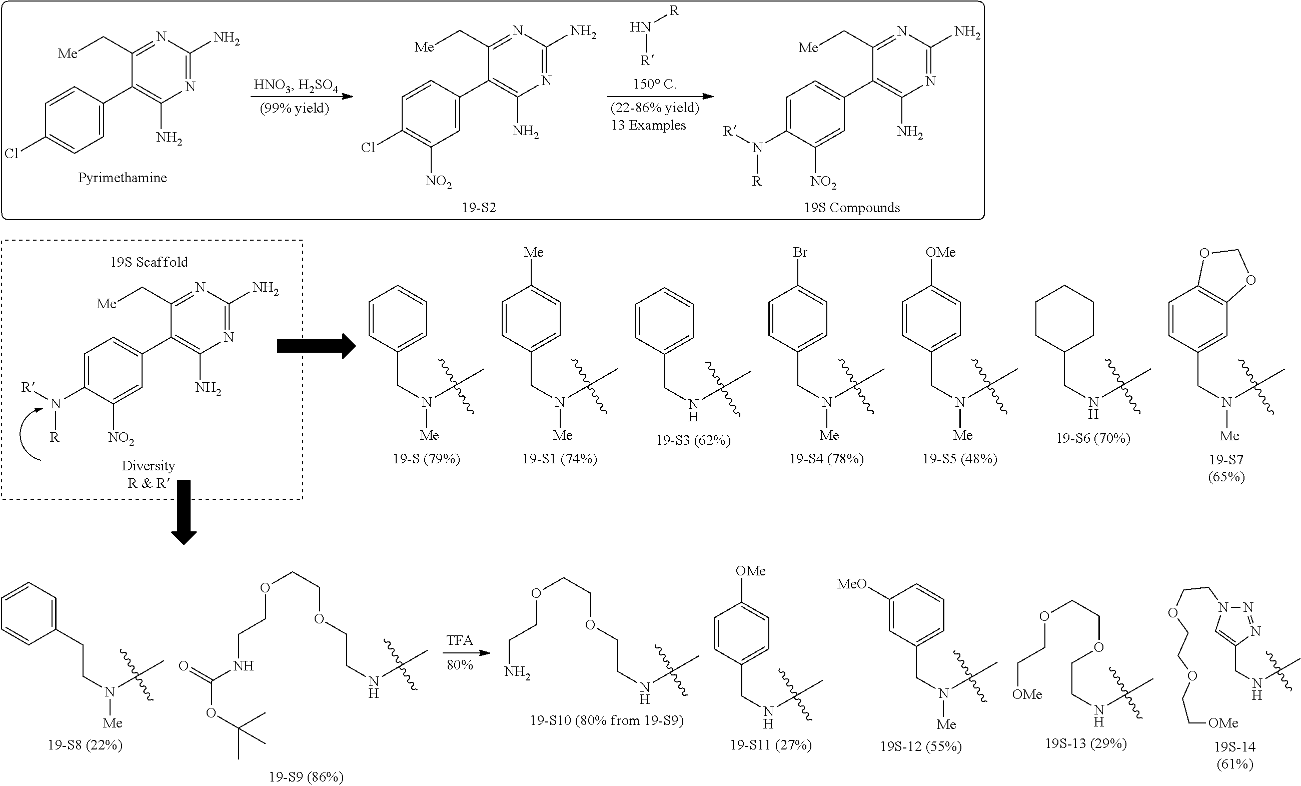

This short synthetic sequence was used to access 13 analogues of 19-S in 22-86% yield. In addition, 19-S10 was prepared using trifluoroacetic acid (TFA) for removal of the Boc-group of 19-S9. This robust synthetic route enabled rapid access to sufficient material (300 milligrams to 1 gram of several analogues) for both in vitro and in vivo studies.

The exemplary compounds synthesized via Scheme 1 are shown in Table 1 and Table A below.

TABLE 1

| Com- | |
|---|---|
| pound | |
| Number | Structure |
| 19-S6 | 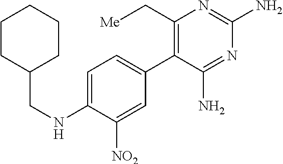 [SMILES: CCc1nc(N)nc(N)c1-c1ccc(NCC2CCCCC2)c([N+](=O)[O-])c1] |
| 19-S7 | 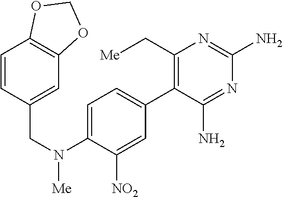 [SMILES: CCc1nc(N)nc(N)c1-c1ccc(N(C)Cc2ccc3c(c2)OCO3)c([N+](=O)[O-])c1] |
| 19-S10 | 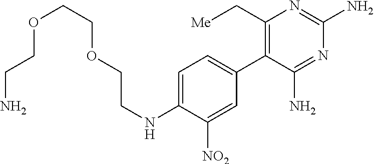 [SMILES: CCc1nc(N)nc(N)c1-c1ccc(NCCOCCOCCN)c([N+](=O)[O-])c1] |
| 19-S13 | 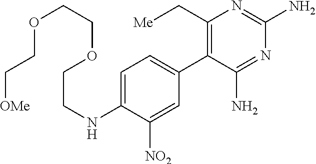 [SMILES: CCc1nc(N)nc(N)c1-c1ccc(NCCOCCOCCOC)c([N+](=O)[O-])c1] |
| 19-S14 | 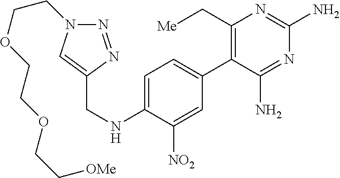 [SMILES: CCc1nc(N)nc(N)c1-c1ccc(NCc2cn(CCOCCOCCOC)nn2)c([N+](=O)[O-])c1] |

TABLE A

| Compound | |
|---|---|
| Number | Structure |
| 19-S4 | 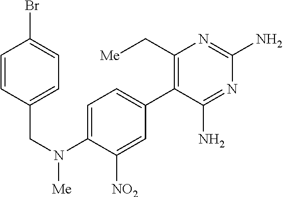 [SMILES: CCc1nc(N)nc(N)c1-c1ccc(N(C)Cc2ccc(Br)cc2)c([N+](=O)[O-])c1] |
| 19-S8 | 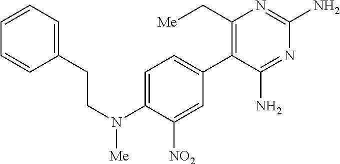 [SMILES: CCc1nc(N)nc(N)c1-c1ccc(N(C)CCc2ccccc2)c([N+](=O)[O-])c1] |
| 19-S12 | 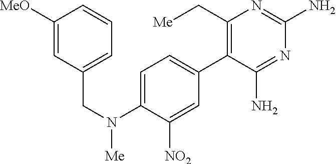 [SMILES: CCc1nc(N)nc(N)c1-c1ccc(N(C)Cc2cccc(OC)c2)c([N+](=O)[O-])c1] |

The precursor molecule pyrimethamine, used in the synthesis of the 19-S and its analogues, is a disclosed inhibitor of the protozoan DHFR enzyme³⁴. Therefore, exploration of a possible dual and/or complementary mechanism of action was conducted for this class of 19-S inhibitors, since TS and DHFR are both folate dependent enzymes.

### Example 3

### Compound 19-S and its Analogues 19-S5 and 19-57 Show Dual TS and DHFR Inhibition

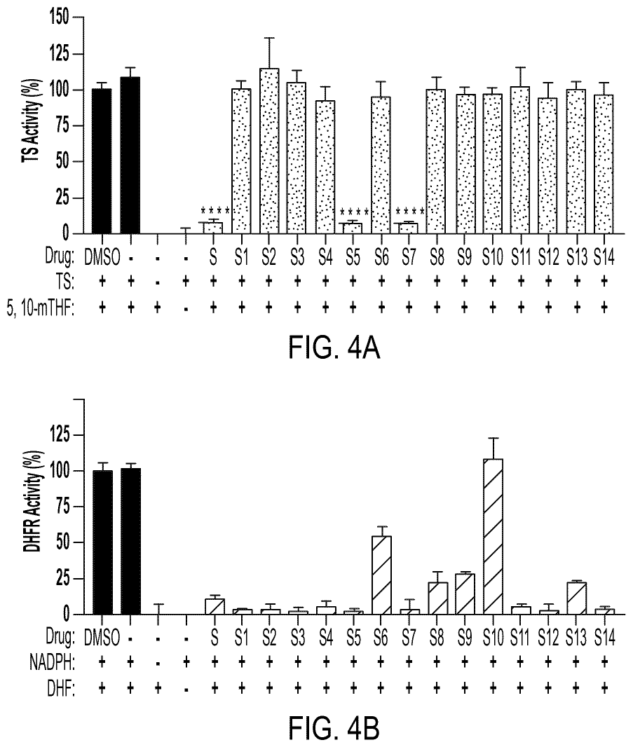

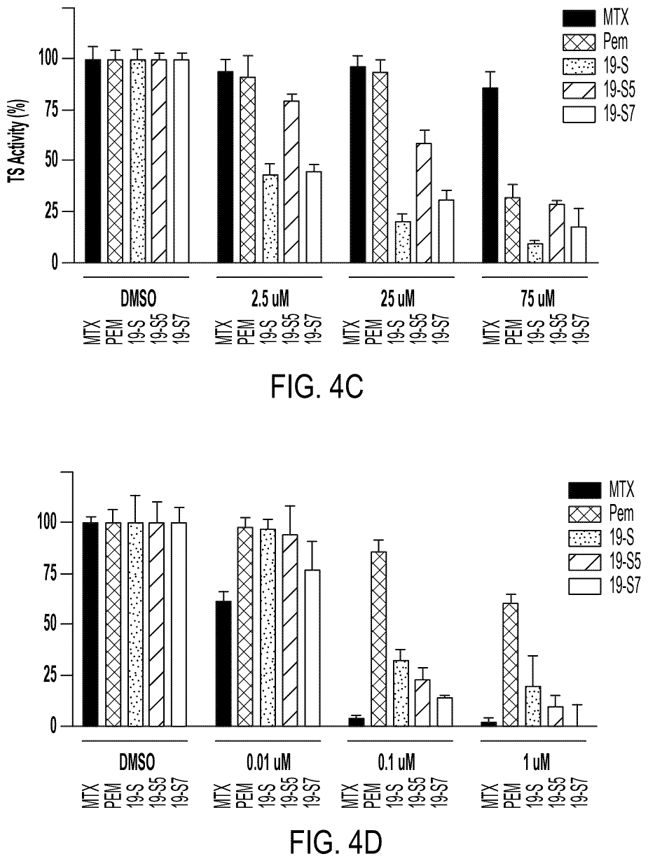

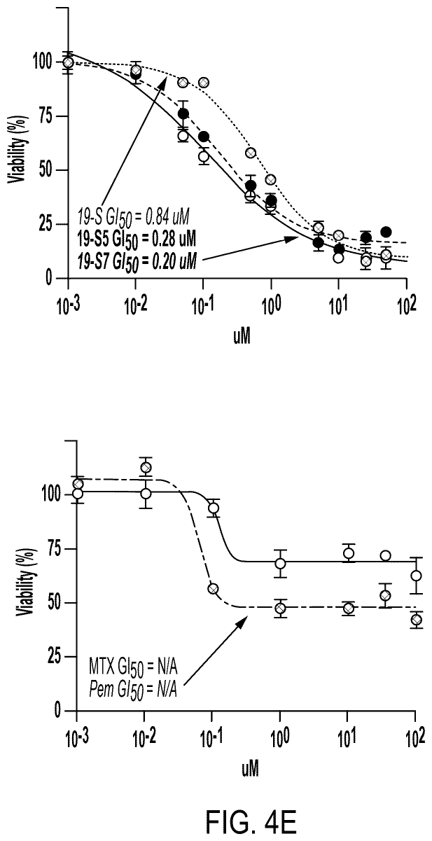

The structurally diverse 19-S series compound library with 15 compounds including the lead compound 19-S and its 14 analogues, 19-S1 through 19-S14, were screened to establish each compound's ability to inhibit TS catalytic activity (FIG. 4A). In addition to compound 19-S, 19-55 and 19-S7 also inhibited TS activity. The initial drug screen with the tritium-based TS activity assay utilized a higher threshold for the drug concentration (250 uM), which was designed to identify compounds showing even weak TS inhibition. It was observed that compounds either showed near complete inhibition of TS activity or no inhibition at all (FIG. 4A). This distinct binary difference suggests there could be a stringent binding criterion for TS inhibition dependent upon the benzylmethylamine moiety.

Since the 19-S series compounds also inhibit DHFR activity, it is suggested that the folate site as the target for inhibition by these compounds. The 15-compound library of the 19-S series was used to initially screen for DHFR inhibition using the standard absorbance-based activity assay (FIG. 4B). With the exception of compound 19-S10, all compounds in the 19-S series library were able to inhibit DHFR activity at 1 μM concentrations (FIG. 4B).

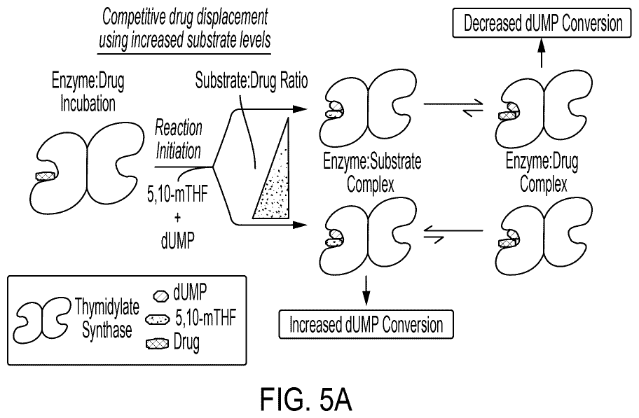

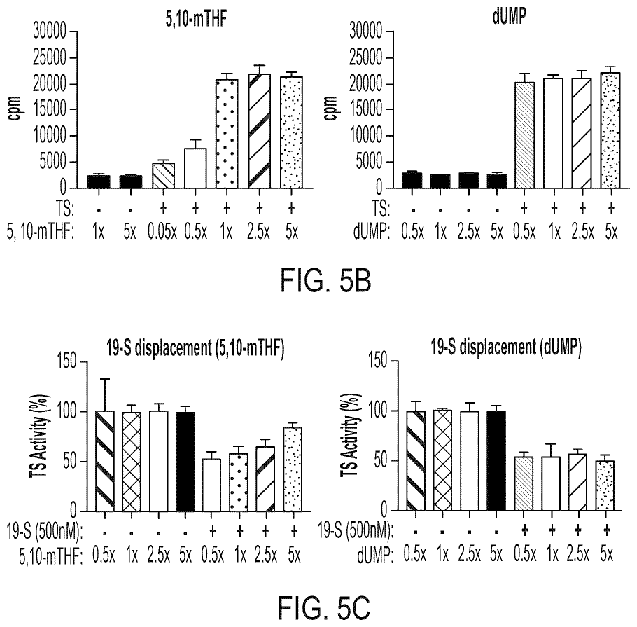

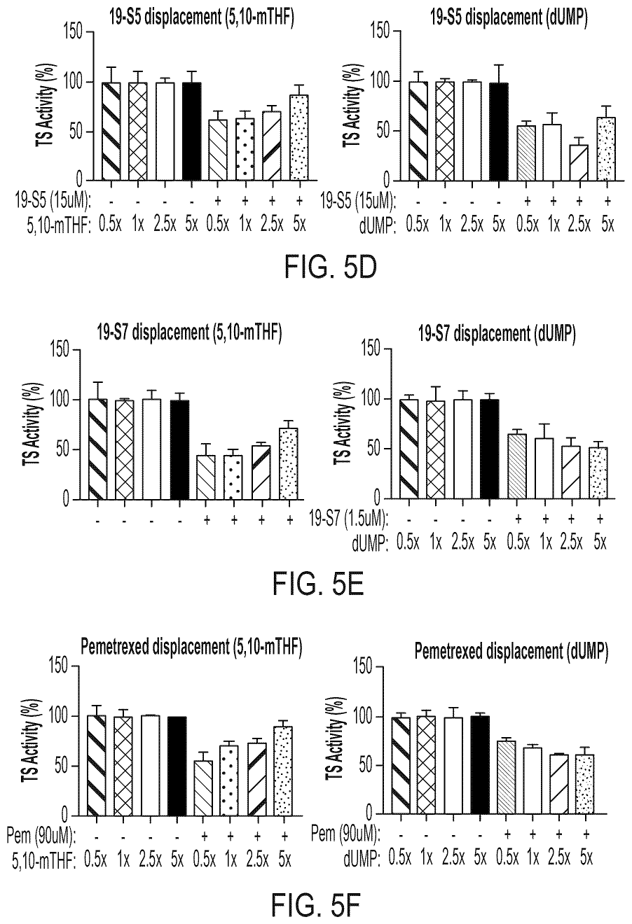

To further discriminate the inhibitory activity for the dual TS/DHFR inhibitor compounds 19-S, 19-55, and 19-S7, the tritium-based TS catalytic activity and DHFR absorbance-based activity assays were repeated at decreasing drug concentrations. Additionally, the classical antifolate compounds pemetrexed (Pem) and methotrexate (MTX) were included as controls to provide a reference for comparison given the different assays utilized to determine TS and DHFR activity. Pem displayed greater TS inhibition than the MTX antifolate while MTX was a more potent inhibitor of DHFR (FIGS. 4C and 4D). Compounds 19-S, 19-55, and 19-S7 demonstrated greater TS inhibition than both MTX and Pen (FIG. 5C). When DHFR activity was determined using reduced concentrations of each inhibitor, as expected, MTX retained the expected potent inhibition while Pern was less effective against DHFR (FIG. 5D). In contrast, compounds 19-S, 19-85, and 19-S7 showed both strong TS and DHFR inhibition (FIG. 5D).

While compound 19-S, 19-S5, and 19-S7 demonstrated potent dual TS and DHFR inhibitory activity, these compounds are structurally distinct from classical antifolates such as Pen and MTX. The cytotoxicity of the analog compounds disclosed herein depends on folate transport and metabolic pathways. These include factors such as the energy dependent membrane transport into the cells by the reduced folate carrier, and the intracellular conversion to its polyglutamated metabolite by FPGS^{25,26,35,36}. The purified enzyme assay results were subsequently checked using cell-based assays that are dependent on factors such as cellular uptake and transport as well as drug metabolism. Cell viability assays were then performed with compounds 19-S, 19-S5, 19-S7, and the classical antifolates Pern and MTX using the PDAC cell line MIA PaCa-2 (FIG. 4E). Following the 72-hour treatment, compounds 19-S, 19-S5, and 19-S7 resulted in potent cytotoxic effects with GI50 values ranging from 0.20 μM to 0.84 μM, while the classical antifolates had a cytostatic effect and were unable to reduce viability below 50% at the highest 350 μM concentration (FIG. 4E). These data further highlight this new class of dual TS/DHFR inhibitors exemplified by compounds 19-S, 19-S5, and 19-S7 that are structurally distinct from classic folate antimetabolites and are independent of obstacles related to folate transport and metabolism.

### Mechanism of TS Inhibition Confirms Non-Classical Antifolate Inhibitors

To further elucidate the non-classical antifolate mechanism of dual TS/DHFR inhibition observed with compounds 19-S, 19-S5, and 19-S7, a series of drug displacement experiments based on the established tritium TS activity assay was designed. For these experiments the TS protein was first incubated with each drug, the reaction was then initiated by addition of 5,10-mTHF and dUMP using different concentrations of either 5,10-mTHF or dUMP to change the substrate:drug ratio. If there was competition between one of the substrates and the drug for the same binding site, increasing the substrate concentration will displace more of the pre-bound drug and shift the equilibrium towards the enzyme: substrate complex, thus allowing the conversion of dUMP to proceed (FIG. 5A).

First, it was confirmed that increasing the concentration of either the dUMP substrate or the 5,10-mTHF cofactor did not affect the amount of dUMP converted during the reaction, and therefore did not increase the scintillation counts (FIG. 513). The different substrate concentrations are expressed relative to the standard concentrations used for the tritium assay. These standard saturating substrate concentrations were previously determined when the assay was optimized to ensure that either of the substrates were not depleted during the reaction. Since TS activity is quantified by the conversion of the tritiated dUMP tracer, maintaining the same tritiated dUMP:regular dUMP ratio is essential for comparing the reactions performed with increased concentrations of dUMP. Increasing the regular dUMP concentration, thus causing a reduction in the tritiated dUMP:regular dUMP ratio, could be perceived as a reduction in TS activity given less tritiated dUMP would be converted due to the increased regular dUMP available, and vice-versa. As observed from the raw scintillation count data, increasing the 5,10-mTHF concentration by 2.5-fold or 5-fold did not have an impact on the conversion of dUMP (FIG. 5B). As expected, when the tritiated dUMP:regular dUMP ratio is maintained and the total dUMP concentration was increased, there was no impact on the total amount of tritiated dUMP converted during the reaction (FIG. 5B). These data confirm the amount dUMP conversion during the reaction does not increase with increasing 5,10-mTHF or dUMP concentrations.

The drug displacement assay for compounds 19-S, 19-S5, and 19-S7 was performed. It was determined that increasing the 5,10-mTHF concentration increased the amount of dUMP conversion during the reaction for all three of the 19-S series compounds (FIGS. 5C-5E). This demonstrates 5,10-mTHF can displace the pre-bound 19-S series compounds shifting the equilibrium toward the enzyme:substrate complex allowing increased conversion of dUMP. When the reactions with the three 19-S series compounds were performed with increasing dUMP concentrations, the amount of dUMP conversion did not increase (FIGS. 5C-5E), demonstrating dUMP is not competing for the same binding site as the 19-S series compounds. Since Pem is a classical antifolate known to bind in the TS folate site, the drug displacement assay was performed with Pem as an additional control. As expected, increasing the 5,10-mTHF concentration increased the dUMP conversion due to displacement of Pem from the folate site, and increasing dUMP concentration did not increase dUMP conversion (FIG. 5F). In summary, this data provides evidence that the mechanism of TS inhibition for compounds 19-S, 19-85, and 19-S7 is by blocking the folate binding site and therefore acting as “non-classical” antifolates.

### Structural Basis for Dual TS/DHFR Inhibition

Molecular docking simulation results suggest that compound 19-S binds the active site of TS occupying the folate-binding pocket (FIG. 6A and FIG. 6C). Therefore, a binding mode in which the aminopyrimidine group establishes the main interactions, involving a hydrogen bond with Asp21S was proposed. The benzyl group lies on an adjacent hydrophobic region establishing several non-polar interactions. The nitro group is exposed to the solvent and apparently does not interact with any residue.

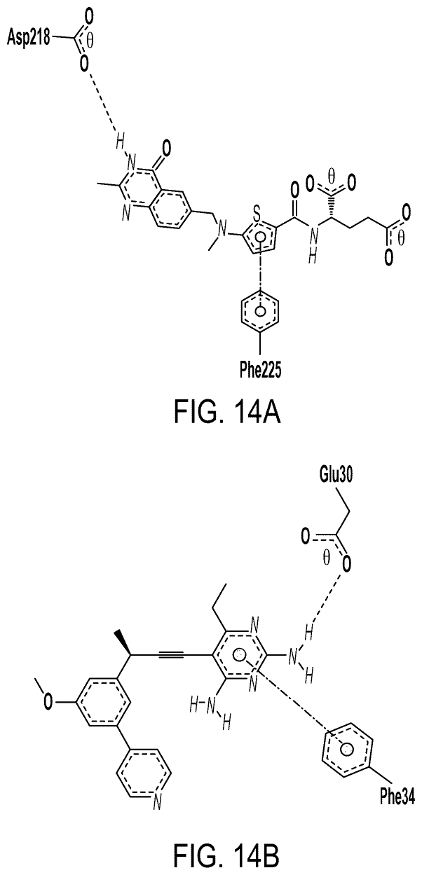

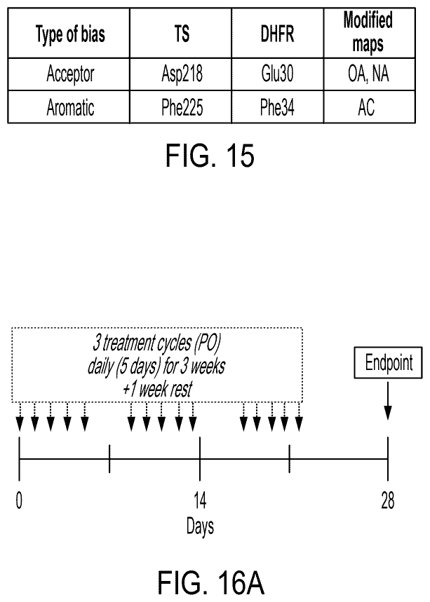

On the other hand, compound 19-S also binds the active site of DHFR occupying the folate-binding pocket (FIGS. 6B and 6D). The proposed ligand binding mode is in agreement with previous reported structures (PDB ID: 4KAK, FIGS. 14A-14B and FIG. 15) in which the aminopyrimidine group also establishes the main interactions with the protein. The model suggests that compound 19-S established more polar and aromatic interactions with DHFR than TS. The nitro group is involved in a hydrogen bond with Ser59 and the aminopyrimidine group could interact with the Glu30 sidechain and Val8 backbone. Finally, as observed in TS, the benzyl group is placed on an adjacent hydrophobic region establishing several non-polar interactions.

### Compound 19-S and 19-S7 Prevent Tumor Progression Following Oral Delivery

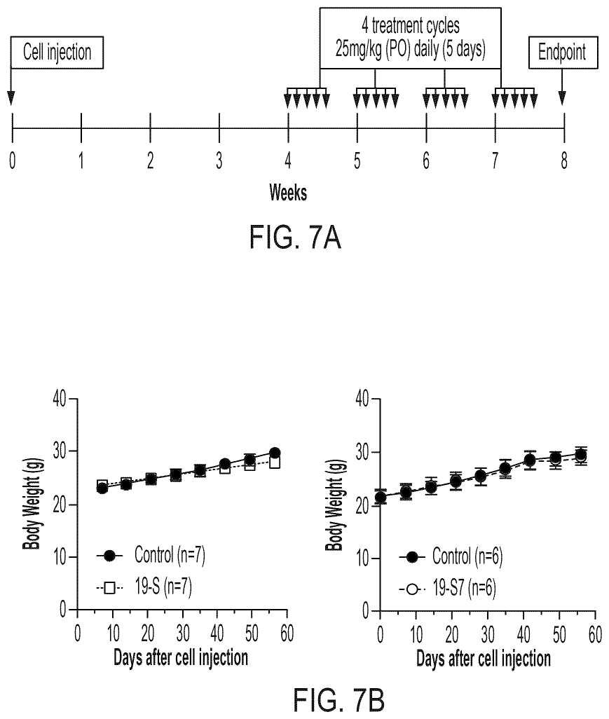

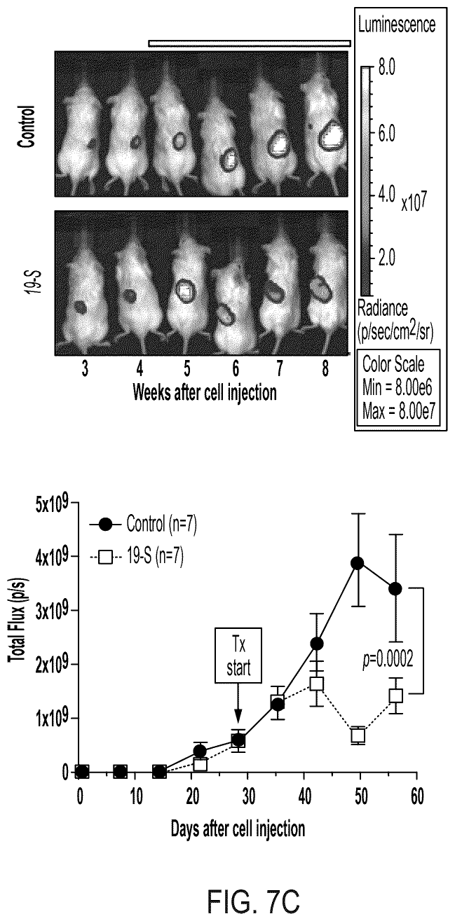

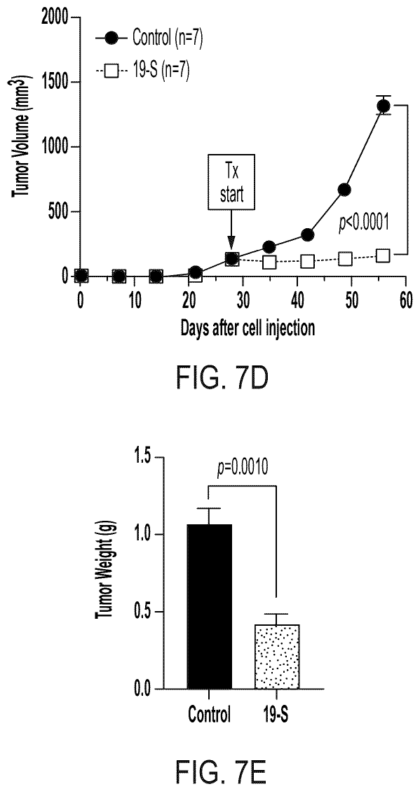

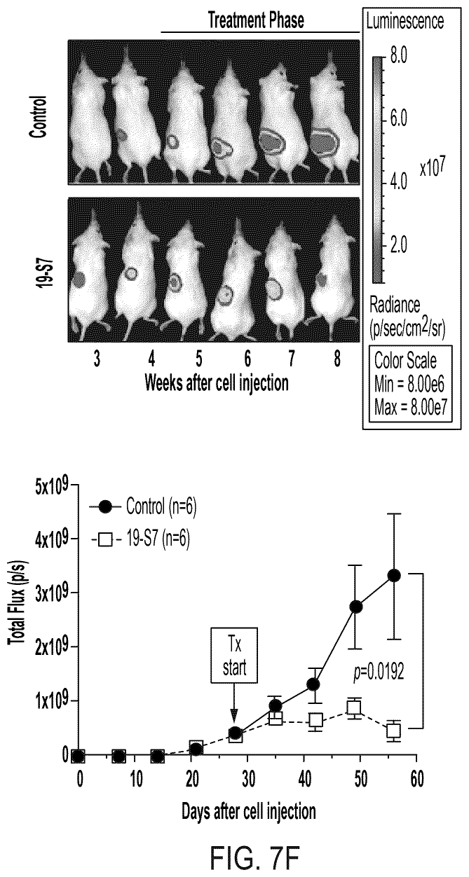

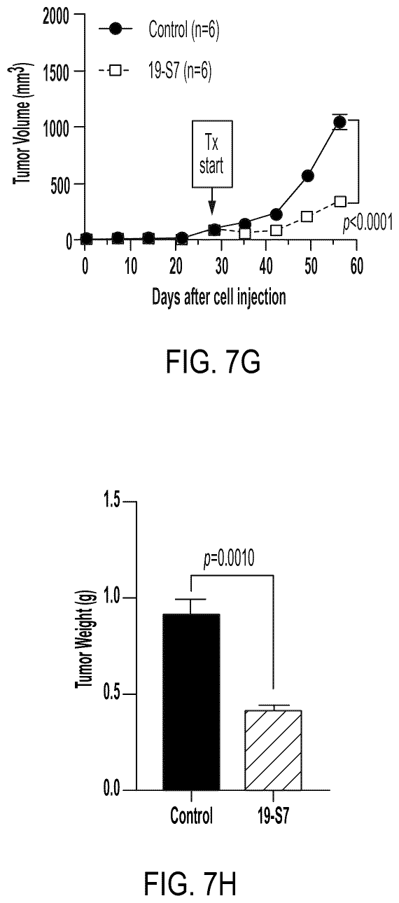

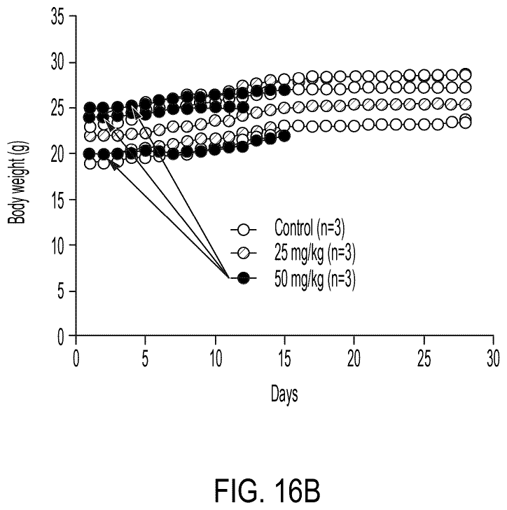

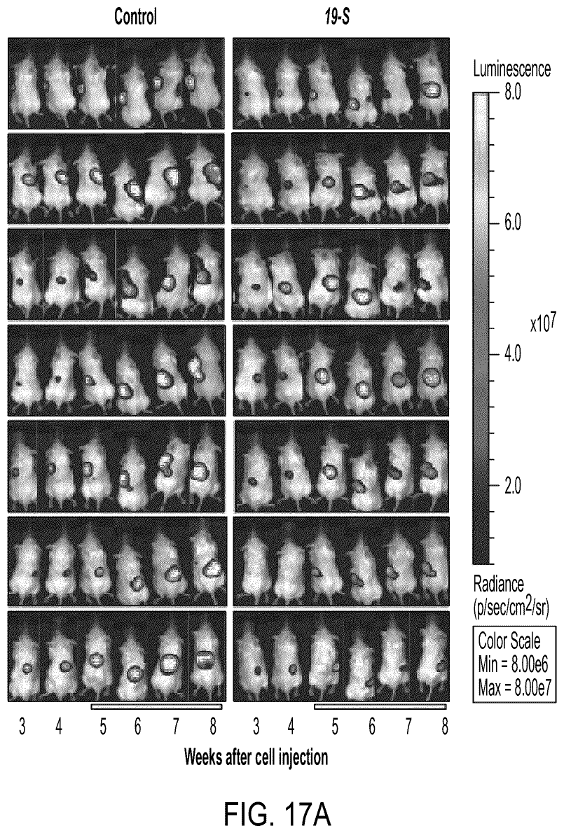

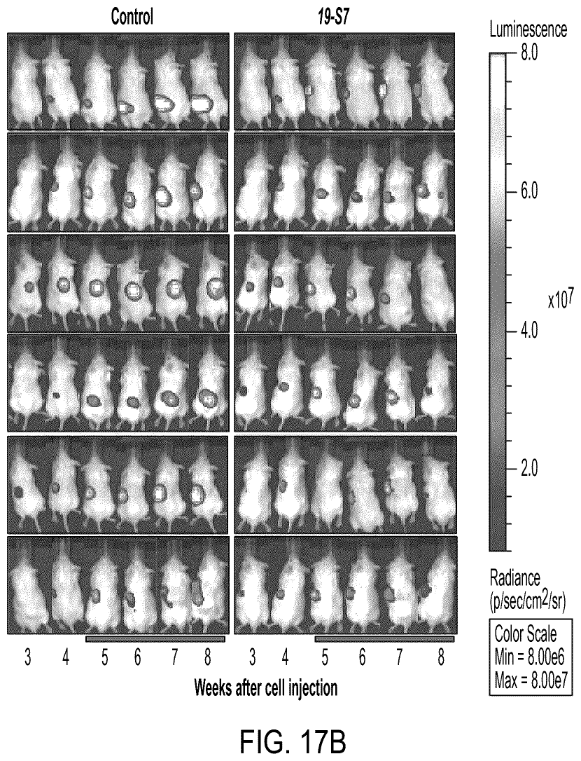

Classical antifolates such as Pem have shown improvements in disease survival when continued in the maintenance phase of cancer treatment³⁷. Due to the poor bioavailability of classical antifolates treatment requires intravenous infusions at MTD dosing every 21 days^{37,38}. However, oral administration of chemotherapy treatment is preferred in clinical management, especially for maintenance therapy allowing for more continuous, metronomic treatment protocols. Therefore, the toxicity and anti-tumor activity following oral administration of compound 19-S and the potent analogue compound 19-S7 using the Luc-PANC-1 derived subcutaneous tumor model (FIGS. 7A-7H) was tested. The experimental timeline (FIG. 7A) illustrates the treatment cycles used for both compounds. For each cycle, animals received daily treatments by oral gavage (25 mg/kg) for 5 continuous days, then allowed to rest for 2 days without treatment, and a total of 4 cycles were administered. The same treatment dose of 25 mg/kg was utilized for both compound 19-S and 19-S7 after confirming the MTD for compound 19-S7 was the same 25 mg/kg observed for compound 19-S (FIGS. 16A-16B). Following oral delivery, tumor bearing animals treated with both compound 19-S and 19-S7 did not show adverse effects or declines in body weight (FIG. 71B). Tumor progression was determined by the bioluminescence photon flux (FIGS. 7C, 7F, and 17A-17B), by direct tumor measurements (FIG. 7) and FIG. 7G), and by measurements of the final excised tumor mass (FIG. 7E and FIG. 7H). Both compounds inhibited tumor progression, as determined by the reduced bioluminescence photon flux from tumor cells, reduced and stable tumor volumes, and reduced mass of the final excised tumors. As a single agent with potent dual TS/DHFR inhibition, with anti-tumor activity and with no observed adverse side effects, and with the ability to be taken orally, the active 19-S series compounds demonstrate their potential to inhibit TS activity without induction of TS expression that may prevent drug resistance.

### Discussion

While TVS inhibition has been a component of combination cytotoxic therapy for difficult-to-treat advanced cancers, current fluoropyrimidines and antifolate TS inhibitors are associated with induction of TS overexpression that confers drug resistance resulting in limited long-term benefit and negligible cure rates in patients with advanced disease1. For example, both the 5-FU prodrug analog, capecitabine1 or compounded agent TAS-102 (trifluridine combined with tipiracil) yields in the active 5-fluoro-2′-deoxyuridine metabolite that exhibits the same potential for TS overexpression as observed with 5-FU 1,39,40. Therefore, the pursuit of more effective TS inhibitors is an important goal.

This example identified compounds structurally distinct from folic acid, where these compounds still compete with the 5,10-nTHF cofactor required for the TS catalyzed conversion of dUMP while maintaining the ability to also inhibit DHFR. These compounds thus act as multifunctional non-classical antifolates. The three 19-S series compounds with dual TS/DHFR inhibitory activity examined in this study show potent biological activity and are predicted to avoid the prototypic drug resistance arising from induction of TS overexpression. In addition, since these compounds are not classical folic acid analogs, they are not dependent on folate transporters or folate metabolism to achieve their full therapeutic potential. Therefore, these non-classical antifolates provide therapeutic benefits of targeted therapy for patients with resistance to the standard classical antifolates related to these folate transport and metabolism pathways.

It was previously reported that aberrant elevated levels of TS have oncogenic activity¹². Therefore, the ability to efficiently lower TS catalytic activity using a continuous maintenance regiment without the risk of inducing reciprocal feedback mechanisms such as TS overexpression is an important strategy in improving outcomes for patients with difficult-to-treat cancers¹. Maintenance-phase therapy using daily, weekly or metronomic therapy could be an effective option for controlling TS activity, reducing side effects, and improving long term outcomes. For this approach to be feasible, oral administration is preferred for improved patient logistics/compliance to maintain a continuous lower dose exposure compared to repeated high dose bolus IV infusions. While current fluoropyrimidine treatment options can also be administered orally^{39,41}, the induction of TS overexpression commonly observed in response to fluoropyrimidines limits the therapeutic benefits of inhibiting TS activity^{1,39}. Maintenance therapy using antifolates was shown to be effective, as observed with Pem for NSCLC, although such maintenance phase treatment still relies on IV infusions every 21-days due to poor oral absorption of the drug^{37,42,43}. Recent preclinical studies reported Pem bioavailability using Pem-bile acid conjugate complexes to enhance absorption⁴⁴⁻⁴⁷. These complexes allow daily oral administration with anti-tumor activity and reductions in new blood vessel formation. While less frequent bi-weekly IV infusions had similar anti-tumor activity there were negligible antiangiogenic properties observed^{44,47}. These preclinical studies further highlight the potential benefits from antifolate therapy using frequent low dose treatment. We have now identified a family of orally administered non-classical antifolates that optimizes inhibition of thymidylate biosynthesis with a favorable safety profile and extends survival in a pancreatic tumor mouse model highlighting potential for improved cancer therapy.

### Methods

### Cell Culture

PANC-1 and MIA PaCa-2 cells were purchased from the American Type Culture Collection (ATCC) and were grown in Dulbecco's modified Eagle's (DMEM) High glucose medium. BON cells, derived from a serotonin-secreting PanNET were grown in DMEM-F12 (50/50) medium. CM cells derived from an insulin-secreting PanNET were grown in RPMI-1640 medium. All were supplemented with 5 U/mil Penicillin/Streptomycin and 10% fetal bovine serum except for CM cells that were supplemented with 5% fetal bovine serum. All cells were grown at a constant temperature of 37° C. in a humidified atmosphere of 5% carbon dioxide and were routinely tested for mycoplasma contamination. PANC-1 and CM cells were further transduced with lentiviral luciferase (Addgene) and maintained in puromycin 8 μg/ml; these luciferase expressing cell lines were designated Luc-PANC-1 and Luc-CM. All cells were tested for mycoplasma before any experiment using a commercially available PCR-based detection kit.

### Cell Treatment, Chemicals, and Compound Synthesis

To prepare lysates, 0.5×106 PA NC-1 cells were seeded in 100 mm dishes in 10 ml DMEM High glucose and 24 hours later, 5-FU (Sigma) or 19-S were added at the specified concentrations. Cells were harvested after 72 hours and cell pellets were stored at −80° C. For GI50 determination, 3000 MIA PaCa-2 cells. 4000 PANC-1 or CM cells, or 5000 BON cells per well were plated in 96 well plates and 16-20 hours after seeding, cells were treated with increasing doses of the indicated drug: 5-FU (Sigma. F6627), MTX (Sigma), Pem (LC Labs), 19-5, 19-55 or 19-S7. After 72 hours, cell viability was assessed by reduction of MTS ((3-4,5-dimethylthiazol-2-yl)-5-(3-carboxymethoxyphenol)-2-(4-sulfophenyl)-2H-tetrazolium) using Cell Titer 96 R Aqueous One Solution Cell Proliferation Assay Kit (Promega), following manufacturer's recommendations. Chemiluminescent output (integration time 1000 ms) was measured on a SpectraMax M3® (Molecular devices). Data were normalized to max/minutes and plotted in GraphPad Prism for GI50 determination. Chemical synthesis details, including characterization of each compound, can be found in the supporting information document associated with this publication.

### Protein Isolation

Protein lysates were generated using RIPA buffer (Santa Cruz) for 20 minutes on ice, followed by 15 minutes centrifugation at 13,000 rpm, at 4° C. Protein-containing supernatant was transferred to microcentrifuge tubes and stored at −80° C. until further use. Protein was quantified using Bradford Assay (BioRad) following manufacturer's recommendations, standard curves were generated with bovine serum albumin (Fisher).

### Immunoblotting

20 μg of total protein lysate was loaded per lane of 10% Tris Glycine Gel (Invitrogen). SDS-PAGE was run at 150V for 1.5 to 2 hours. Proteins were transferred to nitrocellulose membranes using iBlot (Invitrogen). Membranes were blocked with 5% non-fat dry milk (Lab Scientific) in Tris-buffered saline supplemented with Tween20 (0.1%) (TBS-T) for 45-60 minutes at RT. Membranes were incubated on a plate shaker overnight at 4° C. with TS-106 antibody (1:300 as previously described12) or GAPDH (1:1000, Millipore) diluted in blocking buffer. Membranes were washed extensively with TBS-T (minimum 4× for 5 minutes), followed by incubation with horseradish peroxidase-conjugated secondary antibody goat anti-mouse IgG (BioRad) or goat anti-rabbit (BioRad) in blocking buffer 30-60 minutes at RT on a plate shaker. Membranes were washed extensively with TBS-T (minimum 4× for 5 minutes). Signal was detected using West Pico Plus chemiluminescent substrate (Thermo Scientific) following manufacturer's recommendations. Membranes were developed using multiple film (Gene Mate) processed in a Kodak X-Omat 2000A processor with exposures ranging from 2 seconds to 2 minutes. For protein level quantification relative to loading control, densitometric analysis was performed by ImageJ software.

### Mice

NSG (NOD.Cg-Prkdcscid Il2rgtm1Wj1/SzJ strain) mice were bred and maintained at the University of Florida Cancer Genetics Research Center. All animal experiments were conducted in accordance with approved protocols according to national and institutional guidelines. Maximum tolerated dose (MTD) studies were performed as described in the supplementary methods.

### Xenograft Tumor Models

Luc-PANC-1 (5×106) and Luc-CM (0.5×106) cells were resuspended in 200 ul of PBS and injected subcutaneously (SQ) or intraperitonially (IP) to 6-8-week-old NSG mice respectively. Tumor bearing mice were imaged using Xenogen IVIS® Lumina Bioluminescence Imaging System (Perkin Elmer) every week after cell injection. At 4 weeks, animals were randomized based on slope of luciferase signal and treatment was initiated. Mice received daily compound 19-S or 19-S7 at the indicated doses by intraperitoneal (IP) injection or by oral gavage (PO) and vehicle control mice received corn oil. For all animals body weight was recorded weekly and Luc-PANC-1 tumor volume was measured weekly with a caliper. For scheduled sacrifice experiments, animals were euthanized after 4 weeks of treatment. For survival studies, animals were sacrificed when tumors reached 1500 mm3 or when they showed any signs of ulceration. Harvested tumors were excised and weighted, and fixed in alcoholic formalin (67.5% Ethanol, 10% Formaldehyde 37%, 22.5% water) for pathology analysis. Tumor volume was calculated as [volume=0.52 (length×width2)].

### Drug Preparation for Treatment In Vitro and In Vivo

For in vitro studies 50 mM stock solutions of Pem, MTX, 19-S. 19-S5 and 19-S7 were prepared in 100% DMSO and kept protected from light at RT. 50 mM FUrd and FdUrd were prepared in water. Working solutions were prepared dissolving the stock solution in tissue culture media. Stock solutions were stored at RT; no changes in activity were observed for up to 6 months, as determined by repeated viability assays performed using MIA PaCa-2 cell. For all compounds, the dry solid was protected from light and stored at −30° C.

For all in vivo animal treatments, a 5% DMSO stock solution of compound 19-S or 19-S7 was prepared and the stock solution was stored at room temperature for up to 6 months. From this stock, the exact volume needed for daily in vivo delivery was then formulated into 95% corn oil (Mazola) at 2 different concentrations: 1.25 mg/mi (formulation 1) and 3.125 mg/ml (formulation 2). To determine the volume of solution administered to the mice, 200 μl (maximum volume to be delivered in mice via IP or oral gavage) was divided by 25 g of mouse body weight. Therefore, 8 μl of formulation 1 or 2 multiplied by the weight of the mice were used to reach a dose of 10 or 25 mg/kg of body weight, respectively.

### Bioluminescence Imaging

Prior to bioluminescence imaging the region where tumors were located was shaved. Mice were then anesthetized with 2.5% isoflurane in 02 then administered the D-luciferin substrate (150 mg/kg in PBS) by IP injection. Following the injection of the D-luciferin substrate mice were imaged using the Xenogen IVIS® Lumina Bioluminescence Imaging System (Perkin Elmer). The peak of luciferase photon flux was recorded 6 minutes after injection of the D-luciferin substrate. The total photon flux was analyzed and restricted to tumor ROI (region of interest) using Living Image v2.60.1 software (Imaging Systems).

### Human TS and DHFR Preparation

Human Thymidylate Synthase (hTS) and human Dihydrofolate Reductase (hDHFR) were both expressed in BL21 (DE13) E. coli cells, using a standard BL21 transformation protocol, utilizing the expression vector pQE80L-hTS) and pET100/D-TOPO (Invitrogen, K10001) respectively. Both vectors contained hTS and hD1-1FR with a N-terminal 6× Histidine tag. After bacterial transformation, cells were transferred to a Luria Broth culture medium for overnight growth at 37° C. in presence of ampicillin and cell growth was measured via OD600 absorbance. Protein expression was induced via addition of 1 mM Isopropyl β-D-1-thiogalactopyranoside (IPTG) once OD600 reached 0.6, approximately 4 hr. After 3 hours incubation in the presence of IPTG cells were pelleted via centrifugation (4000 RPM for 10 minutes in a Beckman J20 rotor). The resulting pellet(s) were resuspended in Wash Buffer 1 (WB 1) (20 mM NaH2PO4, 30 mM NaCl, 20 mM imidazole, pH 7.8) and lysed via a microfluidizer (LM110, Microfluidics) set to 18,000 PSI. The cell lysate was centrifuged for 75 minutes at 12,000 RPM in a Beckman J10 rotor and subsequent supernatant was collected and passed through a 0.8 μm syringe filter (MilliporeSigma, Burlington, MA, CAT #3 SLAA0335B) and loaded onto a HisTrap HP (GE) 5 mL column. The column was placed on AKT A pure 25 L1 FPLC (Cytiva, Marlborough, MA) and subjected to an imidazole gradient to elute the enzyme using WB1 and hTS Wash Buffer 2 (hTSWB2) (20 mM NaH2PO4, 30 mM NaCl, 500 mM Imidazole, pH 7.4) and hDHFR Wash Buffer 2 (hDHFRWB2) (20 mM NaH2PO4, 30 mM NaCl, 200 mM Imidazole, pH 7.8). Fractions collected were then subjected to 10% SDS-PAGE to identify hTS and hDHFR high purity fractions which were pooled and resuspended in hTS Storage Buffer (hTSSB) (20 mM NaH2PO4, 30 mM NaCl, pH 7.4) and hDHFR SEC Buffer (hDFRSEC) (10 mM Tris, 1 mM EDTA. pH 8.0) using AMICON Ultra-15 Centrifugal Filters (MilliporeSigma, Burlington, MA, CAT #UFC901008) respectively. hDHFR was subjected to further purification via size exclusion chromatography utilizing a HiLoad 16/600 Superdex 75pg column (MilliporeSigma, Burlington, MA. CAT #28989333). The major peak fractions were collected and buffer exchanged into hDHFR Storage Buffer (hDHFRS13, 10 mM Tris, 1 mM EDTA, 1 mM DTT, 50% Glycerol, pH 8.0). Final purity was determined via 10% SDS-Page and concentration was determined by UV/Vis spectroscopy at 280 nm. hTS was stored in hTSSB at 4° C. hDHFR was stored in hDHFRSB at −20° C.

### Tritium Based TS Catalytic Activity Assay

The catalytic activity of TS was determined by measuring the release of [31] from [5-3H]dUMP (ViTrax, VT122) resulting from the conversion of dUMP to dTMP as previously described12,33. The reaction conditions were optimized to determine the amount of TS protein needed to establish the range of TS catalytic activity assay and confirm the background signal from negative control reactions performed without TS or without the 5,10-methylene tetrahydrofolate (5,10-mTHF) cofactor (FIGS. 9A-9C). Reactions were performed in 50 mM KH2PO4 (Sigma Aldrich, P0662) at pH 7.2 using 2 μg of purified human TS. Reactions were performed in a final volume of 200 μL with 100 mM β-mercaptoethanol (Sigma Aldrich, M7522), 40 μM dUMP (Sigma Aldrich, D3876), 100 μM 5,10-mTHF, and the [5-3H]dUMP tracer (ViTrax, VT122) accounting for 0.06% of the final dUMP concentration in the reaction. The 5,10-mTHF used in the reaction was separately generated from tetrahydrofolic acid (Sigma Aldrich, T3125) by dissolving the solid in 10 mM KH12PO4 with 143 mM β-mercaptoethanol (1:100 from 14.3 M pure liquid), and 6.35 mM formaldehyde (Sigma Aldrich, 47608) to yield a 100 mM stock solution of 5,10-mTHF. Reactions were initiated by the addition of the dUMP substrate and 5,10-mTHF cofactor, then incubated at 37° C. for 30 minutes. Reactions performed in the presence of compound 19-S series analogues, FUrd (Sigma), FdUrd (Sigma), Pem (Sigma Aldrich, PHR1596), or MTX (Sigma Aldrich, CS340) were pre-incubated with each drug for 30 minutes before the reaction was initiated by the addition of the dUMP substrate and 5,10-mTHF cofactor. Reactions were terminated by the addition of 100 μL of ice-cold 20% trichloroacetic acid (Sigma Aldrich, T6399), then excess [5-31-]dUMP was removed by the addition of 200 μL of an albumin-coated activated charcoal solution. The albumin-coated activated charcoal solution was prepared by mixing 10 g of acid-washed activated dextran coated charcoal (Sigma Aldrich, C6241) with 2.5 g of bovine albumin (Sigma Aldrich, A1933) in 100 mL of H2O. The suspension was incubated at room temperature for 30 minutes and then centrifuged for 30 minutes at 10,000×g. Then 150 μL of the supernatant was collected and added to 5 mL of ultima gold liquid scintillation cocktail (Perkin Elmer, 6013326, lot: 77-19085), and scintillation measurements were perform using a liquid scintillation counter (Beckman Coulter LS6500) to determine [3H]H₂O levels in each sample. For all experiments, positive control reactions were performed with no drug representing 100% TS activity, and negative control reactions were performed with no TS enzyme or no 5,10-mTHF substrate representing 0% TS activity. All assays were performed in biological duplicate and technical duplicate. Results were analyzed using GraphPad Prism 8.

### Competitive Drug Displacement Assay

The tritium-based Ts catalytic activity assay was modified to determine if TS inhibition from 19-S, 19-S5, and 19-S7 was the result of competitive binding with either the dUMP substrate or the 5,10-mTHF cofactor. The competitive drug displacement assay utilized the standard reaction conditions for the tritium based TS catalytic activity assay including 40 μM dUMP (Sigma Aldrich, D3876) with the [5-3H]dUMP tracer (ViTrax, VT122) accounting for 0.06% of the final dUMP concentration and 100 μM 5,10-methylene tetrahydrofolate (defined as 1× dUMP and 1×5,10-mTHF). Concentrations of total dUMP and 5,10-mTHF were then changed relative to these 1× concentrations to concisely indicate the changes in either the substrate or cofactor concentration. The highest 5× concentration of 5,10-mTHF was 500 M which was limited by the solubility of the cofactor generated from THF. The highest 5× concentration of total dUMP was not limited by solubility, although increases were performed in the same increments as 5,10-mTHF for comparison and 0.06% of the [5-3H]dUMP tracer was maintained at all concentrations. The remaining components in each 200 μL reaction were consistent in all reactions and included 2 μg of purified human TS in 50 mM KH2PO4 (Sigma Aldrich, P0662) and 100 mM β-mercaptoethanol (Sigma Aldrich, M7522). Control antifolate inhibitor Pent (Sigma Aldrich, PHR1596) was performed for reference of a TS specific classical antifolate and to illustrate the increased activity as a result of displacement with the increasing ratios of the 5,10-mTHF cofactor. While FdUrd is a disclosed inhibitor targeting the dUMP binding site, its binding results in an irreversible covalent bond rather than the reversible binding required for the competitive drug displacement assay, although, increasing dUMP did not show drug displacement.

### DHFR Activity Assay

Compound 19-S series analogues activity screen against DHFR activity was determined by using a DHFR assay kit (Sigma Aldrich, CS0340) as described by the manufacturer. 0.1 unit DHFR enzyme (Sigma, D6566) was added to 1× assay buffer (Sigma, A5603), compound 19-S series analogues or control drugs. 6 μL NADPH solution and 5 μL of dihydrofolic acid (DHFR substrate) (Sigma, D7006). The reaction was then read on the spectrophotometer at 340 nm at 22° C. every 15 seconds for 2.5 minutes.

### Initial Computational Screening for Potential Inhibitors

DOCK6.5 with AMBER scoring to computationally screen a curated library within the constraints of the selected docking site in TS. (See onlinelibrary.wiley.com/doi/abs/10.1002/jcc.23905). Compounds were each positioned into the target region in 1000 orientations and ranked based on their predicted energy scores. Molecular docking experiments were performed by using Ut High Performance Computing Cluster resources. The top 1000 hits were visualized in PyMOL and manually checked for consistency of molecular docking predictions and overall fit. Using the ZINC database and ALOPGS (see vcclab.org/lab/alogps/), compounds with favorable lipophilicity (log P≤5), solubility (log S≥−4), and polar surface area (PSA) under ~140 Å² were selected, as these parameters are shown to be good predictors for absorption, distribution and oral bioavailability⁴⁸. The better-scoring 26 compounds from this final curated set were ordered and were tested, as described in the main text.

### Molecular Modeling of the Proposed Inhibition Modes

The 19-S ligand was built and optimized using the VMD Molefacture plugin49. PDB IDs 1 HVY and 4KAK were used as initial receptor structures for TS and D1 FR, respectively. Only protein residues and cofactors (dUMP and NADPH in each case) were kept, and all structural waters were removed. The receptor structure was prepared following the standard AutoDock protocol⁵⁰ using the prepare_receptor4.py script from AutoDock Tools. All non-polar hydrogens were merged, and Gasteiger charges and atom types were added. The ligand PDBQT was prepared using the prepare_ligand4.py script and modified to include de AC atom with prepare_bias.py script51, both available in AutoDock Tools. The grid size and position were chosen to include the whole ligand-binding site (including all protein atoms at a distance lower than 5 Å from all crystallized ligands). The spacing between grid points was set at 0.375 Å. AutoDock Bias protocol⁵¹ was applied to perform a biased docking experiment taking into consideration previous information from the targets. Briefly, considering the main interactions FIG. 15) of the co-crystallized ligands (D16 and 06U ligands from PDB IDs 1HVY and 4KAK, respectively), a hydrogen bond acceptor pharmacophoric restraint was added by modifying their respective energy grids (OA and NA maps) using prepare_bias.py script. Additionally, aromatic biases were handled by creating a new AC atom type in the center of aromatic rings and placing the grid (AC map) modification in the location of ligand interactors (FIG. 15), a strategy thoroughly validated by Arcon et al^{52,53}.

For each system, 100 different docking runs were performed and the results were clustered according to the ligand heavy atom RMSD using a cut-off of 2 Å. The Lamarckian Genetic Algorithm (LGA) parameters for each conformational search run were kept at their default values (150 for initial population size, 1×107 as the maximum number of energy evaluations, and 2.7×104 as the maximum number of generations). The docking results for 19-S were further analyzed by visual inspection.

Images of these molecules were prepared using the Visual Molecular Dynamics VMD) program⁴⁹. The 2D diagram of the protein-ligand interactions was generated using the PoseView server⁵⁴.

### Additional Methods

### Maximum Tolerated Dose (MTD)

For IP treatment, 6-8 weeks old NSG mice were IP injected with 10, 25, 50 or 100 mg/kg of compound 19-S either daily during 5 consecutive days. After 3 treatment cycles, animals rested (no treatment) for one more week before euthanasia. Body weight and physical status of all mice were closely monitored. Tissues were collected and fixed in alcoholic formalin to test for toxicity.

For PO treatment, 6-8 weeks old NSG were administered PO 25 or 50 mg/kg of compound 19-S daily during 5 consecutive days for 3 weeks. After 3 treatment cycles, animals rested (no treatment) for one more week before euthanasia. Body weight and physical status of all mice were closely monitored. Tissues were collected and fixed in alcoholic formalin to test for toxicity.

### Optimization of Tritium-Based TS Catalytic Assay

The tritium based TS catalytic activity assay was optimized to determine the amount of purified TS protein for each reaction. Additionally, these data showing the actual CPM values obtained highlight the range of the assay with low background signal observed when either TS or 5,10-mTHF is absent in the reaction compared to control reaction with all components and 2 ug of TS protein (FIGS. 9A-9C). The reactions performed without 5.10-mTHF resulted in a similar signal as reactions performed without the TS protein (FIGS. 9A-9C). This demonstrates the dependence of the reaction on the 5,10-mTHF cofactor and how the increased signal is due to the conversion of dUMP by TS and not influenced by other factor like potential oxidation, as observed in assays using the conversion 5,10-mTHF to THF as a readout for the TS reaction. Due to the sensitivity and range of the tritium based assay a large decrease in dUMP conversion is observed in the concentration range where the TS dimer begins transitioning to the inactive monomer form (FIGS. 9A-9C), this concentration dependent TS dimer to TS monomer transition is not detectable at the higher concentrations required for absorbance based assays.

First the hTS catalytic activity of different amounts of hTS was determined by measuring the release of [3H] from [5-3H]dUMP (ViTrax) during the conversion of dUMP to dTMP (Figure S2a and S2b). The total volume for each reaction was 200 μL and reactions were performed using purified hTS ranging from 0.00625 ug to 100 ug (FIG. 9A and FIG. 9B). All reactions were performed in 50 mM KH2PO4 (Sigma Aldrich, P0662) at pH 7.2 with 100 mM J3-mercaptoethanol (Sigma Aldrich, M7522), 40 LEM dUMP (Sigma Aldrich, D3876), 100 μM 5,10-methylene tetrahydrofolate, and the [5-3H]dUMP tracer (ViTrax, VT122) accounting for 0.06% of the final dUMP concentration in the reaction. Each reaction was incubated at 37° C. for 30 minutes, then the reaction is terminated by the addition of 100 μL of ice-cold 20% trichloroacetic acid (Sigma Aldrich, T6399). Excess [5-3H]dUMP was removed by the addition of 200 μL of an albumin-coated activated charcoal solution (prepared by mixing 10 g of acid-washed activated dextran coated charcoal (Sigma Aldrich, C6241), 15 g of bovine albumin (Sigma Aldrich, A1933) and 100 mL of water). The suspension was incubated at room temperature for 30 min and then centrifugated for 30 min at 10,000×g. Then 150 μL of the supernatant was collected and added to 5 mL of ultima gold liquid scintillation cocktail (Perkin Elmer, 6013326, lot: 77-19085), and scintillation measurements were performed using a liquid scintillation counter (Beckman Coulter LS6500) to determine [3H]H2O levels in each sample. For each 200 μL reaction 2 μg of purified TS protein was selected for future assays. Then reactions were performed with all components except the 5,10-mTHF cofactor, all reaction components except 2 μg of TS protein, and a positive control with all components including 2 μg TS protein to show the range of the assay, the background signal, and the dependence on 5,10-mTHF (FIG. 9C). To determine the signal from the [5-3H]dUMP tracer used in each reaction the same amount of the dUMP substrate with the [5-3H]dUMP tracer accounting for 0.06% of the final dUMP concentration was added directly to 5 mL of ultima gold liquid scintillation cocktail (FIG. 9C). The resulting signal was over 8-fold higher than the positive control reaction demonstrating all the [5-3H]dUMP tracer is not converted in the reaction and demonstrates the efficiency of the albumin-coated activated charcoal solution removing the [5-3H]dUMP that was not converted during the reaction. All reactions were performed in biological duplicate and technical duplicate. Results were analyzed using GraphPad Prism 8.

### Example 4. Chemical Synthesis, Characterization Data, and NMR Spectra

### Chemical Synthesis and Characterization Data

All reagents for chemical synthesis were purchased at ≥95% purity from commercial sources and used without further purification. Analytical thin layer chromatography (TLC) was performed using 250 μm Silica Gel 60 F254 pre-coated plates (EMD Chemicals Inc.) and used to monitor all reactions. Flash column chromatography was performed using 230-400 Mesh 60 Å Silica Gel from Sorbent Technologies. Melting points were obtained, uncorrected, using a Mel-Temp capillary melting point apparatus from Laboratory Services, Inc.

NMR experiments were recorded using broadband probes on a Varian Mercury-Plus-400 spectrometer via VNMR-J software (400 MHz for ¹H and 101 MHz for ¹³C) Varian Mercury-Plus-500 spectrometer via VNMR-J software (500 MHz for 1H and 126 MHz for ¹³C), and Bruker Avance II (500 MHz for ¹H: 126 MHz for ¹³C). All spectra are presented using MestReNova 11.0 (Mnova) software and are displayed without the use of the signal suppression function. Spectra were obtained at room temperature in the following solvents (reference peaks for 1H and 13C NMRs are included): CDCl3 (¹H NMR, 7.26 ppm; ¹³C NMR, 77.23 ppm) and DMSO-d₆ (¹H NMR, 2.50 ppm; ¹³C NMR, 39.52 ppm). Chemical shift values (6) are reported in parts per mil lion (ppm) for all ¹H NMR and ¹³C NMR spectra. ¹H NMR multiplicities are reported as: s=singlet, br. s=broad singlet, d=doublet, t triplet, q=quartet, m=multiplet. HSQC was used to identify a few challenging ¹³C signals and those spectra are reported in the supporting information. High-Resolution Mass Spectrometry (IRMS) were obtained for all new compounds.

### Synthesis Overview

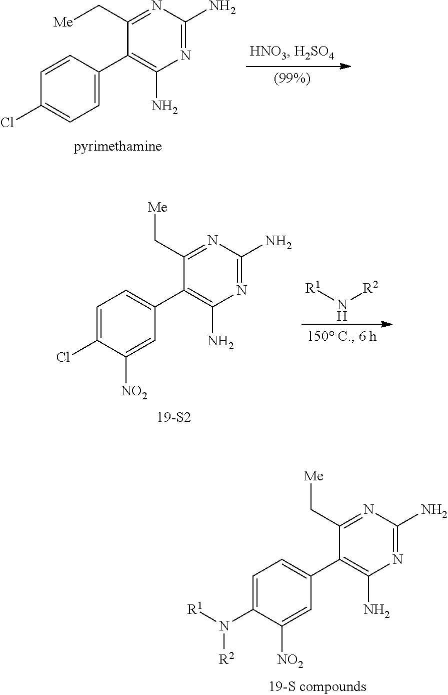

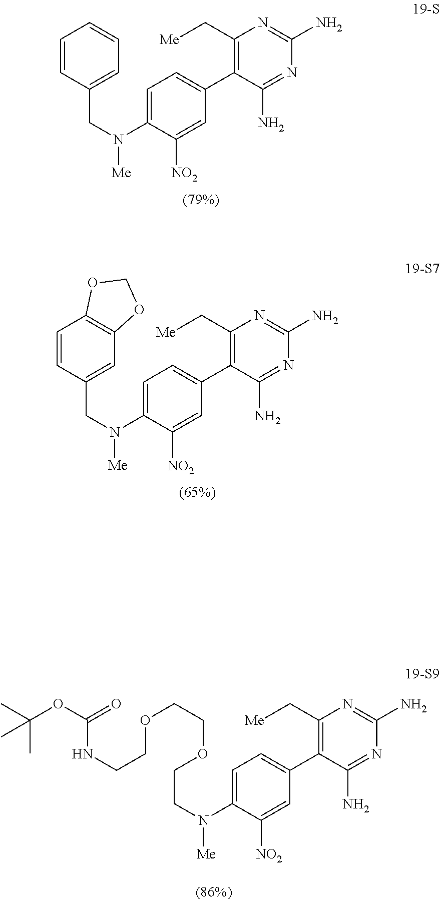 [SMILES: CCc1nc(N)nc(N)c1-c1ccc(N(C)Cc2ccccc2)c([N+](=O)[O-])c1 ; CCc1nc(N)nc(N)c1-c1ccc(N(C)Cc2ccc3c(c2)OCO3)c([N+](=O)[O-])c1 ; CCc1nc(N)nc(N)c1-c1ccc(N(C)CCOCCOCCNC(=O)OC(C)(C)C)c([N+](=O)[O-])c1]

Compound 19-S and derivatives were synthesized through a two-step route. First, commercially available pyrimethamine was selectively ortho-nitrated using nitric acid in sulfuric acid (99% yield). The nitro precursor was subjected to a nucleophilic aromatic substitution reaction with various amines to produce Compound S and derivatives in a rapid and high yielding synthetic sequence (12 examples, average yield=58%). Additionally, both the nitration reaction as well as several analogues were able to scale to several hundreds of milligrams with negligible loss in yield, which provided more than enough material for substantial in vivo testing in mice tumor models (e.g., second step scales for select analogues of interest include: 391 mg 19-S, 461 mg 19-S7).

### Procedure for the Synthesis of 19-S2:

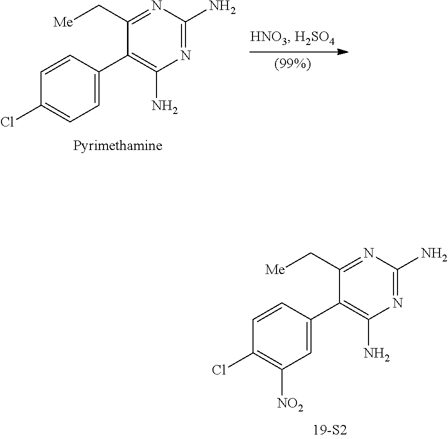 [SMILES: CCc1nc(N)nc(N)c1-c1ccc(Cl)cc1 ; CCc1nc(N)nc(N)c1-c1ccc(Cl)c([N+](=O)[O-])c1]

A round-bottom flask was added concentrated sulfuric acid (2.0 mL) and cooled to 0° C. Concentrated nitric acid was added dropwise and allowed to stir for 5 minutes. Pyrimethamine (500 mg, 2.01 mmol) was added in several portions and the reaction was stirred at 0° C. for 15 minutes, then allowed to warm to room temperature, then heated to 50° C. for 1 hour. The resulting yellow solution was cooled to room temperature, then slowly added to a flask containing ice:ammonium hydroxide:water which produced a yellow precipitate. The precipitate was filtered, washed with additional water, and dried to afford pure 19-S2 (589 mg, 99%) as a yellow solid. Note: 19-S2 is a disclosed compound (CAS number: 21813-35-4).

¹H NMR: (400 MHz, d₆-DMSO) δ 7.87 (d, J=2.1 Hz, 11H), 7.78 (d, J=8.3 Hz, 11H), 7.51 (dd, J=8.3, 2.1 Hz, 1H), 5.99 (br. s, 21H), 5.86 (br. s, 2H). 2.12 (q, J=7.5 Hz, 2H), 0.98 (t, J=7.5 Hz, 3H).

¹³C NMR: (101 MHz, d₆-DMSO) δ 166.4, 162.3, 162.0, 148.0, 137.0, 136.6, 132.0, 127.9, 123.7, 103.6, 27.4, 12.9.

### General Procedure for the Synthesis of 19-S Compounds:

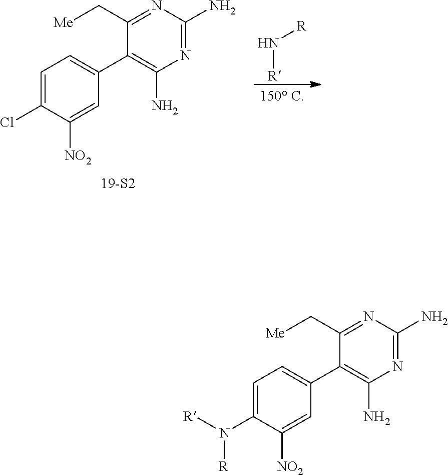

19-S2 (293 mg, 1.00 mmol) was added to a screw-top vessel, purged with argon and added benzylmethylamine (1 mL, 7.75 mmol). The vessel was sealed and heated to 150° C. for 6 hours. The resulting dark-red solution was cooled to room temperature and diethyl ether was added (35 mL). The precipitate was collected, washed with additional ether and dried. The crude precipitate was purified via recrystallization from 2-ethoxyethanol:water 3.5:1 to afford compound 19-S (300 mg, 79%) as a red, crystalline solid.

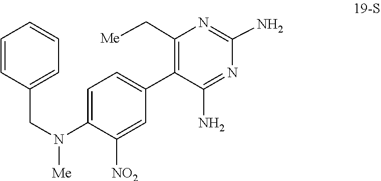 [SMILES: CCc1nc(N)nc(N)c1-c1ccc(N(C)Cc2ccccc2)c([N+](=O)[O-])c1]

Yield: 79%; 300 mg of 19-S isolated as a red, crystalline solid. Note: 19-S is a disclosed compound (CAS number: 118344-71-1); however, no published spectra were found for comparison.

¹H NMR: (400 MHz, d₆-DMSO) δ 7.55 (s, 1H), 7.39-7.33 (m, 2H), 7.33-7.24 (m, 51), 5.90 (br. s, 2H), 5.72 (br. s, 2H), 4.42 (br. s, 2H), 2.72 (s, 3H), 2.12 (q, j=7.5 Hz, 2H), 0.97 (t, J=7.5 Hz, 3H).

¹³C NMR: (101 MHz, d₆-DMSO) δ 166.7, 162.3, 162.1, 144.3, 140.1, 137.4, 135.9, 128.5, 127.7, 127.5, 127.2, 126.7, 120.6, 104.4, 57.8, 40.3 27.4, 13.1.

MP: 208-210° C., lit: 210-211° C.

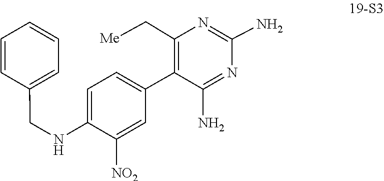 [SMILES: CCc1nc(N)nc(N)c1-c1ccc(NCc2ccccc2)c([N+](=O)[O-])c1]

Yield: 74%; 580 mg of 19-S1 isolated as an orange, crystalline solid.

¹H NMR: (400 MHz, d₆-DMSO) δ 7.54 (d, J=1.7 Hz, 1H), 7.26 (d, J=1.9 Hz, 2H), 7.22-7.03 (m, 4H), 5.89 (br s, 2H), 5.70 (br s, 2H), 4.36 (br s, 2H), 2.70 (s, 3H), 2.28 (s, 31H), 2.12 (q, J=7.5 Hz, 2H), 0.97 (t, J=7.5 Hz, 3H).

¹³C NMR: (101 MHz, d₆-DMSO) δ 166.9, 162.3, 162.1, 144.3, 140.1, 136.3, 135.9, 134.2, 129.0, 127.7, 127.6, 126.6, 120.6, 104.4, 57.5, 40.2, 27.5, 20.7, 13.1.

HRMS (ESI): calc. for C₂₁H₂₅N₆O₂ [M+H]⁺: 393.2034, found: 393.2046.

MP: 232-233° C.

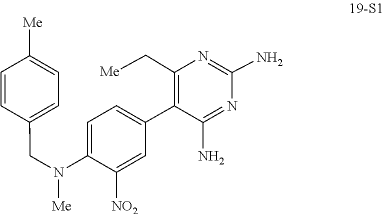 [SMILES: CCc1nc(N)nc(N)c1-c1ccc(N(C)Cc2ccc(C)cc2)c([N+](=O)[O-])c1]

Yield: 62% 78.3 mg of 19-S3 isolated as a red, crystalline solid. Note: 19-S3 is a disclosed compound (CAS number: 118344-70-0); however, no published spectra were found for comparison.

¹H NMR: (400 MHz, d₆-DMSO) δ 872 (t, J=6.1 Hz, 1H), 7.82 (d, J=2.1 Hz, 1H), 7.45-7.40 (m, 2H) 7.0-7.33 (m, 2H), 7.31-7.23 (m, 2H), 6.99 (d, J=8.9 Hz, 1H), 5.84 (br. s. 2H), 5.66 (br. s, 2H), 4.65 (d, J=6.0 Hz, 2H), 2.10 (q, 1=7.5 Hz, 2H), 0.95 (t, J=7.5 Hz, 3H).

¹³C NMR: (101 MHz, d₆-DMSO) δ 167.0, 162.5, 162.1, 144.2, 139.3, 138.6, 131.4, 128.6, 127.8, 127.1 (2), 122.9, 115.4, 104.4, 45.9, 27.5, 13.1.

HRMS (ESI): calc. for C₁₉H₂₁N₆O₂ M+[H]⁺: 365.1721, found: 365.1719.

MP: >250° C., lit: 253-255° C.

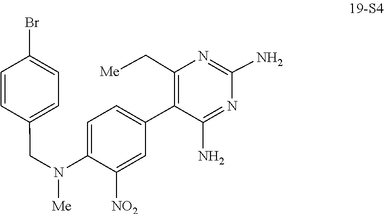 [SMILES: CCc1nc(N)nc(N)c1-c1ccc(N(C)Cc2ccc(Br)cc2)c([N+](=O)[O-])c1]

Yield: 78%; 204 mg of 19-S4 isolated as a red-orange solid

¹H NMR: (400 MHz, d₆-DMSO) δ 7.60-7.51 (m, 3H), 7.32-7.22 (m, 4H), 5.88 (br. s, 2H), 5.69 (br. s, 2H), 4.38 (s. 2H), 2.71 (s, 3H), 2.11 (q, J=7.5 Hz, 2H), 0.97 (t, J=7.5 Hz, 3H).

¹³C NMR: (101 MHz, d₆-DMSO) δ 166.9, 162.3, 162.1, 144.1, 140.4, 136.9, 136.0, 131.3, 129.8, 127.7, 127.2, 120.8, 120.2, 104.3, 57.2, 40.4, 27.5, 13.1.

HRMS (ESI): calc. for C₂₀H₂₂BrN₆O₂ [M+H]⁺: 457.0982, found: 457.0996.

MP: 219-221° C.

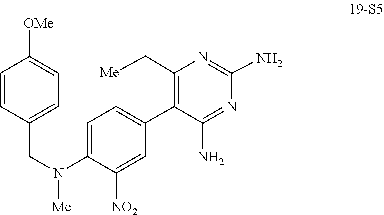 [SMILES: CCc1nc(N)nc(N)c1-c1ccc(N(C)Cc2ccc(OC)cc2)c([N+](=O)[O-])c1]

Yield: 48%; 107 mg of 19-S5 isolated as an orange solid. Note: 19-S5 has an assigned CAS number (118344-80-2), but no published spectra were found for comparison

¹H NMR: (400 MHz, d₆-DMSO) δ 7.54 (s, 1H), 7.21 (d, J=8.3 Hz, 2H), 7.21 (d, J=8.5 Hz, 2H), 6.91 (d, J=8.3 Hz, 2H), 5.88 (br. s, 2H), 5.69 (br. s, 2H), 4.32 (s, 2H), 3.74 (s, 3H), 2.68 (s, 3H), 2.12 (q, J=7.5 Hz, 2H), 0.97 (t, J=7.5 Hz, 3H).

¹³C NMR: (101 MHz, d₆-DMSO) δ 166.9, 162.3, 162.1, 158.5, 144.3, 140.3, 135.9, 129.0, 1289, 127.6, 126.8, 120.7, 113.8, 104.4, 573, 55.0, 40.0, 27.5, 13.1.

HRMS (ESI): calc. for C₂₁H₂₅N₆O₃ [M+H]⁺: 409.1983, found: 409.1999.

MP: 195-197° C., lit: 201-203° C.

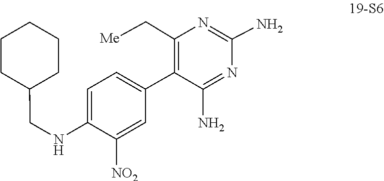 [SMILES: CCc1nc(N)nc(N)c1-c1ccc(NCC2CCCCC2)c([N+](=O)[O-])c1]

Yield: 70%; 136 mg of 19-S6 isolated as an orange crystalline solid.

¹H NMR: (400 MHz, d₆-DNMSO) δ 8.26 (t, J=5.2 Hz, 1H), 7.79 (d, J=1.6 Hz, j H), 7.31 (dd, J=8.5, 2.1 Hz, 1H), 7.11 (d, J=8.9 Hz, 1H), 5.84 (br. s, 2H), 5.68 (br. s, 2H), 3.24 (t, J=6.3 Hz, 2H), 2.13 (q, J=7.5 Hz, 2H), 1.85-1.58 (m, 6H), 1.31-1.10 (m, 3H), 1.10-1.01 (m. 2H), 0.97 (t, J=7.5 Hz, 3H).

¹³C NMR: (101 MHz, d₆-DMSO) δ 167.0, 162.5, 162.1, 144.7, 139.5, 130.9, 127.7, 122.5, 115.2, 104.5, 48.5, 36.7, 30.4, 27.5, 26.0, 25.4, 13.1.

HRMS (ESI): calc. for C₁₉H₂₇N₆O₂ [M+H]⁺: 371.2190, found: 271.2196.

MP: >250° C.

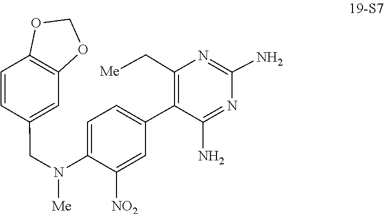 [SMILES: CCc1nc(N)nc(N)c1-c1ccc(N(C)Cc2ccc3c(c2)OCO3)c([N+](=O)[O-])c1]

Yield: 65%; 148 mg of 19-S7 isolated as an orange solid

¹H NMR: (400 MHz, d₆-DMSO) δ 7.54 (t, J=1.2 Hz, 1H), 7.28 (d, J=1.2 Hz, 21H), 6.88 (d, J=7.9 Hz, 1H), 6.83 (d, J=1.6 Hz, 1H), 6.77 (dd, J=7.9, 1.7 Hz, 1H), 6.00 (s, 2H), 5.88 (s, 21H), 5.70 (br. s, 2H), 4.29 (hr. s, 2H), 2.69 (s, 3H), 2.12 (q, J=7.5 Hz, 2H), 0.97 (t, J=7.5 Hz, 3H).

¹³C NMR: (101 MHz, d₆-DMSO) δ 166.8, 162.3, 162.1, 147.4, 146.4, 144.2, 140.4, 135.9, 131.1, 127.6, 126.9, 120.9, 120.8, 108.1, 107.9, 104.4, 100.9, 57.6, 40.2 (buried under d₆-DMSO), 27.5, 13.0.

HRMS (ESI): calc. for C₂₁H₂₃N₆O₄ [M+H]⁺: 423.1775, found: 423.1783.

MP: 194-196° C.

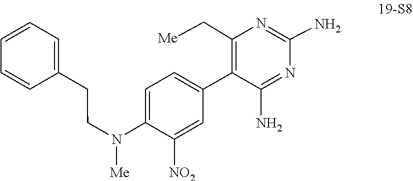 [SMILES: CCc1nc(N)nc(N)c1-c1ccc(N(C)CCc2ccccc2)c([N+](=O)[O-])c1]

Yield: 22%; 45.5 mg of 19-88 isolated as an orange solid

¹H NMR: (400 MHz, d₆-DMSO) δ 7.47 (s, 1H), 7.29-7.14 (m, 7H), 5.87 (br. s, 2H), 5.64 (br. s. 2H), 3.37 (m, 2H), 2.92-2.82 (m, 5H), 2.11 (q, J=7.5 Hz, 2H), 0.97 (t, J=7.5 Hz, 3H).

¹³C NMR: (101 MHz, d₆-DMSO) δ 166.9, 162.3, 162.1, 143.8, 139.8, 139.1, 135.8, 128.7, 128.3, 127.7, 126.2, 126.0, 120.1, 104.4, 55.9, 39.7 (buried under d₆-DMSO), 32.9, 27.4, 13.1.

HRMS (ESI): calc. for C₂₁H₂₅N₆O₂ [M+H]⁺: 393.2034. found: 393.2022.

MP: 184-186° C.

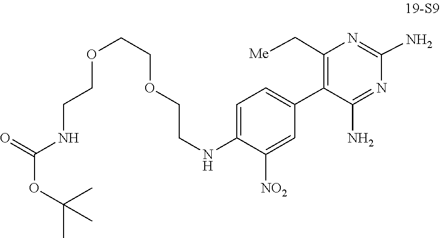 [SMILES: CCc1nc(N)nc(N)c1-c1ccc(NCCOCCOCCNC(=O)OC(C)(C)C)c([N+](=O)[O-])c1]

Yield: 86%; 447 mg of 19-S9 isolated as a red-orange solid. Note: 19-S9 was purified via column chromatography using a gradient of 99:1 hexanes:triethylanine to 98:1:1 ethyl acetate:methanol:trimethylamine

¹H NMR: (400 MHz, CDCl3) δ 8.19 (t, Jr 5.0 Hz, 1H), 7.96 (d, J=2.1 Hz, 1H), 7.24 (dd, J=8.8, 2.1 Hz, 1H), 6.88 (d, J=8.9 Hz, 1H), 5.55 (t, J=5.9 Hz, 1H), 5.30 (s, 2H), 5.17-4.94 (m, 2H), 3.73 (t, J=5.3 Hz, 2H), 3.67-3.56 (m, 4H), 3.54-3.39 (m, 4H), 3.30-3.19 (m, 2H), 2.18 (q, J=7.6 Hz, 2H), 1.36 (s, 9H), 0.97 (t, J=7.6 Hz, 3H).

¹³C NMR: (101 MHz, CDCl3) δ 168.8, 162.7, 161.9, 156.1, 144.8, 138.9, 132.1, 128.5, 122.2, 115.0, 106.0, 79.2, 70.6, 70.5, 70.3, 68.9, 42.9, 40.4, 28.5, 28.1, 13.4

HRMS (ESI): calc. for C₂₃H₃₆N₇O₆ [M+H]⁺: 506.2722, found: 506.2742.

MP: 87-89° C.

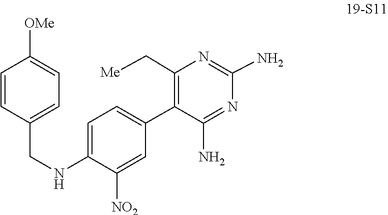 [SMILES: CCc1nc(N)nc(N)c1-c1ccc(NCc2ccc(OC)cc2)c([N+](=O)[O-])c1]

Yield: 27%; 57.8 mg of 19-S11 isolated as a red, crystalline solid. Note: 19-511 is a disclosed compound (CAS number: 118344-94-8); however, no published spectra were found for comparison.

¹H NMR: (400 MHz, d₆-DMSO) δ 8.63 (t, J=5.7 Hz, 1H), 7.81 (s, 1H), 7.36 (d, J=8.2 Hz, 2H), 7.27 (d, J=8.8 Hz, 1H), 7.03 (d, J=8.8 Hz, 1H), 6.93 (d, J=8.2 Hz, 2H), 5.84 (hr. s, 2H), 5.65 (br. s, 2H), 4.56 (d, J=5.8 Hz, 2H), 3.73 (s, 3H), 2.10 (q, J=7.5 Hz, 2H), 0.95 (t, J=7.5 Hz, 3H).

¹³C NMR: (101 MHz, d₆-DMSO) δ 166.9, 162.5, 162.0, 158.5, 144.2, 1393, 131.3, 130.3, 128.5, 127.8, 1228, 115.4, 114.0, 104.4, 55.1, 45.4, 27.4, 13.1.

HRMS (ESI): calc. for C₂₀H₂₃N₆O₃ [M+H]⁺: 395.1826, found: 395.1826.

MP: 242-244° C., lit: 241-242° C.

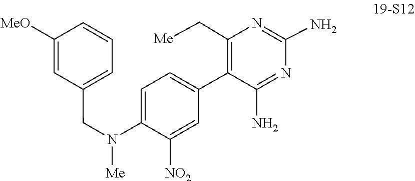 [SMILES: CCc1nc(N)nc(N)c1-c1ccc(N(C)Cc2cccc(OC)c2)c([N+](=O)[O-])c1]

Yield: 55° %; 121 mg of 19-S12 isolated as a yellow-orange solid

¹H NMR: (400 MHz, d₆-DMSO) δ 7.54 (s, 1H), 7.30-7.22 (m, 3H), 6.90-6.80 (m, 3H), 5.87 (br. s, 2H), 5.68 (br. s, 2H), 4.40 (s, 2H), 3.73 (s, 3H), 2.72 (s, 3H), 2.11 (q, J=7.5 Hz, 2H), 0.97 (t, J=7.5 Hz, 3H).

¹³C NMR: (10 MHz, d₆-DMSO) δ 166.9, 162.3, 162.1, 159.4, 144.2, 140.2, 139.1, 135.9, 129.5, 127.7, 126.8, 120.6, 119.6, 113.0, 112.6, 104.3, 57.6, 54.9, 40.5, 27.5, 13.0.

HRMS (ESI): calc. for C₂₁H₂₅N₆O₃ [M+H]⁺: 409.1983, found: 409.1991.

MP: 163-165° C.

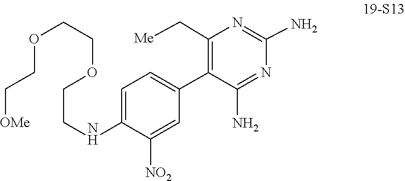 [SMILES: CCc1nc(N)nc(N)c1-c1ccc(NCCOCCOCCOC)c([N+](=O)[O-])c1]

Yield: 29%; 61.2 mg of 19-S13 isolated as an orange solid. Note: 19-S13 was purified via column chromatography using a gradient of 99:1 hexanes:triethylamine to 98:1:1 ethyl acetate:methanol:trimethylamine

¹H NMR: (400 MHz, CDCl₃) δ 8.27 (t, J=5.2 Hz, 11H), 8.04 (d, J=2.1 Hz, 1H), 7.28 (dd, J=8.8, 2.1 Hz, 1H), 6.94 (d, J=8.8 Hz, 1H), 5.04 (br. s, 2H), 4.76 (br. s, 2H). 3.79 (I, J=5.3 Hz, 2H), 3.73-3.60 (m, 6H), 3.56-3.48 (m, 41H), 3.35 (s, 3H), 2.25 (q, J=7.6 Hz, 2H), 1.04 (t, J=7.6 Hz, 3H).

¹³C NMR: (101 MHz, CDCl₃) δ 168.9, 162.7, 161.7, 145.0, 138.8, 132.4, 128.7, 121.9, 115.1, 106.4, 72.1, 70.9, 70.8, 70.8, 69.2, 59.2, 43.0, 28.2, 13.5.

HRMS (ESI): calc. for C₁₉H₂₈N₆O₅Na [M+Na]⁺: 443.2013. found: 443.2024.

MP: 131-133° C.

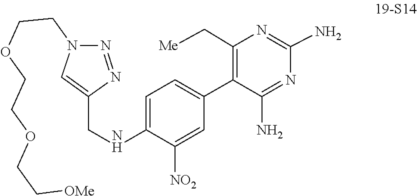 [SMILES: CCc1nc(N)nc(N)c1-c1ccc(NCc2cn(CCOCCOCCOC)nn2)c([N+](=O)[O-])c1]

Yield: 61%; 41.5 mg of 19-S14 isolated as an orange-red, amorphous solid. Note: 19-S14 was purified via silica gel, column chromatography using a gradient of 99:1 ethyl acetate:triethylamine to 97:2:1 ethyl acetate:methanol:triethylamine. After the first column, an unidentified impurity coeluted with the desired product. A second purification was performed via column chromatography, using alumina neutral act I and a gradient of 100% dichloromethane to 2% methanol:dichloromethane to afford pure product.

¹H NMR: (500 MHz, CDCl3) δ 8.46 (t, J=5.8 Hz, 1H), 8.09 (s, 1H), 7.81 (s, 1H), 7.32 (d, J=8.7 Hz, 1H), 7.16 (d, J=8.7 Hz, 1H), 4.78 (br. s, 2H), 4.70 (d, J=5.6 Hz, 2H), 4.56 (t, J=5.0 Hz, 2H), 4.50 (br. s. 2H), 3.87 (t, J=5.0 Hz, 2H), 3.65-3.57 (m, 6H), 3.57-3.51 (n, 2H), 3.36 (s, 3H), 2.29 (q, J=7.6 Hz, 2H), 1.08 (t, J=7.6 Hz, 3H).

¹³C NMR: (126 MHz, CDCl3) δ 168.2, 162.8, 161.4, 144.5, 144.3, 138.9, 132.6, 128.6, 123.5, 122.3, 115.5, 106.3, 72.0, 70.6 (3), 69.5, 59.1, 50.6, 39.1, 28.0, 13.5.

HRMS (ESI): calc. for C₂₂H₃₂N₉O₅[M+H]⁺: 502.2521, found: 502.2537.

### Procedure for the Synthesis of 19-S10

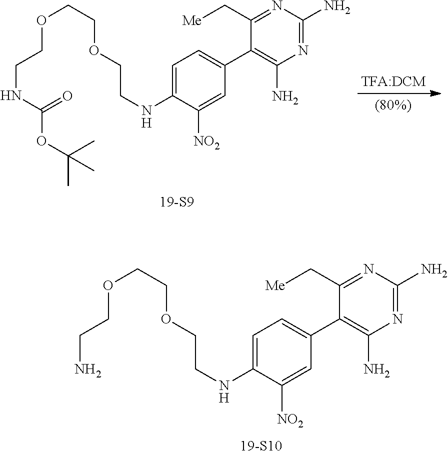 [SMILES: CCc1nc(N)nc(N)c1-c1ccc(NCCOCCOCCNC(=O)OC(C)(C)C)c([N+](=O)[O-])c1 ; CCc1nc(N)nc(N)c1-c1ccc(NCCOCCOCCN)c([N+](=O)[O-])c1]

Compound 19-S9 (179 mg, 0.35 mmol) was added to a flame-dry round-bottom flask and dissolved in dichloromethane (1.5 mL). The solution was cooled to 0° C. and added trifluoroacetic acid (1 mL) dropwise. The reaction was stirred at 0° C. for 0.5 hours then warmed to room temperature and concentrated in vacuo. The residue was dissolved in dichloromethane (30 mL) and extracted with saturated sodium bicarbonate solution (4×150 mL). The organics were pooled, dried with sodium sulfate, filtered and concentrated to afford pure compound 19-S10 (115 mg, 80%) as a red-orange solid.

¹H NMR: (400 MHz, d₆-DMSO) δ 8.26 (t, J=5.3 Hz, 1H), 7.80 (d, J=2.1 Hz, 1H), 7.33 (dd, J=8.8, 2.2 Hz, 1H), 7.15 (d, J=8.8 Hz, 1H), 5.85 (s, 2H), 5.68 (s, 2H), 3.71 (t, J=5.4 Hz, 2H), 3.63-3.58 (m, 2H), 3.58-3.49 (m, 4H), 3.38-3.34 (m, 4H), 2.63 (t, J=5.8 Hz, 2H), 2.13 (q, J=7.5 Hz, 2H), 0.97 (t, J=7.5 Hz, 3H).

¹³C NMR: (101 MHz, d₆-DMSO) δ 167.0, 162.5, 162.1, 144.4, 139.5, 131.2, 127.7, 122.7, 115.4, 104.5, 72.9, 69.7, 69.6, 68.4, 42.2, 41.2, 27.5, 13.2.

HRMS (ESI): calc. for C₁₈H₂₈N₇O₄ [M+H]⁺: 406.2197, found: 406.2192.

MP: 134-136° C.

### REFERENCES

1. Wilson, P. M., Danenberg, P. V., Johnston, P. G., Lenz, H. J. & Ladner, R. D. Standing the test of time: targeting thymidylate biosynthesis in cancer therapy. Nat Rev Clin Oncol 11, 282-298, doi:10.1038/nrclinonc.2014.51 (2014).
2. Longley, D. B. et al. Characterization of a thymidylate synthase (TS)-inducible cell line: a model system for studying sensitivity to TS- and non-TS-targeted chemotherapies. Clin Cancer Res 7, 3533-3539 (2001).
3. Johnston, P. G. et al. Thymidylate synthase gene and protein expression correlate and are associated with response to 5-fluorouracil in human colorectal and gastric tumors. Cancer Res 55, 1407-1412 (1995).
4. Lenz, H. J. et al. p53 and thymidylate synthase expression in untreated stage II colon cancer: associations with recurrence, survival, and site. Clin Cancer Res 4, 1227-1234 (1998).
5. Salonga, D. et al. Colorectal tumors responding to 5-fluorouracil have low gene expression levels of dihydropyrimidine dehydrogenase, thymidylate synthase, and thymidine phosphorylase. Clin Cancer Res 6, 1322-1327 (2000).
6. Qiu, L. X. et al. Predictive value of thymidylate synthase expression in advanced colorectal cancer patients receiving fluoropyrimidine-based chemotherapy: evidence from 24 studies. Int J Cancer 123, 2384-2389, doi:10.1002/ijc.23822 (2008).
7. Liu, Y. et al. Expression of thymidylate synthase predicts clinical outcomes of pemetrexed-containing chemotherapy for non-small-cell lung cancer: a systemic review and meta-analysis. Cancer Chemother Pharmacol 72, 1125-1132, doi:10.1007/s00280-013-2299-2 (2013).
8. Christoph, D. C. et al. Significance of folate receptor alpha and thymidylate synthase protein expression in patients with non-small-cell lung cancer treated with pemetrexed. J Thorac Oncol 8, 19-30, doi:10.1097/JTO.0b013e31827628ff (2013).
9. Sun, J. M., Han, J., Ahn, J. S., Park, K. & Ahn, M. J. Significance of thymidylate synthase and thyroid transcription factor 1 expression in patients with nonsquamous non-small cell lung cancer treated with pemetrexed-based chemotherapy. J Thorac Oncol 6, 1392-1399, doi:10.1097/JTO.0b013e3182208ea8 (2011).
10. Zucali, P. A. et al. Thymidylate synthase and excision repair cross-complementing group-1 as predictors of responsiveness in mesothelioma patients treated with pemetrexed/carboplatin. Clin Cancer Res 17, 2581-2590, doi:10.1158/1078-0432.CCR-10-2873 (2011).
11. Righi, L. et al. Thymidylate synthase but not excision repair cross-complementation group 1 tumor expression predicts outcome in patients with malignant pleural mesothelioma treated with pemetrexed-based chemotherapy. J Clin Oncol 28, 1534-1539, doi:10.1200/JCO.2009.25.9275 (2010).
12. Rahman, L. et al. Thymidylate synthase as an oncogene: a novel role for an essential DNA synthesis enzyme. Cancer Cell 5, 341-351, doi:10.1016/s1535-6108(04)00080-7 (2004).
13. Scagliotti, G. V. et al. Phase III study comparing cisplatin plus gemcitabine with cisplatin plus pemetrexed in chemotherapy-naive patients with advanced-stage non-small-cell lung cancer. J Clin Oncol 26, 3543-3551, doi:10.1200/JCO.2007.15.0375 (2008).
14. Longley, D. B., Harkin, D. P. & Johnston, P. G. 5-fluorouracil: mechanisms of action and clinical strategies. Nat Rev Cancer 3, 330-338, doi:10.1038/nrc1074 (2003).
15. Goulian, M. et al. Mechanism of thymineless death. Adv Exp Med Biol 195 Pt B, 89-95, doi:10.1007/978-1-4684-1248-2_15 (1986).
16. Sommer, H. & Santi, D. V. Purification and amino acid analysis of an active site peptide from thymidylate synthetase containing covalently bound 5-fluoro-2′-deoxyuridylate and methylenetetrahydrofolate. Biochem Biophys Res Commun 57, 689-695, doi:10.1016/0006-291x(74)90601-9 (1974).
17. Santi, D. V., McHenry, C. S. & Sommer, H. Mechanism of interaction of thymidylate synthetase with 5-fluorodeoxyuridylate. Biochemistry 13, 471-481, doi:10.1021/bi00700a012 (1974).
18. Welsh, S. J. et al. Comparison of thymidylate synthase (TS) protein up-regulation after exposure to TS inhibitors in normal and tumor cell lines and tissues. Clin Cancer Res 6, 2538-2546 (2000).
19. Chu, E. et al. Induction of thymidylate synthase associated with multidrug resistance in human breast and colon cancer cell lines. Mol Pharmacol 39, 136-143 (1991).
20. Farber, S. & Diamond, L. K. Temporary remissions in acute leukemia in children produced by folic acid antagonist, 4-aminopteroyl-glutamic acid. N Engl J Med 238, 787-793, doi:10.1056/NEJM194806032382301 (1948).
21. Visentin, M., Zhao, R. & Goldman, I. D. The antifolates. Hematol Oncol Clin North Am 26, 629-648, ix, doi:10.1016/j.hoc.2012.02.002 (2012).
22. Hryniuk, W. M. The mechanism of action of methotrexate in cultured L5178Y leukemia cells. Cancer Res 35, 1085-1092 (1975).
23. Rustum, Y. M. et al. Thymidylate synthase inhibitors in cancer therapy: direct and indirect inhibitors. J Clin Oncol 15, 389-400, doi:10.1200/JCO.1997.15.1.389 (1997).
24. Assaraf, Y. G. Molecular basis of antifolate resistance. Cancer Metastasis Rev 26, 153-181, doi:10.1007/s10555-007-9049-z (2007).
25. Gonen, N. & Assaraf, Y. G. Antifolates in cancer therapy: structure, activity and mechanisms of drug resistance. Drug Resist Updat 15, 183-210, doi:10.1016/j.drup.2012.07.002 (2012).
26. Banerjee, D. et al. Molecular mechanisms of resistance to antifolates, a review. Acta Biochim Pol 42, 457-464 (1995).
27. Bertino, J. R., Goker, E., Gorlick, R., Li, W. W. & Banerjee, D. Resistance mechanisms to methotrexate in tumors. Stem Cells 14, 5-9, doi:10.1002/stem.140005 (1996).
28. O'Connor, C. et al. Folate transporter dynamics and therapy with classic and tumor-targeted antifolates. Sci Rep 11, 6389, doi:10.1038/s41598-021-85818-x (2021).
29. Kim, S. E. Enzymes involved in folate metabolism and its implication for cancer treatment. Nutr Res Pract 14, 95-101, doi:10.4162/nrp.2020.14.2.95 (2020).
30. Assaraf, Y. G. The role of multidrug resistance efflux transporters in antifolate resistance and folate homeostasis. Drug Resist Updat 9, 227-246, doi:10.1016/j.drup.2006.09.001 (2006).
31. Liani, E. et al. Loss of folylpoly-gamma-glutamate synthetase activity is a dominant mechanism of resistance to polyglutamylation-dependent novel antifolates in multiple human leukemia sublines. Int J Cancer 103, 587-599, doi:10.1002/ijc.10829 (2003).
32. Raz, S., Stark, M. & Assaraf, Y. G. Folylpoly-gamma-glutamate synthetase: A key determinant of folate homeostasis and antifolate resistance in cancer. Drug Resist Updat 28, 43-64, doi:10.1016/j.drup.2016.06.004 (2016).
33. Allegra, C. J. et al. Enhanced inhibition of thymidylate synthase by methotrexate polyglutamates. J Biol Chem 260, 9720-9726 (1985).
34. Gregson, A. & Plowe, C. V. Mechanisms of resistance of malaria parasites to antifolates. Pharmacol Rev 57, 117-145, doi:10.1124/pr.57.1.4 (2005).
35. Zhao, R., Babani, S., Gao, F., Liu, L. & Goldman, I. D. The mechanism of transport of the multitargeted antifolate (MTA) and its cross-resistance pattern in cells with markedly impaired transport of methotrexate. Clin Cancer Res 6, 3687-3695 (2000).
36. Matherly, L. H., Wilson, M. R. & Hou, Z. The major facilitative folate transporters solute carrier 19A1 and solute carrier 46A1: biology and role in antifolate chemotherapy of cancer. Drug Metab Dispos 42, 632-649, doi:10.1124/dmd.113.055723 (2014).
37. Paz-Ares, L. et al. Maintenance therapy with pemetrexed plus best supportive care versus placebo plus best supportive care after induction therapy with pemetrexed plus cisplatin for advanced non-squamous non-small-cell lung cancer (PARAMOUNT): a double-blind, phase 3, randomised controlled trial. Lancet Oncol 13, 247-255, doi:10.1016/S1470-2045(12)70063-3 (2012).
38. Hanauske, A. R., Chen, V., Paoletti, P. & Niyikiza, C. Pemetrexed disodium: a novel antifolate clinically active against multiple solid tumors. Oncologist 6, 363-373, doi:10.1634/theoncologist.6-4-363 (2001).
39. Lam, S. W., Guchelaar, H. J. & Boven, E. The role of pharmacogenetics in capecitabine efficacy and toxicity. Cancer Treat Rev 50, 9-22, doi:10.1016/j.ctrv.2016.08.001 (2016).
40. Emura, T., Suzuki, N., Yamaguchi, M., Ohshimo, H. & Fukushima, M. A novel combination antimetabolite, TAS-102, exhibits antitumor activity in FU-resistant human cancer cells through a mechanism involving FTD incorporation in DNA. Int J Oncol 25, 571-578 (2004).
41. Siddiqui, N. S., Godara, A., Byrne, M. M. & Saif, M. W. Capecitabine for the treatment of pancreatic cancer. Expert Opin Pharmacother 20, 399-409, doi:10.1080/14656566.2018.1560422 (2019).
42. Sun, J. M. et al. Pemetrexed Plus Cisplatin Versus Gemcitabine Plus Cisplatin According to Thymidylate Synthase Expression in Nonsquamous Non-Small-Cell Lung Cancer: A Biomarker-Stratified Randomized Phase II Trial. J Clin Oncol 33, 2450-2456, doi:10.1200/JCO.2014.59.9324 (2015).
43. Minami, S. & Kijima, T. Pemetrexed in maintenance treatment of advanced non-squamous non-small-cell lung cancer. Lung Cancer (Auckl) 6, 13-25, doi:10.2147/LCTT.S73268 (2015).
44. Mahmud, F. et al. Oral pemetrexed facilitates low-dose metronomic therapy and enhances antitumor efficacy in lung cancer. J Control Release 284, 160-170, doi:10.1016/j.jconrel.2018.06.018 (2018).
45. Pangeni, R. et al. Intestinal transport mechanism and in vivo anticancer efficacy of a solid oral formulation incorporating an ion-pairing complex of pemetrexed with deoxycholic acid derivative. Int J Nanomedicine 14, 6339-6356, doi:10.2147/IJN.S209722 (2019).
46. Pangeni, R., Choi, J. U., Panthi, V. K., Byun, Y. & Park, J. W. Enhanced oral absorption of pemetrexed by ion-pairing complex formation with deoxycholic acid derivative and multiple nanoemulsion formulations: preparation, characterization, and in vivo oral bioavailability and anticancer effect. Int J Nanomedicine 13, 3329-3351, doi:10.2147/IJN.S167958 (2018).
47. Maharjan, R. et al. Anti-Angiogenic Effect of Orally Available Pemetrexed for Metronomic Chemotherapy. Pharmaceutics 11, doi:10.3390/pharmaceutics11070332 (2019).
48. Tetko, I. V., Tanchuk, V. Y., Kasheva, T. N. & Villa, A. E. Estimation of aqueous solubility of chemical compounds using E-state indices. J Chem Inf Comput Sci 41, 1488-1493, doi:10.1021/ci000392t (2001).
49. Humphrey, W., Dalke, A. & Schulten, K. VMD: visual molecular dynamics. J Mol Graph 14, 33-38, 27-38, doi:10.1016/0263-7855(96)00018-5 (1996).
50. Morris, G. M. et al. AutoDock4 and AutoDockTools4: Automated docking with selective receptor flexibility. J Comput Chem 30, 2785-2791, doi:10.1002/jcc.21256 (2009).
51. Arcon, J. P. et al. AutoDock Bias: improving binding mode prediction and virtual screening using known protein-ligand interactions. Bioinformatics 35, 3836-3838, doi:10.1093/bioinformatics/btz152 (2019).
52. Arcon, J. P. et al. Molecular Dynamics in Mixed Solvents Reveals Protein-Ligand Interactions, Improves Docking, and Allows Accurate Binding Free Energy Predictions. J Chem Inf Model 57, 846-863, doi:10.1021/acs.jcim.6b00678 (2017).
53. Arcon, J. P. et al. Cosolvent-Based Protein Pharmacophore for Ligand Enrichment in Virtual Screening. J Chem Inf Model 59, 3572-3583, doi:10.1021/acs.jcim.9b00371 (2019).
54. Stierand, K., Maass, P. C. & Rarey, M. Molecular complexes at a glance: automated generation of two-dimensional complex diagrams. Bioinformatics 22, 1710-1716, doi:10.1093/bioinformatics/btl150 (2006).
55. Griffin, R. J.; Meek, M. A.; Schwalbe, C. H.; Stevens, M. F. G. J. Med. Chem. 1989, 32, 2468-2474.

### EQUIVALENTS AND SCOPE

In the claims articles such as “a,” “an,” and “the” may mean one or more than one unless indicated to the contrary or otherwise evident from the context. Claims or descriptions that include “or” between one or more members of a group are considered satisfied if one, more than one, or all of the group members are present in, employed in, or otherwise relevant to a given product or process unless indicated to the contrary or otherwise evident from the context. The disclosure includes embodiments in which exactly one member of the group is present in, employed in, or otherwise relevant to a given product or process. The disclosure includes embodiments in which more than one, or all of the group members are present in, employed in, or otherwise relevant to a given product or process.

Furthermore, the disclosure encompasses all variations, combinations, and permutations in which one or more limitations, elements, clauses, and descriptive terms from one or more of the listed claims is introduced into another claim. For example, any claim that is dependent on another claim can be modified to include one or more limitations found in any other claim that is dependent on the same base claim. Where elements are presented as lists, e.g., in Markush group format, each subgroup of the elements is also disclosed, and any element(s) can be removed from the group. It should it be understood that, in general, where the disclosure, or aspects described herein, is/are referred to as comprising particular elements and/or features, certain embodiments described herein or aspects described herein consist, or consist essentially of, such elements and/or features. For purposes of simplicity, those embodiments have not been specifically set forth in haec verba herein. It is also noted that the terms “comprising” and “containing” are intended to be open and permits the inclusion of additional elements or steps. Where ranges are given, endpoints are included. Furthermore, unless otherwise indicated or otherwise evident from the context and understanding of one of ordinary skill in the art, values that are expressed as ranges can assume any specific value or sub-range within the stated ranges in different embodiments described herein, to the tenth of the unit of the lower limit of the range, unless the context clearly dictates otherwise.

This application refers to various issued patents, published patent applications, journal articles, and other publications, all of which are incorporated herein by reference. If there is a conflict between any of the incorporated references and the instant specification, the specification shall control. In addition, any particular embodiment of the present disclosure that falls within the prior art may be explicitly excluded from any one or more of the claims. Because such embodiments are deemed to be known to one of ordinary skill in the art, they may be excluded even if the exclusion is not set forth explicitly herein. Any particular embodiment described herein can be excluded from any claim, for any reason, whether or not related to the existence of prior art.

Those skilled in the art will recognize or be able to ascertain using no more than routine experimentation many equivalents to the specific embodiments described herein. The scope of the present embodiments described herein is not intended to be limited to the above Description, but rather is as set forth in the appended claims. Those of ordinary skill in the art will appreciate that various changes and modifications to this description may be made without departing from the spirit or scope of the present disclosure, as defined in the following claims.

1. A compound of Formula (I): 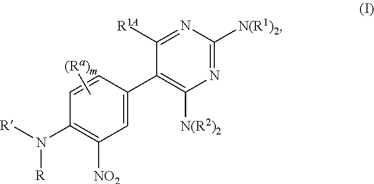
or a pharmaceutically acceptable salt, solvate, hydrate, stereoisomer, polymorph, tautomer, isotopically enriched form, or prodrug thereof, wherein:
R^{1A} is hydrogen or optionally substituted alkyl;
each instance of R¹ is independently hydrogen, optionally substituted acyl, optionally substituted alkyl, or a nitrogen protecting group; or optionally two instances of R¹ are taken together with their intervening atoms to form a substituted or unsubstituted heterocyclic or substituted or unsubstituted heteroaryl ring;
each instance of R² is independently hydrogen, optionally substituted acyl, optionally substituted alkyl, or a nitrogen protecting group; or optionally two instances of R² are taken together with their intervening atoms to form a substituted or unsubstituted heterocyclic or substituted or unsubstituted heteroaryl ring;
each instance of R^{a} is independently halogen, optionally substituted acyl, optionally substituted alkyl, optionally substituted alkenyl, optionally substituted alkynyl, optionally substituted carbocyclyl, optionally substituted heterocyclyl, optionally substituted aryl, optionally substituted heteroaryl, —CN, —NO₂, —OR^{D1}, —N(R^{D1a})₂, or —SR^{D1}, wherein R^{D1} is independently hydrogen, optionally substituted acyl, optionally substituted alkyl, optionally substituted alkenyl, optionally substituted alkynyl, optionally substituted carbocyclyl, optionally substituted heterocyclyl, optionally substituted aryl, optionally substituted heteroaryl, or an oxygen protecting group when attached to an oxygen atom, or a sulfur protecting group when attached to a sulfur atom;
wherein each occurrence of R^{D1a} is independently hydrogen, optionally substituted acyl, optionally substituted alkyl, optionally substituted alkenyl, optionally substituted alkynyl, optionally substituted carbocyclyl, optionally substituted heterocyclyl, optionally substituted aryl, optionally substituted heteroaryl, or a nitrogen protecting group; or optionally two instances of R^{D1a} are taken together with their intervening atoms to form a substituted or unsubstituted heterocyclic or substituted or unsubstituted heteroaryl ring;
R is hydrogen, unsubstituted alkyl, or a nitrogen protecting group;
R′ is 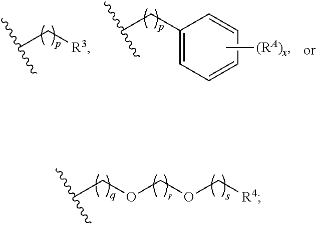
R³ is optionally substituted carbocyclyl, optionally substituted heterocyclyl, or optionally substituted heteroaryl;
each occurrence of R^{A} is independently —SO₂, —SR^{D1}, —NO₂, —N₃, or —CN, or optionally two instances of R^{A} are taken together with their intervening atoms to form a substituted or unsubstituted heterocyclic or substituted or unsubstituted heteroaryl ring;
R⁴ is —OR^{4A} or —N(R^{4B})₂;
R^{4A} is hydrogen, optionally substituted acyl, optionally substituted alkyl, optionally substituted alkenyl, optionally substituted alkynyl, optionally substituted carbocyclyl, optionally substituted heterocyclyl, optionally substituted aryl, optionally substituted heteroaryl, or an oxygen protecting group;
each occurrence of R^{4B} is independently hydrogen, optionally substituted acyl, optionally substituted alkyl, optionally substituted alkenyl, optionally substituted alkynyl, optionally substituted carbocyclyl, optionally substituted heterocyclyl, optionally substituted aryl, optionally substituted heteroaryl, or a nitrogen protecting group; or optionally two instances of R^{4B} are taken together with their intervening atoms to form a substituted or unsubstituted heterocyclic or substituted or unsubstituted heteroaryl ring;
m is 0, 1, 2, or 3;
p is 1, 2, 3, 4, 5, or 6;
q is 1, 2, 3, 4, 5, or 6;
r is 1, 2, 3, 4, 5, or 6;
s is 1, 2, 3, 4, 5, or 6; and
x is 1, 2, 3, 4, or 5.

2. The compound of claim 1, wherein the compound is of Formula (I-A): 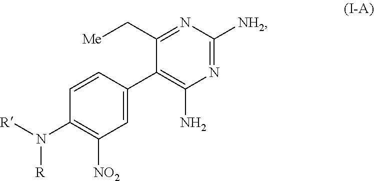
or a pharmaceutically acceptable salt, solvate, hydrate, stereoisomer, polymorph, tautomer, isotopically enriched form, or prodrug thereof.

3. A compound of Formula (II): 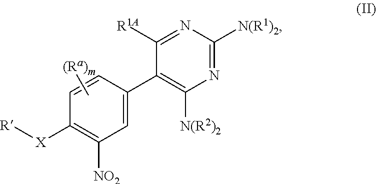
or a pharmaceutically acceptable salt, solvate, hydrate, stereoisomer, polymorph, tautomer, isotopically enriched form, or prodrug thereof, wherein:
X is —O— or —S—;
R^{1A} is hydrogen or optionally substituted alkyl;
each instance of R¹ is independently hydrogen, optionally substituted acyl, optionally substituted alkyl, or a nitrogen protecting group; or optionally two instances of R¹ are taken together with their intervening atoms to form a substituted or unsubstituted heterocyclic or substituted or unsubstituted heteroaryl ring;
each instance of R² is independently hydrogen, optionally substituted acyl, optionally substituted alkyl, or a nitrogen protecting group; or optionally two instances of R² are taken together with their intervening atoms to form a substituted or unsubstituted heterocyclic or substituted or unsubstituted heteroaryl ring;
each instance of R^{a} is independently halogen, optionally substituted acyl, optionally substituted alkyl, optionally substituted alkenyl, optionally substituted alkynyl, optionally substituted carbocyclyl, optionally substituted heterocyclyl, optionally substituted aryl, optionally substituted heteroaryl, —CN, —NO₂, —OR^{D1}, —N(R^{D1a})₂, or —SR^{D1}, wherein R^{D1} is independently hydrogen, optionally substituted acyl, optionally substituted alkyl, optionally substituted alkenyl, optionally substituted alkynyl, optionally substituted carbocyclyl, optionally substituted heterocyclyl, optionally substituted aryl, optionally substituted heteroaryl, or an oxygen protecting group when attached to an oxygen atom, or a sulfur protecting group when attached to a sulfur atom;
wherein each occurrence of R^{D1a} is independently hydrogen, optionally substituted acyl, optionally substituted alkyl, optionally substituted alkenyl, optionally substituted alkynyl, optionally substituted carbocyclyl, optionally substituted heterocyclyl, optionally substituted aryl, optionally substituted heteroaryl, or a nitrogen protecting group; or optionally two instances of R^{D1a} are taken together with their intervening atoms to form a substituted or unsubstituted heterocyclic or substituted or unsubstituted heteroaryl ring;
R^{1′} is 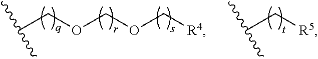
optionally substituted carbocyclyl, optionally substituted heterocyclyl, optionally substituted aryl, or optionally substituted heteroaryl;
R⁴ is —OR^{4A} or —N(R^{4B})₂;
R⁵ is hydrogen, —OR^{D1}, optionally substituted acyl, optionally substituted alkyl, optionally substituted alkenyl, optionally substituted alkynyl, optionally substituted carbocyclyl, optionally substituted heterocyclyl, optionally substituted aryl, or optionally substituted heteroaryl;
R^{4A} is hydrogen, optionally substituted acyl, optionally substituted alkyl, optionally substituted alkenyl, optionally substituted alkynyl, optionally substituted carbocyclyl, optionally substituted heterocyclyl, optionally substituted aryl, optionally substituted heteroaryl, or an oxygen protecting group;
each occurrence of R^{4B} is independently hydrogen, optionally substituted acyl, optionally substituted alkyl, optionally substituted alkenyl, optionally substituted alkynyl, optionally substituted carbocyclyl, optionally substituted heterocyclyl, optionally substituted aryl, optionally substituted heteroaryl, or a nitrogen protecting group; or optionally two instances of R^{4B} are taken together with their intervening atoms to form a substituted or unsubstituted heterocyclic or substituted or unsubstituted heteroaryl ring;
m is 0, 1, 2, or 3;
q is 1, 2, 3, 4, 5, or 6;
r is 1, 2, 3, 4, 5, or 6;
s is 1, 2, 3, 4, 5, or 6; and
t is 1, 2, 3, 4, 5, or 6.

4-5. (canceled)

6. The compound of claim 1, or a pharmaceutically acceptable salt, solvate, hydrate, stereoisomer, polymorph, tautomer, isotopically enriched form, or prodrug thereof, wherein at least one instance of R¹ is hydrogen.

7. (canceled)

8. The compound of claim 1, or a pharmaceutically acceptable salt, solvate, hydrate, stereoisomer, polymorph, tautomer, isotopically enriched form, or prodrug thereof, wherein at least one instance of R² is hydrogen.

9-13. (canceled)

14. The compound of claim 1, or a pharmaceutically acceptable salt, solvate, hydrate, stereoisomer, polymorph, tautomer, isotopically enriched form, or prodrug thereof, wherein R′ is of the formula: 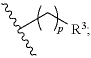
p is 1, 2, or 3; and
R³ is optionally substituted carbocyclyl, optionally substituted heterocyclyl, or optionally substituted heteroaryl.

15-20. (canceled)

21. The compound of claim 1, or a pharmaceutically acceptable salt, solvate, hydrate, stereoisomer, polymorph, tautomer, isotopically enriched form, or prodrug thereof, wherein R′ is of the formula: 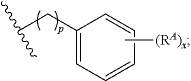
p is 1, 2, or 3;
x is 2; and
two instances of R^{A} are taken together with their intervening atoms to form a substituted or unsubstituted heterocyclic or substituted or unsubstituted heteroaryl ring.

22. (canceled)

23. The compound of claim 1, or a pharmaceutically acceptable salt, solvate, hydrate, stereoisomer, polymorph, tautomer, isotopically enriched form, or prodrug thereof, wherein R′ is of the formula:

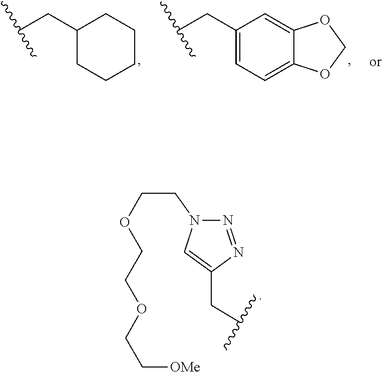

24. The compound of claim 1, or a pharmaceutically acceptable salt, solvate, hydrate, stereoisomer, polymorph, tautomer, isotopically enriched form, or prodrug thereof, wherein R′ is of the formula: 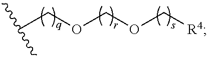
q is 1, 2, or 3;
r is 1, 2, or 3;
s is 1, 2, or 3;
R⁴ is —OR^{4A} or —N(R^{4B})₂;
R^{4A} is hydrogen, optionally substituted acyl, optionally substituted alkyl, optionally substituted alkenyl, optionally substituted alkynyl, optionally substituted carbocyclyl, optionally substituted heterocyclyl, optionally substituted aryl, or optionally substituted heteroaryl; and
each occurrence of R^{4B} is independently hydrogen, optionally substituted acyl, optionally substituted alkyl, optionally substituted alkenyl, optionally substituted alkynyl, optionally substituted carbocyclyl, optionally substituted heterocyclyl, optionally substituted aryl, or optionally substituted heteroaryl; or optionally two instances of R^{4B} are taken together with their intervening atoms to form a substituted or unsubstituted heterocyclic or substituted or unsubstituted heteroaryl ring.

25-30. (canceled)

31. The compound of claim 24, or a pharmaceutically acceptable salt, solvate, hydrate, stereoisomer, polymorph, tautomer, isotopically enriched form, or prodrug thereof, wherein R⁴ is —OMe.

32-33. (canceled)

34. The compound of claim 1, or a pharmaceutically acceptable salt, solvate, hydrate, stereoisomer, polymorph, tautomer, isotopically enriched form, or prodrug thereof, wherein R is hydrogen or unsubstituted C₁₋₆ alkyl; and
R′ is of the formula: 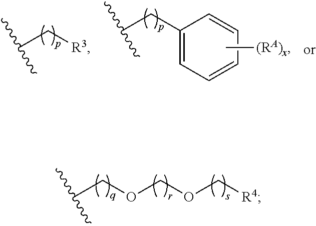
p is 1, 2, or 3;
x is 2;
q is 1, 2, or 3;
r is 1, 2, or 3;
s is 1, 2, or 3;
two instances of R^{A} are taken together with their intervening atoms to form a substituted or unsubstituted heterocyclic or substituted or unsubstituted heteroaryl ring;
R³ is optionally substituted carbocyclyl, optionally substituted heterocyclyl, or 5-membered or 6-membered optionally substituted heteroaryl;
R⁴ is —OR^{4A} or —N(R^{4B})₂;
R^{4A} is hydrogen, optionally substituted acyl, optionally substituted alkyl, optionally substituted alkenyl, or optionally substituted alkynyl; and
each occurrence of R^{4B} is independently hydrogen, optionally substituted acyl, optionally substituted alkyl, optionally substituted alkenyl, or optionally substituted alkynyl; or optionally two instances of R^{4B} are taken together with their intervening atoms to form a substituted or unsubstituted heterocyclic or substituted or unsubstituted heteroaryl ring.

35. The compound of claim 1, or a pharmaceutically acceptable salt, solvate, hydrate, stereoisomer, polymorph, tautomer, isotopically enriched form, or prodrug thereof, wherein R′ is of the formula:

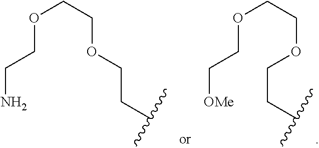 [SMILES: *CCOCCOCCN ; *CCOCCOCCOC]

36. The compound of claim 1, wherein the compound is of formula: 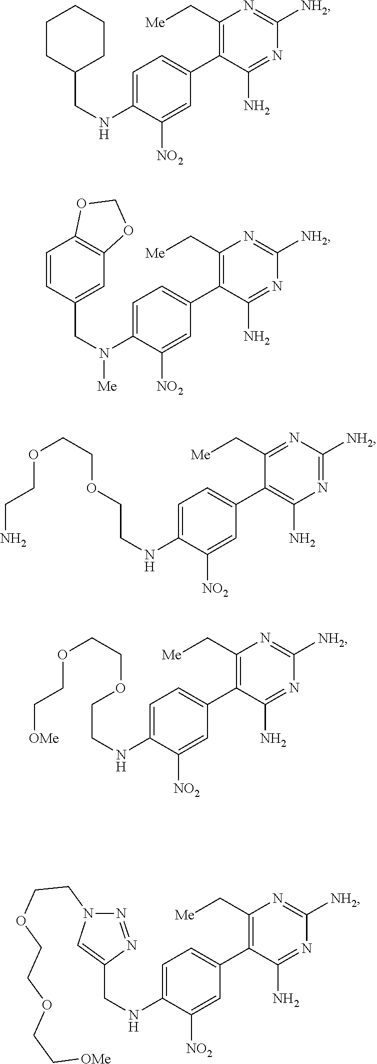 [SMILES: CCc1nc(N)nc(N)c1-c1ccc(NCC2CCCCC2)c([N+](=O)[O-])c1 ; CCc1nc(N)nc(N)c1-c1ccc(N(C)Cc2ccc3c(c2)OCO3)c([N+](=O)[O-])c1 ; CCc1nc(N)nc(N)c1-c1ccc(NCCOCCOCCN)c([N+](=O)[O-])c1 ; CCc1nc(N)nc(N)c1-c1ccc(NCCOCCOCCOC)c([N+](=O)[O-])c1 ; CCc1nc(N)nc(N)c1-c1ccc(NCc2cn(CCOCCOCCOC)nn2)c([N+](=O)[O-])c1]
or a pharmaceutically acceptable salt, solvate, hydrate, stereoisomer, polymorph, tautomer, isotopically enriched form, or prodrug thereof.

37-42. (canceled)

43. A compound of formula: 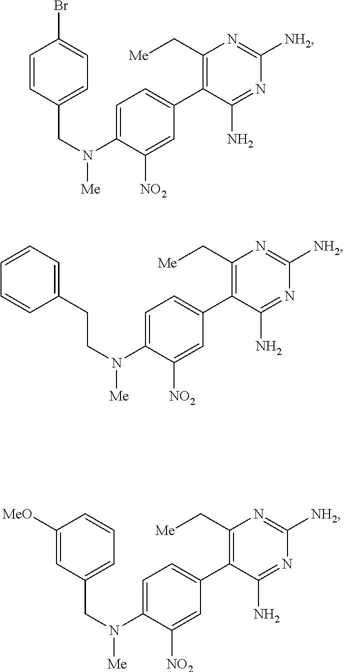 [SMILES: CCc1nc(N)nc(N)c1-c1ccc(N(C)Cc2ccc(Br)cc2)c([N+](=O)[O-])c1 ; CCc1nc(N)nc(N)c1-c1ccc(N(C)CCc2ccccc2)c([N+](=O)[O-])c1 ; CCc1nc(N)nc(N)c1-c1ccc(N(C)Cc2cccc(OC)c2)c([N+](=O)[O-])c1]
or a pharmaceutically acceptable salt, solvate, hydrate, stereoisomer, polymorph, tautomer, isotopically enriched form, or prodrug thereof.

44. A pharmaceutical composition comprising a compound of claim 43, or a pharmaceutically acceptable salt, solvate, hydrate, stereoisomer, polymorph, tautomer, isotopically enriched form, or prodrug thereof, and a pharmaceutically acceptable excipient.

45. The pharmaceutical composition of claim 44, wherein the pharmaceutical composition comprises a therapeutically effective amount of the compound for use in treating a proliferative disease in a subject in need thereof.

46. (canceled)

47. A method of treating a disease in a subject in need thereof, the method comprising administering to the subject a therapeutically effective amount of the compound of claim 1, or a pharmaceutically acceptable salt, solvate, hydrate, stereoisomer, polymorph, tautomer, isotopically enriched form, or prodrug thereof.

48. (canceled)

49. The method of claim 47, wherein the disease is cancer.

50. The method of claim 49, wherein the cancer is:
(a) a carcinoma;
(b) lung cancer;
(c) breast cancer;
(d) liver cancer;
(e) pancreatic cancer;
(f) gastric cancer;
(g) ovarian cancer;
(h) cervical cancer;
(i) colon cancer;
(j) colorectal cancer;
(k) bladder cancer;
(l) hematopoietic cancer;
(m) plasmacytoma or multiple myeloma; or
(n) leukemia or lymphoma.

51-68. (canceled)

69. A method of inhibiting the activity of one or more folate dependent enzymes in a biological sample or subject, the method comprising administering to the subject or contacting the biological sample with a therapeutically effective amount of a compound of claim 1, or a pharmaceutically acceptable salt, solvate, hydrate, stereoisomer, polymorph, tautomer, isotopically enriched form, or prodrug thereof.

70. (canceled)
